SEQUENCE LISTING (194 sequences)

SEQ ID NO: 1 - protein, 5 aa, synthetic construct
  REGION 1..5: note = Synthetic Sequence
EYTIH

SEQ ID NO: 2 - protein, 17 aa, synthetic construct
  REGION 1..17: note = Synthetic Sequence
GINPNNGIPNYNQKFKG

SEQ ID NO: 3 - protein, 15 aa, synthetic construct
  REGION 1..15: note = Synthetic Sequence
RRIAYGYDEGHAMDY

SEQ ID NO: 4 - protein, 17 aa, synthetic construct
  REGION 1..17: note = Synthetic Sequence
KSSQSLLYSRNQKNYLA

SEQ ID NO: 5 - protein, 7 aa, synthetic construct
  REGION 1..7: note = Synthetic Sequence
WASTRES

SEQ ID NO: 6 - protein, 9 aa, synthetic construct
  REGION 1..9: note = Synthetic Sequence
QQYYSYPLT

SEQ ID NO: 7 - protein, 124 aa, synthetic construct
  REGION 1..124: note = Synthetic Sequence
QVQLVQSGAEVKKPGASVKVSCKTSRYTFTEYTIHWVRQAPGQRLEWIGGINPNNGIPNYNQKFKGRVTITVDTSASTAYMELSSLRSEDTAVYYCARRRIAYGYDEGHAMDYWGQGTLVTVSS

SEQ ID NO: 8 - protein, 113 aa, synthetic construct
  REGION 1..113: note = Synthetic Sequence
DIVMTQSPDSLAVSLGERATINCKSSQSLLYSRNQKNYLAWYQQKPGQPPKLLIFWASTRESGVPDRFSGSGFGTDFTLTISSLQAEDFAVYYCQQYFSYPLTFGQGTKVEIK

SEQ ID NO: 9 - protein, 113 aa, synthetic construct
  REGION 1..113: note = Synthetic Sequence
DIVMTQSPDSLAVSLGERATINCKSSQSLLYSRNQKNYLAWYQQKPGQPPKLLIFWASTRESGVPDRFSGSGSGTDFTLTISRLEPEDFAVYYCQQYYSYPLTFGQGTKVEIK

SEQ ID NO: 10 - protein, 124 aa, synthetic construct
  REGION 1..124: note = Synthetic Sequence
EVQLQQSGPELVKPGASVKMSCKTSRYTFTEYTIHWVRQSHGKSLEWIGGINPNNGIPNYNQKFKGRATLTVGKSSSTAYMELRSLTSEDSAVYFCARRRIAYGYDEGHAMDYWGQGTSVTVSS

SEQ ID NO: 11 - protein, 113 aa, synthetic construct
  REGION 1..113: note = Synthetic Sequence
DIVMSQSPSSLAVSVGEKVTMSCKSSQSLLYSRNQKNYLAWFQQKPGQSPKLLIFWASTRESGVPDRFTGSGFGTDFNLTISSVQAEDLAVYDCQQYFSYPLTFGAGTKLELK

SEQ ID NO: 12 - protein, 376 aa, synthetic construct
  REGION 1..376: note = Synthetic Sequence
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKGSTSGGGSGGGSGGGGSSEVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSEPKSSDKTHTCPPCPPCGGGSSGGGSGGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 13 - protein, 376 aa, synthetic construct
  REGION 1..376: note = Synthetic Sequence
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSGSTSGGGSGGGSGGGGSSEIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKEPKSSDKTHTCPPCPPCGGGSSGGGSGGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 14 - protein, 239 aa, synthetic construct
  REGION 1..239: note = Synthetic Sequence
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSSGGGGEIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKGGCPPCPPC

SEQ ID NO: 15 - protein, 242 aa, synthetic construct
  REGION 1..242: note = Synthetic Sequence
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSGGGSGGGGEIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKGGCPPCPPC

SEQ ID NO: 16 - protein, 239 aa, synthetic construct
  REGION 1..239: note = Synthetic Sequence
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKSGGGGEVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSGGCPPCPPC

SEQ ID NO: 17 - protein, 242 aa, synthetic construct
  REGION 1..242: note = Synthetic Sequence
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKGGGSGGGGEVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSGGCPPCPPC

SEQ ID NO: 18 - protein, 375 aa, synthetic construct
  REGION 1..375: note = Synthetic Sequence
EIVLTQSPGTLSLSPGERATLSCRASQSVSRSYLAWYQQKPGQAPRLLIIGASTRATGIPDRFSGSGSGTDFTLTISRLEPEDFAVYYCQQGQVIPPTFGQGTKVEIKGSTSGGGSGGGSGGGGSSEVQLLESGGGLVQPGGSLRLSCAASGFTFSSHAMSWVRQAPGKGLEWVSAIWASGEQYYADSVKGRFTISRDNSKNTLYLQMNSLRAEDTAVYYCAKGWLGNFDYWGQGTLVTVSSEPKSSDKTHTCPPCPPCGGGSSGGGSGGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 19 - protein, 375 aa, synthetic construct
  REGION 1..375: note = Synthetic Sequence
EVQLLESGGGLVQPGGSLRLSCAASGFTFSSHAMSWVRQAPGKGLEWVSAIWASGEQYYADSVKGRFTISRDNSKNTLYLQMNSLRAEDTAVYYCAKGWLGNFDYWGQGTLVTVSSGSTSGGGSGGGSGGGGSSEIVLTQSPGTLSLSPGERATLSCRASQSVSRSYLAWYQQKPGQAPRLLIIGASTRATGIPDRFSGSGSGTDFTLTISRLEPEDFAVYYCQQGQVIPPTFGQGTKVEIKEPKSSDKTHTCPPCPPCGGGSSGGGSGGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 20 - protein, 238 aa, synthetic construct
  REGION 1..238: note = Synthetic Sequence
EIVLTQSPGTLSLSPGERATLSCRASQSVSRSYLAWYQQKPGQAPRLLIIGASTRATGIPDRFSGSGSGTDFTLTISRLEPEDFAVYYCQQGQVIPPTFGQGTKVEIKSGGGGEVQLLESGGGLVQPGGSLRLSCAASGFTFSSHAMSWVRQAPGKGLEWVSAIWASGEQYYADSVKGRFTISRDNSKNTLYLQMNSLRAEDTAVYYCAKGWLGNFDYWGQGTLVTVSSGGCPPCPPC

SEQ ID NO: 21 - protein, 238 aa, synthetic construct
  REGION 1..238: note = Synthetic Sequence
EVQLLESGGGLVQPGGSLRLSCAASGFTFSSHAMSWVRQAPGKGLEWVSAIWASGEQYYADSVKGRFTISRDNSKNTLYLQMNSLRAEDTAVYYCAKGWLGNFDYWGQGTLVTVSSSGGGGEIVLTQSPGTLSLSPGERATLSCRASQSVSRSYLAWYQQKPGQAPRLLIIGASTRATGIPDRFSGSGSGTDFTLTISRLEPEDFAVYYCQQGQVIPPTFGQGTKVEIKGGCPPCPPC

SEQ ID NO: 22 - protein, 241 aa, synthetic construct
  REGION 1..241: note = Synthetic Sequence
EIVLTQSPGTLSLSPGERATLSCRASQSVSRSYLAWYQQKPGQAPRLLIIGASTRATGIPDRFSGSGSGTDFTLTISRLEPEDFAVYYCQQGQVIPPTFGQGTKVEIKGGGSGGGGEVQLLESGGGLVQPGGSLRLSCAASGFTFSSHAMSWVRQAPGKGLEWVSAIWASGEQYYADSVKGRFTISRDNSKNTLYLQMNSLRAEDTAVYYCAKGWLGNFDYWGQGTLVTVSSGGCPPCPPC

SEQ ID NO: 23 - protein, 241 aa, synthetic construct
  REGION 1..241: note = Synthetic Sequence
EVQLLESGGGLVQPGGSLRLSCAASGFTFSSHAMSWVRQAPGKGLEWVSAIWASGEQYYADSVKGRFTISRDNSKNTLYLQMNSLRAEDTAVYYCAKGWLGNFDYWGQGTLVTVSSGGGSGGGGEIVLTQSPGTLSLSPGERATLSCRASQSVSRSYLAWYQQKPGQAPRLLIIGASTRATGIPDRFSGSGSGTDFTLTISRLEPEDFAVYYCQQGQVIPPTFGQGTKVEIKGGCPPCPPC

SEQ ID NO: 24 - protein, 388 aa, synthetic construct
  REGION 1..388: note = Synthetic Sequence
DIVMTQSPDSLAVSLGERATINCKSSQSLLYSRNQKNYLAWYQQKPGQPPKLLIFWASTRESGVPDRFSGSGFGTDFTLTISSLQAEDVAVYYCQQYFSYPLTFGQGTKVEIKGSTSGGGSGGGSGGGGSSQVQLVQSGAEVKKPGASVKVSCKTSRYTFTEYTIHWVRQAPGQRLEWIGGINPNNGIPNYNQKFKGRVTITVDTSASTAYMELSSLRSEDTAVYYCARRRIAYGYDEGHAMDYWGQGTLVTVSSEPKSSDKTHTCPPCPPCGGGSSGGGSGGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 25 - protein, 388 aa, synthetic construct
  REGION 1..388: note = Synthetic Sequence
QVQLVQSGAEVKKPGASVKVSCKTSRYTFTEYTIHWVRQAPGQRLEWIGGINPNNGIPNYNQKFKGRVTITVDTSASTAYMELSSLRSEDTAVYYCARRRIAYGYDEGHAMDYWGQGTLVTVSSGSTSGGGSGGGSGGGGSSDIVMTQSPDSLAVSLGERATINCKSSQSLLYSRNQKNYLAWYQQKPGQPPKLLIFWASTRESGVPDRFSGSGFGTDFTLTISSLQAEDVAVYYCQQYFSYPLTFGQGTKVEIKEPKSSDKTHTCPPCPPCGGGSSGGGSGGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 26 - protein, 251 aa, synthetic construct
  REGION 1..251: note = Synthetic Sequence
QVQLVQSGAEVKKPGASVKVSCKTSRYTFTEYTIHWVRQAPGQRLEWIGGINPNNGIPNYNQKFKGRVTITVDTSASTAYMELSSLRSEDTAVYYCARRRIAYGYDEGHAMDYWGQGTLVTVSSSGGGGDIVMTQSPDSLAVSLGERATINCKSSQSLLYSRNQKNYLAWYQQKPGQPPKLLIFWASTRESGVPDRFSGSGFGTDFTLTISSLQAEDVAVYYCQQYFSYPLTFGQGTKVEIKGGCPPCPPC

SEQ ID NO: 27 - protein, 254 aa, synthetic construct
  REGION 1..254: note = Synthetic Sequence
QVQLVQSGAEVKKPGASVKVSCKTSRYTFTEYTIHWVRQAPGQRLEWIGGINPNNGIPNYNQKFKGRVTITVDTSASTAYMELSSLRSEDTAVYYCARRRIAYGYDEGHAMDYWGQGTLVTVSSGGGSGGGGDIVMTQSPDSLAVSLGERATINCKSSQSLLYSRNQKNYLAWYQQKPGQPPKLLIFWASTRESGVPDRFSGSGFGTDFTLTISSLQAEDVAVYYCQQYFSYPLTFGQGTKVEIKGGCPPCPPC

SEQ ID NO: 28 - protein, 251 aa, synthetic construct
  REGION 1..251: note = Synthetic Sequence
DIVMTQSPDSLAVSLGERATINCKSSQSLLYSRNQKNYLAWYQQKPGQPPKLLIFWASTRESGVPDRFSGSGFGTDFTLTISSLQAEDVAVYYCQQYFSYPLTFGQGTKVEIKSGGGGQVQLVQSGAEVKKPGASVKVSCKTSRYTFTEYTIHWVRQAPGQRLEWIGGINPNNGIPNYNQKFKGRVTITVDTSASTAYMELSSLRSEDTAVYYCARRRIAYGYDEGHAMDYWGQGTLVTVSSGGCPPCPPC

SEQ ID NO: 29 - protein, 254 aa, synthetic construct
  REGION 1..254: note = Synthetic Sequence
DIVMTQSPDSLAVSLGERATINCKSSQSLLYSRNQKNYLAWYQQKPGQPPKLLIFWASTRESGVPDRFSGSGFGTDFTLTISSLQAEDVAVYYCQQYFSYPLTFGQGTKVEIKGGGSGGGGQVQLVQSGAEVKKPGASVKVSCKTSRYTFTEYTIHWVRQAPGQRLEWIGGINPNNGIPNYNQKFKGRVTITVDTSASTAYMELSSLRSEDTAVYYCARRRIAYGYDEGHAMDYWGQGTLVTVSSGGCPPCPPC

SEQ ID NO: 30 - protein, 388 aa, synthetic construct
  REGION 1..388: note = Synthetic Sequence
DIVMTQSPDSLAVSLGERATINCKSSQSLLYSRNQKNYLAWYQQKPGQPPKLLIFWASTRESGVPDRFSGSGSGTDFTLTISSLQAEDFAVYYCQQYFSYPLTFGQGTKVEIKGSTSGGGSGGGSGGGGSSQVQLVQSGAEVKKPGASVKVSCKASGYTFTEYTIHWVRQAPGQRLEWIGGINPNNGIPNYNQKFKGRVTITVDTSASTAYMELSSLRSEDTAVYYCARRRIAYGYDEGHAMDYWGQGTLVTVSSEPKSSDKTHTCPPCPPCGGGSSGGGSGGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 31 - protein, 388 aa, synthetic construct
  REGION 1..388: note = Synthetic Sequence
QVQLVQSGAEVKKPGASVKVSCKASGYTFTEYTIHWVRQAPGQRLEWIGGINPNNGIPNYNQKFKGRVTITVDTSASTAYMELSSLRSEDTAVYYCARRRIAYGYDEGHAMDYWGQGTLVTVSSGSTSGGGSGGGSGGGGSSDIVMTQSPDSLAVSLGERATINCKSSQSLLYSRNQKNYLAWYQQKPGQPPKLLIFWASTRESGVPDRFSGSGSGTDFTLTISSLQAEDFAVYYCQQYFSYPLTFGQGTKVEIKEPKSSDKTHTCPPCPPCGGGSSGGGSGGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 32 - protein, 388 aa, synthetic construct
  REGION 1..388: note = Synthetic Sequence
DIVMTQSPDSLAVSLGERATINCKSSQSLLYSRNQKNYLAWYQQKPGQPPKLLIFWASTRESGVPDRFSGSGSGTDFTLTISSLQAEDFAVYYCQQYYSYPLTFGQGTKVEIKGSTSGGGSGGGSGGGGSSQVQLVQSGAEVKKPGASVKVSCKASGYTFTEYTIHWVRQAPGQRLEWIGGINPNNGIPNYNQKFKGRVTITVDTSASTAYMELSSLRSEDTAVYYCARRRIAYGYDEGHAMDYWGQGTLVTVSSEPKSSDKTHTCPPCPPCGGGSSGGGSGGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 33 - protein, 388 aa, synthetic construct
  REGION 1..388: note = Synthetic Sequence
QVQLVQSGAEVKKPGASVKVSCKASGYTFTEYTIHWVRQAPGQRLEWIGGINPNNGIPNYNQKFKGRVTITVDTSASTAYMELSSLRSEDTAVYYCARRRIAYGYDEGHAMDYWGQGTLVTVSSGSTSGGGSGGGSGGGGSSDIVMTQSPDSLAVSLGERATINCKSSQSLLYSRNQKNYLAWYQQKPGQPPKLLIFWASTRESGVPDRFSGSGSGTDFTLTISSLQAEDFAVYYCQQYYSYPLTFGQGTKVEIKEPKSSDKTHTCPPCPPCGGGSSGGGSGGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 34 - protein, 388 aa, synthetic construct
  REGION 1..388: note = Synthetic Sequence
DIVMTQSPDSLAVSLGERATINCKSSQSLLYSRNQKNYLAWYQQKPGQPPKLLIFWASTRESGVPDRFSGSGFGTDFTLTISRLEPEDFAVYYCQQYFSYPLTFGQGTKVEIKGSTSGGGSGGGSGGGGSSQVQLVQSGAEVKKPGASVKVSCKTSRYTFTEYTIHWVRQAPGQRLEWIGGINPNNGIPNYNQKFKGRVTITVDTSASTAYMELSSLRSEDTAVYYCARRRIAYGYDEGHAMDYWGQGTLVTVSSEPKSSDKTHTCPPCPPCGGGSSGGGSGGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 35 - protein, 388 aa, synthetic construct
  REGION 1..388: note = Synthetic Sequence
QVQLVQSGAEVKKPGASVKVSCKTSRYTFTEYTIHWVRQAPGQRLEWIGGINPNNGIPNYNQKFKGRVTITVDTSASTAYMELSSLRSEDTAVYYCARRRIAYGYDEGHAMDYWGQGTLVTVSSGSTSGGGSGGGSGGGGSSDIVMTQSPDSLAVSLGERATINCKSSQSLLYSRNQKNYLAWYQQKPGQPPKLLIFWASTRESGVPDRFSGSGFGTDFTLTISRLEPEDFAVYYCQQYFSYPLTFGQGTKVEIKEPKSSDKTHTCPPCPPCGGGSSGGGSGGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 36 - protein, 388 aa, synthetic construct
  REGION 1..388: note = Synthetic Sequence
DIVMTQSPDSLAVSLGERATINCKSSQSLLYSRNQKNYLAWYQQKPGQPPKLLIFWASTRESGVPDRFSGSGFGTDFTLTISRLEPEDFAVYYCQQYYSYPLTFGQGTKVEIKGSTSGGGSGGGSGGGGSSQVQLVQSGAEVKKPGASVKVSCKASGYTFTEYTIHWVRQAPGQRLEWIGGINPNNGIPNYNQKFKGRVTITVDTSASTAYMELSSLRSEDTAVYYCARRRIAYGYDEGHAMDYWGQGTLVTVSSEPKSSDKTHTCPPCPPCGGGSSGGGSGGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 37 - protein, 388 aa, synthetic construct
  REGION 1..388: note = Synthetic Sequence
QVQLVQSGAEVKKPGASVKVSCKASGYTFTEYTIHWVRQAPGQRLEWIGGINPNNGIPNYNQKFKGRVTITVDTSASTAYMELSSLRSEDTAVYYCARRRIAYGYDEGHAMDYWGQGTLVTVSSGSTSGGGSGGGSGGGGSSDIVMTQSPDSLAVSLGERATINCKSSQSLLYSRNQKNYLAWYQQKPGQPPKLLIFWASTRESGVPDRFSGSGFGTDFTLTISRLEPEDFAVYYCQQYYSYPLTFGQGTKVEIKEPKSSDKTHTCPPCPPCGGGSSGGGSGGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 38 - protein, 388 aa, synthetic construct
  REGION 1..388: note = Synthetic Sequence
DIVMTQSPDSLAVSLGERATINCKSSQSLLYSRNQKNYLAWYQQKPGQPPKLLIFWASTRESGVPDRFSGSGSGTDFTLTISRLEPEDFAVYYCQQYYSYPLTFGQGTKVEIKGSTSGGGSGGGSGGGGSSQVQLVQSGAEVKKPGASVKVSCKASGYTFTEYTIHWVRQAPGQRLEWIGGINPNNGIPNYNQKFKGRVTITVDTSASTAYMELSSLRSEDTAVYYCARRRIAYGYDEGHAMDYWGQGTLVTVSSEPKSSDKTHTCPPCPPCGGGSSGGGSGGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 39 - protein, 388 aa, synthetic construct
  REGION 1..388: note = Synthetic Sequence
QVQLVQSGAEVKKPGASVKVSCKASGYTFTEYTIHWVRQAPGQRLEWIGGINPNNGIPNYNQKFKGRVTITVDTSASTAYMELSSLRSEDTAVYYCARRRIAYGYDEGHAMDYWGQGTLVTVSSGSTSGGGSGGGSGGGGSSDIVMTQSPDSLAVSLGERATINCKSSQSLLYSRNQKNYLAWYQQKPGQPPKLLIFWASTRESGVPDRFSGSGSGTDFTLTISRLEPEDFAVYYCQQYYSYPLTFGQGTKVEIKEPKSSDKTHTCPPCPPCGGGSSGGGSGGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 40 - protein, 251 aa, synthetic construct
  REGION 1..251: note = Synthetic Sequence
DIVMTQSPDSLAVSLGERATINCKSSQSLLYSRNQKNYLAWYQQKPGQPPKLLIFWASTRESGVPDRFSGSGSGTDFTLTISSLQAEDFAVYYCQQYYSYPLTFGQGTKVEIKSGGGGQVQLVQSGAEVKKPGASVKVSCKASGYTFTEYTIHWVRQAPGQRLEWIGGINPNNGIPNYNQKFKGRVTITVDTSASTAYMELSSLRSEDTAVYYCARRRIAYGYDEGHAMDYWGQGTLVTVSSGGCPPCPPC

SEQ ID NO: 41 - protein, 251 aa, synthetic construct
  REGION 1..251: note = Synthetic Sequence
QVQLVQSGAEVKKPGASVKVSCKASGYTFTEYTIHWVRQAPGQRLEWIGGINPNNGIPNYNQKFKGRVTITVDTSASTAYMELSSLRSEDTAVYYCARRRIAYGYDEGHAMDYWGQGTLVTVSSSGGGGDIVMTQSPDSLAVSLGERATINCKSSQSLLYSRNQKNYLAWYQQKPGQPPKLLIFWASTRESGVPDRFSGSGSGTDFTLTISSLQAEDFAVYYCQQYYSYPLTFGQGTKVEIKGGCPPCPPC

SEQ ID NO: 42 - protein, 254 aa, synthetic construct
  REGION 1..254: note = Synthetic Sequence
DIVMTQSPDSLAVSLGERATINCKSSQSLLYSRNQKNYLAWYQQKPGQPPKLLIFWASTRESGVPDRFSGSGSGTDFTLTISSLQAEDFAVYYCQQYYSYPLTFGQGTKVEIKGGGSGGGGQVQLVQSGAEVKKPGASVKVSCKASGYTFTEYTIHWVRQAPGQRLEWIGGINPNNGIPNYNQKFKGRVTITVDTSASTAYMELSSLRSEDTAVYYCARRRIAYGYDEGHAMDYWGQGTLVTVSSGGCPPCPPC

SEQ ID NO: 43 - protein, 254 aa, synthetic construct
  REGION 1..254: note = Synthetic Sequence
QVQLVQSGAEVKKPGASVKVSCKASGYTFTEYTIHWVRQAPGQRLEWIGGINPNNGIPNYNQKFKGRVTITVDTSASTAYMELSSLRSEDTAVYYCARRRIAYGYDEGHAMDYWGQGTLVTVSSGGGSGGGGDIVMTQSPDSLAVSLGERATINCKSSQSLLYSRNQKNYLAWYQQKPGQPPKLLIFWASTRESGVPDRFSGSGSGTDFTLTISSLQAEDFAVYYCQQYYSYPLTFGQGTKVEIKGGCPPCPPC

SEQ ID NO: 44 - protein, 388 aa, synthetic construct
  REGION 1..388: note = Synthetic Sequence
DIVMSQSPSSLAVSVGEKVTMSCKSSQSLLYSRNQKNYLAWFQQKPGQSPKLLIFWASTRESGVPDRFTGSGFGTDFNLTISSVQAEDLAVYDCQQYFSYPLTFGAGTKLELKGSTSGGGSGGGSGGGGSSEVQLQQSGPELVKPGASVKMSCKTSRYTFTEYTIHWVRQSHGKSLEWIGGINPNNGIPNYNQKFKGRATLTVGKSSSTAYMELRSLTSEDSAVYFCARRRIAYGYDEGHAMDYWGQGTSVTVSSEPKSSDKTHTCPPCPPCGGGSSGGGSGGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 45 - protein, 388 aa, synthetic construct
  REGION 1..388: note = Synthetic Sequence
EVQLQQSGPELVKPGASVKMSCKTSRYTFTEYTIHWVRQSHGKSLEWIGGINPNNGIPNYNQKFKGRATLTVGKSSSTAYMELRSLTSEDSAVYFCARRRIAYGYDEGHAMDYWGQGTSVTVSSGSTSGGGSGGGSGGGGSSDIVMSQSPSSLAVSVGEKVTMSCKSSQSLLYSRNQKNYLAWFQQKPGQSPKLLIFWASTRESGVPDRFTGSGFGTDFNLTISSVQAEDLAVYDCQQYFSYPLTFGAGTKLELKEPKSSDKTHTCPPCPPCGGGSSGGGSGGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 46 - protein, 20 aa, synthetic construct
  REGION 1..20: note = Synthetic Sequence
METDTLLLWVLLLWVPGSTG

SEQ ID NO: 47 - DNA, 1191 bp, synthetic construct
  misc_feature 1..1191: note = Synthetic Sequence
  source 1..1191: mol_type = other DNA
atggagaccgatacactgctgctgtgggtgctgctgctgtgggtccccggatccaccggcgagatcgtgctgacacagagccccggcacactgtctctgtcccccggcgagagagccacactgagctgcagagccagccagagcgtgaccagcagctatctggcttggtaccagcaaaagcccggacaagcccctaggctgctgattaacgtgggatctaggagagccaccggcatccccgacagattttccggcagcggctccggcaccgatttcacactgaccatcagcagactggagcccgaggacttcgctgtgtactactgccagcaaggcatcatgctgccccccacatttggccaaggcaccaaggtggagatcaagggctccacctccggaggaggaagcggcggaggctccggaggcggaggatccagcgaggtgcagctgctggaaagcggaggaggactggtgcaacccggcggctctctgagactgagctgtgccgccagcggcttcaccttcagcagctacgccatgagctgggtgagacaagcccccggaaagggactggaatgggtgtccgccatcatcggcagcggagccagcacctactacgccgacagcgtgaagggaagattcaccatctctagggacaacagcaagaacacactgtatctgcagatgaactctctgagggccgaagacacagccgtgtactattgcgccaagggctggttcggcggctttaactactggggccaaggcacactggtgaccgtcagctccgaacccaagagctccgacaagacccacacgtgtcccccttgccctccttgtggcggaggaagctccggaggcggaagcggaggacagcctagggagccccaggtgtataccctccccccctccagggaagagatgaccaagaaccaggtgagcctgacctgcctcgtgaagggcttttatccctccgatatcgccgtggagtgggagagcaacggccagcctgagaacaattacaagaccaccccccctgtgctggactccgatggcagcttcttcctgtattccaagctgaccgtcgacaagtccaggtggcaacagggcaacgtcttcagctgcagcgtgatgcacgaggccctgcacaatcactacacccagaagtccctctccctgagccccggctga

SEQ ID NO: 48 - DNA, 1191 bp, synthetic construct
  misc_feature 1..1191: note = Synthetic Sequence
  source 1..1191: mol_type = other DNA
atggagaccgatacactgctgctgtgggtgctgctgctgtgggtgcccggcagcaccggcgaagtgcagctgctggagagcggaggcggactggtgcagcccggcggaagcctcagactgagctgcgccgcttccggcttcacctttagctcctacgccatgagctgggtgaggcaagcccccggcaaaggactggaatgggtgtccgccatcattggctccggcgctagcacctactacgccgacagcgtgaagggaagattcaccatctccagagacaacagcaagaacacactgtacctccagatgaactctctgagggccgaggacaccgctgtgtattactgcgccaagggctggttcggcggcttcaattactggggccaaggcaccctcgtgaccgtgagcagcggaagcacctccggaggaggatccggcggaggaagcggcggcggaggcagcagcgaaattgtgctgacccagtcccccggcacactgtctctgtcccccggcgagagagccacactgagctgcagagcctcccagtccgtgacaagcagctatctggcttggtatcagcagaagcccggccaagcccctagactgctgatcaacgtgggcagcagaagagccaccggcatccccgacagattttccggcagcggctccggcacagacttcacactgaccatctctaggctggagcccgaggatttcgccgtgtactactgccagcaaggcatcatgctgcctcctaccttcggacaaggcaccaaggtggaaatcaaagaacccaagagctccgacaagacccacacgtgtcccccttgccctccttgtggcggaggaagctccggaggcggaagcggaggacagcctagggagccccaggtgtataccctccccccctccagggaagagatgaccaagaaccaggtgagcctgacctgcctcgtgaagggcttttatccctccgatatcgccgtggagtgggagagcaacggccagcctgagaacaattacaagaccaccccccctgtgctggactccgatggcagcttcttcctgtattccaagctgaccgtcgacaagtccaggtggcaacagggcaacgtcttcagctgcagcgtgatgcacgaggccctgcacaatcactacacccagaagtccctctccctgagccccggctga

SEQ ID NO: 49 - DNA, 780 bp, synthetic construct
  misc_feature 1..780: note = Synthetic Sequence
  source 1..780: mol_type = other DNA
atggagaccgacacactgctgctgtgggtgctgctgctgtgggtccccggctccaccggagaggtgcagctgctcgagagcggaggcggactcgtgcaacccggcggctctctgagactgagctgtgccgccagcggcttcaccttctccagctacgccatgagctgggtgagacaagcccccggcaaaggactggagtgggtgagcgccattatcggcagcggcgccagcacatactacgccgactccgtgaaaggaagattcacaatctctagggacaactccaagaacacactgtatctgcagatgaactctctgagagccgaggacaccgctgtctactattgcgccaagggatggttcggaggcttcaattactggggccaaggcacactcgtgacagtcagctcctccggaggaggaggagagatcgtgctgacccagagccccggcaccctctctctgagccccggcgaaagggccacactgagctgtagggccagccagagcgtgaccagcagctatctggcttggtaccagcagaagcccggacaagcccctagactgctgatcaacgtgggcagcagaagagccaccggcatccccgacagattctccggaagcggcagcggcaccgactttacactgaccatctctaggctggagcccgaggatttcgccgtctactactgccagcaaggcatcatgctgcctcccacctttggccaaggcaccaaggtggagatcaagggaggctgtcctccttgccctccttgctga

SEQ ID NO: 50 - DNA, 789 bp, synthetic construct
  misc_feature 1..789: note = Synthetic Sequence
  source 1..789: mol_type = other DNA
atggagaccgacacactgctgctgtgggtgctgctgctgtgggtccccggctccaccggagaggtgcagctgctcgagagcggaggcggactcgtgcaacccggcggctctctgagactgagctgtgccgccagcggcttcaccttctccagctacgccatgagctgggtgagacaagcccccggcaaaggactggagtgggtgagcgccattatcggcagcggcgccagcacatactacgccgactccgtgaaaggaagattcacaatctctagggacaactccaagaacacactgtatctgcagatgaactctctgagagccgaggacaccgctgtctactattgcgccaagggatggttcggaggcttcaattactggggccaaggcacactcgtgacagtcagctccggcggaggatccggaggaggaggagagatcgtgctgacccagagccccggcaccctctctctgagccccggcgaaagggccacactgagctgtagggccagccagagcgtgaccagcagctatctggcttggtaccagcagaagcccggacaagcccctagactgctgatcaacgtgggcagcagaagagccaccggcatccccgacagattctccggaagcggcagcggcaccgactttacactgaccatctctaggctggagcccgaggatttcgccgtctactactgccagcaaggcatcatgctgcctcccacctttggccaaggcaccaaggtggagatcaagggaggctgtcctccttgccctccttgctga

SEQ ID NO: 51 - DNA, 780 bp, synthetic construct
  misc_feature 1..780: note = Synthetic Sequence
  source 1..780: mol_type = other DNA
atggagacagacacactgctgctgtgggtgctgctgctgtgggtgcccggcagcaccggcgagatcgtgctgacccagtcccccggaacactgagcctcagccccggcgagagagccacactgagctgcagagccagccagtccgtgacaagcagctatctggcttggtaccagcagaagcccggacaagcccccagactgctgatcaacgtgggcagcagaagagccaccggcattcccgacagattcagcggaagcggcagcggcacagacttcacactgaccatcagcagactggagcccgaggactttgctgtgtactactgccagcaaggcatcatgctgccccccacctttggccaaggcaccaaggtcgagatcaagagcggaggaggaggagaggtccagctgctggaaagcggcggaggactcgtccagcccggcggatctctgaggctgagctgcgccgccagcggatttaccttctccagctacgccatgagctgggtgagacaagcccccggcaagggactggaatgggtgagcgccatcattggaagcggcgcctccacatactacgccgattccgtgaagggaaggttcaccatctctagggacaactccaagaacacactgtatctgcagatgaactctctgagggccgaggataccgccgtgtattactgcgccaagggctggtttggcggcttcaactactggggacaaggcacactggtgacagtgagcagcggaggctgccctccttgtcctccttgctga

SEQ ID NO: 52 - DNA, 789 bp, synthetic construct
  misc_feature 1..789: note = Synthetic Sequence
  source 1..789: mol_type = other DNA
atggagacagacacactgctgctgtgggtgctgctgctgtgggtgcccggcagcaccggcgagatcgtgctgacccagtcccccggaacactgagcctcagccccggcgagagagccacactgagctgcagagccagccagtccgtgacaagcagctatctggcttggtaccagcagaagcccggacaagcccccagactgctgatcaacgtgggcagcagaagagccaccggcattcccgacagattcagcggaagcggcagcggcacagacttcacactgaccatcagcagactggagcccgaggactttgctgtgtactactgccagcaaggcatcatgctgccccccacctttggccaaggcaccaaggtcgagatcaagggaggcggaagcggaggaggaggagaggtccagctgctggaaagcggcggaggactcgtccagcccggcggatctctgaggctgagctgcgccgccagcggatttaccttctccagctacgccatgagctgggtgagacaagcccccggcaagggactggaatgggtgagcgccatcattggaagcggcgcctccacatactacgccgattccgtgaagggaaggttcaccatctctagggacaactccaagaacacactgtatctgcagatgaactctctgagggccgaggataccgccgtgtattactgcgccaagggctggtttggcggcttcaactactggggacaaggcacactggtgacagtgagcagcggaggctgccctccttgtcctccttgctga

SEQ ID NO: 53 - DNA, 1188 bp, synthetic construct
  misc_feature 1..1188: note = Synthetic Sequence
  source 1..1188: mol_type = other DNA
atggaaaccgacacactgctgctgtgggtgctgctgctctgggtgcccggcagcaccggcgagatcgtgctgacccagtcccccggaacactcagcctctcccccggcgagagagccacactgagctgcagagccagccagtccgtgtctaggagctatctggcttggtaccagcagaagcccggacaagcccccagactgctgatcattggcgccagcaccagagccaccggcatccccgacagattctccggctccggcagcggcacagacttcacactgaccatttctaggctggagcccgaggacttcgctgtgtactattgccagcaaggccaagtgatcccccccacattcggccaaggcaccaaagtggagatcaaaggcagcacctccggcggaggatccggaggaggaagcggaggcggaggctccagcgaagtccagctgctcgaaagcggcggaggactggtgcaacccggcggaagcctcagactgagctgcgccgcctccggatttaccttctccagccacgccatgagctgggtgagacaagcccccggcaaaggactggagtgggtgtccgccatttgggcctccggcgagcagtactacgccgactccgtgaagggaagattcaccatctctagggacaacagcaagaacacactgtatctgcagatgaactctctgagggccgaggatacagccgtgtattattgcgccaagggctggctgggcaacttcgactattggggccaaggcacactggtgacagtgagctccgaacccaagagctccgacaagacccacacgtgtcccccttgccctccttgtggcggaggaagctccggaggcggaagcggaggacagcctagggagccccaggtgtataccctccccccctccagggaagagatgaccaagaaccaggtgagcctgacctgcctcgtgaagggcttttatccctccgatatcgccgtggagtgggagagcaacggccagcctgagaacaattacaagaccaccccccctgtgctggactccgatggcagcttcttcctgtattccaagctgaccgtcgacaagtccaggtggcaacagggcaacgtcttcagctgcagcgtgatgcacgaggccctgcacaatcactacacccagaagtccctctccctgagccccggctga

SEQ ID NO: 54 - DNA, 1188 bp, synthetic construct
  misc_feature 1..1188: note = Synthetic Sequence
  source 1..1188: mol_type = other DNA
atggagacagacacactgctgctgtgggtgctgctgctctgggtgcccggatccaccggcgaggtccaactgctggagtccggcggaggactggtgcaacccggaggatctctgagactcagctgtgccgccagcggcttcacctttagctcccacgccatgagctgggtgagacaagcccccggaaagggactcgagtgggtgagcgctatttgggccagcggcgagcagtattacgccgacagcgtgaagggaagattcacaatcagcagagacaacagcaagaacacactgtatctgcagatgaactctctgagagccgaggacaccgccgtctactactgtgccaagggctggctgggcaacttcgattactggggacaaggcacactggtcaccgtctccagcggaagcacatccggcggcggaagcggaggaggaagcggcggcggaggcagctccgagatcgtgctgacacagagccccggcacactgtctctgagccccggcgagagggctacactgagctgtagagcctcccagagcgtgtctaggtcctatctggcttggtaccagcagaaacccggacaagcccctagactgctgatcatcggagccagcacaagggccaccggcattcccgacagattctccggatccggctccggaaccgacttcacactgaccatctccagactggagcccgaggactttgccgtgtactactgtcagcaaggccaagtgatccctcctaccttcggccaaggcaccaaggtggaaattaaggaacccaagagctccgacaagacccacacgtgtcccccttgccctccttgtggcggaggaagctccggaggcggaagcggaggacagcctagggagccccaggtgtataccctccccccctccagggaagagatgaccaagaaccaggtgagcctgacctgcctcgtgaagggcttttatccctccgatatcgccgtggagtgggagagcaacggccagcctgagaacaattacaagaccaccccccctgtgctggactccgatggcagcttcttcctgtattccaagctgaccgtcgacaagtccaggtggcaacagggcaacgtcttcagctgcagcgtgatgcacgaggccctgcacaatcactacacccagaagtccctctccctgagccccggctga

SEQ ID NO: 55 - DNA, 777 bp, synthetic construct
  misc_feature 1..777: note = Synthetic Sequence
  source 1..777: mol_type = other DNA
atggagaccgatacactgctgctgtgggtgctgctgctctgggtgcccggctccaccggcgagatcgtgctgacccagagccccggcacactgtctctgtcccccggcgagagggccacactgagctgcagagcctcccagagcgtgtctaggagctatctggcttggtatcagcagaagcccggccaagcccctagactgctgattatcggcgctagcacaagagccaccggcatccccgatagatttagcggcagcggaagcggcaccgatttcacactgaccatttccagactggagcccgaggattttgccgtgtactactgtcagcaaggccaagtgatccctcccaccttcggacaaggcaccaaggtggagatcaaatccggaggaggaggagaggtgcaactgctggaaagcggcggaggactggtgcaacccggcggatctctgagactgagctgtgccgccagcggcttcacatttagcagccacgccatgagctgggtgagacaagcccccggcaagggactggagtgggtgagcgccatttgggcctccggagagcagtactatgccgacagcgtgaagggcagattcaccatctctagggacaactccaagaacaccctctacctccagatgaattctctgagagccgaggacaccgccgtctactactgtgccaagggctggctgggcaacttcgactactggggccaaggcacactggtgacagtgagcagcggaggctgccctccttgtcctccttgctga

SEQ ID NO: 56 - DNA, 777 bp, synthetic construct
  misc_feature 1..777: note = Synthetic Sequence
  source 1..777: mol_type = other DNA
atggaaacagacacactgctgctctgggtgctgctgctgtgggtgcccggcagcaccggcgaggtgcagctgctggaatccggaggcggactggtgcaacccggcggatctctgagactgagctgtgccgccagcggcttcacctttagcagccacgccatgagctgggtgaggcaagcccccggcaagggactcgagtgggtgagcgccatttgggctagcggcgagcagtactacgccgacagcgtcaaaggaagattcaccatctctagggacaacagcaagaacacactgtacctccagatgaactctctgagggccgaggataccgccgtgtactattgcgctaagggatggctgggcaacttcgactactggggacaaggcacactggtgacagtgagcagctccggaggaggaggcgagatcgtgctgacccaatcccccggcacactgtctctgagccccggcgagagagccacactgagctgcagagccagccagtccgtgtctaggagctatctggcttggtaccagcagaaacccggacaagcccctagactgctgatcatcggcgccagcacaagagccaccggcattcccgacagattttccggcagcggaagcggcacagacttcacactgaccatctctaggctggagcccgaggactttgccgtgtactactgccagcaaggccaagtgatccctcctacctttggccaaggcacaaaggtggagatcaaaggaggatgccctccttgccccccttgctga

SEQ ID NO: 57 - DNA, 786 bp, synthetic construct
  misc_feature 1..786: note = Synthetic Sequence
  source 1..786: mol_type = other DNA
atggagaccgatacactgctgctgtgggtgctgctgctctgggtgcccggctccaccggcgagatcgtgctgacccagagccccggcacactgtctctgtcccccggcgagagggccacactgagctgcagagcctcccagagcgtgtctaggagctatctggcttggtatcagcagaagcccggccaagcccctagactgctgattatcggcgctagcacaagagccaccggcatccccgatagatttagcggcagcggaagcggcaccgatttcacactgaccatttccagactggagcccgaggattttgccgtgtactactgtcagcaaggccaagtgatccctcccaccttcggacaaggcaccaaggtggagatcaaaggcggaggatccggaggaggaggagaggtgcaactgctggaaagcggcggaggactggtgcaacccggcggatctctgagactgagctgtgccgccagcggcttcacatttagcagccacgccatgagctgggtgagacaagcccccggcaagggactggagtgggtgagcgccatttgggcctccggagagcagtactatgccgacagcgtgaagggcagattcaccatctctagggacaactccaagaacaccctctacctccagatgaattctctgagagccgaggacaccgccgtctactactgtgccaagggctggctgggcaacttcgactactggggccaaggcacactggtgacagtgagcagcggaggctgccctccttgtcctccttgctga

SEQ ID NO: 58 - DNA, 786 bp, synthetic construct
  misc_feature 1..786: note = Synthetic Sequence
  source 1..786: mol_type = other DNA
atggaaacagacacactgctgctctgggtgctgctgctgtgggtgcccggcagcaccggcgaggtgcagctgctggaatccggaggcggactggtgcaacccggcggatctctgagactgagctgtgccgccagcggcttcacctttagcagccacgccatgagctgggtgaggcaagcccccggcaagggactcgagtgggtgagcgccatttgggctagcggcgagcagtactacgccgacagcgtcaaaggaagattcaccatctctagggacaacagcaagaacacactgtacctccagatgaactctctgagggccgaggataccgccgtgtactattgcgctaagggatggctgggcaacttcgactactggggacaaggcacactggtgacagtgagcagcggaggaggatccggaggaggaggcgagatcgtgctgacccaatcccccggcacactgtctctgagccccggcgagagagccacactgagctgcagagccagccagtccgtgtctaggagctatctggcttggtaccagcagaaacccggacaagcccctagactgctgatcatcggcgccagcacaagagccaccggcattcccgacagattttccggcagcggaagcggcacagacttcacactgaccatctctaggctggagcccgaggactttgccgtgtactactgccagcaaggccaagtgatccctcctacctttggccaaggcacaaaggtggagatcaaaggaggatgccctccttgccccccttgctga

SEQ ID NO: 59 - DNA, 1227 bp, synthetic construct
  misc_feature 1..1227: note = Synthetic Sequence
  source 1..1227: mol_type = other DNA
atggagaccgacacactgctgctgtgggtgctgctgctctgggtgcccggctccaccggcgatatcgtgatgacacagtcccccgattctctggctgtgtctctgggagagagagccaccattaactgcaagtcctcccagtctctgctgtactctaggaaccagaagaactatctggcttggtaccagcagaagcccggacagcctcccaagctgctgatcttttgggccagcaccagagaaagcggcgtgcccgacagatttagcggcagcggcttcggcaccgactttacactgaccatctcctctctgcaagccgaggacgtggccgtgtactactgccagcagtacttcagctaccctctgaccttcggacaaggcaccaaggtggaaatcaagggcagcaccagcggaggaggaagcggcggaggcagcggaggcggaggaagcagccaagtgcaactggtgcagagcggcgccgaggtcaaaaaacccggcgccagcgtcaaagtgagctgcaagacctctaggtacaccttcaccgagtacaccattcactgggtgaggcaagcccccggccaaagactggagtggatcggcggcatcaaccccaacaacggcatccctaactacaaccaaaagtttaagggcagagtgaccatcaccgtggacaccagcgccagcaccgcctacatggaactgtcctctctgaggagcgaagacaccgccgtctactactgcgctagaaggaggatcgcctacggctacgacgagggccatgccatggactactggggccaaggcacactggtgaccgtctccagcgaacccaagagctccgacaagacccacacgtgtcccccttgccctccttgtggcggaggaagctccggaggcggaagcggaggacagcctagggagccccaggtgtataccctccccccctccagggaagagatgaccaagaaccaggtgagcctgacctgcctcgtgaagggcttttatccctccgatatcgccgtggagtgggagagcaacggccagcctgagaacaattacaagaccaccccccctgtgctggactccgatggcagcttcttcctgtattccaagctgaccgtcgacaagtccaggtggcaacagggcaacgtcttcagctgcagcgtgatgcacgaggccctgcacaatcactacacccagaagtccctctccctgagccccggctga

SEQ ID NO: 60 - DNA, 1227 bp, synthetic construct
  misc_feature 1..1227: note = Synthetic Sequence
  source 1..1227: mol_type = other DNA
atggagaccgacacactgctgctgtgggtgctgctgctgtgggtccccggcagcaccggccaagtccagctggtccagtccggcgctgaagtgaaaaagcccggagccagcgtgaaggtgagctgcaagacctctaggtacaccttcaccgagtataccatccactgggtcagacaagcccccggccagaggctcgagtggatcggcggcatcaaccccaacaacggaatccccaactacaaccagaagttcaagggaagagtgaccattacagtggataccagcgcctccacagcctatatggaactgagctctctgaggagcgaagacaccgccgtgtactactgtgctagaaggagaatcgcctacggctacgacgagggacatgccatggattactggggacaaggcacactggtgaccgtgagcagcggctccaccagcggaggaggatccggcggaggcagcggaggcggcggatccagcgacatcgtgatgacacagagccccgactctctggctgtgtctctgggagagagggccacaatcaactgcaagagcagccagtctctgctgtattctaggaaccagaagaactatctggcttggtaccagcagaaacccggccagcctcccaagctgctgatcttttgggcctccacaagagagagcggcgtgcccgacagattcagcggaagcggcttcggcacagactttacactgaccatcagctctctgcaagccgaagatgtcgccgtctactactgccagcagtacttcagctaccctctgaccttcggccaaggcaccaaggtggaaatcaaagaacccaagagctccgacaagacccacacgtgtcccccttgccctccttgtggcggaggaagctccggaggcggaagcggaggacagcctagggagccccaggtgtataccctccccccctccagggaagagatgaccaagaaccaggtgagcctgacctgcctcgtgaagggcttttatccctccgatatcgccgtggagtgggagagcaacggccagcctgagaacaattacaagaccaccccccctgtgctggactccgatggcagcttcttcctgtattccaagctgaccgtcgacaagtccaggtggcaacagggcaacgtcttcagctgcagcgtgatgcacgaggccctgcacaatcactacacccagaagtccctctccctgagccccggctga

SEQ ID NO: 61 - DNA, 816 bp, synthetic construct
  misc_feature 1..816: note = Synthetic Sequence
  source 1..816: mol_type = other DNA
atggagacagatacactgctgctgtgggtgctgctgctctgggtgcccggcagcaccggccaagtgcagctggtgcagagcggagccgaggtgaaaaaacccggcgcttccgtgaaagtgagctgcaagacatctaggtacaccttcaccgagtacaccatccattgggtgaggcaagcccccggccagagactcgagtggatcggcggaatcaaccctaacaacggcatccccaactacaaccagaagttcaagggaagagtgaccatcaccgtggatacaagcgccagcaccgcttatatggagctgagctctctgaggtccgaggacaccgccgtgtattactgcgctagaaggaggattgcctacggctacgacgaaggccacgctatggactactggggccaaggcaccctcgtgacagtgagcagcagcggcggaggaggagatatcgtgatgacccagagccccgactctctggctgtgagcctcggagagagggccaccatcaactgcaagagcagccaatctctgctgtactctaggaaccagaagaactatctggcttggtaccaacagaaacccggccaacctcccaagctgctgatcttttgggctagcaccagagagtccggcgtccccgatagattcagcggcagcggcttcggcacagactttacactcaccatcagctctctgcaagccgaagacgtggccgtgtactactgccagcagtacttcagctaccctctgaccttcggccaaggcaccaaggtggagatcaaaggcggctgccccccttgccctccttgctga

SEQ ID NO: 62 - DNA, 825 bp, synthetic construct
  misc_feature 1..825: note = Synthetic Sequence
  source 1..825: mol_type = other DNA
atggagacagatacactgctgctgtgggtgctgctgctctgggtgcccggcagcaccggccaagtgcagctggtgcagagcggagccgaggtgaaaaaacccggcgcttccgtgaaagtgagctgcaagacatctaggtacaccttcaccgagtacaccatccattgggtgaggcaagcccccggccagagactcgagtggatcggcggaatcaaccctaacaacggcatccccaactacaaccagaagttcaagggaagagtgaccatcaccgtggatacaagcgccagcaccgcttatatggagctgagctctctgaggtccgaggacaccgccgtgtattactgcgctagaaggaggattgcctacggctacgacgaaggccacgctatggactactggggccaaggcaccctcgtgacagtgagcagcggaggaggcagcggcggaggaggagatatcgtgatgacccagagccccgactctctggctgtgagcctcggagagagggccaccatcaactgcaagagcagccaatctctgctgtactctaggaaccagaagaactatctggcttggtaccaacagaaacccggccaacctcccaagctgctgatcttttgggctagcaccagagagtccggcgtccccgatagattcagcggcagcggcttcggcacagactttacactcaccatcagctctctgcaagccgaagacgtggccgtgtactactgccagcagtacttcagctaccctctgaccttcggccaaggcaccaaggtggagatcaaaggcggctgccccccttgccctccttgctga

SEQ ID NO: 63 - DNA, 816 bp, synthetic construct
  misc_feature 1..816: note = Synthetic Sequence
  source 1..816: mol_type = other DNA
atggagaccgacacactgctgctgtgggtgctgctgctgtgggtccccggctccaccggcgacattgtgatgacccagagccccgattctctggctgtgtctctgggagagagggccaccatcaactgcaagtccagccagtctctgctctattctaggaaccagaagaactatctggcttggtaccagcaaaagcccggccagccccccaagctgctgattttctgggcttccacaagggagagcggcgtgcccgacagattcagcggcagcggctttggcaccgacttcacactgacaatttcctctctgcaagccgaggacgtggctgtgtactactgccagcagtacttcagctaccctctgacatttggacaaggcacaaaagtggagatcaaaagcggcggaggaggacaagtgcagctcgtgcagagcggagccgaagtgaagaaacccggcgctagcgtcaaggtgagctgcaagacctctaggtacaccttcaccgagtacaccattcactgggtgagacaagcccccggccagagactggaatggatcggcggcattaaccccaacaacggcatccccaactacaaccagaagttcaagggaagagtgacaatcacagtggacacatccgcctccacagcctacatggagctgtcctctctgagaagcgaagacaccgccgtgtattactgcgctagaagaaggatcgcctacggctatgacgaaggccacgccatggattattggggccaaggcacactggtcaccgtcagcagcggaggatgccctccttgtcccccttgctga

SEQ ID NO: 64 - DNA, 825 bp, synthetic construct
  misc_feature 1..825: note = Synthetic Sequence
  source 1..825: mol_type = other DNA
atggagaccgacacactgctgctgtgggtgctgctgctgtgggtccccggctccaccggcgacattgtgatgacccagagccccgattctctggctgtgtctctgggagagagggccaccatcaactgcaagtccagccagtctctgctctattctaggaaccagaagaactatctggcttggtaccagcaaaagcccggccagccccccaagctgctgattttctgggcttccacaagggagagcggcgtgcccgacagattcagcggcagcggctttggcaccgacttcacactgacaatttcctctctgcaagccgaggacgtggctgtgtactactgccagcagtacttcagctaccctctgacatttggacaaggcacaaaagtggagatcaaaggaggaggaagcggcggaggaggacaagtgcagctcgtgcagagcggagccgaagtgaagaaacccggcgctagcgtcaaggtgagctgcaagacctctaggtacaccttcaccgagtacaccattcactgggtgagacaagcccccggccagagactggaatggatcggcggcattaaccccaacaacggcatccccaactacaaccagaagttcaagggaagagtgacaatcacagtggacacatccgcctccacagcctacatggagctgtcctctctgagaagcgaagacaccgccgtgtattactgcgctagaagaaggatcgcctacggctatgacgaaggccacgccatggattattggggccaaggcacactggtcaccgtcagcagcggaggatgccctccttgtcccccttgctga

SEQ ID NO: 65 - DNA, 1227 bp, synthetic construct
  misc_feature 1..1227: note = Synthetic Sequence
  source 1..1227: mol_type = other DNA
atggagaccgacacactgctgctgtgggtgctgctgctctgggtgcccggcagcaccggcgatattgtgatgacacagtcccccgattctctggctgtgtctctgggagagagagccaccattaactgcaagtcctcccagtctctgctctactctaggaaccagaagaactatctggcttggtaccagcagaagcccggccagccccctaagctgctgatcttctgggcctccacaagagagtccggcgtgcccgacagattctccggctccggatccggcaccgatttcacactgaccatttcctctctgcaagccgaggactttgccgtctactactgccagcagtacttcagctatcctctgaccttcggccaaggcaccaaggtggaaatcaagggcagcaccagcggaggaggaagcggcggaggcagcggaggcggaggaagcagccaagtgcagctggtgcagagcggagctgaggtgaagaaacccggcgctagcgtgaaggtgagctgcaaggccagcggctacaccttcaccgagtataccatccactgggtgaggcaagcccccggacagagactcgagtggatcggcggcatcaaccccaataacggcatccctaactacaaccagaaattcaagggaagagtgaccatcaccgtcgataccagcgcctccacagcttacatggagctctcctctctgagaagcgaggacaccgccgtgtactactgcgctagaagaagaatcgcctatggctatgacgagggacatgccatggattactggggccaaggcacactggtgaccgtcagcagcgaacccaagagctccgacaagacccacacgtgtcccccttgccctccttgtggcggaggaagctccggaggcggaagcggaggacagcctagggagccccaggtgtataccctccccccctccagggaagagatgaccaagaaccaggtgagcctgacctgcctcgtgaagggcttttatccctccgatatcgccgtggagtgggagagcaacggccagcctgagaacaattacaagaccaccccccctgtgctggactccgatggcagcttcttcctgtattccaagctgaccgtcgacaagtccaggtggcaacagggcaacgtcttcagctgcagcgtgatgcacgaggccctgcacaatcactacacccagaagtccctctccctgagccccggctga

SEQ ID NO: 66 - DNA, 1227 bp, synthetic construct
  misc_feature 1..1227: note = Synthetic Sequence
  source 1..1227: mol_type = other DNA
atggagaccgacacactgctgctgtgggtgctgctgctgtgggtccccggcagcaccggacaagtgcagctggtgcaaagcggcgccgaggtgaaaaagcccggcgcttccgtgaaggtcagctgcaaagccagcggctacaccttcacagagtacaccatccactgggtgagacaagcccccggacagagactcgagtggatcggcggcatcaaccccaacaacggcatccccaactacaaccagaaattcaaaggaagagtgaccatcaccgtggatacctccgccagcaccgcctatatggagctgagctctctgagatccgaggatacagccgtgtactactgcgccagaagaaggatcgcctacggctacgacgaaggccacgccatggattactggggacaaggcacactggtcacagtcagcagcggctccacctccggaggaggaagcggaggaggatccggaggaggcggatcctccgacattgtgatgacacagagccccgactctctggccgtgtctctgggagagagggctaccatcaactgcaagagcagccagtctctgctctactctaggaaccagaagaactatctggcttggtaccagcagaagcccggccagcctcctaagctgctcattttctgggctagcacaagagaaagcggcgtgcccgatagatttagcggatccggctccggcaccgacttcacactcaccatcagctctctgcaagccgaagacttcgccgtctactactgccagcagtacttcagctaccccctcacattcggacaaggcaccaaggtggagatcaaggaacccaagagctccgacaagacccacacgtgtcccccttgccctccttgtggcggaggaagctccggaggcggaagcggaggacagcctagggagccccaggtgtataccctccccccctccagggaagagatgaccaagaaccaggtgagcctgacctgcctcgtgaagggcttttatccctccgatatcgccgtggagtgggagagcaacggccagcctgagaacaattacaagaccaccccccctgtgctggactccgatggcagcttcttcctgtattccaagctgaccgtcgacaagtccaggtggcaacagggcaacgtcttcagctgcagcgtgatgcacgaggccctgcacaatcactacacccagaagtccctctccctgagccccggctga

SEQ ID NO: 67 - DNA, 1227 bp, synthetic construct
  misc_feature 1..1227: note = Synthetic Sequence
  source 1..1227: mol_type = other DNA
atggagaccgacacactgctgctgtgggtgctgctgctctgggtgcccggcagcaccggcgatattgtgatgacacagtcccccgattctctggctgtgtctctgggagagagagccaccattaactgcaagtcctcccagtctctgctctactctaggaaccagaagaactatctggcttggtaccagcagaagcccggccagccccctaagctgctgatcttctgggcctccacaagagagtccggcgtgcccgacagattctccggctccggatccggcaccgatttcacactgaccatttcctctctgcaagccgaggactttgccgtctactactgccagcagtactacagctatcctctgaccttcggccaaggcaccaaggtggaaatcaagggcagcaccagcggaggaggaagcggcggaggcagcggaggcggaggaagcagccaagtgcagctggtgcagagcggagctgaggtgaagaaacccggcgctagcgtgaaggtgagctgcaaggccagcggctacaccttcaccgagtataccatccactgggtgaggcaagcccccggacagagactcgagtggatcggcggcatcaaccccaataacggcatccctaactacaaccagaaattcaagggaagagtgaccatcaccgtcgataccagcgcctccacagcttacatggagctctcctctctgagaagcgaggacaccgccgtgtactactgcgctagaagaagaatcgcctatggctatgacgagggacatgccatggattactggggccaaggcacactggtgaccgtcagcagcgaacccaagagctccgacaagacccacacgtgtcccccttgccctccttgtggcggaggaagctccggaggcggaagcggaggacagcctagggagccccaggtgtataccctccccccctccagggaagagatgaccaagaaccaggtgagcctgacctgcctcgtgaagggcttttatccctccgatatcgccgtggagtgggagagcaacggccagcctgagaacaattacaagaccaccccccctgtgctggactccgatggcagcttcttcctgtattccaagctgaccgtcgacaagtccaggtggcaacagggcaacgtcttcagctgcagcgtgatgcacgaggccctgcacaatcactacacccagaagtccctctccctgagccccggctga

SEQ ID NO: 68 - DNA, 1227 bp, synthetic construct
  misc_feature 1..1227: note = Synthetic Sequence
  source 1..1227: mol_type = other DNA
atggagaccgacacactgctgctgtgggtgctgctgctgtgggtccccggcagcaccggacaagtgcagctggtgcaaagcggcgccgaggtgaaaaagcccggcgcttccgtgaaggtcagctgcaaagccagcggctacaccttcacagagtacaccatccactgggtgagacaagcccccggacagagactcgagtggatcggcggcatcaaccccaacaacggcatccccaactacaaccagaaattcaaaggaagagtgaccatcaccgtggatacctccgccagcaccgcctatatggagctgagctctctgagatccgaggatacagccgtgtactactgcgccagaagaaggatcgcctacggctacgacgaaggccacgccatggattactggggacaaggcacactggtcacagtcagcagcggctccacctccggaggaggaagcggaggaggatccggaggaggcggatcctccgacattgtgatgacacagagccccgactctctggccgtgtctctgggagagagggctaccatcaactgcaagagcagccagtctctgctctactctaggaaccagaagaactatctggcttggtaccagcagaagcccggccagcctcctaagctgctcattttctgggctagcacaagagaaagcggcgtgcccgatagatttagcggatccggctccggcaccgacttcacactcaccatcagctctctgcaagccgaagacttcgccgtctactactgccagcagtactacagctaccccctcacattcggacaaggcaccaaggtggagatcaaggaacccaagagctccgacaagacccacacgtgtcccccttgccctccttgtggcggaggaagctccggaggcggaagcggaggacagcctagggagccccaggtgtataccctccccccctccagggaagagatgaccaagaaccaggtgagcctgacctgcctcgtgaagggcttttatccctccgatatcgccgtggagtgggagagcaacggccagcctgagaacaattacaagaccaccccccctgtgctggactccgatggcagcttcttcctgtattccaagctgaccgtcgacaagtccaggtggcaacagggcaacgtcttcagctgcagcgtgatgcacgaggccctgcacaatcactacacccagaagtccctctccctgagccccggctga

SEQ ID NO: 69 - DNA, 1227 bp, synthetic construct
  misc_feature 1..1227: note = Synthetic Sequence
  source 1..1227: mol_type = other DNA
atggagaccgacacactgctgctgtgggtgctgctgctctgggtgcccggcagcaccggcgacatcgtgatgacccagtcccccgattctctggctgtgtctctgggagagagagccaccattaactgcaagagcagccagtctctgctgtactctaggaaccagaagaactatctggcttggtatcagcagaagcccggacagccccccaaactgctgatcttttgggccagcacaagggagtccggcgtgcccgacagattctccggcagcggctttggcaccgacttcacactgaccatttctaggctggagcccgaggactttgctgtgtactactgccagcagtacttcagctatcctctgaccttcggccaaggcaccaaagtggagatcaagggctccaccagcggcggaggaagcggaggcggcagcggaggaggaggcagcagccaagtgcagctggtgcagagcggcgccgaagtgaagaaacccggcgctagcgtgaaggtgagctgcaagacctctaggtacaccttcaccgagtacaccatccactgggtgaggcaagcccccggccagagactggaatggatcggcggcatcaaccccaacaacggcatccccaactacaaccagaaattcaagggaagagtgaccatcacagtcgacaccagcgccagcaccgcctacatggaactgtcctctctgaggagcgaagacacagccgtgtattactgcgctagaaggagaatcgcctacggctacgacgagggccacgctatggactactggggacaaggcacactggtgaccgtgagctccgaacccaagagctccgacaagacccacacgtgtcccccttgccctccttgtggcggaggaagctccggaggcggaagcggaggacagcctagggagccccaggtgtataccctccccccctccagggaagagatgaccaagaaccaggtgagcctgacctgcctcgtgaagggcttttatccctccgatatcgccgtggagtgggagagcaacggccagcctgagaacaattacaagaccaccccccctgtgctggactccgatggcagcttcttcctgtattccaagctgaccgtcgacaagtccaggtggcaacagggcaacgtcttcagctgcagcgtgatgcacgaggccctgcacaatcactacacccagaagtccctctccctgagccccggctga

SEQ ID NO: 70 - DNA, 1227 bp, synthetic construct
  misc_feature 1..1227: note = Synthetic Sequence
  source 1..1227: mol_type = other DNA
atggagaccgacacactgctgctgtgggtgctgctgctctgggtgcccggcagcaccggccaagtgcagctggtccagagcggcgctgaagtgaaaaagcccggagccagcgtgaaggtgagctgcaagacctctaggtacaccttcacagagtacaccatccactgggtgagacaagcccccggacagaggctcgagtggatcggaggcatcaaccccaacaacggcatccccaactacaaccagaagttcaagggaagagtgacaatcaccgtggacacctccgccagcaccgcctacatggagctgtccagcctcagaagcgaggacaccgctgtctactactgcgccagaaggaggattgcctacggatacgacgaaggacacgccatggattactggggacaaggcacactggtcacagtgagctccggaagcacatccggaggaggaagcggaggaggatccggaggcggaggatccagcgacatcgtgatgacacagagccccgactctctggctgtctctctgggagagagggctacaatcaactgcaagagcagccagtctctgctgtactctaggaaccagaagaactacctcgcttggtaccagcagaagcccggacagcctcccaagctgctgatcttctgggccagcacaagagaaagcggagtgcccgacagattctccggcagcggattcggaacagacttcacactgaccatctccagactggagcccgaggacttcgccgtgtactactgccagcagtacttcagctaccctctgacctttggccaaggcaccaaggtggagatcaaggaacccaagagctccgacaagacccacacgtgtcccccttgccctccttgtggcggaggaagctccggaggcggaagcggaggacagcctagggagccccaggtgtataccctccccccctccagggaagagatgaccaagaaccaggtgagcctgacctgcctcgtgaagggcttttatccctccgatatcgccgtggagtgggagagcaacggccagcctgagaacaattacaagaccaccccccctgtgctggactccgatggcagcttcttcctgtattccaagctgaccgtcgacaagtccaggtggcaacagggcaacgtcttcagctgcagcgtgatgcacgaggccctgcacaatcactacacccagaagtccctctccctgagccccggctga

SEQ ID NO: 71 - DNA, 1227 bp, synthetic construct
  misc_feature 1..1227: note = Synthetic Sequence
  source 1..1227: mol_type = other DNA
atggagaccgacacactgctgctgtgggtgctgctgctctgggtgcccggcagcaccggcgatatcgtgatgacacagagccccgattctctggctgtgtctctgggagagagagccaccatcaactgcaagtccagccaatctctgctgtactctaggaaccagaagaactatctggcttggtatcagcagaagcccggacagccccccaagctgctgatcttttgggctagcaccagagagtccggcgtgcccgacagattctccggctccggattcggcaccgacttcacactgaccatcagcagactggaacccgaggacttcgccgtctactactgccagcagtactacagctaccccctcaccttcggacaaggcaccaaggtggaaatcaagggcagcacatccggaggaggaagcggaggaggatccggaggcggaggaagcagccaagtgcagctggtgcagtccggagctgaagtgaagaaacccggagccagcgtgaaggtgagctgcaaagcttccggctacacctttaccgagtacaccatccactgggtgaggcaagcccccggccagaggctcgagtggatcggcggcatcaaccccaataacggcatccccaactacaaccagaagttcaagggaagagtgaccatcacagtggacaccagcgccagcaccgcctacatggagctgtcctctctgaggagcgaggacacagccgtgtactactgcgctagaagaagaattgcctacggatacgacgagggccacgctatggactactggggccaaggcacactggtgaccgtgtccagcgaacccaagagctccgacaagacccacacgtgtcccccttgccctccttgtggcggaggaagctccggaggcggaagcggaggacagcctagggagccccaggtgtataccctccccccctccagggaagagatgaccaagaaccaggtgagcctgacctgcctcgtgaagggcttttatccctccgatatcgccgtggagtgggagagcaacggccagcctgagaacaattacaagaccaccccccctgtgctggactccgatggcagcttcttcctgtattccaagctgaccgtcgacaagtccaggtggcaacagggcaacgtcttcagctgcagcgtgatgcacgaggccctgcacaatcactacacccagaagtccctctccctgagccccggctga

SEQ ID NO: 72 - DNA, 1227 bp, synthetic construct
  misc_feature 1..1227: note = Synthetic Sequence
  source 1..1227: mol_type = other DNA
atggaaaccgacacactgctgctgtgggtgctgctgctctgggtgcccggcagcaccggccaagtgcagctggtgcagtccggagccgaagtgaaaaaacccggcgccagcgtgaaagtgagctgcaaggccagcggctacaccttcaccgagtacacaatccactgggtgagacaagcccccggacagagactggaatggatcggcggcatcaaccccaacaacggcatccccaactacaaccagaagttcaagggaagagtgaccatcaccgtggacacctccgccagcaccgcctatatggagctgagctctctgagatccgaggacacagccgtgtactactgcgctagaagaagaatcgcctatggctacgacgagggccatgccatggattactggggccaaggcacactcgtgacagtgtcctccggcagcacctccggaggaggaagcggaggcggatccggaggcggaggctcctccgacatcgtgatgacacagagccccgactctctggccgtgtctctgggagagagggctacaatcaactgcaagagcagccagtctctgctgtattctaggaaccagaagaactacctcgcttggtaccagcagaaacccggccagcctcccaagctgctgatcttctgggcttccacaagggagagcggcgtgcccgacagattcagcggctccggcttcggaaccgacttcaccctcaccatcagcagactggagcccgaggacttcgccgtctactactgccagcagtactacagctaccctctgacattcggccaaggcaccaaggtggagatcaaggaacccaagagctccgacaagacccacacgtgtcccccttgccctccttgtggcggaggaagctccggaggcggaagcggaggacagcctagggagccccaggtgtataccctccccccctccagggaagagatgaccaagaaccaggtgagcctgacctgcctcgtgaagggcttttatccctccgatatcgccgtggagtgggagagcaacggccagcctgagaacaattacaagaccaccccccctgtgctggactccgatggcagcttcttcctgtattccaagctgaccgtcgacaagtccaggtggcaacagggcaacgtcttcagctgcagcgtgatgcacgaggccctgcacaatcactacacccagaagtccctctccctgagccccggctga

SEQ ID NO: 73 - DNA, 1227 bp, synthetic construct
  misc_feature 1..1227: note = Synthetic Sequence
  source 1..1227: mol_type = other DNA
atggagaccgacacactgctgctgtgggtgctgctgctctgggtgcccggcagcaccggcgatatcgtgatgacacagagccccgattctctggctgtgtctctgggagagagagccaccatcaactgcaagtccagccaatctctgctgtactctaggaaccagaagaactatctggcttggtatcagcagaagcccggacagccccccaagctgctgatcttttgggctagcaccagagagtccggcgtgcccgacagattctccggctccggaagcggcaccgacttcacactgaccatcagcagactggaacccgaggacttcgccgtctactactgccagcagtactacagctaccccctcaccttcggacaaggcaccaaggtggaaatcaagggcagcacatccggaggaggaagcggaggaggatccggaggcggaggaagcagccaagtgcagctggtgcagtccggagctgaagtgaagaaacccggagccagcgtgaaggtgagctgcaaagcttccggctacacctttaccgagtacaccatccactgggtgaggcaagcccccggccagaggctcgagtggatcggcggcatcaaccccaataacggcatccccaactacaaccagaagttcaagggaagagtgaccatcacagtggacaccagcgccagcaccgcctacatggagctgtcctctctgaggagcgaggacacagccgtgtactactgcgctagaagaagaattgcctacggatacgacgagggccacgctatggactactggggccaaggcacactggtgaccgtgtccagcgaacccaagagctccgacaagacccacacgtgtcccccttgccctccttgtggcggaggaagctccggaggcggaagcggaggacagcctagggagccccaggtgtataccctccccccctccagggaagagatgaccaagaaccaggtgagcctgacctgcctcgtgaagggcttttatccctccgatatcgccgtggagtgggagagcaacggccagcctgagaacaattacaagaccaccccccctgtgctggactccgatggcagcttcttcctgtattccaagctgaccgtcgacaagtccaggtggcaacagggcaacgtcttcagctgcagcgtgatgcacgaggccctgcacaatcactacacccagaagtccctctccctgagccccggctga

SEQ ID NO: 74 - DNA, 1227 bp, synthetic construct
  misc_feature 1..1227: note = Synthetic Sequence
  source 1..1227: mol_type = other DNA
atggaaaccgacacactgctgctgtgggtgctgctgctctgggtgcccggcagcaccggccaagtgcagctggtgcagtccggagccgaagtgaaaaaacccggcgccagcgtgaaagtgagctgcaaggccagcggctacaccttcaccgagtacacaatccactgggtgagacaagcccccggacagagactggaatggatcggcggcatcaaccccaacaacggcatccccaactacaaccagaagttcaagggaagagtgaccatcaccgtggacacctccgccagcaccgcctatatggagctgagctctctgagatccgaggacacagccgtgtactactgcgctagaagaagaatcgcctatggctacgacgagggccatgccatggattactggggccaaggcacactcgtgacagtgtcctccggcagcacctccggaggaggaagcggaggcggatccggaggcggaggctcctccgacatcgtgatgacacagagccccgactctctggccgtgtctctgggagagagggctacaatcaactgcaagagcagccagtctctgctgtattctaggaaccagaagaactacctcgcttggtaccagcagaaacccggccagcctcccaagctgctgatcttctgggcttccacaagggagagcggcgtgcccgacagattcagcggctccggcagcggaaccgacttcaccctcaccatcagcagactggagcccgaggacttcgccgtctactactgccagcagtactacagctaccctctgacattcggccaaggcaccaaggtggagatcaaggaacccaagagctccgacaagacccacacgtgtcccccttgccctccttgtggcggaggaagctccggaggcggaagcggaggacagcctagggagccccaggtgtataccctccccccctccagggaagagatgaccaagaaccaggtgagcctgacctgcctcgtgaagggcttttatccctccgatatcgccgtggagtgggagagcaacggccagcctgagaacaattacaagaccaccccccctgtgctggactccgatggcagcttcttcctgtattccaagctgaccgtcgacaagtccaggtggcaacagggcaacgtcttcagctgcagcgtgatgcacgaggccctgcacaatcactacacccagaagtccctctccctgagccccggctga

SEQ ID NO: 75 - DNA, 816 bp, synthetic construct
  misc_feature 1..816: note = Synthetic Sequence
  source 1..816: mol_type = other DNA
atggagaccgatacactgctgctctgggtgctgctgctgtgggtgcccggcagcaccggcgacattgtgatgacccagagccccgactctctggccgtgtctctgggagagagagccaccatcaactgcaagagctcccagtctctgctctactctaggaaccagaagaactatctggcttggtaccagcagaagcccggacagcctcctaagctgctcatcttctgggctagcacaagagaaagcggagtgcccgacagattttccggctccggcagcggcaccgactttacactgaccatcagctctctgcaagccgaagattttgccgtgtactactgccagcagtactacagctaccctctgacatttggccaaggcacaaaggtggagatcaaaagcggcggaggaggacaagtgcagctcgtgcagtccggcgccgaagtgaaaaagcccggcgcttccgtgaaggtgagctgcaaagccagcggctacaccttcaccgagtataccatccactgggtgaggcaagcccccggccagagactggagtggatcggcggcatcaaccccaacaacggcattcccaactataaccagaagttcaagggaagagtgaccatcaccgtggacaccagcgctagcacagcttacatggagctgtcctctctgaggagcgaagacaccgccgtctactactgcgctaggaggagaattgcctacggctacgatgaaggccacgccatggactactggggacaaggcacactcgtgaccgtgagctccggaggatgtcctccttgccccccttgctga

SEQ ID NO: 76 - DNA, 816 bp, synthetic construct
  misc_feature 1..816: note = Synthetic Sequence
  source 1..816: mol_type = other DNA
atggaaaccgatacactgctgctgtgggtgctgctgctgtgggtgcccggcagcaccggccaagtgcagctggtgcagagcggagccgaggtgaagaaacccggcgccagcgtgaaggtgagctgcaaggccagcggctacaccttcaccgagtacaccatccactgggtgaggcaagcccccggccagagactggagtggattggcggcatcaaccccaacaacggcatccccaactacaatcagaagttcaagggaagagtgaccatcaccgtggataccagcgctagcacagcctacatggaactgagctctctgaggagcgaggacacagccgtgtactactgcgctagaagaaggatcgcctatggctatgacgaaggccacgctatggactactggggacaaggcacactggtgacagtcagcagctccggaggcggaggcgacatcgtgatgacacagtcccccgattctctggctgtgtctctgggagagagggctaccatcaattgcaagagcagccagtctctgctgtactctaggaaccagaagaactatctggcttggtaccagcagaagcccggccagcctcctaagctgctcatcttctgggcctccacaagagaaagcggcgtgcccgatagattctccggcagcggcagcggcaccgattttacactgaccatcagctctctgcaagccgaagacttcgccgtctactactgccagcagtactactcctaccctctgaccttcggccaaggcaccaaggtggagatcaaaggcggctgccccccttgccctccttgctga

SEQ ID NO: 77 - DNA, 825 bp, synthetic construct
  misc_feature 1..825: note = Synthetic Sequence
  source 1..825: mol_type = other DNA
atggagaccgatacactgctgctctgggtgctgctgctgtgggtgcccggcagcaccggcgacattgtgatgacccagagccccgactctctggccgtgtctctgggagagagagccaccatcaactgcaagagctcccagtctctgctctactctaggaaccagaagaactatctggcttggtaccagcagaagcccggacagcctcctaagctgctcatcttctgggctagcacaagagaaagcggagtgcccgacagattttccggctccggcagcggcaccgactttacactgaccatcagctctctgcaagccgaagattttgccgtgtactactgccagcagtactacagctaccctctgacatttggccaaggcacaaaggtggagatcaaaggaggaggaagcggcggaggaggacaagtgcagctcgtgcagtccggcgccgaagtgaaaaagcccggcgcttccgtgaaggtgagctgcaaagccagcggctacaccttcaccgagtataccatccactgggtgaggcaagcccccggccagagactggagtggatcggcggcatcaaccccaacaacggcattcccaactataaccagaagttcaagggaagagtgaccatcaccgtggacaccagcgctagcacagcttacatggagctgtcctctctgaggagcgaagacaccgccgtctactactgcgctaggaggagaattgcctacggctacgatgaaggccacgccatggactactggggacaaggcacactcgtgaccgtgagctccggaggatgtcctccttgccccccttgctga

SEQ ID NO: 78 - DNA, 825 bp, synthetic construct
  misc_feature 1..825: note = Synthetic Sequence
  source 1..825: mol_type = other DNA
atggaaaccgatacactgctgctgtgggtgctgctgctgtgggtgcccggcagcaccggccaagtgcagctggtgcagagcggagccgaggtgaagaaacccggcgccagcgtgaaggtgagctgcaaggccagcggctacaccttcaccgagtacaccatccactgggtgaggcaagcccccggccagagactggagtggattggcggcatcaaccccaacaacggcatccccaactacaatcagaagttcaagggaagagtgaccatcaccgtggataccagcgctagcacagcctacatggaactgagctctctgaggagcgaggacacagccgtgtactactgcgctagaagaaggatcgcctatggctatgacgaaggccacgctatggactactggggacaaggcacactggtgacagtcagcagcggaggaggatccggaggcggaggcgacatcgtgatgacacagtcccccgattctctggctgtgtctctgggagagagggctaccatcaattgcaagagcagccagtctctgctgtactctaggaaccagaagaactatctggcttggtaccagcagaagcccggccagcctcctaagctgctcatcttctgggcctccacaagagaaagcggcgtgcccgatagattctccggcagcggcagcggcaccgattttacactgaccatcagctctctgcaagccgaagacttcgccgtctactactgccagcagtactactcctaccctctgaccttcggccaaggcaccaaggtggagatcaaaggcggctgccccccttgccctccttgctga

SEQ ID NO: 79 - DNA, 1227 bp, synthetic construct
  misc_feature 1..1227: note = Synthetic Sequence
  source 1..1227: mol_type = other DNA
atggagaccgacacactgctgctctgggtgctgctgctgtgggtgcccggctccaccggagacatcgtgatgtcccagagccctagctccctcgctgtgagcgtgggagagaaggtgaccatgagctgcaagagcagccagtctctgctgtactctaggaaccagaagaactatctggcttggtttcagcagaagcccggccaatcccccaagctgctgatcttttgggctagcacaagggagtccggcgtgcccgacagattcaccggcagcggctttggcaccgatttcaatctgaccatcagcagcgtccaagccgaggatctggccgtgtacgactgccagcagtatttcagctatcctctgaccttcggcgccggaaccaagctggaactgaagggcagcacatccggaggaggaagcggaggaggatccggaggcggaggaagcagcgaagtgcagctgcagcagagcggccccgagctcgtgaaacccggcgcctccgtgaagatgagctgtaagacctctaggtacaccttcaccgagtacaccatccactgggtgagacagagccacggaaaatctctggagtggatcggcggcatcaaccccaacaacggcatccccaactacaaccagaaattcaagggaagagccaccctcacagtgggcaagtccagctccaccgcctacatggagctcagatctctgaccagcgaggacagcgccgtgtacttttgcgctagaaggagaatcgcctatggctacgacgagggccacgccatggattactggggccaaggcacatccgtgaccgtctcctccgaacccaagagctccgacaagacccacacgtgtcccccttgccctccttgtggcggaggaagctccggaggcggaagcggaggacagcctagggagccccaggtgtataccctccccccctccagggaagagatgaccaagaaccaggtgagcctgacctgcctcgtgaagggcttttatccctccgatatcgccgtggagtgggagagcaacggccagcctgagaacaattacaagaccaccccccctgtgctggactccgatggcagcttcttcctgtattccaagctgaccgtcgacaagtccaggtggcaacagggcaacgtcttcagctgcagcgtgatgcacgaggccctgcacaatcactacacccagaagtccctctccctgagccccggctga

SEQ ID NO: 80 - DNA, 1227 bp, synthetic construct
  misc_feature 1..1227: note = Synthetic Sequence
  source 1..1227: mol_type = other DNA
atggagaccgacacactgctgctgtgggtgctgctgctgtgggtgcccggcagcaccggcgaggtgcagctgcagcaaagcggacccgagctggtgaaacccggagccagcgtgaagatgagctgcaagacctctaggtacaccttcaccgagtacacaatccactgggtgaggcagtcccacggaaagtccctcgagtggatcggcggaatcaaccctaacaacggcatccccaactacaaccagaaattcaagggaagagccacactgaccgtgggcaaaagcagcagcacagcctacatggagctgaggtctctgaccagcgaggattccgccgtgtacttctgcgccagaagaaggatcgcctacggctacgatgagggccacgctatggattactggggccaaggcaccagcgtcacagtgagctccggaagcaccagcggaggaggaagcggaggcggatccggaggcggaggctccagcgacatcgtgatgtcccagagcccttcctctctggctgtgagcgtgggagagaaggtgaccatgagctgcaagagcagccagtctctgctctactctaggaaccagaagaactacctcgcttggttccagcagaaacccggccagtcccccaagctgctgatcttctgggccagcacaagagagagcggcgtgcccgacagatttaccggcagcggcttcggcaccgacttcaatctgacaatcagcagcgtgcaagccgaagatctggccgtgtacgactgccagcagtacttcagctaccctctgacatttggcgccggaaccaaactggagctgaaggaacccaagagctccgacaagacccacacgtgtcccccttgccctccttgtggcggaggaagctccggaggcggaagcggaggacagcctagggagccccaggtgtataccctccccccctccagggaagagatgaccaagaaccaggtgagcctgacctgcctcgtgaagggcttttatccctccgatatcgccgtggagtgggagagcaacggccagcctgagaacaattacaagaccaccccccctgtgctggactccgatggcagcttcttcctgtattccaagctgaccgtcgacaagtccaggtggcaacagggcaacgtcttcagctgcagcgtgatgcacgaggccctgcacaatcactacacccagaagtccctctccctgagccccggctga

SEQ ID NO: 81 - protein, 5 aa, synthetic construct
  REGION 1..5: note = Synthetic Construct
NYDIN

SEQ ID NO: 82 - protein, 9 aa, synthetic construct
  REGION 1..9: note = Synthetic Construct
LIWTGGGTN

SEQ ID NO: 83 - protein, 11 aa, synthetic construct
  REGION 1..11: note = Synthetic Construct
GGPLVWYALDY

SEQ ID NO: 84 - protein, 11 aa, synthetic construct
  REGION 1..11: note = Synthetic Construct
KASQDVSTAVA

SEQ ID NO: 85 - protein, 7 aa, synthetic construct
  REGION 1..7: note = Synthetic Construct
SASYRYT

SEQ ID NO: 86 - protein, 9 aa, synthetic construct
  REGION 1..9: note = Synthetic Construct
QQHYSNPRT

SEQ ID NO: 87 - protein, 119 aa, synthetic construct
  REGION 1..119: note = Synthetic Construct
QVQLKESGPGLVAPSQSLSITCTVSGFSLTNYDINWIRQPPGKGLEWLGLIWTGGGTNYNSAFMSRLSISKDNSKSQVFLKMNSLQTDDTAIYYCVRGGPLVWYALDYWGQGTSVTVSS

SEQ ID NO: 88 - protein, 107 aa, synthetic construct
  REGION 1..107: note = Synthetic Construct
DIVMTQSHKFMSTSVGDRVSITCKASQDVSTAVAWYQQKPGQSPKLLIYSASYRYTGVPDRFTGSGSGTDFTFTISSVQAEDLAVYYCQQHYSNPRTFGGGTKLEIK

SEQ ID NO: 89 - protein, 97 aa, synthetic construct
  REGION 1..97: note = Synthetic Construct
QVQLQESGPGLVKPSETLSLTCTVSGGSISSYYWSWIRQPPGKGLEWIGYIYTSGSTNYNPSLKSRVTISVDTSKNQFSLKLSSVTAADTAVYYCAR

SEQ ID NO: 90 - protein, 93 aa, synthetic construct
  REGION 1..93: note = Synthetic Construct
DIQMTQSPSSLSASVGDRVTITCQASQDISNYLNWYQQKPGKAPKLLIYDASNLETGVPSRFSGSGSGTDFTFTISSLQPEDIATYYCQQYDN

SEQ ID NO: 91 - protein, 119 aa, synthetic construct
  REGION 1..119: note = Synthetic Construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARYDGIYGELDFWGQGTLVTVSS

SEQ ID NO: 92 - protein, 107 aa, synthetic construct [sequence omitted]

SEQ ID NO: 93 - protein, 119 aa, synthetic construct
  REGION 1..119: note = Synthetic Construct
QVQLQESGPGLVKPSETLSLTCTVSGFSLTNYDINWIRQPPGKGLEWIGLIWTGGGTNYNPSLKSRVTISKDTSKNQFSLKLSSVTAADTAVYYCVRGGPLVWYALDYWGQGTLVTVSS

SEQ ID NO: 94 - protein, 107 aa, synthetic construct
  REGION 1..107: note = Synthetic Construct
DIQMTQSPSSLSASVGDRVTITCKASQDVSTAVAWYQQKPGKAPKLLIYSASYRYTGVPSRFSGSGSGTDFTFTISSLQPEDIATYYCQQHYSNPRTFGGGTKVEIK

SEQ ID NO: 95 - protein, 119 aa, synthetic construct
  REGION 1..119: note = Synthetic Construct
QVQLVQSGAEVKKPGSSVKVSCKASGFSLTNYDINWVRQAPGQGLEWMGLIWTGGGTNYNQKFQGRVTITKDNSTSTAYMELSSLRSEDTAVYYCVRGGPLVWYALDYWGQGTLVTVSS

SEQ ID NO: 96 - protein, 107 aa, synthetic construct
  REGION 1..107: note = Synthetic Construct
DIQMTQSPSSLSASVGDRVTITCKASQDVSTAVAWYQQKPGKAPKLLIYSASYRYTGVPSRFSGSGSGTDFTLTISSLQPEDFATYYCQQHYSNPRTFGQGTKVEIK

SEQ ID NO: 97 - protein, 226 aa, synthetic construct
  REGION 1..226: note = Synthetic Construct
QVQLKESGPGLVAPSQSLSITCTVSGFSLTNYDINWIRQPPGKGLEWLGLIWTGGGTNYNSAFMSRLSISKDNSKSQVFLKMNSLQTDDTAIYYCVRGGPLVWYALDYWGQGTSVTVSSDIVMTQSHKFMSTSVGDRVSITCKASQDVSTAVAWYQQKPGQSPKLLIYSASYRYTGVPDRFTGSGSGTDFTFTISSVQAEDLAVYYCQQHYSNPRTFGGGTKLEIK

SEQ ID NO: 98 - protein, 377 aa, synthetic construct
  REGION 1..377: note = Synthetic Construct
DIQMTQSPSSLSASVGDRVTITCKASQDVSTAVAWYQQKPGKAPKLLIYSASYRYTGVPSRFSGSGSGTDFTFTISSLQPEDIATYYCQQHYSNPRTFGGGTKVEIKGSTSGGGSGGGSGGGGSSQVQLQESGPGLVKPSETLSLTCTVSGFSLTNYDINWIRQPPGKGLEWIGLIWTGGGTNYNPSLKSRVTISKDTSKNQFSLKLSSVTAADTAVYYCVRGGPLVWYALDYWGQGTLVTVSSEPKSSDKTHTCPPCPPCGGGSSGGGSGGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 99 - protein, 377 aa, synthetic construct
  REGION 1..377: note = Synthetic Construct
QVQLQESGPGLVKPSETLSLTCTVSGFSLTNYDINWIRQPPGKGLEWIGLIWTGGGTNYNPSLKSRVTISKDTSKNQFSLKLSSVTAADTAVYYCVRGGPLVWYALDYWGQGTLVTVSSGSTSGGGSGGGSGGGGSSDIQMTQSPSSLSASVGDRVTITCKASQDVSTAVAWYQQKPGKAPKLLIYSASYRYTGVPSRFSGSGSGTDFTFTISSLQPEDIATYYCQQHYSNPRTFGGGTKVEIKEPKSSDKTHTCPPCPPCGGGSSGGGSGGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 100 - protein, 244 aa, synthetic construct
  REGION 1..244: note = Synthetic Construct
QVQLVQSGAEVKKPGSSVKVSCKASGFSLTNYDINWVRQAPGQGLEWMGLIWTGGGTNYNQKFQGRVTITKDNSTSTAYMELSSLRSEDTAVYYCVRGGPLVWYALDYWGQGTLVTVSSGSTSGGGSGGGSGGGGSSDIQMTQSPSSLSASVGDRVTITCKASQDVSTAVAWYQQKPGKAPKLLIYSASYRYTGVPSRFSGSGSGTDFTLTISSLQPEDFATYYCQQHYSNPRTFGQGTKVEIK

SEQ ID NO: 101 - protein, 377 aa, synthetic construct
  REGION 1..377: note = Synthetic Construct
QVQLQESGPGLVKPSETLSLTCTVSGFSLTNYDINWIRQPPGKGLEWIGLIWTGGGTNYNPSLKSRVTISKDNSKNQFSLKLSSVTAADTAVYYCVRGGPLVWYALDYWGQGTLVTVSSGSTSGGGSGGGSGGGGSSDIQMTQSPSSLSASVGDRVTITCKASQDVSTAVAWYQQKPGKAPKLLIYSASYRYTGVPSRFSGSGSGTDFTFTISSLQPEDIATYYCQQHYSNPRTFGGGTKVEIKEPKSSDKTHTCPPCPPCGGGSSGGGSGGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 102 - protein, 377 aa, synthetic construct
  REGION 1..377: note = Synthetic Construct
QVQLQESGPGLVKPSETLSLTCTVSGFSLTNYDINWIRQPPGKGLEWIGLIWTGGGTNYNPSLKSRVTISKDTSKNQFSLKLSSVTAADTAVYYCVRGGPLVWYALDYWGQGTLVTVSSGSTSGGGSGGGSGGGGSSDIVMTQSPSSLSASVGDRVTITCKASQDVSTAVAWYQQKPGKAPKLLIYSASYRYTGVPSRFSGSGSGTDFTFTISSLQPEDIATYYCQQHYSNPRTFGGGTKVEIKEPKSSDKTHTCPPCPPCGGGSSGGGSGGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 103 - protein, 377 aa, synthetic construct
  REGION 1..377: note = Synthetic Construct
QVQLQESGPGLVKPSETLSLTCTVSGFSLTNYDINWIRQPPGKGLEWIGLIWTGGGTNYNPSLKSRVTISKDTSKSQFSLKLSSVTAADTAVYYCVRGGPLVWYALDYWGQGTLVTVSSGSTSGGGSGGGSGGGGSSDIQMTQSPSSLSASVGDRVTITCKASQDVSTAVAWYQQKPGKAPKLLIYSASYRYTGVPSRFSGSGSGTDFTFTISSLQPEDIATYYCQQHYSNPRTFGGGTKVEIKEPKSSDKTHTCPPCPPCGGGSSGGGSGGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 104 - protein, 5 aa, synthetic construct
  REGION 1..5: note = Synthetic Construct
SYVMH

SEQ ID NO: 105 - protein, 10 aa, synthetic construct
  REGION 1..10: note = Synthetic Construct
YINPHNDGTK

SEQ ID NO: 106 - protein, 15 aa, synthetic construct
  REGION 1..15: note = Synthetic Construct
ARWGIYYGYGAWFAY

SEQ ID NO: 107 - protein, 11 aa, synthetic construct
  REGION 1..11: note = Synthetic Construct
KASQNVGTYVA

SEQ ID NO: 108 - protein, 8 aa, synthetic construct
  REGION 1..8: note = Synthetic Construct
YSASNRYS

SEQ ID NO: 109 - protein, 9 aa, synthetic construct
  REGION 1..9: note = Synthetic Construct
QQYNTYPYT

SEQ ID NO: 110 - protein, 122 aa, synthetic construct
  REGION 1..122: note = Synthetic Construct
EVQLQQSGPELVKPGASVKMSCKASGYTFTSYVMHWLKQKPGQGLEWIGYINPHNDGTKYNEKFKGKATLTSDKSSSTAYMELSSLTSEDSAVYYCARWGIYYGYGAWFAYWGQGTLVTVSA

SEQ ID NO: 111 - protein, 107 aa, synthetic construct
  REGION 1..107: note = Synthetic Construct
DIVMTQSQKFMSTSVGDRVSVTCKASQNVGTYVAWYQQKPGQSPKALIYSASNRYSGVPDRFTGSGSGTDFTLTISNVQSEDLAEYFCQQYNTYPYTFGGGTKLEIK

SEQ ID NO: 112 - protein, 98 aa, synthetic construct
  REGION 1..98: note = Synthetic Construct
QVQLVQSGAEVKKPGASVKVSCKASGYTFTSYAMHWVRQAPGQRLEWMGWINAGNGNTKYSQKFQGRVTITRDTSASTAYMELSSLRSEDTAVYYCAR

SEQ ID NO: 113 - protein, 95 aa, synthetic construct
  REGION 1..95: note = Synthetic Construct
DIQMTQSPSSLSASVGDRVTITCRASQGISNYLAWFQQKPGKAPKSLIYAASSLQSGVPSRFSGSGSGTDFTLTISSLQPEDFATYYCQQYNSYP

SEQ ID NO: 114 - protein, 122 aa, synthetic construct
  REGION 1..122: note = Synthetic Construct
QVQLVQSGAEVKKPGASVKVSCKASGYTFTSYVMHWVRQAPGQRLEWMGYINPHNDGTKYSQKFQGRVTITSDKSASTAYMELSSLRSEDTAVYYCARWGIYYGYGAWFAYWGQGTLVTVSS

SEQ ID NO: 115 - protein, 107 aa, synthetic construct
  REGION 1..107: note = Synthetic Construct
DIQMTQSPSSLSASVGDRVTITCKASQNVGTYVAWFQQKPGKAPKSLIYSASNRYSGVPSRFSGSGSGTDFTLTISSLQPEDFATYYCQQYNTYPYTFGQGTKLEIK

SEQ ID NO: 116 - protein, 229 aa, synthetic construct
  REGION 1..229: note = Synthetic Construct
EVQLQQSGPELVKPGASVKMSCKASGYTFTSYVMHWLKQKPGQGLEWIGYINPHNDGTKYNEKFKGKATLTSDKSSSTAYMELSSLTSEDSAVYYCARWGIYYGYGAWFAYWGQGTLVTVSADIVMTQSQKFMSTSVGDRVSVTCKASQNVGTYVAWYQQKPGQSPKALIYSASNRYSGVPDRFTGSGSGTDFTLTISNVQSEDLAEYFCQQYNTYPYTFGGGTKLEIK

SEQ ID NO: 117 - protein, 380 aa, synthetic construct
  REGION 1..380: note = Synthetic Construct
DIQMTQSPSSLSASVGDRVTITCKASQNVGTYVAWFQQKPGKAPKSLIYSASNRYSGVPSRFSGSGSGTDFTLTISSLQPEDFATYYCQQYNTYPYTFGQGTKLEIKGSTSGGGSGGGSGGGGSSQVQLVQSGAEVKKPGASVKVSCKASGYTFTSYVMHWVRQAPGQRLEWMGYINPHNDGTKYSQKFQGRVTITSDKSASTAYMELSSLRSEDTAVYYCARWGIYYGYGAWFAYWGQGTLVTVSSEPKSSDKTHTCPPCPPCGGGSSGGGSGGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 118 - protein, 380 aa, synthetic construct
  REGION 1..380: note = Synthetic Construct
QVQLVQSGAEVKKPGASVKVSCKASGYTFTSYVMHWVRQAPGQRLEWMGYINPHNDGTKYSQKFQGRVTITSDKSASTAYMELSSLRSEDTAVYYCARWGIYYGYGAWFAYWGQGTLVTVSSGSTSGGGSGGGSGGGGSSDIQMTQSPSSLSASVGDRVTITCKASQNVGTYVAWFQQKPGKAPKSLIYSASNRYSGVPSRFSGSGSGTDFTLTISSLQPEDFATYYCQQYNTYPYTFGQGTKLEIKEPKSSDKTHTCPPCPPCGGGSSGGGSGGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 119 - protein, 380 aa, synthetic construct
  REGION 1..380: note = Synthetic Construct
DIQMTQSPSSLSASVGDRVTITCKASQNVGTYVNWYQQKPGKAPKLLIYSASNRYSGVPSRFSGSGSGTDFTLTISSLQPEDFATYYCQQYNTYPYTFGQGTKLEIKGSTSGGGSGGGSGGGGSSQVQLVQSGAEVKKPGSSVKVSCKASGYTFTSYVMHWVRQAPGQGLEWMGYINPHNDGTKYAQKFQGRVTITADESTSTAYMELSSLRSEDTAVYYCARWGIYYGYGAWFAYWGQGTLVTVSSEPKSSDKTHTCPPCPPCGGGSSGGGSGGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 120 - protein, 380 aa, synthetic construct
  REGION 1..380: note = Synthetic Construct
QVQLVQSGAEVKKPGSSVKVSCKASGYTFTSYVMHWVRQAPGQGLEWMGYINPHNDGTKYAQKFQGRVTITADESTSTAYMELSSLRSEDTAVYYCARWGIYYGYGAWFAYWGQGTLVTVSSGSTSGGGSGGGSGGGGSSDIQMTQSPSSLSASVGDRVTITCKASQNVGTYVNWYQQKPGKAPKLLIYSASNRYSGVPSRFSGSGSGTDFTLTISSLQPEDFATYYCQQYNTYPYTFGQGTKLEIKEPKSSDKTHTCPPCPPCGGGSSGGGSGGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 121 - protein, 20 aa, synthetic construct
  REGION 1..20: note = Synthetic Construct
METDTLLLWVLLLWVPGSTG

SEQ ID NO: 122 - protein, 15 aa, synthetic construct
  REGION 1..15: note = Synthetic Construct
YFDYWGQGTLVTVSS

SEQ ID NO: 123 - protein, 12 aa, synthetic construct
  REGION 1..12: note = Synthetic Construct
LTFGGGTKVEIK

SEQ ID NO: 124 - protein, 16 aa, synthetic construct
  REGION 1..16: note = Synthetic Construct
NWFDSWGQGTLVTVSS

SEQ ID NO: 125 - protein, 12 aa, synthetic construct
  REGION 1..12: note = Synthetic Construct
YTFGQGTKLEIK

SEQ ID NO: 126 - protein, 377 aa, synthetic construct
  REGION 1..377: note = Synthetic Construct
QVQLVQSGAEVKKPGSSVKVSCKASGFSLTNYDINWVRQAPGQGLEWMGLIWTGGGTNYNQKFQGRVTITKDESTSTAYMELSSLRSEDTAVYYCVRGGPLVWYALDYWGQGTLVTVSSGSTSGGGSGGGSGGGGSSDIQMTQSPSSLSASVGDRVTITCKASQDVSTAVAWYQQKPGKAPKLLIYSASYRYTGVPSRFSGSGSGTDFTLTISSLQPEDFATYYCQQHYSNPRTFGQGTKVEIKEPKSSDKTHTCPPCPPCGGGSSGGGSGGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 127 - protein, 380 aa, synthetic construct
  REGION 1..380: note = Synthetic Construct
DIQMTQSPSSLSASVGDRVTITCKASQNVGTYVNWYQQKPGKAPKLLIYSASNRYSGVPSRFSGSGSGTDFTLTISSLQPEDFATYYCQQYNTYPYTFGQGTKLEIKGSTSGGGSGGGSGGGGSSQVQLVQSGAEVKKPGSSVKVSCKASGYTFTSYVMHWVRQAPGQGLEWMGYINPHNDGTKYAQKFQGRVTITADKSTSTAYMELSSLRSEDTAVYYCARWGIYYGYGAWFAYWGQGTLVTVSSEPKSSDKTHTCPPCPPCGGGSSGGGSGGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 128 - protein, 380 aa, synthetic construct
  REGION 1..380: note = Synthetic Construct
DIQMTQSPSSLSASVGDRVTITCKASQNVGTYVNWYQQKPGKAPKLLIYSASNRYSGVPSRFSGSGSGTDFTLTISSLQPEDFATYYCQQYNTYPYTFGQGTKLEIKGSTSGGGSGGGSGGGGSSQVQLVQSGAEVKKPGSSVKVSCKASGYTFTSYVMHWVRQAPGQGLEWMGYINPHNDGTKYAQKFQGRVTITADTSTSTAYMELSSLRSEDTAVYYCARWGIYYGYGAWFAYWGQGTLVTVSSEPKSSDKTHTCPPCPPCGGGSSGGGSGGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 129 - protein, 380 aa, synthetic construct
  REGION 1..380: note = Synthetic Construct
DIQMTQSPSSLSASVGDRVTITCKASQNVGTYVAWYQQKPGKAPKLLIYSASNRYSGVPSRFSGSGSGTDFTLTISSLQPEDFATYYCQQYNTYPYTFGQGTKLEIKGSTSGGGSGGGSGGGGSSQVQLVQSGAEVKKPGSSVKVSCKASGYTFTSYVMHWVRQAPGQGLEWMGYINPHNDGTKYAQKFQGRVTITADESTSTAYMELSSLRSEDTAVYYCARWGIYYGYGAWFAYWGQGTLVTVSSEPKSSDKTHTCPPCPPCGGGSSGGGSGGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 130 - protein, 380 aa, synthetic construct
  REGION 1..380: note = Synthetic Construct
DIQMTQSPSSLSASVGDRVTITCKASQNVGTYVAWFQQKPGKAPKSLIYSASNRYSGVPSRFSGSGSGTDFTLTISSLQPEDFATYYCQQYNTYPYTFGQGTKLEIKGSTSGGGSGGGSGGGGSSQVQLVQSGAEVKKPGASVKVSCKASGYTFTSYVMHWVRQAPGQGLEWMGYINPHNDGTKYAQKFQGRVTMTRDTSTSTVYMELSSLRSEDTAVYYCARWGIYYGYGAWFAYWGQGTLVTVSSEPKSSDKTHTCPPCPPCGGGSSGGGSGGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 131 - protein, 380 aa, synthetic construct
  REGION 1..380: note = Synthetic Construct
QVQLVQSGAEVKKPGASVKVSCKASGYTFTSYVMHWVRQAPGQGLEWMGYINPHNDGTKYAQKFQGRVTMTRDTSTSTVYMELSSLRSEDTAVYYCARWGIYYGYGAWFAYWGQGTLVTVSSGSTSGGGSGGGSGGGGSSDIQMTQSPSSLSASVGDRVTITCKASQNVGTYVAWFQQKPGKAPKSLIYSASNRYSGVPSRFSGSGSGTDFTLTISSLQPEDFATYYCQQYNTYPYTFGQGTKLEIKEPKSSDKTHTCPPCPPCGGGSSGGGSGGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 132 - protein, 380 aa, synthetic construct
  REGION 1..380: note = Synthetic Construct
DIQMTQSPSSLSASVGDRVTITCKASQNVGTYVAWFQQKPGKAPKSLIYSASNRYSGVPSRFSGSGSGTDFTLTISSLQPEDFATYYCQQYNTYPYTFGQGTKLEIKGSTSGGGSGGGSGGGGSSQVQLVQSGAEVKKPGSSVKVSCKASGYTFTSYVMHWVRQAPGQGLEWMGYINPHNDGTKYAQKFQGRVTMTRDTSTSTVYMELSSLRSEDTAVYYCARWGIYYGYGAWFAYWGQGTLVTVSSEPKSSDKTHTCPPCPPCGGGSSGGGSGGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 133 - protein, 380 aa, synthetic construct
  REGION 1..380: note = Synthetic Construct
DIQMTQSPSSLSASVGDRVTITCKASQNVGTYVAWFQQKPGKAPKLLIYSASNRYSGVPSRFSGSGSGTDFTLTISSLQPEDFATYYCQQYNTYPYTFGQGTKLEIKGSTSGGGSGGGSGGGGSSQVQLVQSGAEVKKPGASVKVSCKASGYTFTSYVMHWVRQAPGQGLEWMGYINPHNDGTKYAQKFQGRVTMTRDTSTSTVYMELSSLRSEDTAVYYCARWGIYYGYGAWFAYWGQGTLVTVSSEPKSSDKTHTCPPCPPCGGGSSGGGSGGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 134 - protein, 5 aa, synthetic construct
  REGION 1..5: note = Synthetic Construct
SYTMS

SEQ ID NO: 135 - protein, 10 aa, synthetic construct
  REGION 1..10: note = Synthetic Construct
TISSGGSYTY

SEQ ID NO: 136 - protein, 11 aa, synthetic construct
  REGION 1..11: note = Synthetic Construct
TRDQVGYAMDY

SEQ ID NO: 137 - protein, 11 aa, synthetic construct
  REGION 1..11: note = Synthetic Construct
KASQDVGIAVA

SEQ ID NO: 138 - protein, 8 aa, synthetic construct
  REGION 1..8: note = Synthetic Construct
YWASTRHT

SEQ ID NO: 139 - protein, 9 aa, synthetic construct
  REGION 1..9: note = Synthetic Construct
QQYSRYPYT

SEQ ID NO: 140 - protein, 118 aa, synthetic construct
  REGION 1..118: note = Synthetic Construct
DVKLVESGGGFVKPGGSLRLSYTVSGFTFSSYTMSWVRQTPEKRLEWVATISSGGSYTYYLDSVRGRFTISRDNAKKTLYLQMSSLKSEDTAIYYCTRDQVGYAMDYWGQGTSVTVSS

SEQ ID NO: 141 - protein, 107 aa, synthetic construct
  REGION 1..107: note = Synthetic Construct
DIVMTQSHKFMSTSVGDRVSITCKASQDVGIAVAWYQQKPGQSPKLLIYWASTRHTGVPDRCTGSGSGTDFTLTISNVQSEDLADYFCQQYSRYPYTFGGGTKLEIK

SEQ ID NO: 142 - protein, 98 aa, synthetic construct
  REGION 1..98: note = Synthetic Construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCAR

SEQ ID NO: 143 - protein, 118 aa, synthetic construct
  REGION 1..118: note = Synthetic Construct
DVQLVESGGGLVKPGGSLRLSCTVSGFTFSSYTMSWVRQAPGKGLEWVSTISSGGSYTYYADSVKGRFTISRDNAKNSLYLQMNSLRAEDTAVYYCTRDQVGYAMDYWGQGTLVTVSS

SEQ ID NO: 144 - protein, 225 aa, synthetic construct
  REGION 1..225: note = Synthetic Construct
DVKLVESGGGFVKPGGSLRLSYTVSGFTFSSYTMSWVRQTPEKRLEWVATISSGGSYTYYLDSVRGRFTISRDNAKKTLYLQMSSLKSEDTAIYYCTRDQVGYAMDYWGQGTSVTVSSDIVMTQSHKFMSTSVGDRVSITCKASQDVGIAVAWYQQKPGQSPKLLIYWASTRHTGVPDRCTGSGSGTDFTLTISNVQSEDLADYFCQQYSRYPYTFGGGTKLEIK

SEQ ID NO: 145 - protein, 376 aa, synthetic construct
  REGION 1..376: note = Synthetic Construct
DIQMTQSPSSLSASVGDRVTITCKASQDVGIAVAWYQQKPGKAPKLLIYWASTRHTGVPSRFSGSGSGTDFTFTISSLQPEDIATYYCQQYSRYPYTFGQGTKLEIKGSTSGGGSGGGSGGGGSSDVQLVESGGGLVKPGGSLRLSCTVSGFTFSSYTMSWVRQAPGKGLEWVSTISSGGSYTYYADSVKGRFTISRDNAKNSLYLQMNSLRAEDTAVYYCTRDQVGYAMDYWGQGTLVTVSSEPKSSDKTHTCPPCPPCGGGSSGGGSGGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 146 - protein, 376 aa, synthetic construct
  REGION 1..376: note = Synthetic Construct
DVQLVESGGGLVKPGGSLRLSCTVSGFTFSSYTMSWVRQAPGKGLEWVSTISSGGSYTYYADSVKGRFTISRDNAKNSLYLQMNSLRAEDTAVYYCTRDQVGYAMDYWGQGTLVTVSSGSTSGGGSGGGSGGGGSSDIQMTQSPSSLSASVGDRVTITCKASQDVGIAVAWYQQKPGKAPKLLIYWASTRHTGVPSRFSGSGSGTDFTFTISSLQPEDIATYYCQQYSRYPYTFGQGTKLEIKEPKSSDKTHTCPPCPPCGGGSSGGGSGGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 147 - protein, 741 aa, synthetic construct
HHHHHHLRPSRVHNSEENTMRALTLKDILNGTFSYKTFFPNWISGQEYLHQSADNNIVLYNIETGQSYTILSNRTMKSVNASNYGLSPDRQFVYLESDYSKLWRYSYTATYYIYDLSNGEFVRGNELPRPIQYLCWSPVGSKLAYVYQNNIYLKQRPGDPPFQITFNGRENKIFNGIPDWVYEEEMLATKYALWWSPNGKFLAYAEFNDTDIPVIAYSYYGDEQYPRTINIPYPKAGAKNPVVRIFIIDTTYPAYVGPQEVPVPAMIASSDYYFSWLTWVTDERVCLQWLKRVQNVSVLSICDFREDWQTWDCPKTQEHIEESRTGWAGGFFVSTPVFSYDAISYYKIFSDKDGYKHIHYIKDTVENAIQITSGKWEAINIFRVTQDSLFYSSNEFEEYPGRRNIYRISIGSYPPSKKCVTCHLRKERCQYYTASFSDYAKYYALVCYGPGIPISTLHDGRTDQEIKILEENKELENALKNIQLPKEEIKKLEVDEITLWYKMILPPQFDRSKKYPLLIQVYGGPCSQSVRSVFAVNWISYLASKEGMVIALVDGRGTAFQGDKLLYAVYRKLGVYEVEDQITAVRKFIEMGFIDEKRIAIWGWSYGGYVSSLALASGTGLFKCGIAVAPVSSWEYYASVYTERFMGLPTKDDNLEHYKNSTVMARAEYFRNVDYLLIHGTADDNVHFQNSAQIAKALVNAQVDFQAMWYSDQNHGLSGLSTNHLYTHMTHFLKQCFSLSD

SEQ ID NO: 148 - protein, 760 aa, synthetic construct
MKTWVKIVFGVATSAVLALLVMCIVLRPSRVHNSEENTMRALTLKDILNGTFSYKTFFPNWISGQEYLHQSADNNIVLYNIETGQSYTILSNRTMKSVNASNYGLSPDRQFVYLESDYSKLWRYSYTATYYIYDLSNGEFVRGNELPRPIQYLCWSPVGSKLAYVYQNNIYLKQRPGDPPFQITFNGRENKIFNGIPDWVYEEEMLATKYALWWSPNGKFLAYAEFNDTDIPVIAYSYYGDEQYPRTINIPYPKAGAKNPVVRIFIIDTTYPAYVGPQEVPVPAMIASSDYYFSWLTWVTDERVCLQWLKRVQNVSVLSICDFREDWQTWDCPKTQEHIEESRTGWAGGFFVSTPVFSYDAISYYKIFSDKDGYKHIHYIKDTVENAIQITSGKWEAINIFRVTQDSLFYSSNEFEEYPGRRNIYRISIGSYPPSKKCVTCHLRKERCQYYTASFSDYAKYYALVCYGPGIPISTLHDGRTDQEIKILEENKELENALKNIQLPKEEIKKLEVDEITLWYKMILPPQFDRSKKYPLLIQVYGGPCSQSVRSVFAVNWISYLASKEGMVIALVDGRGTAFQGDKLLYAVYRKLGVYEVEDQITAVRKFIEMGFIDEKRIAIWGWSYGGYVSSLALASGTGLFKCGIAVAPVSSWEYYASVYTERFMGLPTKDDNLEHYKNSTVMARAEYFRNVDYLLIHGTADDNVHFQNSAQIAKALVNAQVDFQAMWYSDQNHGLSGLSTNHLYTHMTHFLKQCFSLSD

SEQ ID NO: 149 - protein, 244 aa, synthetic construct
QVQLVQSGAEVKKPGSSVKVSCKASGFSLTNYDINWVRQAPGQGLEWMGLIWTGGGTNYNQKFQGRVTITKDESTSTAYMELSSLRSEDTAVYYCVRGGPLVWYALDYWGQGTLVTVSSGSTSGGGSGGGSGGGGSSDIQMTQSPSSLSASVGDRVTITCKASQDVSTAVAWYQQKPGKAPKLLIYSASYSYTGVPSRFSGSGSGTDFTLTISSLQPEDFATYYCQQHYSNPRTFGQGTKVEIK

SEQ ID NO: 150 - protein, 244 aa, synthetic construct
QVQLVQSGAEVKKPGSSVKVSCKASGFSLTNYDINWVRQAPGQGLEWMGLIWTGGGTNYNQKFQGRVTITKDESTSTAYMELSSLRSEDTAVYYCVRGGPLVWYALDYWGQGTLVTVSSGSTSGGGSGGGSGGGGSSDIQMTQSPSSLSASVGDRVTITCKASQDVSTAVAWYQQKPGQAPKLLIYSASYSYTGVPSRFSGSGSGTDFTLTISSLQPEDFATYYCQQHYSNPRTFGQGTKVEIK

SEQ ID NO: 151 - protein, 15 aa, synthetic construct
WYQQKPGKAPKLLIY

SEQ ID NO: 152 - protein, 15 aa, synthetic construct
WYQQKPGKAPKLLIY

SEQ ID NO: 153 - protein, 15 aa, synthetic construct
WYQQKPGQAPRLLIY

SEQ ID NO: 154 - protein, 15 aa, synthetic construct
WYQQKPGQAPRLLIY

SEQ ID NO: 155 - protein, 15 aa, synthetic construct
WYQQKPGQPPKLLIY

SEQ ID NO: 156 - protein, 15 aa, synthetic construct
WYQQKPGKAPKLLLY

SEQ ID NO: 157 - protein, 15 aa, synthetic construct
WYQQKPGKAPKLLLY

SEQ ID NO: 158 - protein, 15 aa, synthetic construct
WYQQKPGKAPKLLIY

SEQ ID NO: 159 - protein, 15 aa, synthetic construct
WYLQKPGQSPQLLIY

SEQ ID NO: 160 - protein, 15 aa, synthetic construct
WYQQKPGKAPKLLIY

SEQ ID NO: 161 - protein, 15 aa, synthetic construct
WFQQKPGKAPKSLIY

SEQ ID NO: 162 - protein, 15 aa, synthetic construct
WYQQHPGKAPKLMIY

SEQ ID NO: 163 - protein, 15 aa, synthetic construct
WYQQKPGQAPRLLIY

SEQ ID NO: 164 - protein, 16 aa, synthetic construct
WYQQKPDQSPKLLIKY

SEQ ID NO: 165 - protein, 15 aa, synthetic construct
WYQQKPGKAPKLLIY

SEQ ID NO: 166 - protein, 15 aa, synthetic construct
WYQQLPGTAPKLLIY

SEQ ID NO: 167 - protein, 15 aa, synthetic construct
WYLQKPGQSPQLLIY

SEQ ID NO: 168 - protein, 15 aa, synthetic construct
WYQQKPGQSPVLVIY

SEQ ID NO: 169 - protein, 15 aa, synthetic construct
WYQQKPGQAPRLLIY

SEQ ID NO: 170 - protein, 15 aa, synthetic construct
WYQQKPEKAPKSLIY

SEQ ID NO: 171 - protein, 15 aa, synthetic construct
WYQQKPGKAPKLLIY

SEQ ID NO: 172 - protein, 15 aa, synthetic construct
WYQQKPGQAPVLVIY

SEQ ID NO: 173 - protein, 15 aa, synthetic construct
WYQQKPGKAPKLLIY

SEQ ID NO: 174 - protein, 15 aa, synthetic construct
WYQQKPGKAPKLLIY

SEQ ID NO: 175 - protein, 15 aa, synthetic construct
WYLQKPGQSPQLLIY

SEQ ID NO: 176 - protein, 15 aa, synthetic construct
WYQQRPGSSPTTVIY

SEQ ID NO: 177 - protein, 15 aa, synthetic construct
WYQQHPGKAPKLMIY

SEQ ID NO: 178 - protein, 15 aa, synthetic construct
WYQQLPGTAPKLLIY

SEQ ID NO: 179 - protein, 15 aa, synthetic construct
WFQQRPGQSPRRLIY

SEQ ID NO: 180 - protein, 15 aa, synthetic construct
WYQQKPGKAPKRLIY

SEQ ID NO: 181 - protein, 15 aa, synthetic construct
WYQQKPGQAPRLLIY

SEQ ID NO: 182 - protein, 15 aa, synthetic construct
WYQQKPGQPPKLLIY

SEQ ID NO: 183 - protein, 15 aa, synthetic construct
WFQQKPGQAPRALIY

SEQ ID NO: 184 - protein, 15 aa, synthetic construct
WYQQKPGQAPVLVVY

SEQ ID NO: 185 - protein, 15 aa, synthetic construct
WFQQRPGQSPRRLIY

SEQ ID NO: 186 - protein, 15 aa, synthetic construct
WYQQKPGQAPVLVVY

SEQ ID NO: 187 - protein, 113 aa, synthetic construct
DIVMTQSPDSLAVSLGERATINCKSSQSLLYSRNQKNYLAWYQQKPGQPPKLLIFWASTRESGVPDRFSGSGFGTDFTLTISSLQAEDVAVYYCQQYFSYPLTFGQGTKVEIK

SEQ ID NO: 188 - protein, 24 aa, synthetic construct
EPKSCDKTHTCPPCGGGSSGGGSG

SEQ ID NO: 189 - protein, 24 aa, synthetic construct
EPKSSDKTHTCPPCGGGSSGGGSG

SEQ ID NO: 190 - protein, 22 aa, synthetic construct
ERKCCVECPPCPGGGSSGGGSG

SEQ ID NO: 191 - protein, 22 aa, synthetic construct
ERKSCVECPPCPGGGSSGGGSG

SEQ ID NO: 192 - protein, 19 aa, synthetic construct
ERKCCVECPPCPAPPVAGP

SEQ ID NO: 193 - protein, 19 aa, synthetic construct
ERKSCVECPPCPAPPVAGP

SEQ ID NO: 194 - protein, 766 aa, synthetic construct
MKTPWKVLLGLLGAAALVTIITVPVVLLNKGTDDATADSRKTYTLTDYLKNTYRLKLYSLRWISDHEYLYKQENNILVFNAEYGNSSVFLENSTFDEFGHSINDYSISPDGQFILLEYNYVKQWRHSYTASYDIYDLNKRQLITEERIPNNTQWVTWSPVGHKLAYVWNNDIYVKIEPNLPSYRITWTGKEDIIYNGITDWVYEEEVFSAYSALWWSPNGTFLAYAQFNDTEVPLIEYSFYSDESLQYPKTVRVPYPKAGAVNPTVKFFVVNTDSLSSVTNATSIQITAPASMLIGDHYLCDVTWATQERISLQWLRRIQNYSVMDICDYDESSGRWNCLVARQHIEMSTTGWVGRFRPSEPHFTLDGNSFYKIISNEEGYRHICYFQIDKKDCTFITKGTWEVIGIEALTSDYLYYISNEYKGMPGGRNLYKIQLSDYTKVTCLSCELNPERCQYYSVSFSKEAKYYQLRCSGPGLPLYTLHSSVNDKGLRVLEDNSALDKMLQNVQMPSKKLDFIILNETKFWYQMILPPHFDKSKKYPLLLDVYAGPCSQKADTVFRLNWATYLASTENIIVASFDGRGSGYQGDKIMHAINRRLGTFEVEDQIEAARQFSKMGFVDNKRIAIWGWSYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDVGVDFQAMWYTDEDHGIASSTAHQHIYTHMSHFIKQCFSLP

(19) United States
(12) Patent Application Publication
(10) Pub. No.: US 2024/0390531 A1
(43) Pub. Date: Nov. 28, 2024

(54) NOVEL ANTIBODY SEQUENCES FOR DIAGNOSTICS AND THERAPEUTICS

(71) Applicant: IMAGINAB, INC., Inglewood, CA (US)

(72) Inventors: Alessandro MASCIONI, Los Angeles, CA (US); Fang JIA, Cerritos, CA (US); Leticia Maria DE SOUZA CORDEIRO, Redondo Beach, CA (US); Kelley C. ATKINSON, Anaheim, CA (US); Patrick JOYCE, Gardena, CA (US); Argin AIVAZIAN, Burbank, CA (US); Zheng LIU, Rancho Palos Verdes, CA (US); Ian Andrew WILSON, Venice, CA (US)

(21) Appl. No.: 18/576,566

(22) PCT Filed: Jul. 8, 2022
(86) PCT No.: PCT/US2022/073565

§ 371 Date: Jul. 25, 2024

Related U.S. Application Data
(60) Provisional application No. 63/220,120, filed on Jul. 9, 2021.
(60) Provisional application No. 63/220,146, filed on Jul. 9, 2021.

(51) Int. Cl. A61K 51/10; C07K 16/40

(52) U.S. Cl. CPC A61K 51/1075; C07K 16/40; A61K 2121/00; A61K 2123/00; C07K 2317/33; C07K 2317/565; C07K 2317/92

(57) ABSTRACT

Aspects of the present disclosure relate generally to novel antigen binding sequences and uses thereof. More specifically, certain features of the present disclosure concern antibodies, minibodies, and cys-diabodies for use in targeting the protein FAP. The antigen binding sequences herein may be used as part of treatment to confer health benefits to subjects in need thereof. The antigen binding sequences can also be used for detection, targeting, and imaging purposes.

### CROSS-REFERENCE TO RELATED APPLICATIONS

The present application is a U.S. national phase application under 35 U.S.C. § 371 of International Application No. PCT/US2022/073565, filed Jul. 8, 2022, which claims the benefit of U.S. Provisional Application Nos. 63/220,120 and 63/220,146 filed Jul. 9, 2021. The content of each of these related applications is hereby incorporated by reference in its entirety.

### FIELD OF THE INVENTION

Aspects of the present disclosure relate to novel antigen binding sequences and uses of various antigen binding sequence. Certain features of the present disclosure concern antibodies, minibodies, and cys-diabodies for use in targeting the protein FAP.

### REFERENCE TO SEQUENCE LISTING

The present application is being filed along with a Sequence Listing in electronic format. The Sequence Listing is provided as a file entitled IGNAB055NP.xml, which was created on Jan. 4, 2024, which is 283574 bytes in size. The information in the electronic Sequence Listing is hereby incorporated by reference in its entirety.

### BACKGROUND OF THE INVENTION

Fibroblast activation protein (FAP), is a cell surface glycoprotein serine protease that has been shown to play a role in extracellular matrix degradation, cell invasiveness, cell adhesion, wound healing, tissue remodeling, fibrosis, inflammation, and possibly tumor growth. Both the membrane-bound and soluble forms of FAP exhibit post-proline cleaving endopeptidase activity, with a marked preference for the Ala/Ser-Gly-Pro-Ser/Asn/Ala consensus sequences. FAP also has dipeptidyl peptidase activity, exhibiting the ability to hydrolyze the prolyl bond between a proline and second residue at the N-terminus, with a preference for Ala-Pro, Ile-Pro, Gly-Pro, Arg-Pro and Pro-Pro. The enzyme activity of human FAP is believed to be highest in single reactive fibroblasts, glucagon producing A-cells in pancreatic islets, and endometrial cells in healthy tissues. In mice, the highest FAP enzymatic activity was detected in the uterus, pancreas, submaxillary gland, and skin, whereas the lowest levels were in brain, prostate, leukocytes, and testis. FAP's enzymatic activity is influenced by DPP4 (Dipeptidyl Peptidase IV) on the cell surface, and possibly also by TERT, TWIST1, and TCF15.

### SUMMARY OF THE INVENTION

Some aspects of the present disclosure relate to an isolated antigen binding construct. In some embodiments, the isolated antigen binding construct comprises a HCDR1 comprising amino acids having at least 80% identity with the amino acid sequence of SEQ ID NO: 1 (EYTIH); a HCDR2 comprising amino acids having at least 80% identity with the amino acid sequence of SEQ ID NO: 2 (GINPNNGIPNYNQKFKG); a HCDR3 comprising amino acids having at least 80% identity with the amino acid sequence of SEQ ID NO: 3 (RRIAYGYDEGHAMDY); a LCDR1 comprising amino acids having at least 80% identity with the amino acid sequence of SEQ ID NO: 4 (KSSQSLLYSRNQKNYLA); a LCDR2 comprising amino acids having at least 80% identity with the amino acid sequence of SEQ ID NO: 5 (WASTRES); and a LCDR3 comprising amino acids having the amino acid sequence of SEQ ID NO: 6 (QQYYSYPLT). In some embodiments, the isolated antigen binding construct further comprises a heavy chain having at least 80% identity with the amino acid sequence of SEQ ID NO: 7. In some embodiments, the isolated antigen binding construct comprises a heavy chain having at least 90% identity with the amino acid sequence of SEQ ID NO: 7. In some embodiments, the isolated antigen binding construct comprises a heavy chain having at least 95% identity with the amino acid sequence of SEQ ID NO: 7. In some embodiments, the isolated antigen binding construct comprises a light chain having at least 80% identity with the amino acid sequence of SEQ ID NO: 8. In some embodiments, the isolated antigen binding construct comprises a light chain having at least 90% identity with the amino acid sequence of SEQ ID NO: 8. In some embodiments, the isolated antigen binding construct comprises a light chain having at least 95% identity with the amino acid sequence of SEQ ID NO: 8. In some embodiments, the isolated antigen binding construct comprises a light chain having at least 80% identity with the amino acid sequence of SEQ ID NO: 9. In some embodiments, the isolated antigen binding construct comprises a light chain having at least 90% identity with the amino acid sequence of SEQ ID NO: 9. In some embodiments, the isolated antigen binding construct comprises a light chain having at least 95% identity with the amino acid sequence of SEQ ID NO: 9. In some embodiments, the isolated antigen binding construct comprises at least one VH framework residue selected from the group consisting of: an Alanine at position 24 of the sequence in SEQ ID NO: 7; or a Glycine at position 26 of the sequence in SEQ ID NO: 7. In some embodiments, the isolated antigen binding construct comprises at least one VL framework residue selected from the group consisting of: a Serine at position 73 of the sequence in SEQ ID NO: 8; an Arginine at position 83 of the sequence in SEQ ID NO: 8; a Glutamic acid at position 85 of the sequence in SEQ ID NO:8; a Proline at position 86 of the sequence in SEQ ID NO: 8; a Phenylalanine at position 89 of the sequence in SEQ ID NO: 8; or a Tyrosine at position 98 of the sequence in SEQ ID NO: 8. In some embodiments, the isolated antigen binding construct comprises a phenylalanine at position 89 of the light chain sequence in SEQ ID NO: 8. In some embodiments, the isolated antigen binding construct comprises an alanine at position 24 and a glycine at position 26 of the heavy chain sequence in SEQ ID NO: 7. In some embodiments, the isolated antigen binding construct comprises a phenylalanine at position 89 and a serine at position 73 of the sequence of the light chain sequence in SEQ ID NO: 8. In some embodiments, the isolated antigen binding construct comprises a phenylalanine at position 89, a serine at position 73, and a tyrosine at position 98 of the sequence of the light chain sequence in SEQ ID NO: 8. In some embodiments, the isolated antigen binding construct comprises a phenylalanine at position 89 and a tyrosine at position 98 of the sequence of the light chain sequence in SEQ ID NO: 8. In some embodiments, the isolated antigen binding construct comprises a phenylalanine at position 89, an arginine at position 83, a glutamic acid at position 85, a proline at position 86, and a tyrosine at position 98 of the sequence of the light chain sequence in SEQ ID NO: 8. In some embodiments, the isolated antigen binding construct comprises a phenylalanine at position 89, a serine at position 73, an arginine at position 83, a glutamic acid at position 85, and a proline at position 86 of the sequence of the light chain sequence in SEQ ID NO: 8. In some embodiments, the isolated antigen binding construct comprises a phenylalanine at position 89, a serine at position 73, an arginine at position 83, a glutamic acid at position 85, a proline at position 86, and a tyrosine at position 98 of the sequence of the light chain sequence in SEQ ID NO: 8. In some embodiments, the isolated antigen binding construct comprises a phenylalanine at position 89, an arginine at position 83, a glutamic acid at position 85, and a proline at position 86 of the sequence of the light chain sequence in SEQ ID NO: 8. In some embodiments, the isolated antigen binding construct comprises a variable heavy domain (VH) of SEQ ID NO: 7; and a variable light domain (VL) of SEQ ID NO: 8. In some embodiments, the isolated antigen binding construct comprises a variable heavy domain (VH) of SEQ ID NO: 7; and a variable light domain (VL), of SEQ ID NO: 9.

Some aspects of the present disclosure relate to an isolated antigen binding construct specific against FAP alpha comprising a CDR3 comprising amino acids having at least 90% identity with the amino acid sequence of SEQ ID NO: 6 (QQYYSYPLT). Some aspects of the present disclosure relate to an isolated antigen binding construct comprising a light chain CDR3 comprising amino acids having at least 100% identity with the amino acid sequence of SEQ ID NO: 6. Some aspects of the present disclosure relate to an isolated antigen binding construct thereof, comprising: a LCDR1 comprising amino acids having at least 80% identity with the amino acid sequence of SEQ ID NO: 4 (KSSQSLLYSRNQKNYLA); a LCDR2 comprising amino acids having at least 80% identity with the amino acid sequence of SEQ ID NO: 5 (WASTRES); and a LCDR3 comprising amino acids having the amino acid sequence of SEQ ID NO: 6 (QQYYSYPLT). Some aspects of the present disclosure relate to an isolated antigen binding construct comprising a heavy chain having at least 99% identity with the amino acid sequence of SEQ ID NO: 7. Some aspects of the present disclosure relate to an isolated antigen binding construct comprising a heavy chain having at least 80% identity with the amino acid sequence of SEQ ID NO: 7, comprising at least one VH framework residue selected from the group consisting of: an Alanine at position 24 of the sequence in SEQ ID NO: 7; and a Glycine at position 26 of the sequence in SEQ ID NO: 7. Some aspects of the present disclosure relate to an isolated antigen binding construct comprising a light chain having at least 80% identity with the amino acid sequence of SEQ ID NO: 8, comprising at least one VL framework residue selected from the group consisting of: a Serine at position 73 of the sequence in SEQ ID NO: 8; an Arginine at position 83 of the sequence in SEQ ID NO: 8; a Glutamic acid at position 85 of the sequence in SEQ ID NO:8; a Proline at position 86 of the sequence in SEQ ID NO: 8; a Phenylalanine at position 89 of the sequence in SEQ ID NO: 8; and a Tyrosine at position 98 of the sequence in SEQ ID NO: 8. Some aspects of the present disclosure relate to an isolated antigen binding construct comprising a light chain comprising amino acids having at least 95% identity with the amino acid sequence of SEQ ID NO: 9. Some aspects of the present disclosure relate to an isolated antigen binding construct comprising a light chain comprising amino acids having at least 95% identity with the amino acid sequence of SEQ ID NO: 8. Some aspects of the present disclosure relate to an isolated antigen binding construct comprising: a variable heavy domain (VH) comprising amino acids having at least 95% identity with the amino acid sequence of SEQ ID NO: 7; and a variable light domain (VL) comprising amino acids having at least 95% identity with the amino acid sequence of SEQ ID NO: 8. Some aspects of the present disclosure relate to an isolated humanized antigen binding construct comprising: a variable heavy domain (VH) comprising amino acids having at least 95% identity with the amino acid sequence of SEQ ID NO: 7; and a variable light domain (VL) comprising amino acids having at least 89% identity with the amino acid sequence of SEQ ID NO: 9. In some embodiments, the isolated antigen binding construct comprises at least one VH framework residue selected from the group consisting of: an Alanine at position 24 of the sequence in SEQ ID NO: 7; and a Glycine at position 26 of the sequence in SEQ ID NO: 7. In some embodiments, the isolated antigen binding construct comprises at least one VL framework residue selected from the group consisting of: a Serine at position 73 of the sequence in SEQ ID NO: 8; an Arginine at position 83 of the sequence in SEQ ID NO: 8; a Glutamic acid at position 85 of the sequence in SEQ ID NO:8; a Proline at position 86 of the sequence in SEQ ID NO: 8; a Phenylalanine at position 89 of the sequence in SEQ ID NO: 8; and a Tyrosine at position 98 of the sequence in SEQ ID NO: 8. In some embodiments, the isolated antigen binding construct comprises a phenylalanine at position 89 of the light chain sequence in SEQ ID NO: 8. In some embodiments, the isolated antigen binding construct comprises a phenylalanine at position 89 and a serine at position 73 of the sequence of the light chain sequence in SEQ ID NO: 4. In some embodiments, the isolated antigen binding construct comprises a phenylalanine at position 89, a serine at position 73, and a tyrosine at position 98 of the sequence of the light chain sequence in SEQ ID NO: 8. In some embodiments, the isolated antigen binding construct comprises a phenylalanine at position 89 and a tyrosine at position 98 of the sequence of the light chain sequence in SEQ ID NO: 8. In some embodiments, the isolated antigen binding construct comprises a phenylalanine at position 89, an arginine at position 83, a glutamic acid at position 85, a proline at position 86, and a tyrosine at position 98 of the sequence of the light chain sequence in SEQ ID NO: 8. In some embodiments, the isolated antigen binding construct comprises a phenylalanine at position 89, a serine at position 73, an arginine at position 83, a glutamic acid at position 85, and a proline at position 86 of the sequence of the light chain sequence in SEQ ID NO: 8. In some embodiments, the isolated antigen binding construct comprises a phenylalanine at position 89, a serine at position 73, an arginine at position 83, a glutamic acid at position 85, a proline at position 86, and a tyrosine at position 98 of the sequence of the light chain sequence in SEQ ID NO: 8. In some embodiments, the isolated antigen binding construct comprises a phenylalanine at position 89, an arginine at position 83, a glutamic acid at position 85, and a proline at position 86 of the sequence of the light chain sequence in SEQ ID NO: 8.

Some aspects of the present disclosure relate to an isolated antigen binding construct comprising amino acids having at least 80% identity with an amino acid sequence selected from the group of SEQ ID NOS: 23-44. Some aspects of the present disclosure relate to an isolated antigen binding construct comprising amino acids having at least 90% identity with an amino acid sequence selected from the group of SEQ ID NOS: 23-44. Some aspects of the present disclosure relate to an isolated antigen binding construct comprising amino acids having at least 100% identity with an amino acid sequence selected from the group of SEQ ID NOS: 23-44, 12-43, 12-45, 87-96, 110-115, 122-125, or 140-143.

Some aspects of the present disclosure relate to an isolated antigen binding construct. In some embodiments, the isolated antigen binding construct comprises a HCDR1 comprising the amino acid sequence of SEQ ID NO: 81 (NYDIN), or a sequence that has no more than 3 point mutations thereto, a HCDR2 comprising the amino acid sequence of SEQ ID NO: 82 (LIWTGGGTN), or a sequence that has no more than 3 point mutations thereto, a HCDR3 comprising the amino acid sequence of SEQ ID NO: 83 (GGPLVWYALDY), or a sequence that has no more than 3 point mutations thereto, a LCDR1 comprising the amino acid sequence of SEQ ID NO: 84 (KASQDVSTAVA), or a sequence that has no more than 3 point mutations thereto, a LCDR2 comprising the amino acid sequence of SEQ ID NO: 85 (SASYRYT), or a sequence that has no more than 3 point mutations thereto, and/or a LCDR3 comprising the amino acid sequence of SEQ ID NO: 86 (QQHYSNPRT), or a sequence that has no more than 3 point mutations thereto.

Also disclosed herein is an isolated antigen binding construct thereof comprising a HCDR1 comprising the amino acid sequence of SEQ ID NO: 81 (NYDIN), or a sequence that has no more than 3 point mutations thereto, a HCDR2 comprising the amino acid sequence of SEQ ID NO: 82 (LIWTGGGTN), or a sequence that has no more than 3 point mutations thereto, and/or a HCDR3 comprising the amino acid sequence of SEQ ID NO: 83 (GGPLVWYALDY), or a sequence that has no more than 3 point mutations thereto. Also disclosed herein is an isolated antigen binding construct thereof, comprising a LCDR1 comprising the amino acid sequence of SEQ ID NO: 84 (KASQDVSTAVA), or a sequence that has no more than 3 point mutations thereto, a LCDR2 comprising the amino acid sequence of SEQ ID NO: 85 (SASYRYT), or a sequence that has no more than 3 point mutations thereto, and/or a LCDR3 comprising the amino acid sequence of SEQ ID NO: 86 (QQHYSNPRT), or a sequence that has no more than 3 point mutations thereto. Also disclosed herein is an isolated antigen binding construct specific against FAP comprising a CDR1 comprising the amino acid sequence of SEQ ID NO: 81 (NYDIN), or a sequence that has no more than 3 point mutations thereto. In some embodiments, the CDR1 is part of a heavy chain. Also disclosed herein is an isolated antigen binding construct specific against FAP comprising a CDR2 comprising with the amino acid sequence of SEQ ID NO: 82 (LIWTGGGTN), or a sequence that has no more than 3 point mutations thereto. In some embodiments, the CDR2 is part of a heavy chain. Also disclosed herein is an isolated antigen binding construct specific against FAP comprising a CDR3 comprising the amino acid sequence of SEQ ID NO: 83 (GGPLVWYALDY), or a sequence that has no more than 3 point mutations thereto. In some embodiments, the CDR3 is part of a heavy chain. Also disclosed herein is an isolated antigen binding construct specific against FAP comprising a CDR1 comprising the amino acid sequence of SEQ ID NO: 84 (KASQDVSTAVA), or a sequence that has no more than 3 point mutations thereto. In some embodiments, the CDR1 is part of a light chain. Also disclosed herein is an isolated antigen binding construct specific against FAP comprising a CDR2 comprising the amino acid sequence of SEQ ID NO: 85 (SASYRYT), or a sequence that has no more than 3 point mutations thereto. In some embodiments, the CDR2 is part of a light chain. Also disclosed herein is an isolated antigen binding construct specific against FAP comprising a CDR3 comprising the amino acid sequence of SEQ ID NO: 86 (QQHYSNPRT), or a sequence that has no more than 3 point mutations thereto. In some embodiments, the CDR3 is part of a light chain. In some embodiments, there are no more than 2 point mutations. In some embodiments, there is no more than 1 point mutation. In some embodiments, there are no point mutations. In some embodiments, the isolated antigen binding construct further comprises a heavy chain having at least 80% identity with the amino acid sequence of SEQ ID NO: 87. In some embodiments, the isolated antigen binding construct further comprises a light chain having at least 80% identity with the amino acid sequence of SEQ ID NO: 88. In some embodiments, the antigen binding construct further comprises a variable heavy domain (VH) having at least 80% identity with the amino acid sequence of SEQ ID NO: 87, and/or a variable light domain (VL) chain having at least 80% identity with the amino acid sequence of SEQ ID NO: 88. In some embodiments, the antigen binding construct further comprises a variable heavy domain (VH) having at least 80% identity with the amino acid sequence of SEQ ID NO: 89, and/or a variable light domain (VL) chain having at least 80% identity with the amino acid sequence of SEQ ID NO: 90. In some embodiments, the antigen binding construct further comprises a variable heavy domain (VH) having at least 80% identity with the amino acid sequence of SEQ ID NO: 91, and/or a variable light domain (VL) chain having at least 80% identity with the amino acid sequence of SEQ ID NO: 92. In some embodiments, the antigen binding construct further comprises a variable heavy domain (VH) having at least 80% identity with the amino acid sequence of SEQ ID NO: 93 and/or a variable light domain (VL) chain having at least 80% identity with the amino acid sequence of SEQ ID NO: 94. In some embodiments, the antigen binding construct further comprises a variable heavy domain (VH) having at least 80% identity with the amino acid sequence of SEQ ID NO: 95, and/or a variable light domain (VL) chain having at least 80% identity with the amino acid sequence of SEQ ID NO: 96. In some embodiments, the antigen binding construct further comprises a variable heavy domain (VH) having at least 80% identity with the amino acid sequence of SEQ ID NO: 42, and/or a variable light domain (VL) chain having at least 80% identity with the amino acid sequence of SEQ ID NO: 43.

Also disclosed herein is an isolated antigen binding construct thereof, comprising a variable heavy domain (VH) having at least 80% identity with the amino acid sequence of SEQ ID NO: 87, and/or a variable light domain (VL) chain having at least 80% identity with the amino acid sequence of SEQ ID NO: 88. Also disclosed herein is an isolated antigen binding construct thereof, comprising a variable heavy domain (VH) having at least 80% identity with the amino acid sequence of SEQ ID NO: 89, and/or a variable light domain (VL) chain having at least 80% identity with the amino acid sequence of SEQ ID NO: 90. Also disclosed herein is an isolated antigen binding construct thereof, comprising a variable heavy domain (VH) having at least 80% identity with the amino acid sequence of SEQ ID NO: 91, and/or a variable light domain (VL) chain having at least 80% identity with the amino acid sequence of SEQ ID NO: 92. Also disclosed herein is an isolated antigen binding construct thereof, comprising a variable heavy domain (VH) having at least 80% identity with the amino acid sequence of SEQ ID NO: 93, and/or a variable light domain (VL) chain having at least 80% identity with the amino acid sequence of SEQ ID NO: 94. Also disclosed herein is an isolated antigen binding construct thereof, comprising a variable heavy domain (VH) having at least 80% identity with the amino acid sequence of SEQ ID NO: 95, and/or a variable light domain (VL) chain having at least 80% identity with the amino acid sequence of SEQ ID NO: 96. Also disclosed herein is an isolated antigen binding construct thereof, comprising a variable heavy domain (VH) having at least 80% identity with the amino acid sequence of SEQ ID NO: 42, and/or a variable light domain (VL) chain having at least 80% identity with the amino acid sequence of SEQ ID NO: 43.

Also disclosed herein is an isolated antigen binding construct thereof, comprising a HCDR1 comprising the amino acid sequence of SEQ ID NO: 104 (SYVMH), or a sequence that has no more than 3 point mutations thereto, a HCDR2 comprising the amino acid sequence of SEQ ID NO: 105 (YINPHNDGTK), or a sequence that has no more than 3 point mutations thereto, a HCDR3 comprising the amino acid sequence of SEQ ID NO: 106 (ARWGIYYGYGAWFAY), or a sequence that has no more than 3 point mutations thereto, a LCDR1 comprising the amino acid sequence of SEQ ID NO: 107 (KASQNVGTYVA), or a sequence that has no more than 3 point mutations thereto, a LCDR2 comprising the amino acid sequence of SEQ ID NO: 108 (YSASNRYS), or a sequence that has no more than 3 point mutations thereto, and/or a LCDR3 comprising the amino acid sequence of SEQ ID NO: 109 (QQYNTYPYT), or a sequence that has no more than 3 point mutations thereto. Also disclosed herein is an isolated antigen binding construct thereof, comprising a HCDR1 comprising the amino acid sequence of SEQ ID NO: 104 (SYVMH), or a sequence that has no more than 3 point mutations thereto, a HCDR2 comprising the amino acid sequence of SEQ ID NO: 105 (YINPHNDGTK), or a sequence that has no more than 3 point mutations thereto, and/or a HCDR3 the amino acid sequence of SEQ ID NO: 106 (ARWGIYYGYGAWFAY), or a sequence that has no more than 3 point mutations thereto. Also disclosed herein is an isolated antigen binding construct thereof, comprising a LCDR1 comprising the amino acid sequence of SEQ ID NO: 107 (KASQNVGTYVA), or a sequence that has no more than 3 point mutations thereto, a LCDR2 comprising the amino acid sequence of SEQ ID NO: 108 (YSASNRYS), or a sequence that has no more than 3 point mutations thereto, and/or a LCDR3 comprising the amino acid sequence of SEQ ID NO: 109 (QQYNTYPYT), or a sequence that has no more than 3 point mutations thereto. Also disclosed herein is an isolated antigen binding construct specific against FAP comprising a CDR1 comprising the amino acid sequence of SEQ ID NO: 104 (SYVMH), or a sequence that has no more than 3 point mutations thereto. In some embodiments, the CDR1 is part of a heavy chain. Also disclosed herein is an isolated antigen binding construct specific against FAP comprising a CDR2 comprising the amino acid sequence of SEQ ID NO: 105 (YINPHNDGTK), or a sequence that has no more than 3 point mutations thereto. In some embodiments, the CDR2 is part of a heavy chain. Also disclosed herein is an isolated antigen binding construct specific against FAP comprising a CDR3 comprising the amino acid sequence of SEQ ID NO: 106 (ARWGIYYGYGAWFAY), or a sequence that has no more than 3 point mutations thereto. In some embodiments, the CDR3 is part of a heavy chain. Also disclosed herein is an isolated antigen binding construct specific against FAP comprising a CDR1 comprising the amino acid sequence of SEQ ID NO: 107 (KASQNVGTYVA), or a sequence that has no more than 3 point mutations thereto. In some embodiments, the CDR1 is part of a light chain. Also disclosed herein is an isolated antigen binding construct specific against FAP comprising a CDR2 comprising the amino acid sequence of SEQ ID NO: 108 (YSASNRYS), or a sequence that has no more than 3 point mutations thereto. In some embodiments, the CDR2 is part of a light chain. Also disclosed herein is an isolated antigen binding construct specific against FAP comprising a CDR3 comprising the amino acid sequence of SEQ ID NO: 109 (QQYNTYPYT), or a sequence that has no more than 3 point mutations thereto. In some embodiments, the CDR3 is part of a light chain. In some embodiments, there are no more than 2 point mutations thereto of any of the CDR regions of the antigen binding constructs disclosed herein. In some embodiments, there are no more than 1 point mutation thereto of any of the CDR regions of the antigen binding constructs disclosed herein. In some embodiments, there are no point mutations thereto of any of the CDR regions of the antigen binding constructs disclosed herein. In some embodiments, the antigen binding construct further comprises a heavy chain having at least 80% identity with the amino acid sequence of SEQ ID NO: 110. In some embodiments, the antigen binding construct further comprises a light chain having at least 80% identity with the amino acid sequence of SEQ ID NO: 111. In some embodiments, the antigen binding construct further comprises a variable heavy domain (VH) having at least 80% identity with the amino acid sequence of SEQ ID NO: 110, and/or a variable light domain (VL) chain having at least 80% identity with the amino acid sequence of SEQ ID NO: 111. In some embodiments, the antigen binding construct further comprises a heavy chain having at least 80% identity with the amino acid sequence of SEQ ID NO: 112. In some embodiments, the antigen binding construct further comprises a light chain having at least 80% identity with the amino acid sequence of SEQ ID NO: 113. In some embodiments, the antigen binding construct further comprises a variable heavy domain (VH) having at least 80% identity with the amino acid sequence of SEQ ID NO: 112, and/or a variable light domain (VL) chain having at least 80% identity with the amino acid sequence of SEQ ID NO: 113. In some embodiments, the antigen binding construct further comprises a heavy chain having at least 80% identity with the amino acid sequence of SEQ ID NO: 91. In some embodiments, the antigen binding construct further comprises a light chain having at least 80% identity with the amino acid sequence of SEQ ID NO: 92. In some embodiments, the antigen binding construct further comprises a variable heavy domain (VH) having at least 80% identity with the amino acid sequence of SEQ ID NO: 91, and/or a variable light domain (VL) chain having at least 80% identity with the amino acid sequence of SEQ ID NO: 92. In some embodiments, the antigen binding construct further comprises a heavy chain having at least 80% identity with the amino acid sequence of SEQ ID NO: 114. In some embodiments, the antigen binding construct further comprises a light chain having at least 80% identity with the amino acid sequence of SEQ ID NO: 115. In some embodiments, the antigen binding construct further comprises a variable heavy domain (VH) having at least 80% identity with the amino acid sequence of SEQ ID NO: 114, and/or a variable light domain (VL) chain having at least 80% identity with the amino acid sequence of SEQ ID NO: 115. In some embodiments, the antigen binding construct further comprises a heavy chain having at least 80% identity with the amino acid sequence of SEQ ID NO: 95. In some embodiments, the antigen binding construct further comprises a light chain having at least 80% identity with the amino acid sequence of SEQ ID NO: 96. In some embodiments, the antigen binding construct further comprises a variable heavy domain (VH) having at least 80% identity with the amino acid sequence of SEQ ID NO: 95, and/or a variable light domain (VL) chain having at least 80% identity with the amino acid sequence of SEQ ID NO: 96. In some embodiments, the antigen binding construct further comprises a heavy chain having at least 80% identity with the amino acid sequence of SEQ ID NO: 44. In some embodiments, the antigen binding construct is not the amino acid sequence of SEQ ID NO: 44. In some embodiments, the antigen binding construct further comprises a light chain having at least 80% identity with the amino acid sequence of SEQ ID NO: 45. In some embodiments, the antigen binding construct further comprises a variable heavy domain (VH) having at least 80% identity with the amino acid sequence of SEQ ID NO: 84, and/or a variable light domain (VL) chain having at least 80% identity with the amino acid sequence of SEQ ID NO: 45.

Also disclosed herein is an isolated antigen binding construct thereof, comprising a variable heavy domain (VH) having at least 80% identity with the amino acid sequence of SEQ ID NO: 110; and/or a variable light domain (VL) chain having at least 80% identity with the amino acid sequence of SEQ ID NO: 111. Also disclosed herein is an isolated antigen binding construct thereof, comprising a variable heavy domain (VH) having at least 80% identity with the amino acid sequence of SEQ ID NO: 112, and/or a variable light domain (VL) chain having at least 80% identity with the amino acid sequence of SEQ ID NO: 113. Also disclosed herein is an isolated antigen binding construct thereof, comprising a variable heavy domain (VH) having at least 80% identity with the amino acid sequence of SEQ ID NO: 114, and/or a variable light domain (VL) chain having at least 80% identity with the amino acid sequence of SEQ ID NO: 115. Also disclosed herein is an isolated antigen binding construct thereof, comprising a variable heavy domain (VH) having at least 80% identity with the amino acid sequence of SEQ ID NO: 44, and/or a variable light domain (VL) chain having at least 80% identity with the amino acid sequence of SEQ ID NO: 45.

Also disclosed herein is an isolated antigen binding construct thereof, comprising a HCDR1 comprising the amino acid sequence of SEQ ID NO: 134 (SYTMS), or a sequence that has no more than 3 point mutations thereto, a HCDR2 comprising the amino acid sequence of SEQ ID NO: 135 (TISSGGSYTY), or a sequence that has no more than 3 point mutations thereto, a HCDR3 comprising the amino acid sequence of SEQ ID NO: 136 (TRDQVGYAMDY), or a sequence that has no more than 3 point mutations thereto, a LCDR1 comprising the amino acid sequence of SEQ ID NO: 137 (TRDQVGYAMDY), or a sequence that has no more than 3 point mutations thereto, a LCDR2 comprising the amino acid sequence of SEQ ID NO: 138 (YWASTRHT), or a sequence that has no more than 3 point mutations thereto, and/or a LCDR3 comprising the amino acid sequence of SEQ ID NO: 139 (QQYSRYPYT), or a sequence that has no more than 3 point mutations thereto. Also disclosed herein is an isolated antigen binding construct thereof, comprising a HCDR1 comprising the amino acid sequence of SEQ ID NO: 134 (SYTMS), or a sequence that has no more than 3 point mutations thereto, a HCDR2 comprising the amino acid sequence of SEQ ID NO: 135 (TISSGGSYTY), or a sequence that has no more than 3 point mutations thereto, and/or a HCDR3 comprising the amino acid sequence of SEQ ID NO: 136 (TRDQVGYAMDY), or a sequence that has no more than 3 point mutations thereto. Also disclosed herein is an isolated antigen binding construct thereof, comprising a LCDR1 comprising the amino acid sequence of SEQ ID NO: 137 (TRDQVGYAMDY), or a sequence that has no more than 3 point mutations thereto, a LCDR2 comprising the amino acid sequence of SEQ ID NO: 138 (YWASTRHT), or a sequence that has no more than 3 point mutations thereto, and/or a LCDR3 comprising the amino acid sequence of SEQ ID NO: 139 (QQYSRYPYT), or a sequence that has no more than 3 point mutations thereto. Also disclosed herein is an isolated antigen binding construct specific against FAP comprising a CDR1 comprising the amino acid sequence of SEQ ID NO: 134 (SYTMS), or a sequence that has no more than 3 point mutations thereto. In some embodiments, the CDR1 is part of a heavy chain. Also disclosed herein is an isolated antigen binding construct specific against FAP comprising a CDR2 comprising the amino acid sequence of SEQ ID NO: 135 (TISSGGSYTY), or a sequence that has no more than 3 point mutations thereto. In some embodiments, the CDR2 is part of a heavy chain. Also disclosed herein is an isolated antigen binding construct specific against FAP comprising a CDR3 comprising the amino acid sequence of SEQ ID NO: 136 (TRDQVGYAMDY), or a sequence that has no more than 3 point mutations thereto. In some embodiments, the CDR3 is part of a heavy chain. Also disclosed herein is an isolated antigen binding construct specific against FAP comprising a CDR1 comprising the amino acid sequence of SEQ ID NO: 137 (TRDQVGYAMDY), or a sequence that has no more than 3 point mutations thereto. In some embodiments, the CDR1 is part of a light chain. Also disclosed herein is an isolated antigen binding construct specific against FAP comprising a CDR2 comprising the amino acid sequence of SEQ ID NO: 138 (YWASTRHT), or a sequence that has no more than 3 point mutations thereto. In some embodiments, the CDR2 is part of a light chain. Also disclosed herein is an isolated antigen binding construct specific against FAP comprising a CDR3 comprising the amino acid sequence of SEQ ID NO: 139 (QQYSRYPYT), or a sequence that has no more than 3 point mutations thereto. In some embodiments, the CDR3 is part of a light chain. In some embodiments, there are no more than 2 point mutations thereto. In some embodiments, there are no more than 1 point mutation thereto. In some embodiments, there are no point mutations.

In some embodiments, the isolated antigen binding construct further comprises a heavy chain having at least 80% identity with the amino acid sequence of SEQ ID NO: 140. In some embodiments, the isolated antigen binding construct further comprises a light chain having at least 80% identity with the amino acid sequence of SEQ ID NO: 141. In some embodiments, the isolated antigen binding construct further comprises a variable heavy domain (VH) having at least 80% identity with the amino acid sequence of SEQ ID NO: 140, and/or a variable light domain (VL) chain having at least 80% identity with the amino acid sequence of SEQ ID NO: 141. In some embodiments, the antigen binding construct further comprises a variable heavy domain (VH) having at least 80% identity with the amino acid sequence of SEQ ID NO: 142, and/or a variable light domain (VL) chain having at least 80% identity with the amino acid sequence of SEQ ID NO: 90. In some embodiments, the antigen binding construct further comprises a variable heavy domain (VH) having at least 80% identity with the amino acid sequence of SEQ ID NO: 143 and/or a variable light domain (VL) chain having at least 80% identity with the amino acid sequence of SEQ ID NO: 94.

Also disclosed herein is an isolated antigen binding construct comprising a variable heavy domain (VH) having at least 80% identity with the amino acid sequence of SEQ ID NO: 140, and/or a variable light domain (VL) chain having at least 80% identity with the amino acid sequence of SEQ ID NO: 141. Also disclosed herein is an isolated antigen binding construct comprising a variable heavy domain (VH) having at least 80% identity with the amino acid sequence of SEQ ID NO: 142, and/or a variable light domain (VL) chain having at least 80% identity with the amino acid sequence of SEQ ID NO: 90. Also disclosed herein is an isolated antigen binding construct comprising a variable heavy domain (VH) having at least 80% identity with the amino acid sequence of SEQ ID NO: 143, and/or a variable light domain (VL) chain having at least 80% identity with the amino acid sequence of SEQ ID NO: 94.

In some embodiments, the order of the variable regions, from N terminus to C terminus of the polypeptide is V_{L}, V_{H}. In some embodiments, the order of the variable regions, from N terminus to C terminus of the polypeptide is V_{H}, V_{L}.

Also disclosed herein is an isolated antigen binding construct comprising amino acids having at least 98% identity with at least one amino acid sequence selected from the group of SEQ ID NOS: 97-103 and 126.

Also disclosed herein is an isolated antigen binding construct comprising amino acids having at least 98% identity with at least one amino acid sequence selected from the group of SEQ ID NOS: 116-120, and 127-133.

Also disclosed herein is an isolated antigen binding construct comprising amino acids having at least 98% identity with at least one amino acid sequence selected from the group of SEQ ID NOS: 144-146.

In some embodiments, the antigen binding construct is mammalian. In some embodiments, the antigen binding construct is murine. In some embodiments, the antigen binding construct is human. In some embodiments, the isolated antigen binding construct of any one of the above embodiments is an antibody. In some embodiments, the isolated antigen binding construct is a scFv. In some embodiments, the isolated antigen binding construct is an Fab. In some embodiments, the isolated antigen binding construct is an Fab2. In some embodiments, the isolated antigen binding construct is a nanobody. In some embodiments, the isolated antigen binding construct is a minibody. In some embodiments, the isolated antigen binding construct is a cys-diabody. In some embodiments, the isolated antigen binding construct is part of a composition that comprises one or more of an antibody, scFv, Fab, Fab2, nanobody, minibody, cys-diabody, or any combination thereof.

Some aspects of the present disclosure relate to a minibody antigen binding construct comprising amino acids having at least about 80%, at least about 90%, or at least about 99% identity with an amino acid sequence selected from the group of SEQ ID NOS: 11-22. Some aspects of the present disclosure relate to a cys-diabody antigen binding construct comprising amino acids having at least about 80%, at least about 90%, or at least about 99% identity with an amino acid sequence selected from the group of SEQ ID NOS: 11-22.

Some aspects of the present disclosure relate to a minibody antigen binding construct comprising amino acids having at least about 80%, at least about 90%, or at least about 99% identity with at least one of an amino acid sequence selected from the group of SEQ ID NOS: 97-103, 116-120, 126-133, and 144-146. Also disclosed herein is a cys-diabody antigen binding construct comprising amino acids having at least about 80%, at least about 90%, or at least about 99% identity with at least one of an amino acid sequence selected from the group of SEQ ID NOS: 97-103, 116-120, 126-133, and 144-146.

In some embodiments, the isolated antigen binding construct is specific against human FAP. In some embodiments, the isolated antigen binding construct of any one of the above embodiments is specific against FAP alpha. In some embodiments, the isolated antigen binding construct does not bind DPP4.

In some embodiments, a minibody constructed with the Sibrotuzumab CDR domains is IAB16M1-12 and/or IAB16M2-13. These minibodies are sometimes referred to herein as “Sibrotuzumab minibodies”. In some embodiments, the isolated antigen binding construct has an expression in mammalian cells that is at least 2-fold greater than the expression of Sibrotuzumab minibodies. In some embodiments, the isolated antigen binding construct has an expression in mammalian cells that is at least 6-fold greater than the expression of Sibrotuzumab minibodies (FIG. 1). In some embodiments, the Sibrotuzumab minibody is IAB16M1-12 and/or IAB16M2-13. In some embodiments, the isolated antigen binding construct has a KD of less than 2×10\hat{ }-9 M. In some embodiments, the isolated antigen binding construct has a KD of less than 1×10\hat{ }-9 M. In some embodiments, the isolated antigen binding construct has an on-rate (kₒₙ) greater than 8.0 (l/Ms). In some embodiments, the isolated antigen binding construct has an on-rate (kₒₙ) greater than 9.0 (l/Ms). In some embodiments, the isolated antigen binding construct has an off-rate (k_{off}) lesser than 2.5×10\hat{ }-3 (l/s). In some embodiments, the isolated antigen binding construct has an off-rate (k_{off}) lesser than 1.5×10\hat{ }-3 (l/s).

In some embodiments, the order of the variable regions, from N terminus to C terminus of the polypeptide is V_{L}, V_{H}. In some embodiments, the order of the variable regions, from N terminus to C terminus of the polypeptide is V_{H}, V_{L}.

In some embodiments, the isolated antigen binding construct further comprises a payload. In some embodiments, the payload is fluorescent. In some embodiments, the payload is luminescent. In some embodiments, the payload is colorimetric. In some embodiments, the payload is radioactive. In some embodiments, the payload is non-radioactive. In some embodiments, the payload is chemically reactive. In some embodiments, the payload is a detectable marker. In some embodiments, the combination of the minibody or cys-diabody and the payload is antibody-drug-conjugate (ADC). In some embodiments, the isolated antigen binding construct is humanized.

Some aspects of the present disclosure relate to a composition comprising the amino acid sequence of any one of embodiments 1-66, further comprising at least one payload selected from a group consisting of ¹⁸F, ¹⁸F-FAC, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁵Sc, ⁷⁷As, ⁸⁶Y, ⁹⁰Y, ⁸⁹Sr, ⁸⁹Zr, ⁹⁴Tc, ⁹⁴Tc, ⁹⁹mTc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁴⁹Tb, ¹⁵³Sm, ¹⁵⁴⁻¹⁵⁸Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴I, ¹⁹⁸Au, ¹⁹⁹Au, ²¹¹At, ²¹¹Pb, ²¹²Bi, ²¹²Pb, ²¹³Bi, ²²³Ra, ²²⁷Th and ²²⁵Ac, or any combination thereof.

In some embodiments, the amino acid sequence of the antigen binding construct further comprises at least one metal binding site. In some embodiments, the amino acid sequence of the antigen binding construct further comprises a histidine (His) tag sequence. In some embodiments, the amino acid sequence of the antigen binding construct further comprises at ^{99m}Tc-carbonyl radiolabel.

Some aspects of the present disclosure relate to an expression vector capable of expressing the sequence of any one of the embodiments disclosed herein. Also disclosed herein is an expression vector configured to express the sequence of any one of the antigen binding constructs described herein. In some embodiments, the vector is a viral vector selected from a lentiviral vector or an adenoviral vector. In some embodiments, the vector is a vector for transfection in mammalian cells. In some embodiments, the vector comprises a sequence encoding the cleavable signal peptide having at least 99% identity with the amino acid sequence of SEQ ID NOS: 46 and 121 (METDTLLLWVLLLWVPGSTG).

Some aspects of the present disclosure relate to a method of transferring the amino acid sequence or expression vector of any of the preceding embodiments into a host cell, comprising performing electroporation, viral infection, and/or at least one chemical method. Also disclosed herein is a host cell comprising the amino acid sequence or expression vector of any one of the antigen binding constructs described herein.

Some aspects of the present disclosure relate to a method of use of the amino acid sequence or expression vector of any of the preceding claims as a pre-targeting modality. In some embodiments, the method comprises adding a non-radioactive sequence, vector, or antigen binding construct to a system, and adding a fast-clearing radiolabeled product that recognizes a protein product of the sequence or vector. In some embodiments, the fast-clearing radiolabeled product comprises a small molecule and/or a peptide. Some aspects of the present disclosure relate to a host cell comprising the amino acid sequence or expression vector of any one of the above embodiments disclosed herein.

Some aspects of the present disclosure relate to a composition comprising the amino acid sequence of any one of the above embodiments disclosed herein and at least one chelator. In some embodiments, at least one chelator is selected from a group consisting of ethylenediaminetetraacetic acid (EDTA), diethylenetriaminepentaacetic acid (DTPA), DOTA, NOTA, NOGADA, NETA, deferoxamine (DFO), porphyrins, polyamines, crown ethers, bis-thiosemicarbazones, polyoximes, dimercaprol, penicillamine, trientine, zinc, deferasirox, deferiprone, deferoxamine, succimer, pyrophosphoric acid, tripolyphosphoric acid, citric acid, tartaric acid, glycine, DMPS, DMSA, NTA, calcium, sodium, desferioxamine, dicobalt EDTA, dimercarpol, BAL, and demercaptosuccinic acid, or any combination thereof. In some embodiments, the composition further comprises dual or triple chelators. In some embodiments, the dual or triple chelators are ⁶⁴Cu/⁶⁷Cu, or ⁸⁹Zr/¹⁷⁷Lu or ⁸⁹Zr/²²⁷Th. In some embodiments, the dual chelator is capable of capturing both metals: ⁶⁴Cu and ⁶⁷Cu. In some embodiments, the dual chelator is capable of capturing both metals: ⁸⁹Zr and ¹⁷⁷Lu. In some embodiments, at least one chelator is configured to capture an isotope. In some embodiments, the composition further comprises at least one optical probe. In some embodiments, the composition further comprises at least one payload. In some embodiments, the at least one payload is selected from a group consisting of ¹⁸F, ¹⁸F-FAC, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁵Sc, ⁷⁷As, ⁸⁶Y, ⁹⁰Y, ⁸⁹Sr, ⁸⁹Zr, ⁹⁴Tc, ⁹⁴Tc, ⁹⁹mTc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁴⁹Tb, ¹⁵³Sm, ¹⁵⁴⁻¹⁵⁸Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁷Th and ²²⁵Ac, or any combination thereof.

Some aspects of the present disclosure relate to a use of the composition disclosed herein as a medicament. Some aspects of the present disclosure relate to a use of the composition disclosed herein in the manufacture of a medicament for administration to a subject. Some aspects of the present disclosure relate to a use of the composition disclosed herein for imaging a cell, tissue, organ, and/or subject. Some aspects of the present disclosure relate to a use of the composition disclosed herein for identifying a disease in a subject. In some embodiments, the disease is a cancer or tumor. In some embodiments, the disease is a solid tumor. In some embodiments, the disease is fibrosis. In some embodiments, the disease is an autoimmune disease. In some embodiments, the disease is cardiovascular. In some embodiments, the disease is not a hematological cancer or tumor. In some embodiments, the disease relates to the presence, abundance, and/or variance of FAP.

Some aspects of the present disclosure relate to a method of identifying a disease in a subject, comprising: administering at least one of the antibody, antigen binding construct, minibody, and/or cys-diabody of any one of the embodiments disclosed herein to the subject; screening for the binding of the at least one antibody, antigen binding construct, minibody, and/or cys-diabody to FAP; and determining whether the subject has a disease thereof based upon the presence or absence of binding to FAP. In some embodiments, wherein the disease is a cancer or tumor. In some embodiments, the disease is a solid tumor. In some embodiments, the disease is fibrosis. In some embodiments, the disease is an autoimmune disease. In some embodiments, the disease is cardiovascular. In some embodiments, the subject is mammalian or human. In some embodiments, the at least one of the antibody, antigen binding construct, minibody, and/or cys-diabody binds to a tumor stroma. In some embodiments, the at least one of the antibody, antigen binding construct, minibody, and/or cys-diabody binds to a fibroblast. In some embodiments, the at least one of the antibody, antigen binding construct, minibody, and/or cys-diabody binds to a cancer-associated fibroblast. In some embodiments, the disease is epithelial. In some embodiments, the least one antibody, antigen binding construct, minibody, and/or cys-diabody further comprises a payload. In some embodiments, the payload is selected from a group consisting of ¹⁸F, ¹⁸F-FAC, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁵Sc, ⁷⁷As, ⁸⁶Y, ⁹⁰Y, ⁸⁹Sr, ⁸⁹Zr, ⁹⁴Tc, ⁹⁴Tc, ⁹⁹mTc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁷Th and ²²⁵Ac. In some embodiments, the payload is used to determine the binding of the at least one antibody, antigen binding construct, minibody, and/or cys-diabody to FAP. In some embodiments, the binding of the at least one antibody, antigen binding construct, minibody, and/or cys-diabody to FAP is determined through a PET scan. In some embodiments, the binding of the at least one antibody, antigen binding construct, minibody, and/or cys-diabody to FAP is determined through at least one of a group consisting of: MR imaging, optical probe, magnetic nanoparticles, spectroscopy, and/or photoacoustics. In some embodiments, the binding of the at least one antibody, antigen binding construct, minibody, and/or cys-diabody to FAP is determined through an in vitro testing of a tissue or cell sample removed from the subject. In some embodiments, the method further comprises identifying the subject as negative for having a disease upon the absence of significant binding of the at least one antibody, antigen binding construct, minibody, and/or cys-diabody to FAP. In some embodiments, the method further comprises identifying the subject having a disease upon the significant binding of the at least one antibody, antigen binding construct, minibody, and/or cys-diabody to FAP.

Some aspects of the present disclosure relate to a pharmaceutical composition comprising an amount of the at least one antibody, antigen binding construct, minibody, and/or cys-diabody thereof of any one of the embodiments disclosed herein, that is effective for treating a subject with cancer and/or a tumor; and a pharmaceutically acceptable carrier. In some embodiments, the amount of the at least one antibody, antigen binding construct, minibody, and/or cys-diabody is from about 0.01 mg/kg to about 25 mg/kg. In some embodiments, the amount of the at least one antibody, antigen binding construct, minibody, and/or cys-diabody is from about 1 mg/kg to about 20 mg/kg. In some embodiments, the pharmaceutical composition further comprises an at least one known small molecule, therapeutic, or antigen binding construct effective in treating a disease. In some embodiments, the at least one known small molecule, therapeutic, or antigen binding construct effective in treating a disease is an antibody. In some embodiments, the at least one known small molecule, therapeutic, or antigen binding construct effective in treating a disease is used as part of chemotherapy. In some embodiments, the at least one known small molecule, therapeutic, or antigen binding construct effective in treating a disease is an immuno-oncology drug. In some embodiments, the at least one known small molecule, therapeutic, or antigen binding construct effective in treating a disease is a DNA repair inhibitor. In some embodiments, the at least one known small molecule, therapeutic, or antigen binding construct effective in treating a cancer and/or tumor is selected from the group consisting of an alkylating agent, metabolic inhibitor, a radiosensitizer agent, an anti-tumor antibiotic, a topoisomerase inhibitor, a mitotic inhibitor, a nitrosourea, a corticosteroid, an anti-angiogenic, an apoptosis inducer, an anti-microtubule agent, a vinca alkaloid, a taxane, an anthracycline, an anti-androgen, a VEGF pathway inhibitor, a VEGF pathway inhibitor, a MAPK/Ras/Raf pathway inhibitor, and an EGFR pathway inhibitor, a KRAS pathway inhibitor. In some embodiments, the disease is fibrosis. In some embodiments, the disease is a cancer or tumor. In some embodiments, the disease is a solid tumor. In some embodiments, the disease is an autoimmune disease. In some embodiments, the disease is cardiovascular.

Some aspects of the present disclosure relate to a method of treating, inhibiting, or ameliorating a disease in a subject, comprising: administering the pharmaceutical composition of any one of embodiments disclosed herein to the subject in need thereof. In some embodiments, the disease is fibrosis. In some embodiments, the disease is a cancer or tumor. In some embodiments, the disease is a solid tumor. In some embodiments, the disease is an autoimmune disease. In some embodiments, the disease is cardiovascular. In some embodiments, the method further comprises imaging the disease using the composition of any of embodiments disclosed herein. In some embodiments, the method further comprises imaging the disease using the antigen binding construct of any one of the embodiments disclosed herein.

Some aspects of the present disclosure relate to a method of targeting FAP protein on a fibroblast in a subject, comprising: administering the pharmaceutical composition of any one of embodiments disclosed herein to the subject. In some embodiments, the fibroblast is a cancer-associated fibroblast. In some embodiments, a cancer cell and/or a tumor associated macrophage are damaged or killed following the targeting of the fibroblast.

Some aspects of the present disclosure relate to a method of inhibiting, ameliorating, damaging, or inducing apoptosis in a cancer or tumor associated macrophage in a subject, comprising: administering the pharmaceutical composition of any one of the embodiments disclosed herein to the subject in need thereof. In some embodiments, the subject is mammalian or human. In some embodiments, the disease is a solid tumor. In some embodiments, the disease is epithelial. In some embodiments, the cancer or tumor is selected from the group consisting of bone cancer, bone sarcoma, breast cancer, carcinoid, cervical cancer, colon cancer, colorectal cancer, endometrial carcinoma, epithelial ovarian cancer, esophageal cancer, gastric cancer, gastrointestinal cancer, glioma, head and neck cancer, hepatocellular cancer, kidney cancer, leukemia, liver cancer, lung cancer, lymphoma, medullary thyroid carcinoma, melanoma, non-small cell lung cancer, osteosarcoma, oral squamous cell carcinoma, oral cancer, ovarian carcinoma, ovarian cancer, pancreatic adenocarcinoma, pancreatic cancer, prostate cancer, rectal cancer, renal cancer, skin cancer, stomach cancer, testis cancer, thyroid cancer, and urothelial cancer.

In some embodiments, the antigen binding construct or composition disclosed herein is humanized.

Some aspects of the present disclosure relate to a use of any of the compositions or antigen binding constructs disclosed herein in the manufacturing of a medicament for administration to a subject. Some aspects of the present disclosure relate to a use of any of the compositions or antigen binding constructs disclosed herein for photodynamic therapy. Some aspects of the present disclosure relate to a use of any of the compositions or antigen binding constructs disclosed herein for theranostics. Some aspects of the present disclosure relate to a use of any of the compositions or antigen binding constructs disclosed herein for diagnostics. Some aspects of the present disclosure relate to a use of any of the compositions or antigen binding constructs disclosed herein molecular imaging. In some embodiments, the imaging one of more of a group consisting of: photoacoustics, MR imaging, magnetic nanoparticles, spectroscopy, optical probes, and/or any other standard method of imaging. Some aspects of the present disclosure relate to a use of any of the compositions or antigen binding constructs disclosed herein for therapy. Some aspects of the present disclosure relate to a use of any of the compositions or antigen binding constructs disclosed herein for diagnosing cancer or a tumor in a subject. Some aspects of the present disclosure relate to a use of any of the compositions or antigen binding constructs disclosed herein for determining a subject's stratification for a therapy or treatment. Some aspects of the present disclosure relate to a use of any of the compositions or antigen binding constructs disclosed herein for monitoring a subject's response to a therapy or treatment. Some aspects of the present disclosure relate to a use of any of the compositions or antigen binding constructs disclosed herein to inform on a change of therapy decision for a subject in need thereof. Some aspects of the present disclosure relate to a use of any of the compositions or antigen binding constructs disclosed herein for assisting in surgery or surgical procedures.

### BRIEF DESCRIPTION OF THE DRAWINGS

FIG. 1 illustrates an expression of various antigen binding constructs in Expi293™ cells in mg/L.

FIG. 2 illustrates a crystallized structure of FAP alpha taken from the Protein Data Bank database, 1Z68.

FIG. 3 illustrates embodiment heavy chain (H) and light chain (L) CDR sequences of SEQ ID NOS: 1-6.

FIG. 4 illustrates embodiment heavy chain (VH) and light chain (VL) sequences of SEQ ID NOS: 7-11.

FIG. 5 illustrates a cleavable signal peptide sequence of SEQ ID NOS: 46 and 121.

FIG. 6 illustrates an embodiment of an antigen binding construct IAB16M1-8 amino acid sequence of SEQ ID NO: 12.

FIG. 7 illustrates an embodiment of an antigen binding construct IAB16M2-9 amino acid sequence of SEQ ID NO: 13.

FIG. 8 illustrates an embodiment of an antigen binding construct IAB16C2-18 amino acid sequence of SEQ ID NO: 14.

FIG. 9 illustrates an embodiment of an antigen binding construct IAB16C4-19 amino acid sequence of SEQ ID NO: 15.

FIG. 10 illustrates an embodiment of an antigen binding construct IAB16C1-22 amino acid sequence of SEQ ID NO: 16.

FIG. 11 illustrates an embodiment of an antigen binding construct IAB16C3-23 amino acid sequence of SEQ ID NO: 17.

FIG. 12 illustrates an embodiment of an antigen binding construct IAB16M1-10 amino acid sequence of SEQ ID NO: 18.

FIG. 13 illustrates an embodiment of an antigen binding construct IAB16M2-11 amino acid sequence of SEQ ID NO: 19.

FIG. 14 illustrates an embodiment of an antigen binding construct IAB16C1-24 amino acid sequence of SEQ ID NO: 20.

FIG. 15 illustrates an embodiment of an antigen binding construct IAB16C2-25 amino acid sequence of SEQ ID NO: 21.

FIG. 16 illustrates an embodiment of an antigen binding construct IAB16C3-26 amino acid sequence of SEQ ID NO: 22.

FIG. 17 illustrates an embodiment of an antigen binding construct IAB16C4-27 amino acid sequence of SEQ ID NO: 23.

FIG. 18 illustrates an embodiment of an antigen binding construct IAB16M1-12 amino acid sequence of SEQ ID NO: 24.

FIG. 19 illustrates an embodiment of an antigen binding construct IAB16M2-13 amino acid sequence of SEQ ID NO: 25.

FIG. 20 illustrates an embodiment of an antigen binding construct IAB16C2-16 amino acid sequence of SEQ ID NO: 26.

FIG. 21 illustrates an embodiment of an antigen binding construct IAB16C4-17 amino acid sequence of SEQ ID NO: 27.

FIG. 22 illustrates an embodiment of an antigen binding construct IAB16C1-20 amino acid sequence of SEQ ID NO: 28.

FIG. 23 illustrates an embodiment of an antigen binding construct IAB16C3-21 amino acid sequence of SEQ ID NO: 29.

FIG. 24 illustrates an embodiment of an antigen binding construct IAB16M1-28 amino acid sequence of SEQ ID NO: 30.

FIG. 25 illustrates an embodiment of an antigen binding construct IAB16M2-29 amino acid sequence of SEQ ID NO: 31.

FIG. 26 illustrates an embodiment of an antigen binding construct IAB16M1-30 amino acid sequence of SEQ ID NO: 32.

FIG. 27 illustrates an embodiment of an antigen binding construct IAB16M2-31 amino acid sequence of SEQ ID NO: 33.

FIG. 28 illustrates an embodiment of an antigen binding construct IAB16M1-32 amino acid sequence of SEQ ID NO: 34.

FIG. 29 illustrates an embodiment of an antigen binding construct IAB16M2-33 amino acid sequence of SEQ ID NO: 35.

FIG. 30 illustrates an embodiment of an antigen binding construct IAB16M1-34 amino acid sequence of SEQ ID NO: 36.

FIG. 31 illustrates an embodiment of an antigen binding construct IAB16M2-35 amino acid sequence of SEQ ID NO: 37.

FIG. 32 illustrates an embodiment of an antigen binding construct IAB16M1-36 amino acid sequence of SEQ ID NO: 38.

FIG. 33 illustrates an embodiment of an antigen binding construct IAB16M2-37 amino acid sequence of SEQ ID NO: 39.

FIG. 34 illustrates an embodiment of an antigen binding construct IAB16C1-38 amino acid sequence of SEQ ID NO: 40.

FIG. 35 illustrates an embodiment of an antigen binding construct IAB16C2-39 amino acid sequence of SEQ ID NO: 41.

FIG. 36 illustrates an embodiment of an antigen binding construct IAB16C3-40 amino acid sequence of SEQ ID NO: 42.

FIG. 37 illustrates an embodiment of an antigen binding construct IAB16C4-41 amino acid sequence of SEQ ID NO: 43.

FIG. 38 illustrates an embodiment of an antigen binding construct IAB16M1-14 amino acid sequence of SEQ ID NO: 44.

FIG. 39 illustrates an embodiment of an antigen binding construct IAB16M2-15 amino acid sequence of SEQ ID NO: 45.

FIG. 40 illustrates some embodiments of nucleic acid sequences encoding an antigen binding construct of SEQ ID NOS: 47-80.

FIG. 41 illustrates some embodiments of novel antigen binding constructs, parental constructs, structure class, and the corresponding amino acid and nucleotide sequences.

FIG. 42A is a graph showing the binding kinetics of antigen binding construct IAB16M1-12 to FAP.

FIG. 42B is a graph showing the binding kinetics of deimmunized antigen binding construct IAB16B2-37 to FAP.

FIG. 43 illustrates the sequence alignment of the heavy and light chains of Sibrotuzumab (SEQ ID NOS: 7 and 187) with six example antigen binding construct sequences (SEQ ID NOS: 24, 30, 32, 34, 36 and 38).

FIG. 44 illustrates the sequence alignment of the heavy and light chains of Sibrotuzumab (SEQ ID NOS: 7 and 187) with six example antigen binding construct sequences (SEQ ID NOS: 25, 31, 33, 35, 37 and 39).

FIG. 45 illustrates the sequence alignment of the heavy and light chains of Sibrotuzumab (SEQ ID NOS: 7 and 187) with four example antigen binding construct sequences (SEQ ID NOS: 28, 40, 29 and 42).

FIG. 46 illustrates the sequence alignment of the heavy and light chains of Sibrotuzumab (SEQ ID NOS: 7 and 187) with four example antigen binding construct sequences (SEQ ID NOS: 26, 41, 27 and 43).

FIG. 47 illustrates the sequence alignment of the heavy chains of Sibrotuzumab (SEQ ID NO: 7) and muF19 (SEQ ID NO: 10) with fourteen example antigen binding construct sequences. Highlighted residues indicate predicted mutations or backmutation based on frequency of occurrence in humans.

FIG. 48 illustrates the sequence alignment of the light chains of Sibrotuzumab (SEQ ID NO: 187) and muF19 (SEQ ID NO: 10) with fourteen example antigen binding construct sequences. Highlighted residues indicate predicted mutations or backmutation based on frequency of occurrence in humans.

FIG. 49A illustrates some embodiments of a schematic of a minibody having bivalent binding to CD8.

FIG. 49B illustrates some embodiments of a schematic of a minibody.

FIG. 50A illustrates some embodiments of a schematic of a cys-diabody showing bivalent binding to an antigen.

FIG. 50B illustrates a schematic of a cys-diabody showing bivalent binding to an antigen.

FIG. 51 shows an embodiment illustration of a minibody.

FIG. 52A illustrates an embodiment of a hinge region on an antigen binding construct of SEQ ID NO: 188.

FIG. 52B illustrates an embodiment of a hinge region on an antigen binding construct of SEQ ID NO: 189.

FIG. 52C illustrates an embodiment of a hinge region on an antigen binding construct of SEQ ID NO: 190.

FIG. 52D illustrates an embodiment of a hinge region on an antigen binding construct of SEQ ID NO: 191.

FIG. 52E illustrates an embodiment of a hinge region on an antigen binding construct of SEQ ID NO: 192.

FIG. 52F illustrates an embodiment of a hinge region on an antigen binding construct of SEQ ID NO: 193.

FIG. 53 illustrates an embodiment of the sequence for a His-tagged extracellular domain of human FAP of SEQ ID NO: 147.

FIG. 54 illustrates an embodiment of the sequence of His-tagged FAP alpha of SEQ ID NO: 148

FIG. 55 illustrates an alignment of the wildtype human FAP amino acid sequence (SEQ ID NO: 148) with the wildtype human DPP4 amino acid sequence (SEQ ID NO: 194).

FIG. 56A is a graph showing the binding kinetics of antigen binding constructs IAB16M1-8, IAB16M2-9, IAB16M1-10, IAB16M1-12, and IAB16M2-13 to a purified human FAP antigen using ELISA.

FIG. 56B is a graph showing the binding kinetics of antigen binding constructs IAB16M1-30, IAB16M2-31, IAB16M1-32, and IAB16M2-33 to a purified human FAP antigen using ELISA.

FIG. 56C is a graph showing the binding kinetics of antigen binding constructs IAB16M1-36 and IAB16M2-37 to a purified human FAP antigen using ELISA.

FIG. 56D is a graph showing the binding kinetics of antigen binding construct IAB16C1-38 to a purified human FAP antigen using ELISA.

FIG. 56E is a graph showing the binding kinetics of antigen binding constructs IAB16M2-29, IAB16C3-21, and IAB16C1-20 to a purified human FAP antigen using ELISA.

FIG. 56F is a graph showing the binding kinetics of antigen binding constructs IAB16C1-24, IAB16C2-25, IAB16C3-26, and IAB16C4-27 to a purified human FAP antigen using ELISA.

FIG. 56G is a graph showing the binding kinetics of antigen binding constructs IAB16C2-16, IAB16C4-17, IAB16C2-18, and IAB16C4-19 to a purified human FAP antigen using ELISA.

FIG. 57A is a graph showing the cross-reactivity binding kinetics of antigen binding construct IAB16C3-26 to a purified murine FAP antigen and a purified human FAP antigen using ELISA.

FIG. 57B is a graph showing the cross-reactivity binding kinetics of antigen binding constructs IAB16M2-9, IAB16M1-10, and IAB16M2-37 to a purified murine FAP antigen and a purified human FAP antigen using ELISA.

FIG. 57C is a graph showing the cross-reactivity binding kinetics of antigen binding constructs IAB16M2-9, IAB16M1-10, and IAB16M1-12 to a purified human FAP antigen and a purified human DPP4 antigen using ELISA.

FIG. 58A is a graph showing the binding of antigen binding constructs IAB16M1-8, IAB16M2-9, IAB16M1-10, IAB16M1-12, IAB16M2-13, IAB16M2-7, and IAB16M1-6 to MRC-5 fibroblasts using flow cytometry.

FIG. 58B is a graph showing the binding of antigen binding constructs IAB16M1-8, IAB16M2-9, IAB16M1-10, IAB16M1-12, IAB16M2-13, IAB16M2-7, IAB16M1-6, and Anti-FAP to U87-MG glioblastoma cells using flow cytometry.

FIG. 58C is a graph showing the binding of antigen binding construct IAB16M2-37 to MRC-5 fibroblasts and U87-MG glioblastoma cells using flow cytometry.

FIG. 59A is a visualization of IAB16M2-9 internalization into HT1080 human FAP cells after 1 hour, using fluorescence microscopy. The white arrows depict cells with particularly significant construct internalization.

FIG. 59B is a visualization of IAB16M1-10 internalization into HT1080 human FAP cells after 1 hour, using fluorescence microscopy. The white arrows depict cells with particularly significant construct internalization.

FIG. 59C is a visualization of IAB16M2-37 internalization into HT1080 human FAP cells after 1 hour, using fluorescence microscopy. The white arrows depict cells with particularly significant construct internalization.

FIG. 60 illustrates embodiments of heavy chain (H) and light chain (L) CDR sequences of SEQ ID NOS: 81-86, 104-109, and 134-139.

FIG. 61 illustrates embodiments of heavy chain (VH) and light chain (VL) sequences of SEQ ID NOS: 87-96, 110-115, 122-125, and 140-143.

FIG. 62 illustrates an embodiment of an antigen binding construct 9A5 amino acid sequence of SEQ ID NO: 97.

FIG. 63 illustrates an embodiment of an antigen binding construct IAB16M1-42 amino acid sequence of SEQ ID NO: 98.

FIG. 64 illustrates an embodiment of an antigen binding construct IAB16M2-43 amino acid sequence of SEQ ID NO: 99.

FIG. 65 illustrates an embodiment of an antigen binding construct IAB16M2-48 amino acid sequence of SEQ ID NO: 100.

FIG. 66 illustrates an embodiment of an antigen binding construct IAB16M2-51 (HC-N76S) amino acid sequence of SEQ ID NO: 101.

FIG. 67 illustrates an embodiment of an antigen binding construct IAB16M2-52 amino acid sequence of SEQ ID NO: 102.

FIG. 68 illustrates an embodiment of an antigen binding construct IAB16M2-53 amino acid sequence of SEQ ID NO: 103.

FIG. 69 illustrates an embodiment of an antigen binding construct 9A2 amino acid sequence of SEQ ID NO: 116.

FIG. 70 illustrates an embodiment of an antigen binding construct IAB16M1-44 amino acid sequence of SEQ ID NO: 117.

FIG. 71 illustrates an embodiment of an antigen binding construct IAB16M2-45 amino acid sequence of SEQ ID NO: 118.

FIG. 72 illustrates an embodiment of an antigen binding construct IAB16M1-46 amino acid sequence of SEQ ID NO: 119.

FIG. 73 illustrates an embodiment of an antigen binding construct IAB16M2-47 amino acid sequence of SEQ ID NO: 120.

FIG. 74 illustrates an embodiment of an antigen binding construct IAB16M2-56 amino acid sequence of SEQ ID NO: 126.

FIG. 75 illustrates an embodiment of an antigen binding construct IAB16M1-59 amino acid sequence of SEQ ID NO: 127.

FIG. 76 illustrates an embodiment of an antigen binding construct IAB16M1-60 amino acid sequence of SEQ ID NO: 128.

FIG. 77 illustrates an embodiment of an antigen binding construct IAB16M1-61 amino acid sequence of SEQ ID NO: 129.

FIG. 78 illustrates an embodiment of an antigen binding construct IAB16M1-54 amino acid sequence of SEQ ID NO: 130.

FIG. 79 illustrates an embodiment of an antigen binding construct IAB16M2-55 amino acid sequence of SEQ ID NO: 131.

FIG. 80 illustrates an embodiment of an antigen binding construct IAB16M1-57 amino acid sequence of SEQ ID NO: 132.

FIG. 81 illustrates an embodiment of an antigen binding construct IAB16M1-58 amino acid sequence of SEQ ID NO: 133.

FIG. 82 illustrates an embodiment of an antigen binding construct 3A9 amino acid sequence of SEQ ID NO: 144.

FIG. 83 illustrates an embodiment of an antigen binding construct IAB16M1-49 amino acid sequence of SEQ ID NO: 145.

FIG. 84 illustrates an embodiment of an antigen binding construct IAB16M2-50 amino acid sequence of SEQ ID NO: 146.

FIG. 85 illustrates the sequence alignment of the heavy chain of murine 9A5 construct (SEQ ID NO: 87) with five example antigen binding construct sequences (SEQ ID NOS: 90, 92, 94, 96 and 123). Underlined residues represent CDR regions. Highlighted residues indicate differences in humanized germlines.

FIG. 86 illustrates the sequence alignment of the light chain of murine 9A5 construct (SEQ ID NO: 87) with five example antigen binding construct sequences (SEQ ID NOS: 89, 91, 93, 95 and 122). Underlined residues represent CDR regions. Highlighted residues indicate differences in humanized germlines.

FIG. 87 illustrates the sequence alignment of the heavy chain of murine 9A2 construct (SEQ ID NO: 110) with five example antigen binding construct sequences (SEQ ID NOS: 112, 91, 114, 95 and 124). Underlined residues represent CDR regions. Highlighted residues indicate differences in humanized germlines.

FIG. 88 illustrates the sequence alignment of the light chain of murine 9A2 construct (SEQ ID NO: 110) with five example antigen binding construct sequences (SEQ ID NOS: 113, 92, 115, 96 and 125). Underlined residues represent CDR regions. Highlighted residues indicate differences in humanized germlines.

FIG. 89 illustrates the sequence alignment of the heavy chain of murine 3A9 construct (SEQ ID NO: 140) with four example antigen binding construct sequences (SEQ ID NOS: 142, 91, 122 and 143). Underlined residues represent CDR regions. Highlighted residues indicate differences in humanized germlines.

FIG. 90 illustrates the sequence alignment of the light chain of murine 3A9 construct (SEQ ID NO: 140) with three example antigen binding construct sequences (SEQ ID NOS: 90, 125 and 94). Underlined residues represent CDR regions. Highlighted residues indicate differences in humanized germlines.

FIG. 91A is a graph showing the binding kinetics of antigen binding constructs IAB16M1-54, IAB16M2-55, and 9A2 to a purified human FAP antigen using ELISA.

FIG. 91B is a graph showing the binding kinetics of antigen binding constructs IAB16M1-42 and IAB16M2-43 to a purified human FAP antigen using ELISA.

FIG. 91C is a graph showing the binding kinetics of antigen binding construct 9G5 to a purified human FAP antigen using ELISA.

FIG. 91D is a graph showing the binding kinetics of antigen binding constructs IAB16M2-48 and IAB16M2-51 to a purified human FAP antigen using ELISA.

FIG. 91E is a graph showing the binding kinetics of antigen binding constructs IAB16M2-52 and IAB16M2-53 to a purified human FAP antigen using ELISA.

FIG. 91F is a graph showing the binding kinetics of antigen binding constructs IAB16M1-49 and IAB16M2-50 to a purified human FAP antigen using ELISA.

FIG. 92 is a graph showing the cross-reactivity binding kinetics of antigen binding construct IAB16M2-43 to a purified murine FAP antigen, a cynomolgus-FAP, a human FAP antigen, and a human DPP4 antigen using ELISA.

FIG. 93 is a graph showing the binding of antigen binding constructs 9A2, 3A9, and 9A5 to HT1080 human FAP cells using flow cytometry.

FIG. 94A is a visualization of IAB16M2-43 internalization into HT1080 human FAP cells, using fluorescence microscopy. The white arrows depict cells with particularly significant construct internalization.

FIG. 94B is a visualization of IAB16M2-43 internalization into MRC5 cells, using fluorescence microscopy. The white arrows depict cells with particularly significant construct internalization.

FIG. 94C is a visualization of IAB16M2-43 internalization into U87-MG cells, using fluorescence microscopy. The white arrows depict cells with particularly significant construct internalization.

FIG. 95 is a graph showing the uptake of radiolabeled minibodies IAB16M2-77 and IAB16M2-78 in liver, kidneys and spleen.

FIG. 96 is a graph showing the uptake of radiolabeled minibody IAB16M2-37 in conjugation with LicorIRDye800 in liver, kidneys and spleen.

FIG. 97 is a graph showing the uptake of radiolabeled minibody IAB16M2-56 in conjugation with LicorIRDye800 and metal chelators DTPA in liver, kidneys and spleen.

FIG. 98 is a graph showing the biodistribution of radiolabeled minibody IAB16M2-56 in conjugation with metal chelator DTPA with different Chelator-to-Minibody Ratio (CMR) in liver, kidneys and spleen.

FIG. 99 is a graph showing the biodistribution of radiolabeled minibody IAB16C3-26 in conjugation with metal chelator DTPA in liver, kidneys and spleen.

FIG. 100 illustrates an embodiment of radiolabeled minibody IAB16M2-77 of SEQ ID NO: 149.

FIG. 101 illustrates an embodiment of radiolabeled minibody IAB16M2-78 of SEQ ID NO: 150.

FIG. 102 illustrates an embodiment of framework 2 of germline sequences of antibodies of SEQ ID NOS: 151-186.

### DETAILED DESCRIPTION

Antibodies which bind FAP are known in the art. An antibody termed F19 that specifically binds to FAP was described in U.S. Pat. No. 5,059,523. F19 may be obtained from the hybridoma cell line ATCC Accession No. HB 8269. A minibody targeting FAP named M036 was developed by phage display and is disclosed in Brocks et al., (2001) Phage Display Selection of FAP-Specific scFv. Molec. Med, 7 (7) pp 461-469. An antibody called Sibrotuzumab binds specifically to FAPα, and is disclosed in U.S. Pat. No. 6,455,677. Antibodies 4B9 and 28H1, which also bind FAP, are disclosed in US patent application US20120128591A1.

Described herein are antigen binding constructs, including minibodies and cys-diabodies formats thereof. In some embodiments, the antigen binding constructs bind to FAP. In some embodiments, the antigen binding constructs can be used for targeting FAP for diagnostic and/or therapeutic purposes. In some embodiments, the antigen binding construct may be at least one of an antibody, a minibody, and a cys-diabody. In some embodiments, the antigen binding constructs may be combined with at least one of: a payload, chelator, a label (detectable marker etc.), small molecule, and/or therapeutic. In some embodiments, targeting FAP provides a health benefit to a subject in need thereof.

### Definitions

As used herein, “a” or “an” may mean one or more than one.

As used herein, the term “about” or “approximately” has its usual meaning as understood by those skilled in the art and thus indicates that a value includes the inherent variation of error for the method being employed to determine a value, or the variation that exists among multiple determinations.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

As used herein, “optional” or “optionally” means that the subsequently described event or circumstance does or does not occur, and that the description includes instances where the event or circumstance occurs and instances where it does not. For example, an optionally substituted group means that the group is unsubstituted or is substituted.

The terms “function” and “functional” as used herein have their plain and ordinary meaning as understood in light of the specification, and refer to a biological, enzymatic, or therapeutic function.

The term “inhibit” as used herein has its plain and ordinary meaning as understood in light of the specification, and may refer to the reduction or prevention of a biological activity. The reduction can be by a percentage that is, is about, is at least, is at least about, is not more than, or is not more than about, 10%, 20%, 30%, 40%, 50%, 60%, 70%, 80%, 90%, or 100%, or an amount that is within a range defined by any two of the aforementioned values. As used herein, the term “delay” has its plain and ordinary meaning as understood in light of the specification, and refers to a slowing, postponement, or deferment of a biological event, to a time which is later than would otherwise be expected. The delay can be a delay of a percentage that is, is about, is at least, is at least about, is not more than, or is not more than about, 0%, 10%, 20%, 30%, 40%, 50%, 60%, 70%, 80%, 90%, 100%, or an amount within a range defined by any two of the aforementioned values. Non-limiting examples of inhibition as used herein may refer to the reduction of FAP activity upon contact with an antigen binding construct, such as reduction of dipeptidyl peptidase activity, endopeptidase activity, and/or proteolytic activity. In some embodiments, inhibition is measured in an enzymatic assay, whereby the drop of activity of an enzyme of interest in quantified over an increasing concentration of inhibitor. For example, the amount of fluorogenic substrate cleaved by FAP is measured in the presence of potential FAP inhibitors, such as BR03354. The values of inhibition are typically represented as an IC50, wherein half of the activity of an enzyme is inhibited at that given concentration of inhibitor. In some embodiments, FAP inhibitors have IC50 values between 10-20 nM. The terms inhibit and delay may not necessarily indicate a 100% inhibition or delay. A partial inhibition or delay may be realized.

The term “yield” of any given substance, compound, or material as used herein has its plain and ordinary meaning as understood in light of the specification and refers to the actual overall amount of the substance, compound, or material relative to the expected overall amount. For example, the yield of the substance, compound, or material is, is about, is at least, is at least about, is not more than, or is not more than about, 80, 81, 82, 83, 84, 85, 90, 91, 92, 93, 94, 95, 96, 97, 98, 99, or 100% of the expected overall amount, including all decimals in between. Yield may be affected by the efficiency of a reaction or process, unwanted side reactions, degradation, quality of the input substances, compounds, or materials, or loss of the desired substance, compound, or material during any step of the production.

As used herein, the term “isolated” has its plain and ordinary meaning as understood in light of the specification, and refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from equal to, about, at least, at least about, not more than, or not more than about, 10%, about 20%, about 30%, about 40%, about 50%, about 60%, about 70%, about 80%, about 90%, about 95%, about 98%, about 99%, substantially 100%, or 100% of the other components with which they were initially associated (or ranges including and/or spanning the aforementioned values). In some embodiments, isolated agents are, are about, are at least, are at least about, are not more than, or are not more than about 80%, about 85%, about 90%, about 91%, about 92%, about 93%, about 94%, about 95%, about 96%, about 97%, about 98%, about 99%, substantially 100%, or 100% pure (or ranges including and/or spanning the aforementioned values). As used herein, a substance that is “isolated” may be “pure” (e.g., substantially free of other components). As used herein, the term “isolated cell” may refer to a cell not contained in a multi-cellular organism or tissue.

As used herein, “in vivo” is given its plain and ordinary meaning as understood in light of the specification and refers to the performance of a method inside living organisms, usually animals, mammals, including humans, and plants, or living cells which make up these living organisms, as opposed to a tissue extract or dead organism.

As used herein, “ex vivo” is given its plain and ordinary meaning as understood in light of the specification and refers to the performance of a method outside a living organism with little alteration of natural conditions.

As used herein, “in vitro” is given its plain and ordinary meaning as understood in light of the specification and refers to the performance of a method outside of biological conditions, e.g., in a petri dish or test tube.

As used herein, “nucleic acid”, “nucleic acid molecule”, or “nucleotide” refers to polynucleotides or oligonucleotides such as deoxyribonucleic acid (DNA) or ribonucleic acid (RNA), oligonucleotides, fragments generated by the polymerase chain reaction (PCR), and fragments generated by any of ligation, scission, endonuclease action, exonuclease action, and by synthetic generation. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid molecule” also includes so-called “peptide nucleic acids,” which comprise naturally-occurring or modified nucleic acid bases attached to a polyamide backbone. Nucleic acids can be either single stranded or double stranded.

The terms “peptide”, “polypeptide”, and “protein” as used herein have their plain and ordinary meaning as understood in light of the specification and refer to macromolecules comprised of amino acids linked by peptide bonds. The numerous functions of peptides, polypeptides, and proteins are known in the art, and include but are not limited to enzymes, structure, transport, defense, hormones, or signaling. Peptides, polypeptides, and proteins are often, but not always, produced biologically by a ribosomal complex using a nucleic acid template, although chemical syntheses are also available. By manipulating the nucleic acid template, peptide, polypeptide, and protein mutations such as substitutions, deletions, truncations, additions, duplications, or fusions of more than one peptide, polypeptide, or protein can be performed. These fusions of more than one peptide, polypeptide, or protein can be joined in the same molecule adjacently, or with extra amino acids in between, e.g. linkers, repeats, epitopes, or tags, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths. The term “downstream” on a polypeptide as used herein has its plain and ordinary meaning as understood in light of the specification and refers to a sequence being after the C-terminus of a previous sequence. The term “upstream” on a polypeptide as used herein has its plain and ordinary meaning as understood in light of the specification and refers to a sequence being before the N-terminus of a subsequent sequence.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, gamma-carboxyglutamate, and O-phosphoserine. Amino acid analogs refer to compounds that have the same basic chemical structure as a naturally occurring amino acid, i.e., an .alpha.-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the constructs provided herein.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

“Percentage of sequence identity” can be determined by comparing two optimally aligned sequences over a comparison window, wherein the portion of the polynucleotide sequence in the comparison window may comprise additions or deletions (i.e., gaps) as compared to the reference sequence (e.g., a polypeptide of the constructs provided herein), which does not comprise additions or deletions, for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same sequences. Two sequences are “substantially identical” if two sequences have a specified percentage of amino acid residues or nucleotides that are the same (for example, 70%, 75%, 80%, 85%, 90%, 95%, 96%, 97%, 98% or 99% sequence identity over a specified region, or, when not specified, over the entire sequence of a reference sequence), when compared and aligned for maximum correspondence over a comparison window, or designated region as measured using one of the following sequence comparison algorithms or by manual alignment and visual inspection. Some embodiments provided herein provide polypeptides or polynucleotides that are substantially identical to the polypeptides or polynucleotides, respectively, exemplified herein. Optionally, the identity exists over a region that is at least about 15, 25 or 50 nucleotides in length, or more preferably over a region that is 100 to 500 or 1000 or more nucleotides in length, or over the full length of the reference sequence. With respect to amino acid sequences, identity or substantial identity can exist over a region that is at least 5, 10, 15 or 20 amino acids in length, optionally at least about 25, 30, 35, 40, 50, 75 or 100 amino acids in length, optionally at least about 150, 200 or 250 amino acids in length, or over the full length of the reference sequence. With respect to shorter amino acid sequences, e.g., amino acid sequences of 20 or fewer amino acids, in some embodiments, substantial identity exists when one or two amino acid residues are conservatively substituted, according to the conservative substitutions defined herein.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or embodiment parameters can be designated. The sequence comparison algorithm then calculates the percent sequence identities for the test sequences relative to the reference sequence, based on the program parameters.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, in some embodiments, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The term “gene” as used herein have their plain and ordinary meaning as understood in light of the specification, and generally refers to a portion of a nucleic acid that encodes a protein or functional RNA; however, the term may optionally encompass regulatory sequences. It will be appreciated by those of ordinary skill in the art that the term “gene” may include gene regulatory sequences (e.g., promoters, enhancers, etc.) and/or intron sequences. It will further be appreciated that definitions of gene include references to nucleic acids that do not encode proteins but rather encode functional RNA molecules such as tRNAs and miRNAs. In some cases, the gene includes regulatory sequences involved in transcription, or message production or composition. In other embodiments, the gene comprises transcribed sequences that encode for a protein, polypeptide or peptide. In keeping with the terminology described herein, an “isolated gene” may comprise transcribed nucleic acid(s), regulatory sequences, coding sequences, or the like, isolated substantially away from other such sequences, such as other naturally occurring genes, regulatory sequences, polypeptide or peptide encoding sequences, etc. In this respect, the term “gene” is used for simplicity to refer to a nucleic acid comprising a nucleotide sequence that is transcribed, and the complement thereof. As will be understood by those in the art, this functional term “gene” includes both genomic sequences, RNA or cDNA sequences, or smaller engineered nucleic acid segments, including nucleic acid segments of a non-transcribed part of a gene, including but not limited to the non-transcribed promoter or enhancer regions of a gene. Smaller engineered gene nucleic acid segments may express, or may be adapted to express using nucleic acid manipulation technology, proteins, polypeptides, domains, peptides, fusion proteins, mutants and/or such like.

An “expression vector” or a “vector,” as described herein, is a nucleic acid molecule encoding a gene that is expressed in a host-cell. Typically, an expression vector comprises a transcription promoter, a gene, and a transcription terminator. Gene expression is usually placed under the control of a promoter, and such a gene is said to be “operably linked to” the promoter. Similarly, a regulatory element and a core promoter are operably linked if the regulatory element modulates the activity of the core promoter.

A “ligand” as described herein refers to a small molecule, peptide, or protein that can form a complex with another molecule or biomolecule for a biological purpose such as, for example, signal triggering. Binding can occur through intermolecular forces, for example ionic bonds, hydrogen bonds, and van der walls interactions. Ligand binding to a receptor protein can alter the three dimensional structure and determine its functional state.

By way of example and not of limitation, ligands can include substrates, proteins, small molecules, inhibitors, activators and neurotransmitters. The strength of binding of a ligand is referred to as the binding affinity and can be determined by direct interactions and solvent effects. A ligand can be bound by a “ligand binding domain.” A ligand binding domain can refer to a conserved sequence in a structure that can bind a specific ligand. Without being limiting, a ligand binding domain can be a specific protein domain that is specific for a ligand or ligands.

“Specific” or “Specificity” can refer to the characteristic of a ligand for the binding partner or alternatively, the binding partner for the ligand, and can include complementary shape, charge and hydrophobic specificity for binding. Specificity for binding can include stereospecificity, regioselectivity and chemoselectivity.

“Label”, “detectable label” or “detectable marker” are used interchangeably herein and refer to a detectable compound or composition which is conjugated directly or indirectly associated with the antibody so as to generate a “labeled” antibody. The label may be detectable by itself (e.g., radioisotope labels or fluorescent labels) or, in the case of an enzymatic label, may catalyze chemical alteration of a substrate compound or composition which is detectable.

The term “payload” denotes an atom or molecule or other entity that is associated (covalently or otherwise) to an antigen binding construct. It includes labels or markers for aspects for diagnostics for example, as well as toxins, cytotoxic agents, chemotherapteutic agents for various therapies. In some embodiments, the payload involves as chelator so as to attach the antigen binding construct to the molecule or atom to be delivered via or colocalized via the antigen binding construct.

The term “cytotoxic agent” as used herein refers to a substance that inhibits or prevents a cellular function and/or causes cell death or destruction. The term is intended to include non-radioactive isotopes (ADC), radioactive isotopes (e.g., At.sup.211, I.sup.131, Isup.125, Y.sup.90, Re.sup.186, Re.sup.188, Sm.sup.153, Bi.sup.212, P.sup.32, Pb.sup.212 and radioactive isotopes of Lu), chemotherapeutic agents (e.g., methotrexate, adriamicin, vinca alkaloids (vincristine, vinblastine, etoposide), doxorubicin, melphalan, mitomycin C, chlorambucil, daunorubicin or other intercalating agents, enzymes and fragments thereof such as nucleolytic enzymes, antibiotics, and toxins such as small molecule toxins or enzymatically active toxins of bacterial, fungal, plant or animal origin, including fragments and/or variants thereof, toxins, growth inhibitory agents, drug moieties, and the various antitumor or anticancer agents disclosed below. Other cytotoxic agents are described below. A tumoricidal agent causes destruction of tumor cells.

A “toxin” is any substance capable of having a detrimental effect on the growth or proliferation of a cell.

A “chemotherapeutic agent” is a chemical compound useful in the treatment of cancer. Examples of chemotherapeutic agents include alkylating agents such as thiotepa and CYTOXAN™ cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylolomelamine; acetogenins (especially bullatacin and bullatacinone); delta-9-tetrahydrocannabinol (dronabinol, MARINOL™); beta-lapachone; lapachol; colchicines; betulinic acid; a camptothecin (including the synthetic analogue topotecan (HYCAMTIN™), CPT-11 (irinotecan, CAMPTOSAR™), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e. g., calicheamicin, especially calicheamicin gammall and calicheamicin omegall (see, e.g., Agnew, Chem Intl. Ed. Engl., 33: 183-186 (1994)); dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycins, dactinomycin, daunorubicin, detorubicin, 6-diazo-5-oxo-L-norleucine, ADRIAMYCIN™ doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; metabolic inhibitor such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE™, FILDESIN™); dacarbazine; mannomustine; mitobronitol; mitolactol; pipobroman; gacytosine; arabinoside (“Ara-C”); thiotepa; taxoids, e.g., TAXOL® paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE™ docetaxel (Rhone-Poulenc Rorer, Antony, France); chloranbucil; gemcitabine (GEMZAR™); 6-thioguanine; mercaptopurine; methotrexate; platinum analogs such as cisplatin and carboplatin; vinblastine (VELBAN™); platinum; etoposide (VP-16); ifosfamide; mitoxantrone; vincristine (ONCOVIN™); oxaliplatin; leucovovin; vinorelbine (NAVELBINE™); novantrone; edatrexate; daunomycin; aminopterin; ibandronate; topoisomerase inhibitor RFS 2000; difluoromethylornithine (DMFO); retinoids such as retinoic acid; capecitabine (XELODA™); pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovovin.

“Radiotherapy” means treatment using radiation or a radioisotope with a therapeutic purpose. It includes radiation therapy intended to have abscopal effect as described in Yang Liu, Yinping Dong, Li Kong, Fang Shi, Hui Zhu & Jinming Yu; “Abscopal effect of radiotherapy combined with immune checkpoint inhibitors”; Journal of Hematology & Oncology volume 11, Article number: 104 (2018); and in Melek Tugce Yilmaz, Aysenur Elmali, and Gozde Yazici; “Abscopal Effect, From Myth to Reality: From Radiation Oncologists' Perspective”; Cureus. 2019 January; 11(1).

The terms “Immune checkpoint inhibitor” (sometimes referred to as “ICI”), or “checkpoint inhibitor” (sometimes “CPI”) or “immune checkpoint blockade inhibitor” and all similar terms, denote a subclass of immunotherapies. Examples include molecules that block certain proteins made by some types of immune system cells, such as T cells, and some cancer cells. These proteins help keep immune responses in check and can keep T cells from killing cancer cells. When these proteins are blocked, the immune system is free to be active and T cells are able to kill cancer cells. Some embodiments include anti-PD1 and anti-PD-L1 binding agents, anti-CTLA4 agents, and multi-specific agents including, but not limited to, anti-CTLA-4/B7-1/B7-2. Additional immunotherapies include checkpoint inhibitors such as ipilimumab (Yervoy), pembrolizumab (Keytruda), nivolumab (Opdivo), atezolizumab (Tecentriq), avelumab (Bavencio), and durvalumab (Imfinzi). IOTs also include tremelimumab and pidilizumab, Small molecule ICIs are also in development including BMS-1001, BMS-1116, CA-170, CA-327, Imiquimod, Resiquimod, 852A, VTX-2337, ADU-S100, MK-1454, Ibrutinib, 3AC, Idelalisib, IPI-549, Epacadostat, AT-38, CPI-444, Vipadenant, Preladenant, PBF, AZD4635, Galuniseritib, OTX015/MK-8628, CPI-0610 (c.f. Kerr and Chisolm (2019) The Journal of Immunology, 2019, 202: 11-19.)

IOTs also include other modalities which are not CPIs but which also activate the host immune system against the cancer, or render the tumor vulnerable to CPI therapy. Such embodiment IOTs include but are not limited to: T-cell immunomodulators such as the cytokines IL-2, IL-7, IL-15, IL-21, IL-12, GM-CSF and IFNα (including THOR-707 of Synthorx Therapeutics; and NKTR-214 bempegaldesleukin of Nektar Therapeutics); Various other interferons and interleukins; TGFβ1 inhibitors (such as SRK-181 in development by Scholar Rock); Oncolytic therapy (including oncolytic virus therapy); Adoptive cell therapy such as T cell-therapy (including CAR-T cell therapy); Cancer vaccines (both preventative and treatment based). Immunotherapy also includes strategies that increase the burden of neoantigens in tumour cells, including targeted therapies which cause a tumor cell to express or reveal tumor associated antigens. (c.f. Galon and Bruni (2019) Nature Reviews Drug Discovery v. 18, pages 197-218). Further IOTs include TLR9 ligands (Checkmate Pharmaceuticals), A2A/A2B dual antagonists (Arcus Biosciences) and vaccination peptides directed to endogenous enzymes such as IDO-1 and arginase (IO Biotech). IOTs include HS-110, HS-130 and PTX-35 (Heat Biologics).

Those skilled in the art recognize that immunotherapies may be used in combination with each other. Immunotherapies can also be used before, after, or in combination with other therapies for the disease, including in the case of cancer, radiation therapy, chemotherapy of all types (including the cytotoxic agents, chemotherapeutic agents, anti-hormonal agents, and growth inhibitory agents referred to above) and surgical resection.

The term “antigen binding construct” includes all varieties of antibodies, including binding fragments thereof. Further included are constructs that include 1, 2, 3, 4, 5, and/or 6 CDRs. In some embodiments, these CDRs can be distributed between their appropriate framework regions in a traditional antibody. In some embodiments, the CDRs can be contained within a heavy and/or light chain variable region. In some embodiments, the CDRs can be within a heavy chain and/or a light chain. In some embodiments, the CDRs can be within a single peptide chain. In some embodiments, the CDRs can be within two or more peptides that are covalently linked together. In some embodiments, they can be covalently linked together by a disulfide bond. In some embodiments, they can be linked via a linking molecule or moiety. In some embodiments, the antigen binding proteins are non-covalent, such as a diabody and a monovalent scFv. Unless otherwise denoted herein, the antigen binding constructs described herein bind to the noted target molecule. The term also includes minibodies and cys-diabodies.

The term “antibody” includes, but is not limited to, genetically engineered or otherwise modified forms of immunoglobulins, such as intrabodies, chimeric antibodies, fully human antibodies, humanized antibodies, antibody fragments, and heteroconjugate antibodies (e.g., bispecific antibodies, diabodies, triabodies, tetrabodies, etc.). The term “antibody” includes cys-diabodies and minibodies. Thus, each and every embodiment provided herein in regard to “antibodies” is also envisioned as cys-diabody and/or minibody embodiments, unless explicitly denoted otherwise. The term “antibody” includes a polypeptide of the immunoglobulin family or a polypeptide comprising fragments of an immunoglobulin that is capable of noncovalently, reversibly, and in a specific manner binding a corresponding antigen. An exemplary antibody structural unit comprises a tetramer. In some embodiments, a full-length antibody can be composed of two identical pairs of polypeptide chains, each pair having one “light” and one “heavy” chain, connected through a disulfide bond. The recognized immunoglobulin genes include the kappa, lambda, alpha, gamma, delta, epsilon, and mu constant region genes, as well as the myriad immunoglobulin variable region genes. For full length chains, the light chains are classified as either kappa or lambda. For full length chains, the heavy chains are classified as gamma, mu, alpha, delta, or epsilon, which in turn define the immunoglobulin classes, IgG, IgM, IgA, IgD, and IgE, respectively. The N-terminus of each chain defines a variable region of about 100 to 110 or more amino acids primarily responsible for antigen recognition. The terms variable light chain (VL) and variable heavy chain (VH) refer to these regions of light and heavy chains respectively. As used in this application, an “antibody” encompasses all variations of antibody and fragments thereof. Thus, within the scope of this concept are full length antibodies, chimeric antibodies, humanized antibodies, single chain antibodies (scFv), Fab, Fab′, and multimeric versions of these fragments (e.g., F(ab′)2) with the same binding specificity. In some embodiments, the antibody binds specifically to a desired target.

The term “sibrotuzumab”, or “BIBH1”, as used herein is given its plain and ordinary meaning as understood in light of the specification and refers to a known humanized monoclonal antibody that binds FAP. It was intended and developed by Boehringer Ingelheim Pharma KG for the treatment of cancer. However, in 2003 it failed a phase II clinical trial for metastatic colorectal cancer.

“Complementarity-determining domains” or “complementarity-determining regions (“CDRs”) interchangeably refer to the hypervariable regions of VL and VH. The CDRs are the target protein-binding site of the antibody chains that harbors specificity for such target protein. In some embodiments, there are three CDRs (CDR1-3, numbered sequentially from the N-terminus) in each VL and/or VH, constituting about 15-20% of the variable domains. The CDRs are structurally complementary to the epitope of the target protein and are thus directly responsible for the binding specificity. The remaining stretches of the VL or VH, the so-called framework regions (FRs), exhibit less variation in amino acid sequence (Kuby, Immunology, 4th ed., Chapter 4. W.H. Freeman & Co., New York, 2000).

The positions of the CDRs and framework regions can be determined using various well known definitions in the art, e.g., Kabat (Wu, T. T., E. A. Kabat. 1970. An analysis of the sequences of the variable regions of Bence Jones proteins and myeloma light chains and their implications for antibody complementarity. J. Exp. Med. 132: 211-250; Kabat, E. A., Wu, T. T., Perry, H., Gottesman, K., and Foeller, C. (1991) Sequences of Proteins of Immunological Interest, 5th ed., NIH Publication No. 91-3242, Bethesda, MD), Chothia (Chothia and Lesk, J. Mol. Biol., 196:901-917 (1987); Chothia et al., Nature, 342:877-883 (1989); Chothia et al., J. Mol. Biol., 227:799-817 (1992); Al-Lazikani et al., J. Mol. Biol., 273:927-748 (1997)), ImMunoGeneTics database (IMGT) (on the worldwide web at imgt.org/) Giudicelli, V., Duroux, P., Ginestoux, C., Folch, G., Jabado-Michaloud, J., Chaume, D. and Lefranc, M.-P. IMGT/LIGM-DB, the IMGT® comprehensive database of immunoglobulin and T cell receptor nucleotide sequences Nucl. Acids Res., 34, D781-D784 (2006), PMID: 16381979; Lefranc, M.-P., Pommie, C., Ruiz, M., Giudicelli, V., Foulquier, E., Truong, L., Thouvenin-Contet, V. and Lefranc, G., IMGT unique numbering for immunoglobulin and T cell receptor variable domains and Ig superfamily V-like domains Dev. Comp. Immunol., 27, 55-77 (2003). PMID: 12477501; Brochet, X., Lefranc, M.-P. and Giudicelli, V. IMGT/V-QUEST: the highly customized and integrated system for IG and TR standardized V-J and V-D-J sequence analysis Nucl. Acids Res, 36, W503-508 (2008); AbM (Martin et al., Proc. Natl. Acad. Sci. USA, 86:9268-9272 (1989), North (North B., Lehmann A., Dunbrack R. L., A new clustering of antibody CDR loop conformations, J. Mol. Biol. (2011) 406(2): 228-256), AHo (Honegger A., Pluckthun, Yet another numbering scheme for immunoglobulin variable domains: an automatic modeling and analysis tool, J. Mol. Biol. (2001) 309, 657-670); the contact definition (MacCallum et al., J. Mol. Biol., 262:732-745 (1996)), and/or the automatic modeling and analysis tool Honegger A, Plückthun A. (world wide web at bioc dot uzh dot ch/antibody/Numbering/index dot html). In some embodiments, any one or more of the CDRs in any of the sequences provided herein can be defined by any of these CDR definitions.

An “antibody variable light chain” or an “antibody variable heavy chain” as used herein refers to a polypeptide comprising the VL or VH, respectively. The endogenous VL is encoded by the gene segments V (variable) and J (junctional), and the endogenous VH by V, D (diversity), and J. Each of VL or VH includes the CDRs as well as the framework regions. In this application, antibody variable light chains and/or antibody variable heavy chains may, from time to time, be collectively referred to as “antibody chains.” These terms encompass antibody chains containing mutations that do not disrupt the basic structure of VL or VH, as one skilled in the art will readily recognize. In some embodiments, full length heavy and/or light chains are contemplated. In some embodiments, only the variable region of the heavy and/or light chains are contemplated as being present.

Antibodies can exist as intact immunoglobulins or as a number of fragments produced by digestion with various peptidases. Thus, for example, pepsin digests an antibody below the disulfide linkages in the hinge region to produce F(ab)′2, a dimer of Fab′ which itself is a light chain (VL-CL) joined to VH-CH1 by a disulfide bond. The F(ab)′2 may be reduced under mild conditions to break the disulfide linkage in the hinge region, thereby converting the F(ab)′2 dimer into a Fab′ monomer. The Fab′ monomer is a Fab with part of the hinge region. (Paul, Fundamental Immunology 3d ed. (1993). Likewise, Fab fragments can be derived by enzymatic digestion of full-length antibodies with papain, which cleaves the antibody at the upper hinge region. While various antibody fragments are defined in terms of the digestion of an intact antibody, one of skill will appreciate that such fragments may be synthesized de novo either chemically or by using recombinant DNA methodology. Thus, the term “antibody,” as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

For preparation of monoclonal or polyclonal antibodies, any technique known in the art can be used (see, e.g., Kohler & Milstein, Nature 256:495-497 (1975); Kozbor et al., Immunology Today 4:72 (1983); Cole et al., Monoclonal Antibodies and Cancer Therapy, pp. 77-96. Alan R. Liss, Inc. 1985; Advances in the production of human monoclonal antibodies Shixia Wang, Antibody Technology Journal 2011:1 1-4; J Cell Biochem. 2005 Oct. 1; 96(2):305-13; Recombinant polyclonal antibodies for cancer therapy; Sharon J, Liebman M A, Williams B R; and Drug Discov Today. 2006 July, 11(13-14):655-60, Recombinant polyclonal antibodies: the next generation of antibody therapeutics?, Haurum J S). Techniques for the production of single chain antibodies (U.S. Pat. No. 4,946,778) can be adapted to produce antibodies to polypeptides. Also, transgenic mice, or other organisms such as other mammals, may be used to express fully human monoclonal antibodies. Alternatively, phage display technology can be used to identify high affinity binders to selected antigens (see, e.g., McCafferty et al., supra; Marks et al., Biotechnology, 10:779-783, (1992)). B-cell cloning can be used to identify fully human antibodies directly from human subjects (Wardemann H., Busse E., Expression Cloning of Antibodies from Single Human B Cells, Methods Mol. Biol. (2019) 1956:105-125).

Methods for humanizing or primatizing non-human antibodies are well known in the art. Generally, in this method, an antibody or antibody fragment from a non-human source has one or more amino acid residues introduced into it from a source which is human (or primate, in the case of primatization). In some embodiments, a non-human antibody has one or more amino acid residues introduced into it from a source which is human. These human amino acid residues are often referred to as import residues, which are typically taken from an import variable domain. In some embodiments, the terms “donor” and “acceptor” sequences can be employed. Humanization can be essentially performed following the method of Winter and co-workers (see, e.g., Jones et al., Nature 321:522-525 (1986); Riechmann et al., Nature 332:323-327 (1988); Verhoeyen et al., Science 239:1534-1536 (1988) and Presta, Curr. Op. Struct. Biol. 2:593-596 (1992)), by substituting rodent CDRs or CDR sequences for the corresponding sequences of a human antibody. Accordingly, such humanized antibodies (as described in, e.g., U.S. Pat. No. 4,816,567) have substantially less than an intact non-human variable domain substituted by the corresponding sequence from a human. In practice, humanized antibodies are typically rodent or other mammalian antibodies in which some complementarity determining region (“CDR”) residues and possibly some framework (“FR”) residues are substituted by residues from analogous sites in human antibodies.

Antibodies further include one or more immunoglobulin chains that are chemically conjugated to, or expressed as, fusion proteins with other proteins. In some embodiments, the antigen binding constructs can be a monovalent scFv constructs. In some embodiments, the antigen binding constructs can be a bispecific construct. A bispecific or bifunctional antibody is an artificial hybrid antibody having two different heavy/light chain pairs and two different binding sites. Other antigen-binding fragments or antibody portions include bivalent scFv (diabody), bispecific scFv antibodies where the antibody molecule recognizes two different epitopes, single binding domains (sdAb or nanobodies), and minibodies.

The term “antibody fragment” includes, but is not limited to one or more antigen binding fragments of antibodies alone or in combination with other molecules, including, but not limited to Fab′, F(ab′)2, Fab, Fv, rIgG (reduced IgG), scFv fragments (monovalent, tri-valent, etc.), single domain fragments (nanobodies), peptibodies, minibodies, diabodies, and cys-diabodies. The term “scFv” refers to a single chain Fv (“fragment variable”) antibody in which the variable domains of the heavy chain and of the light chain of a traditional two chain antibody have been joined to form one chain.

A “single chain variable fragment” or scFv is a fusion protein that comprises the variable regions of the heavy chain (VH) and the light chains (VL) of an immunoglobulin that is connected with a short linker peptide. Without being limiting, the linker can comprise glycine for flexibility and hydrophilic amino acids, for example serine or threonine for solubility. The linker can connect the N-terminus of the VH with the C-terminus of the VL or it can connect the C-terminus of the VH with the N-terminus of the VL.

A minibody is an antibody format that has a smaller molecular weight than the full-length antibody while maintaining the bivalent binding property against an antigen. In some embodiments, minibodies are between 50-100 kDa, and most preferably about 80 kDa. In some embodiments, Fab2 is about 100 kDa. In some embodiments, a full-length antibody is about 150 kDa. In some embodiments, FAP-diabodies are about 50 kDa. In some embodiments, cys-diabodies are about 50 kDa. In some embodiments, scFv is about 25 kDa. In some embodiments, VHH is about 12 kDa. Because of its smaller size, the minibody has a faster clearance from the system and enhanced penetration when targeting tumor tissue. With the ability for strong and selective targeting combined with rapid clearance, the minibody is advantageous for diagnostic imaging and delivery of cytotoxic/radioactive payloads for which prolonged circulation times may result in adverse side effects. A “minibody” as described herein includes a homodimer, wherein each monomer is a single-chain variable fragment (scFv) linked to a human IgG1 C_{H}3 domain by a linker, such as a hinge sequence. In some embodiments, the hinge sequence is a human IgG1 or IgG2 hinge sequence. In some embodiments, the CH3 sequence comprises an IgG1 C_{H}3, or IgG2 C_{H}3 sequence.

In some embodiments, the hinge sequence is an artificial hinge sequence. In some embodiments, the hinge sequence can be an IgG hinge from any one or more of the four classes. The artificial hinge sequence may include a portion of a human IgG1 or IgG2 hinge and a GlySer linker (also known as an “extension” when distinguishing this section from the generic linker sequence that links the Vh and Vl regions) sequence. Artificial hinge sequences and linker sequences that may be usefully employed with the invention are set out a PCT Publication WO2017027325A1.

The scFv can have a V_{H}-V_{L} or a V_{L}-V_{H} orientation. In some embodiments, the V_{H} and V_{L} are linked to each other by an amino acid linker sequence. The amino acid linker can be a linker as described herein. In some embodiments, the linker is GlySer-rich and approximately 15-20 amino acids in length. In another embodiment, the linker is GlySer rich and is 18 amino acids in length. In some embodiments, the linker length varies between (and including) about 1 to 50 amino acids, for example, 2 to 30, 3 to 20, 4 to 15, or 5 amino acids to 8 amino acids. In some embodiments, the minibody scFv has a sequence that is at least about 80% identical to a scFv of a cys-diabody described herein, for example at least about 80%, 85%, 86%, 87%, 88%, 89%, 90%, 91%, 92%, 93, 94, 95%, 96%, 97%, 98%, or 99% identity. The scFv can have a V_{H}V_{L} or a V_{L}V_{H} orientation.

In some embodiments, each monomer of the minibody includes the following elements, from N-terminus to C-terminus: (a) an scFv sequence that includes a V_{H} domain linked to a V_{L} domain and that binds to the target molecule, (b) a hinge-extension domain comprising a human IgG1 hinge region, and (c) a human IgG C_{H}3 sequence. In some embodiments, each monomer of the minibody includes the following elements, from N-terminus to C-terminus: (a) an scFv sequence that includes a VH domain linked to a V_{L} domain and that binds to the target molecule, (b) a hinge-extension domain comprising an IgG2 hinge region as described herein, and (c) a human IgG C_{H}3 sequence. In some embodiments, each monomer of the minibody includes an IgG2, an IgG3, or an IgG4 C_{H}3. In some embodiments, each monomer of the minibody can include a CH3 domain of an IgA or IgD and/or a CH4 domain of a IgM and/or an IgE. In some embodiments, the minibody is encoded by a nucleic acid can be expressed by a cell, a cell line or other suitable expression system as described herein. Thus, a signal sequence can be fused to the N-terminus of the scFv to enable secretion of the minibody when expressed in the cell or cell line.

The term “diabody” denotes a dimer that comprises heavy chain (V_{H}) domains and light-chain variable (V_{L}) domains. Each heavy chain domain is connected to a light chain domain through a linker.

The term “linker” denotes a peptide sequence that connects the V_{H} and V_{L} domains. Linkers can orient the V_{H} and V_{L} domains in either a V_{L}-V_{H} or V_{H}-V_{L} orientation. The linker connects V_{H} to V_{L} through its peptide backbone.

The term “extension sequence” denotes a region that connects a first V_{H} domain to a second V_{H} domain or a first V_{L} to a second V_{L} domain, in for example, a diabody. Extension sequences can connect the domains through the C-terminus of each domain. In some embodiments, extension sequences connect the domains through covalent bonds. In some embodiments, the extension sequence will include one or more cysteine, allowing for one or more disulfide bonds to be formed between two such extension sequences. Examples of extension sequences, as applied to the C-terminus of cys-diabodies are disclosed in WIPO Publication WO2018147960A1. An example of a pair of extension sequences is shown as the line with two cysteines connecting either the two heavy chain domains or the two light chain domains. While the extension sequence will be towards the C-terminus of the constructs, it need not be the absolute last amino acid in the variable domain. That is, the linker can be positioned slightly N-terminal to the C-terminus. For example, the extension sequence can be placed within the 10 amino acids at the C-terminus. Similarly, additional sequence can be placed between the native C-terminus and where the extension sequence starts. The extension sequence can connect V_{H} to V_{H} or V_{L} to V_{L} through a disulfide bond.

A cys-diabody denotes a modified protein carrying an added C-terminal cys sequence which can result in a construct that is a disulfide-bonded dimer. In some embodiments, the cys-diabody is mono-specific. In some embodiments, the cys-diabody is bispecific.

As will be appreciated by those of skill in the art, while the present disclosure generally references antigen binding constructs, specific cys-diabodies embodiment arrangements can be employed for various embodiments for specific advantages. In some embodiments, the cysteines are cross-linked with one another. In some embodiments, the cysteines are reduced, and thus, these tails forming cysteines do not form a disulfide bond with one another. In some embodiments, one or more of the “tail forming” cysteines form a covalent bond with one or more detectable marker, such as a fluorescent probe. In some embodiments, one or more of the “tail forming” cysteines can be conjugated through a covalent bond with a half-life extension moiety, such as polyethylene glycols (PEGs) of different molecular weight. In some embodiments, any covalently modifiable moiety can be employed in place of one or more of the cysteines. For example, this can include a GlySer linker, a GlyLeu linker, and/or an insert cysteine after a short tag. In some embodiments, the connection can be established via a coiled coil or a leucine zipper. In some embodiments, the “tail” itself can include functional groups on its end so that it can selectively bind to a desired residue and/or location at the ends of each of the polypeptides, in place of the disulfide bond itself. In some embodiments, rather than the tail providing space between the two polypeptide chains, the covalently modifiable moieties can be attached directly to the end of the heavy or light chain polypeptide, but the two covalently modifiable moieties can be connected by a linker. In such embodiments, the construct can still include the cysteine on the tail, but simply not be cross-linked. In other embodiments, the construct need not have the cysteine in a tail or the tail at all.

The phrase “specifically bind” or “selectively bind,” when used in the context of describing the interaction between an antigen, e.g., a protein, to an antibody or antibody-derived binding agent, refers to a binding reaction that is determinative of the presence of the antigen in a heterogeneous population of proteins and other biologics, e.g., in a biological sample, e.g., a blood, serum, plasma or tissue sample. Thus, under designated immunoassay conditions, in some embodiments, the antibodies or binding agents with a particular binding specificity bind to a particular antigen at least two times the background and do not substantially bind in a significant amount to other antigens present in the sample. Specific binding to an antibody or binding agent under such conditions may require the antibody or agent to have been selected for its specificity for a particular protein. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998), for a description of immunoassay formats and conditions that can be used to determine specific immunoreactivity). Typically, a specific or selective binding reaction will produce a signal at least twice over the background signal and more typically at least 10 to 100 times over the background.

The term “equilibrium dissociation constant (KD, M)” refers to the dissociation rate constant (kd, time⁻¹) divided by the association rate constant (ka, time⁻¹, M⁻¹). Equilibrium dissociation constants can be measured using any known method in the art. The antibodies provided herein can have an equilibrium dissociation constant of less than about 10⁻⁷ or 10⁻⁸ M, for example, less than about 10⁻⁹ M or 10⁻¹⁰ M, in some embodiments, less than about 10⁻¹¹ M, 10⁻¹² M, 10⁻¹³ M, 10⁻¹⁴ M or 10⁻¹⁵ M. In some embodiments, the KD is determined for an antibody dissolved in buffer. In some embodiments, the buffer is a phosphate buffer.

“PET” is a diagnostic technique that can be used to observe functions and metabolism of human organs and tissues at the molecular level. For PET, a positron radioactive drug (e.g., ¹⁸F-FDG) can be injected into a human body. If FDG is used, because the metabolism of fludeoxyglucose (FDG) is similar to glucose, the FDG will gather in cells that digest the glucose. A positron emitted by the decay of ¹⁸F and an electron in tissues will undergo an annihilation reaction to generate two gamma-photons with the same energy in opposite directions. A detector array surrounding the human body can detect the two photons using a coincidence measurement technique, and determine position information of the positron. A tomography image of positrons in the human body can then be constructed by processing the position information using an image reconstruction software. In some situations, Immuno-PET can be employed, where the label (e.g., ¹⁸F) is attached or associated with an antigen binding construct. In such embodiments, the distribution of the antigen binding construct can be monitored, which will depend upon the binding properties and distribution properties of the antigen binding construct. For example, if a CD8-directed minibody is used, then PET can be used to monitor the distribution of the CD8 molecules through the hosts' system. PET systems are known in the art and include, for example U.S. Pat. Pub. No. 20170357015, 20170153337, 20150196266, 20150087974, 20120318988, and 20090159804, the entireties of each of which are incorporated by reference herein for their description regarding PET and the use thereof.

In some embodiments, imaging is conducted through PET scan, a PET/CT scan, or SPECT scan. In some embodiments, imaging is conducted through photoacoustics, optical probes, MR imaging, magnetic nanoparticles for imaging, spectroscopy, and/or any other standard method of imaging. In some embodiments, at least one optical probe is coupled together with an at least one metal chelator. In some embodiments, optical imaging is used for assisted surgery. In some embodiments, photodynamic therapy is used. In some embodiments, photodynamic therapy is used for assisted surgery. In some embodiments, photodynamic therapy is used for theranostics.

In some embodiments, diagnostics are performed in vivo. In some embodiments, the diagnostics comprise imaging using the antigen binding constructs and/or formulations described herein. In some embodiments, the imaging is used in one or more of cancer diagnoses, determining a subject's stratification for therapy, monitoring a subject's response to therapy, assist in surgery, and/or inform on change in therapy decision.

In some embodiments, the antigen binding constructs and/or compositions are used as part of a theranostics approach. The term “theranostics” as used herein has its usual meaning as understood by those skilled in the art and thus refers to the combination of therapeutics and diagnostics. In some embodiments, theranostics comprises the combination of first identifying (diagnosing) a disease using a first compound, and then delivering (therapy) a second compound to treat the disease. In some embodiments, the second compound is the same as the first compound. In some embodiments, the first and second compounds are derivatives of each other. In some embodiments, the first and second compounds are distinct from one another. In some embodiments, the first compound is conjugated to a detectable marker, such as a fluorescent, chemically reactive, luminescent, or radioactive marker. In some embodiments, the detectable marker is monitored through imaging. In some embodiments, the second compound is conjugated to a therapeutic small molecule or payload that is effective as a medicament. In some embodiments, the second compound is conjugated to a toxic radiolabel, cytotoxic agent, or other molecule capable of producing an adverse effect in the targeted cell, tissue, organ, or organ system. In some embodiments, the adverse effect is one or more of cell cycle arrest, apoptosis, growth arrest, stress induction, cytotoxicity, DNA repair inhibition, necrosis, oxidative stress, nitrosative stress, free radical stress, enhanced targeting by subject's immune system, protein degradation, enhanced protein turnover, inhibited protein turnover, metabolic arrest, organelle arrest, transcription arrest, DNA replication arrest, translation arrest, or any combination thereof.

Some embodiments described herein relate to pharmaceutical compositions or dietary supplements that comprise, consist essentially of, or consist of an effective amount of any one or more of the therapeutic agents described herein. Such pharmaceutical compositions and dietary supplements are suitable for human and/or veterinary applications.

The terms “individual”, “subject”, “host,” or “patient” as used herein have their usual meaning as understood by those skilled in the art and thus includes a human or a non-human mammal. The term “mammal” is used in its usual biological sense. Thus, it specifically includes, but is not limited to, primates, including simians (chimpanzees, apes, monkeys), humans, cattle, horses, sheep, goats, swine, rabbits, dogs, cats, rodents, rats, mice, or guinea pigs.

As used herein, “treatment” or “therapy” of a disease or condition refers to reducing the severity, frequency, or occurrence of at least one symptom of that disease or condition, compared to a similar but untreated patient. Treatment can also refer to halting, slowing, or reversing the progression of a disease or condition, compared to a similar but untreated patient. Treatment may further comprise addressing the root cause of the disease and/or one or more symptoms. The term “prevent” does not require the absolute prohibition of the disorder or disease.

The terms “effective amount” or “effective dose” as used herein have their usual meaning as understood by those skilled in the art and refer to that amount of a recited composition or compound that results in an observable biological effect. Actual dosage levels of active ingredients in an active composition of the presently disclosed subject matter can be varied so as to administer an amount of the active composition or compound that is effective to achieve the desired response for a particular subject and/or application. The selected dosage level will depend upon a variety of factors including, but not limited to, the activity of the composition, formulation, route of administration, combination with other drugs or treatments, severity of the condition being treated, and the physical condition and prior medical history of the subject being treated. In some embodiments, a minimal dose is administered, and dose is escalated in the absence of dose-limiting toxicity to a minimally effective amount. Determination and adjustment of an effective dose, as well as evaluation of when and how to make such adjustments, are contemplated herein.

A “therapeutically effective amount” or a “therapeutically effective dose” is an amount that produces a desired therapeutic effect in a subject, such as preventing, treating a target condition, delaying the onset of the disorder and/or symptoms, and/or alleviating symptoms associated with the condition. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and/or the route of administration. One skilled in the clinical and pharmacological arts will be able to determine a therapeutically effective amount through routine experimentation, for example by monitoring a subject's response to administration of a compound and adjusting the dosage accordingly, given the present disclosure. For additional guidance, see Remington: The Science and Practice of Pharmacy 21.sup.st Edition, Univ. of Sciences in Philadelphia (USIP), Lippincott Williams & Wilkins, Philadelphia, Pa., 2005.

The term “biopharmaceutical” refers to biological products such as proteins (including fusion proteins), vaccines, blood and blood components, allergenics, somatic cells, gene therapy components, tissues, and recombinant therapeutic proteins. Biologics can include sugars, proteins, or nucleic acids or complex combinations thereof, or may be living entities such as cells and tissues. Biologics can be isolated from a variety of natural sources such as human, animal, or microorganism and may be produced by a variety of methods including the use of recombinant DNA.

As used herein, “pharmaceutically acceptable” has its plain and ordinary meaning as understood in light of the specification and refers to carriers, excipients, and/or stabilizers that are nontoxic to the cell or mammal being exposed thereto at the dosages and concentrations employed or that have an acceptable level of toxicity. A “pharmaceutically acceptable” “diluent,” “excipient,” and/or “carrier” as used herein have their plain and ordinary meaning as understood in light of the specification and are intended to include any and all solvents, dispersion media, coatings, antibacterial or antifungal agents, isotonic or absorption delaying agents, compatible with administration to humans, cats, dogs, or other vertebrate hosts. Typically, a pharmaceutically acceptable diluent, excipient, and/or carrier is a diluent, excipient, and/or carrier approved by a regulatory agency of a Federal, a state government, or other regulatory agency, or listed in the U.S. Pharmacopeia or other generally recognized pharmacopeia for use in animals, including humans as well as non-human mammals, such as cats and dogs. The term diluent, excipient, and/or “carrier” can refer to a diluent, adjuvant, excipient, or vehicle with which the pharmaceutical composition is administered. Such pharmaceutical diluent, excipient, and/or carriers, which can be incorporated in any one or more of the compositions described herein, include sterile liquids, such as water and oils, including those of petroleum, animal, vegetable or synthetic origin. Water, saline solutions or aqueous dextrose and glycerol solutions can be employed as liquid diluents, excipients, and/or carriers. Suitable pharmaceutical diluents and/or excipients, which can be incorporated in any one or more of the compositions described herein, also include starch, glucose, lactose, sucrose, gelatin, malt, rice, flour, chalk, silica gel, sodium stearate, glycerol monostearate, talc, sodium chloride, dried skim milk, glycerol, propylene, glycol, water, or ethanol. The physiologically acceptable carrier may also comprise one or more of the following: antioxidants, such as ascorbic acid, low molecular weight (less than about 10 residues) polypeptides, proteins, such as serum albumin, gelatin, immunoglobulins, hydrophilic polymers such as polyvinylpyrrolidone, amino acids, carbohydrates such as glucose, mannose, or dextrins, chelating agents such as EDTA, sugar alcohols such as mannitol or sorbitol, salt-forming counterions such as sodium, and nonionic surfactants such as TWEEN®, polyethylene glycol (PEG), PLURONICS® or preservatives such as an essential oil, methyl paraben, propyl paraben, or sodium salt of parabens. Preferably, the preservative is bronidiol. The composition, if desired, can also contain minor amounts of wetting, bulking, emulsifying agents, or pH buffering agents. These compositions can take the form of solutions, suspensions, emulsion, sustained release formulations and the like. The formulation should suit the mode of administration.

Additional excipients with desirable properties include but are not limited to preservatives, adjuvants, stabilizers, solvents, buffers, diluents, solubilizing agents, detergents, surfactants, chelating agents, antioxidants, alcohols, ketones, aldehydes, ethylenediaminetetraacetic acid (EDTA), citric acid, salts, sodium chloride, sodium bicarbonate, sodium phosphate, sodium borate, sodium citrate, potassium chloride, potassium phosphate, magnesium sulfate sugars, dextrose, fructose, mannose, lactose, galactose, sucrose, sorbitol, cellulose, serum, amino acids, polysorbate 20, polysorbate 80, sodium deoxycholate, sodium taurodeoxycholate, magnesium stearate, octylphenol ethoxylate, benzethonium chloride, thimerosal, gelatin, esters, ethers, 2-phenoxyethanol, urea, or vitamins, or any combination thereof. Some excipients may be in residual amounts or contaminants from the process of manufacturing, including but not limited to serum, albumin, ovalbumin, antibiotics, inactivating agents, formaldehyde, glutaraldehyde, β-propiolactone, gelatin, cell debris, nucleic acids, peptides, amino acids, or growth medium components or any combination thereof. The amount of the excipient may be found in the composition at a percentage that is, is about, is at least, is at least about, is not more than, or is not more than about, 0%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 20%, 30%, 40%, 50%, 60%, 70%, 80%, 90%, 95%, 100% w/w or any percentage by weight in a range defined by any two of the aforementioned numbers.

As used herein, a “carrier” has its plain and ordinary meaning as understood in light of the specification and refers to a compound, particle, solid, semi-solid, liquid, or diluent that facilitates the passage, delivery and/or incorporation of a compound to cells, tissues and/or bodily organs.

As used herein, a “diluent” has its plain and ordinary meaning as understood in light of the specification and refers to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

Administered “in combination,” as used herein, means that two (or more) different compositions are delivered to the subject during the course of the subject's affliction with the disorder, e.g., the two or more compositions are delivered after the subject has been diagnosed or selected as one having the disorder and before the disorder has been cured or eliminated. In some embodiments the subject is selected to receive any one or more of the compositions described herein by diagnostic analysis or clinical evaluation or both. For instance, in some embodiments, a subject is screened to determine whether said subject lacks one or more beneficial bacteria or has a reduced amount of said one or more beneficial bacteria prior to receiving an administration of any one or more of the compositions described herein. In some embodiments, the delivery of one therapy is still occurring when the delivery of the second begins, so that there is overlap. This is sometimes referred to herein as “simultaneous” or “concomitant” or “concurrent delivery”. In other embodiments, the delivery of one therapy ends before the delivery of the other therapy begins. This is sometimes referred to herein as “successive” or “sequential delivery.” In embodiments of either case, the therapy is more effective because of combined administration. For example, the second therapy is a more effective, e.g., an equivalent effect is seen with less of the second therapy, or the second therapy reduces symptoms to a greater extent, than would be seen if the second therapy were administered in the absence of the first therapy, or the analogous situation is seen with the first therapy. In some embodiments, delivery is such that the reduction in a symptom, or other parameter related to the disorder is greater than what would be observed with one therapy delivered in the absence of the other. The effect of the two therapies can be partially additive, wholly additive, or greater than additive (e.g., synergistic). The delivery can be such that an effect of the first therapy delivered is still detectable when the second is delivered.

“Tumor,” as used herein, refers to all neoplastic cell growth and proliferation, whether malignant or benign, and all pre-cancerous and cancerous cells and tissues. The terms “cancer,” “cancerous,” “cell proliferative disorder,” “proliferative disorder” and “tumor” are not mutually exclusive as referred to herein. The term “neoplasia” encompasses the term tumor.

The terms “cancer” and “cancerous” refer to or describe the physiological condition in mammals that is typically characterized by unregulated cell growth. Examples of cancer include, but are not limited to, carcinoma, lymphoma, blastoma, sarcoma, and leukemia or lymphoid malignancies. More particular examples of such cancers include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, bone cancer, hepatic carcinoma, anal carcinoma, penile carcinoma, melanoma, multiple myeloma and B-cell lymphoma, brain, as well as head and neck cancer, and associated metastases. The term cancer includes adult and pediatric solid cancers. In some embodiments, the cancer can be a solid tumor. In some embodiments, the cancer is a highly fibrotic tumor or cancer. In some embodiments, the cancer is a desmoplasia.

### Non-Limiting Examples of Novel Antigen Binding Construct Sequences and Properties Thereof

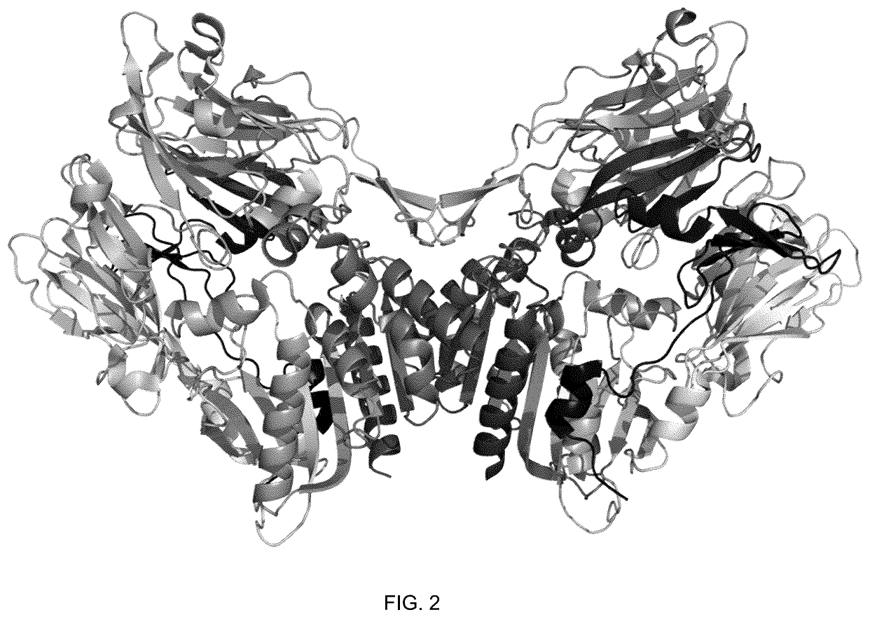

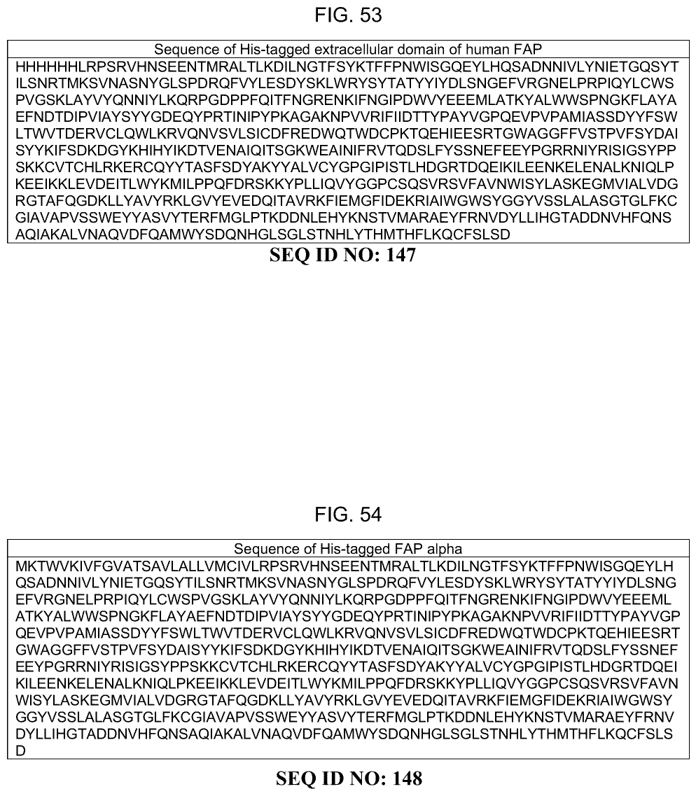

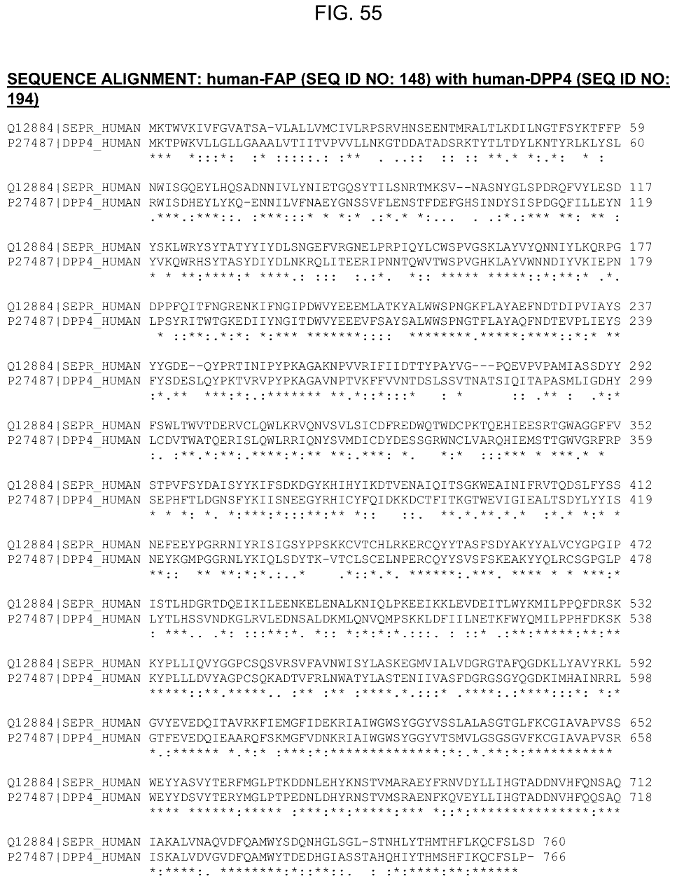

FAP has a molecular weight of 170 kDa and consists of two 97 kDa glycoprotein subunits (FIGS. 2 and 55). Non-limiting examples of FAP sequences are given in FIGS. 53-54. Based on its amino acid homology, FAP is most similar to DPP4 (FIG. 55). The major difference between these two enzymes is that FAP has an Ala657 while DPP4 contains Asp663 at their respective active sites.

Expression of FAP varies by organism and tissue type. FAP appears to be conserved among chordates, with especially high homology in mammals. FAP is usually absent in normal adult tissues. The soluble and enzymatically active form of FAP, antiplasmin-cleaving enzyme (APCE), circulates in human plasma. FAP is expressed on reactive stromal fibroblasts in tumor tissue and wound healing and on synoviocytes in rheumatoid arthritis. The expression of FAP is weak in the cervix and the uterine stroma, although expression reaches the highest levels during the proliferative phase. FAP is also present in multipotent bone marrow stromal cells (BM-MSC) in both mice and humans. It has also been detected in the human placenta and some cases in dermal fibroblasts surrounding hair follicles. Furthermore, FAP expression may change under a variety of pathological conditions, including liver cirrhosis, rheumatoid arthritis, tissue remodeling, tumor formation, and tumor-associated stromal cells.

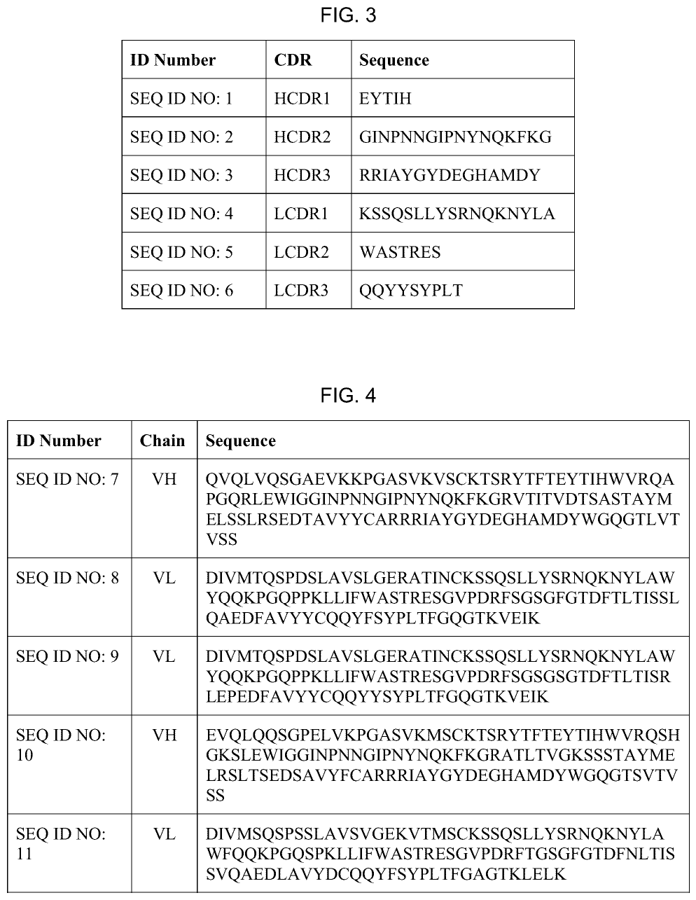

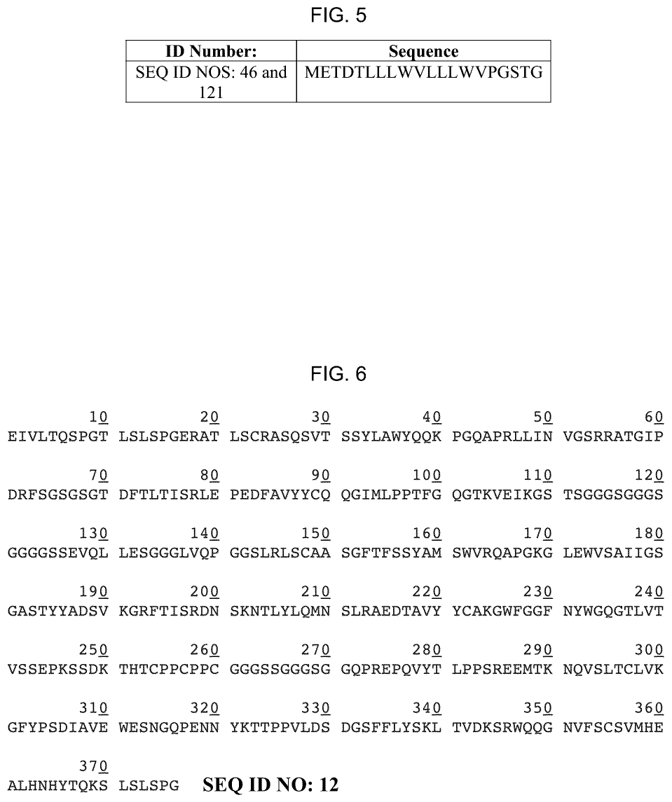

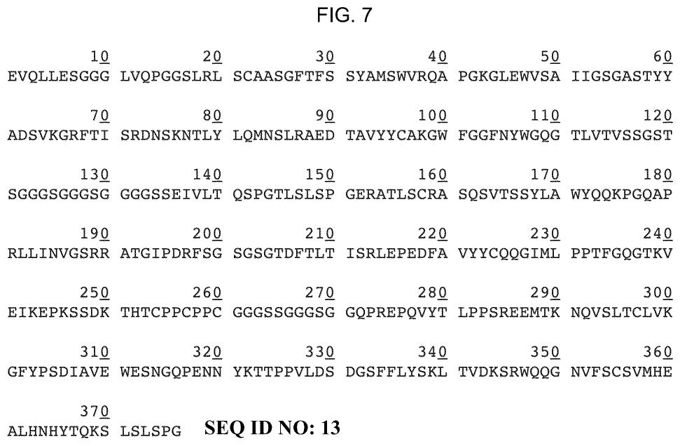

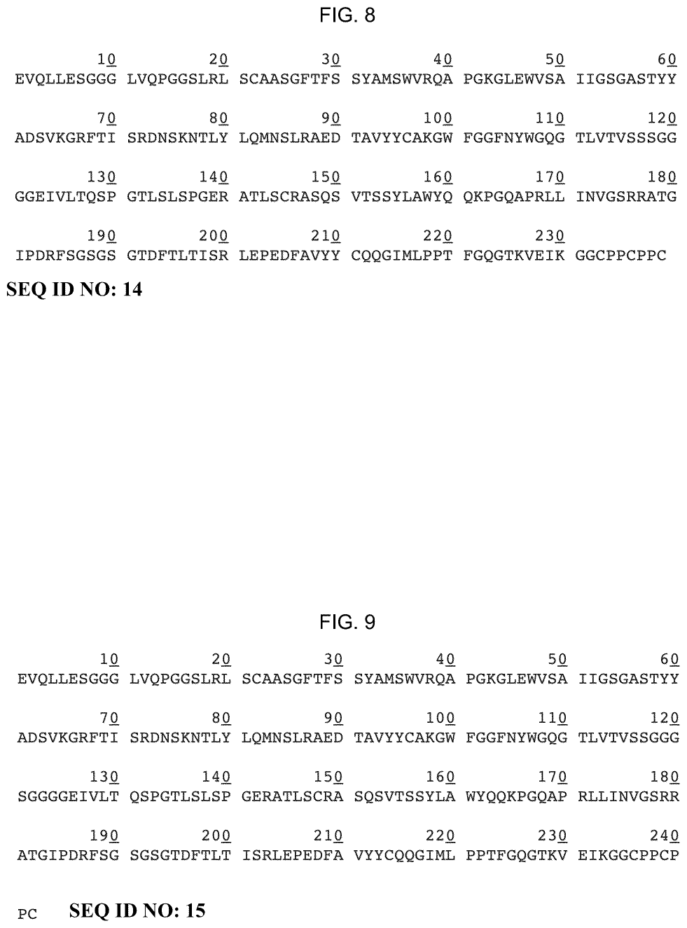

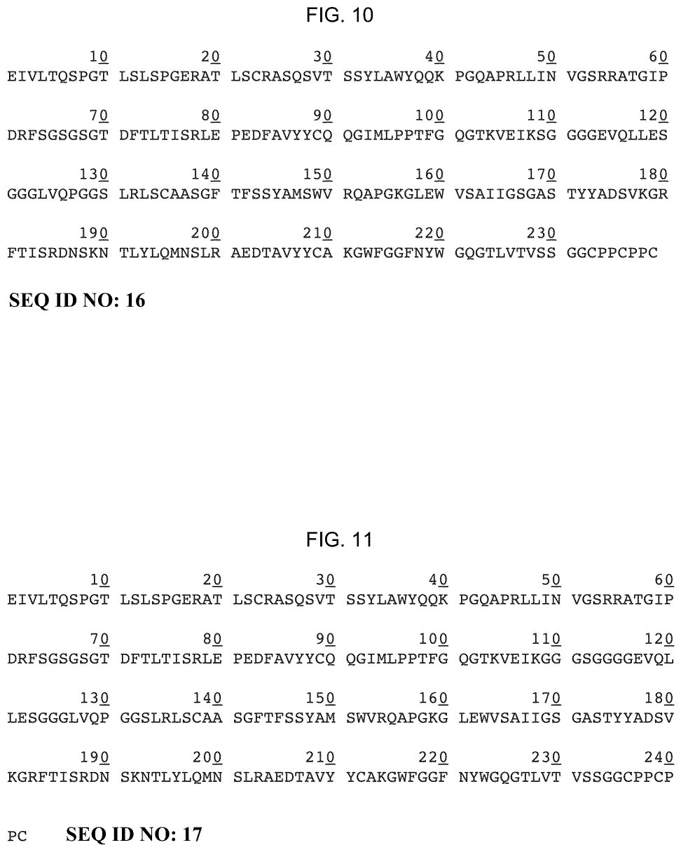

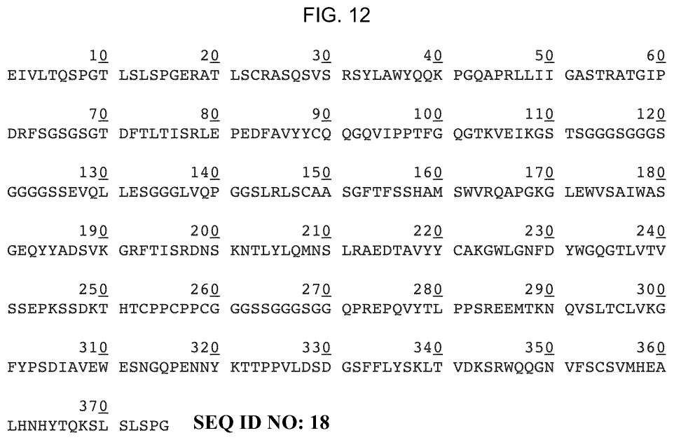

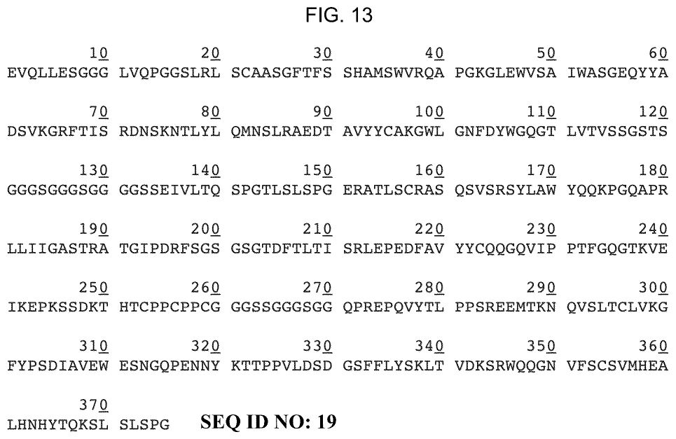

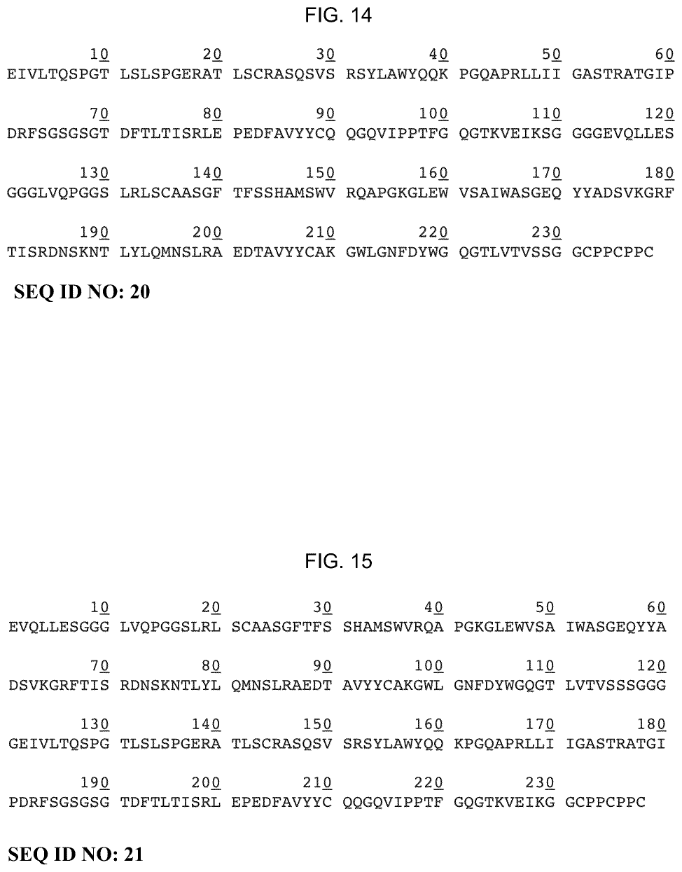

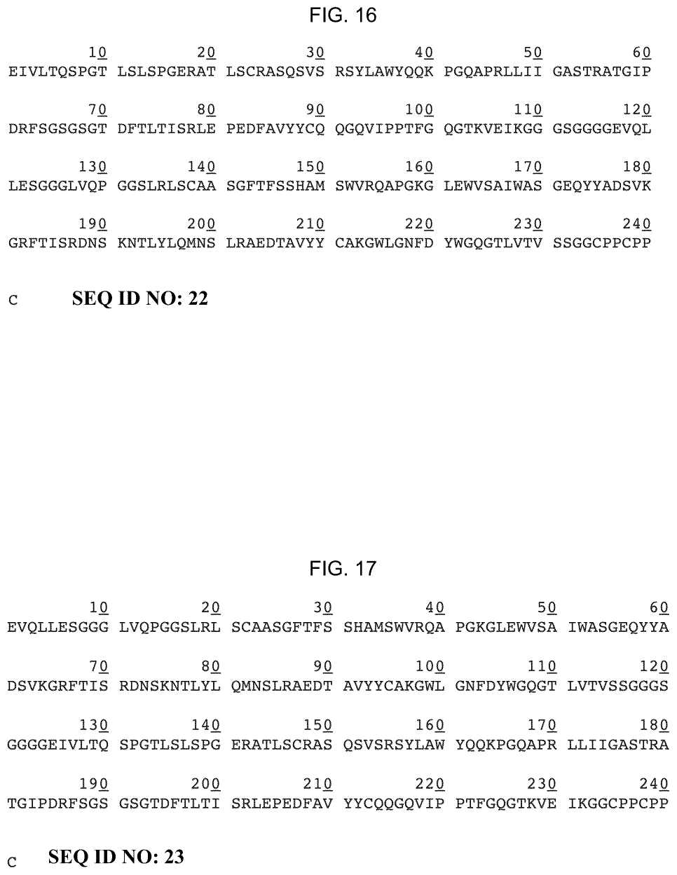

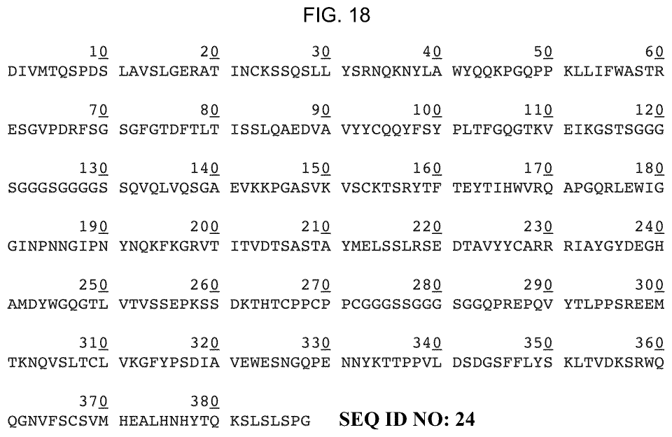

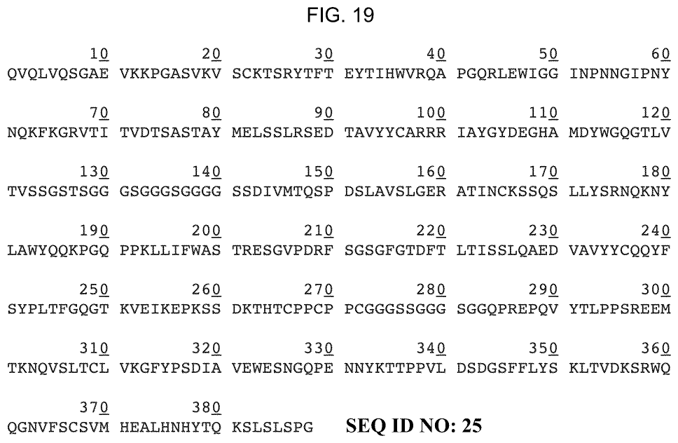

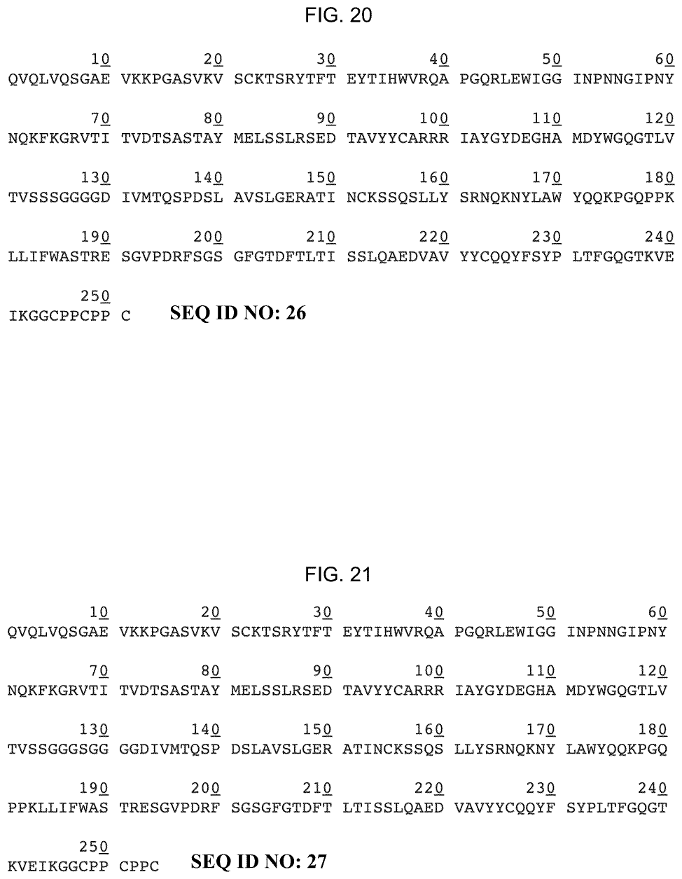

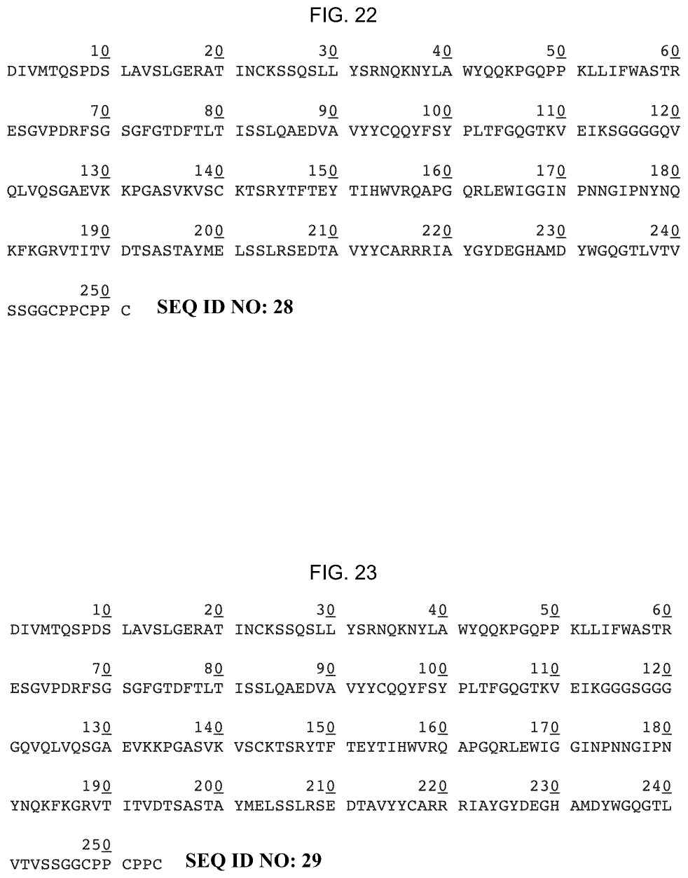

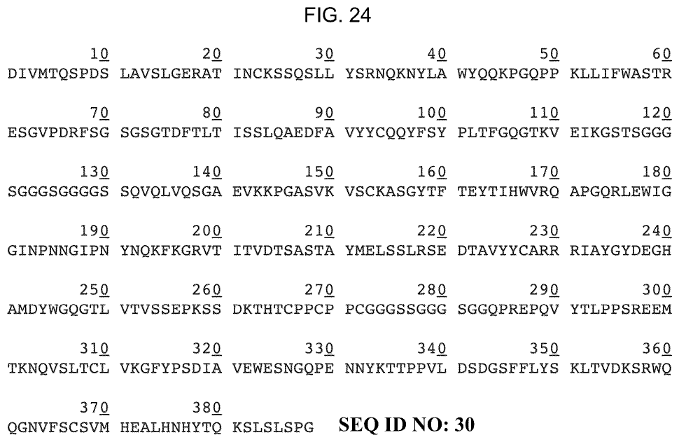

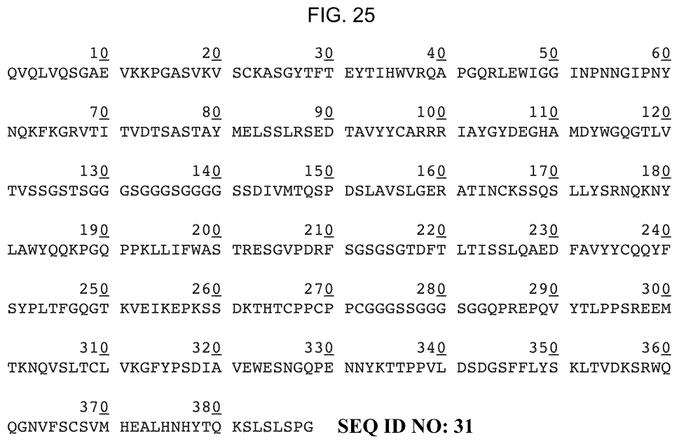

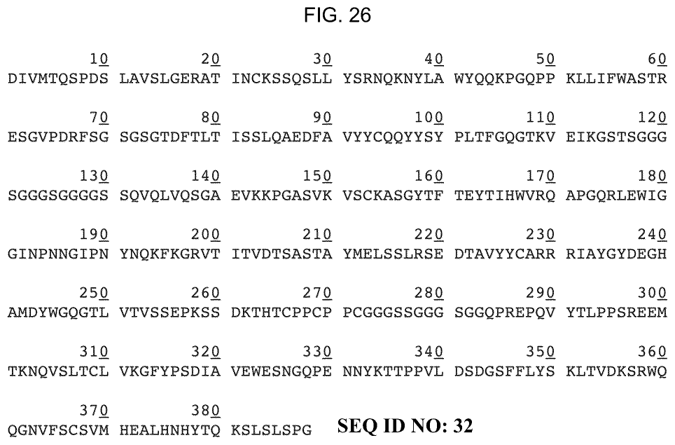

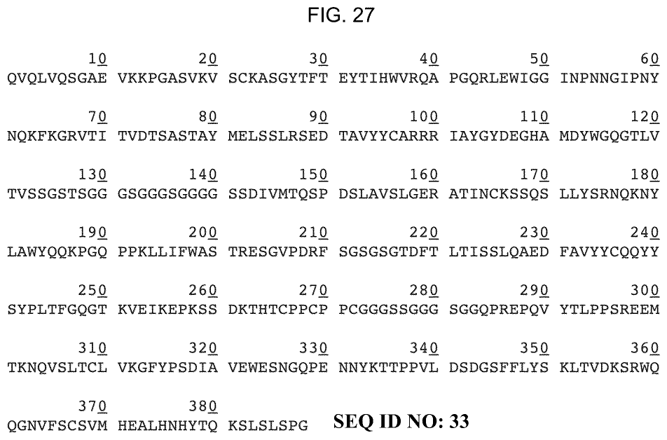

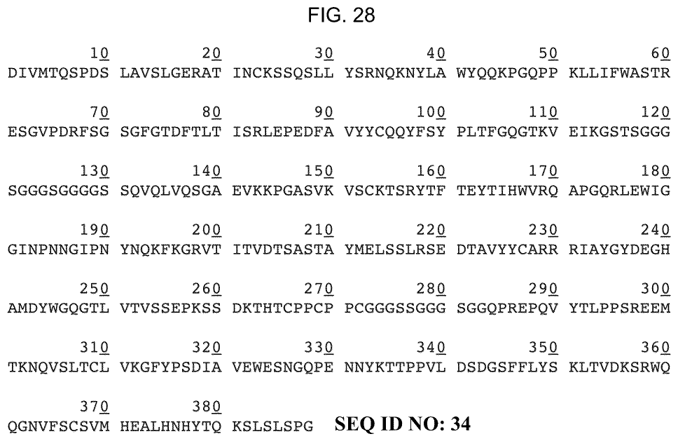

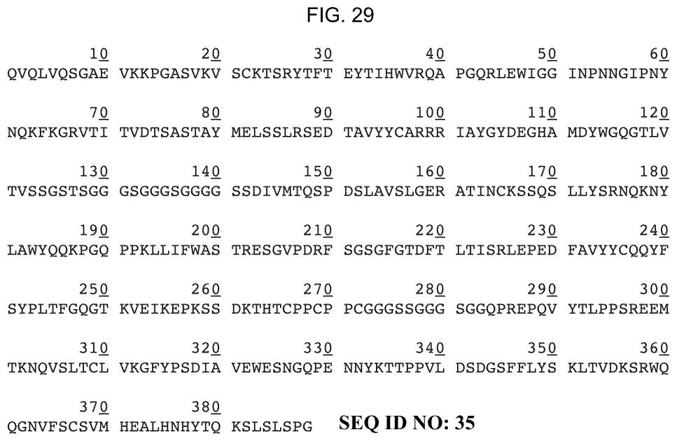

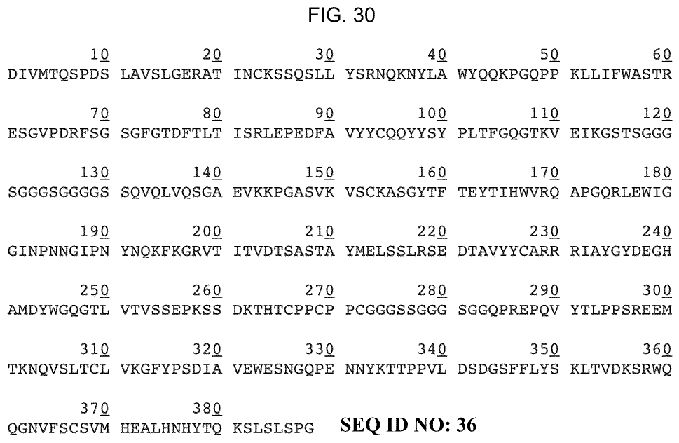

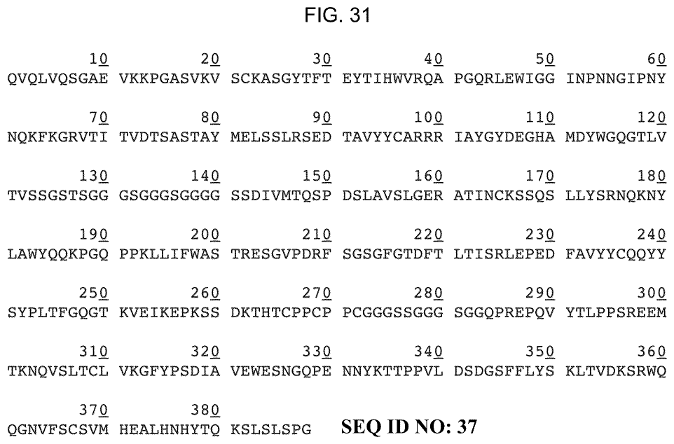

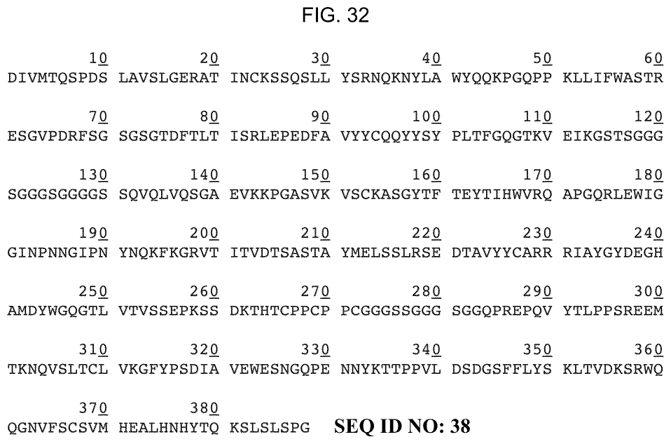

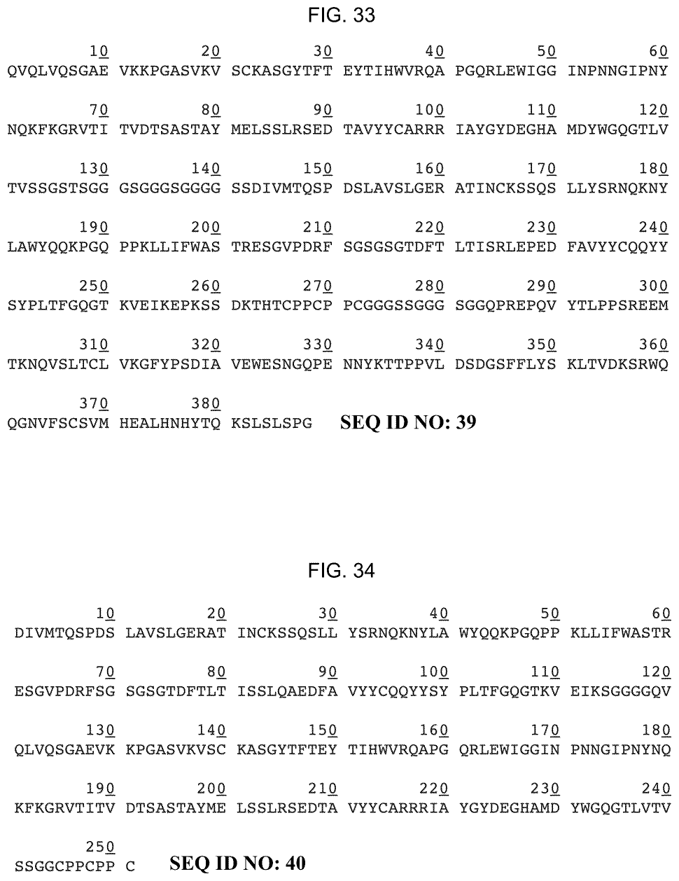

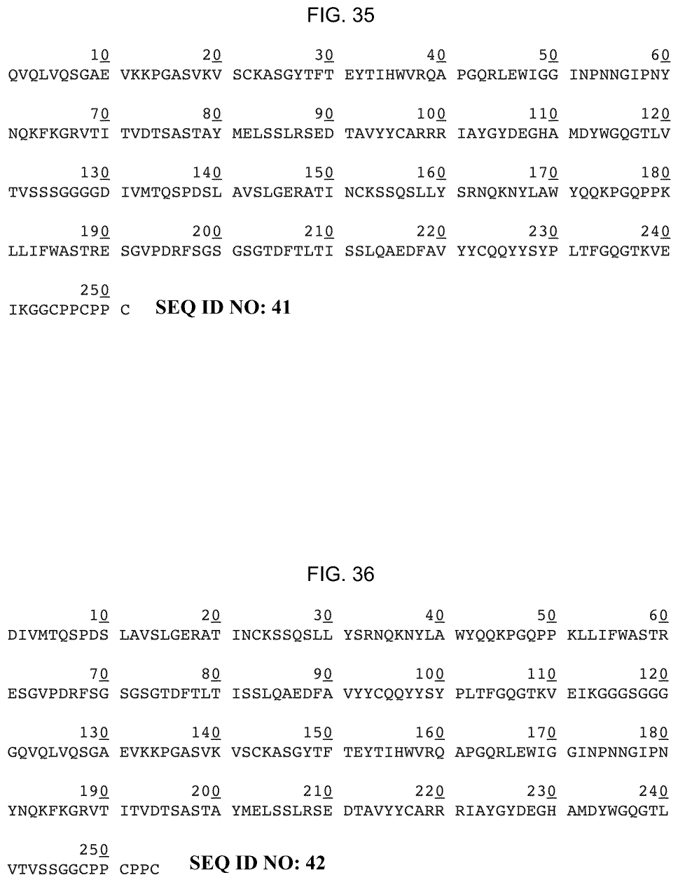

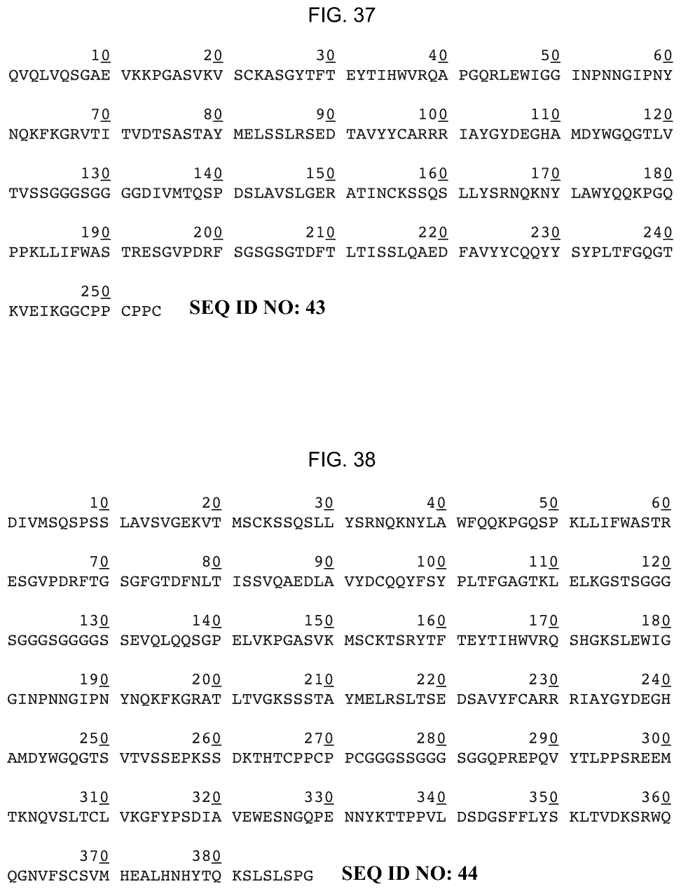

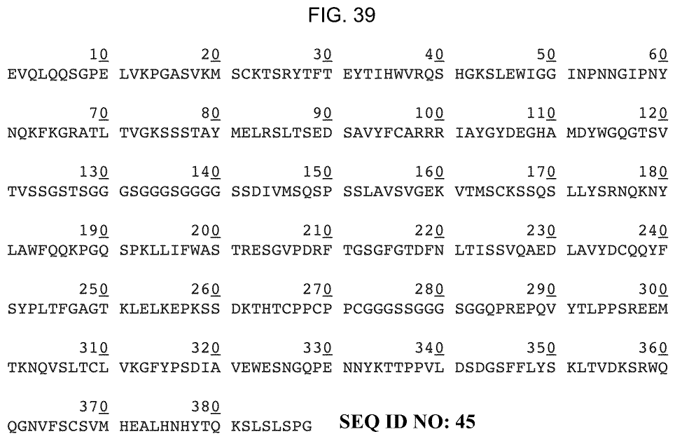

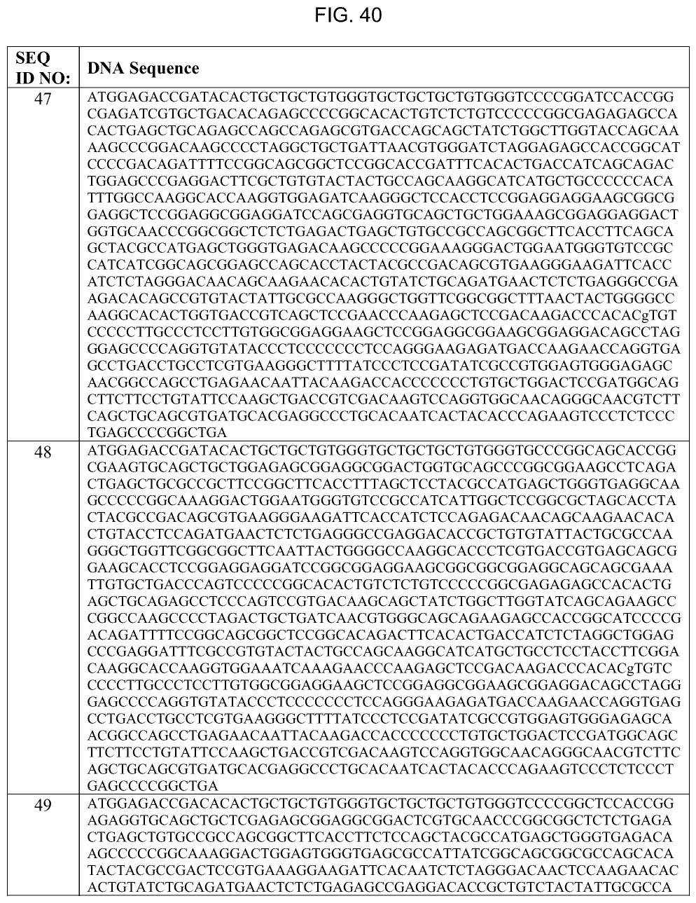

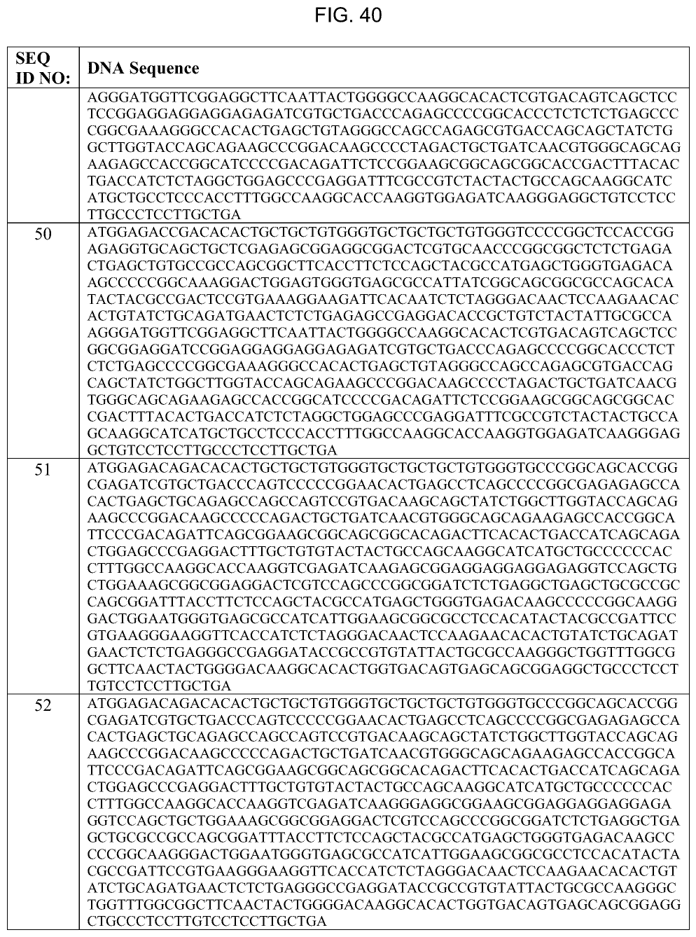

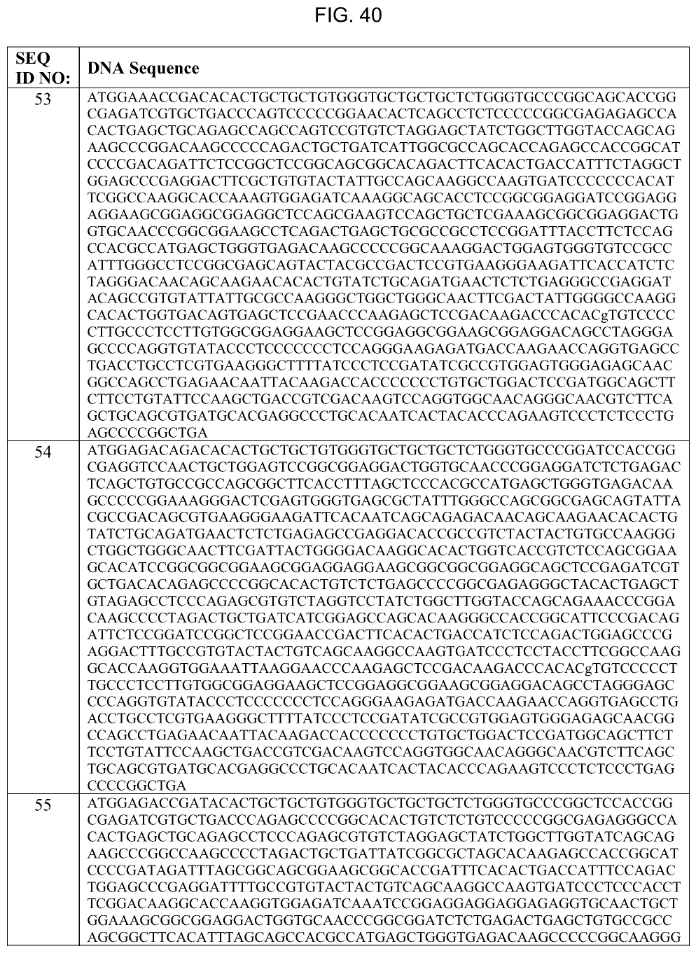

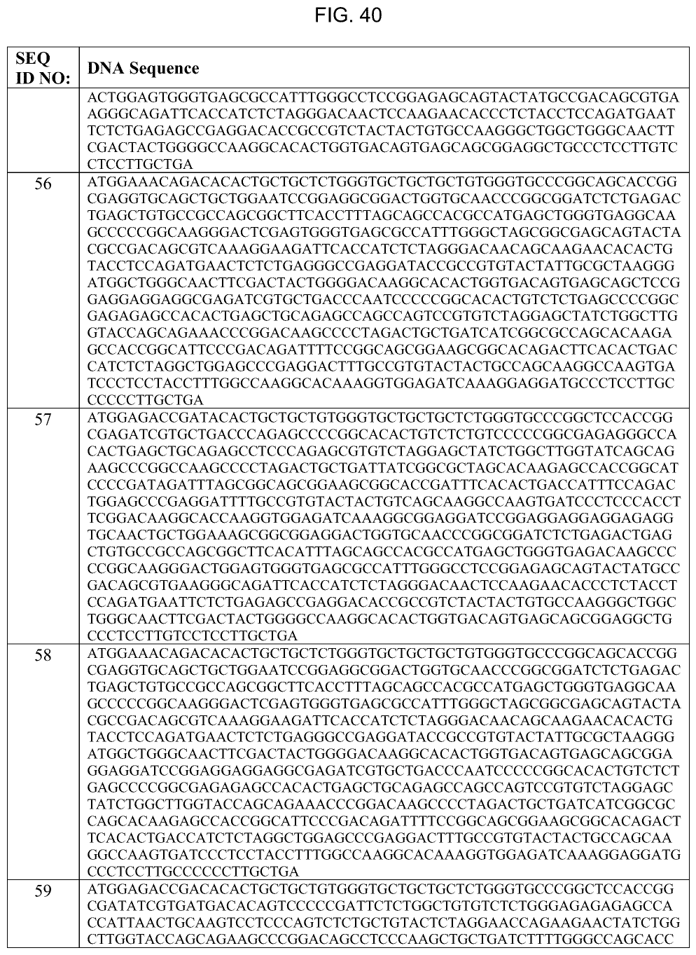

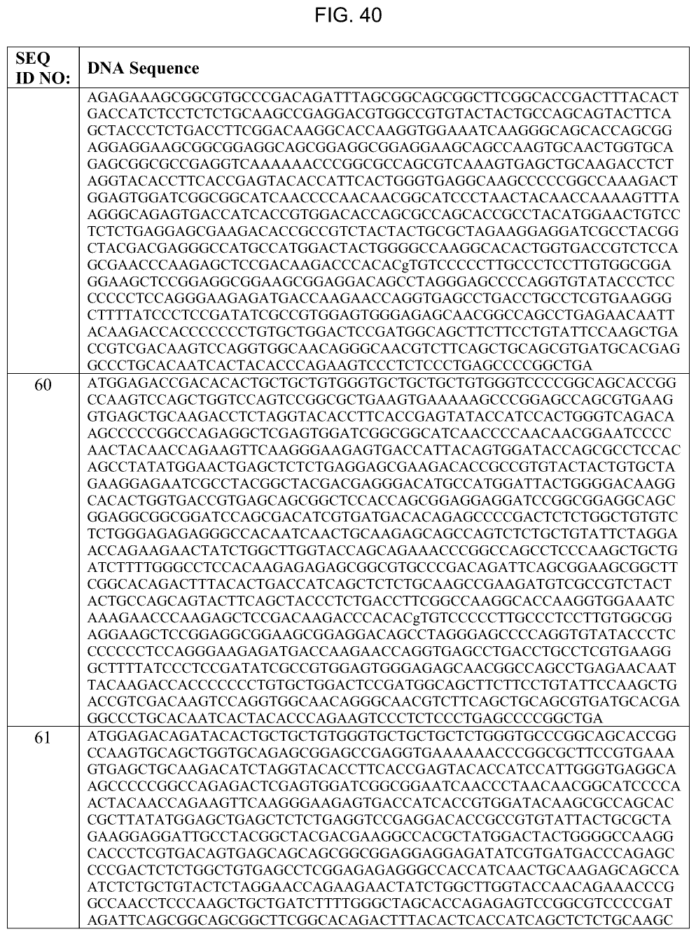

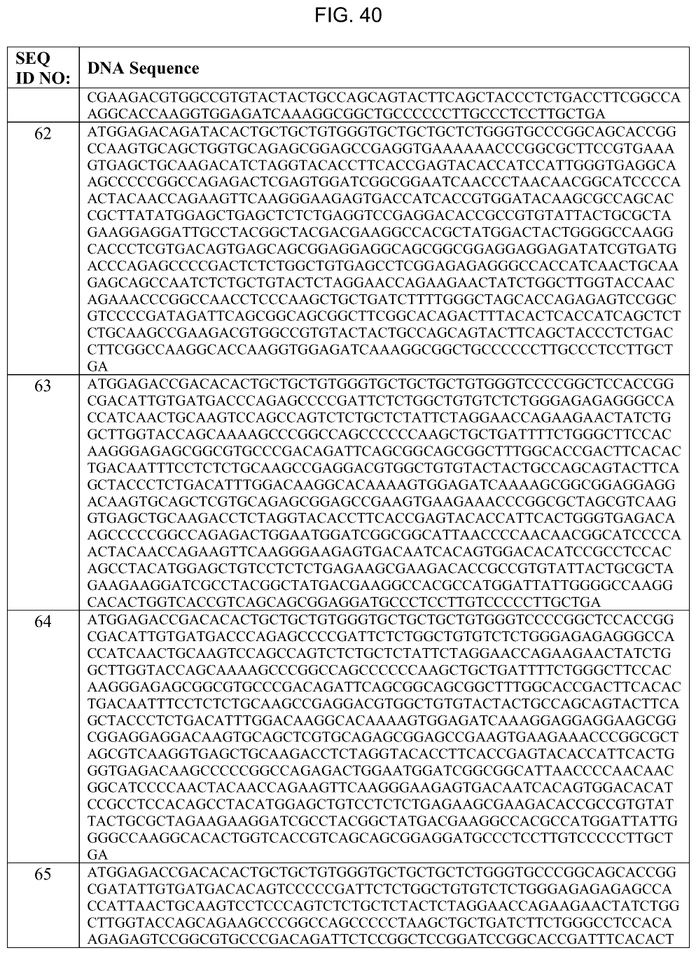

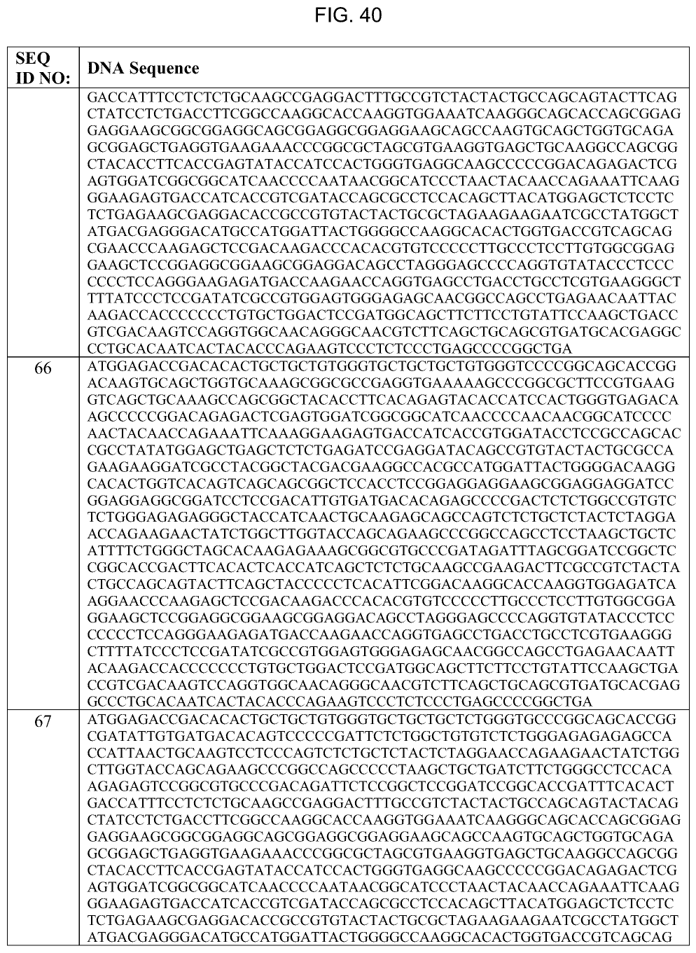

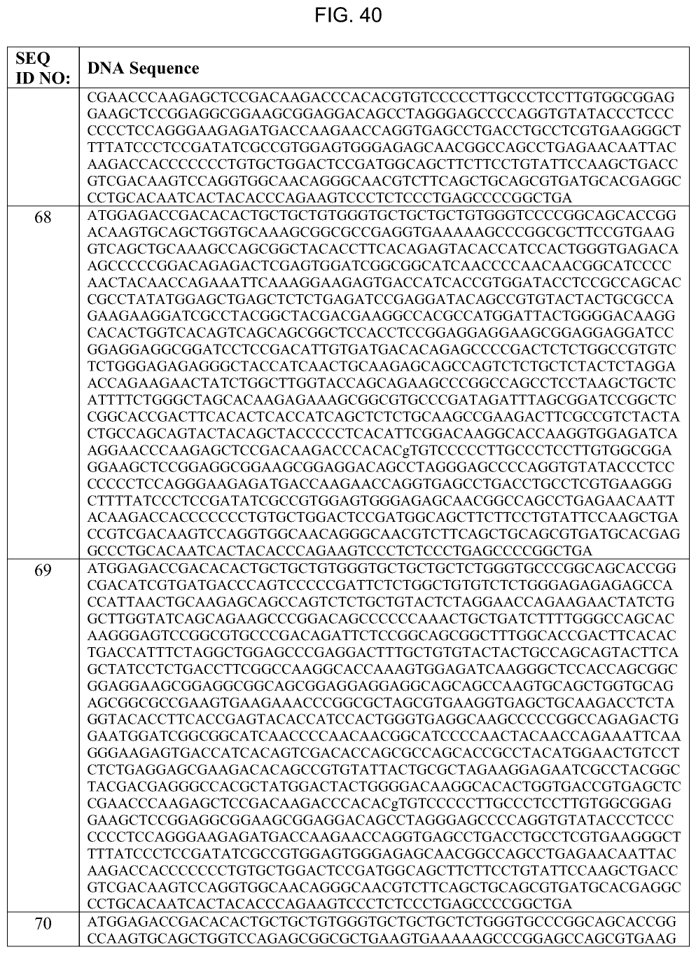

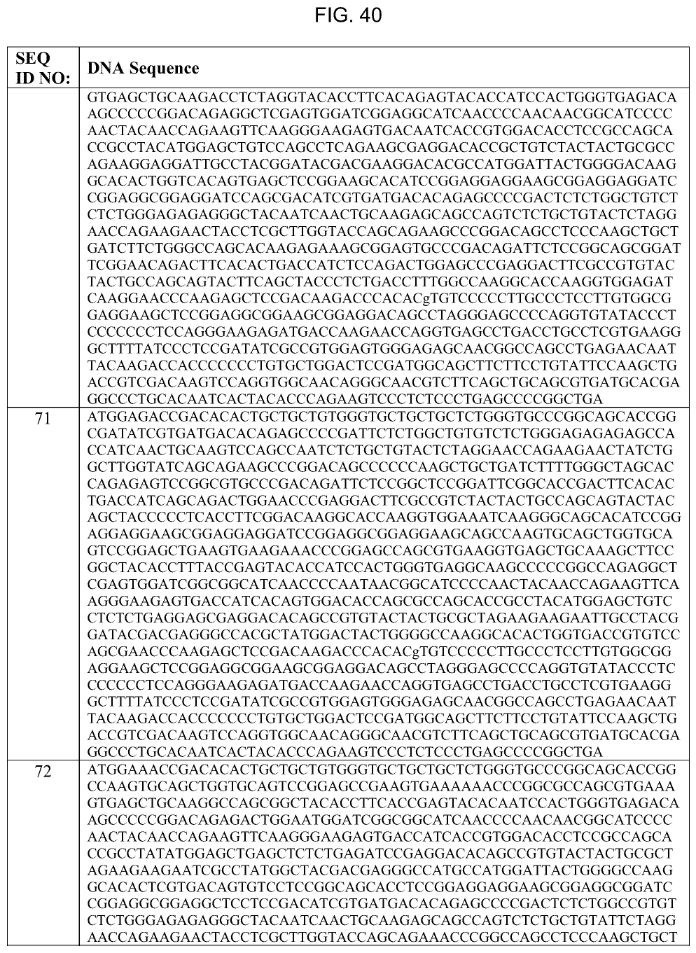

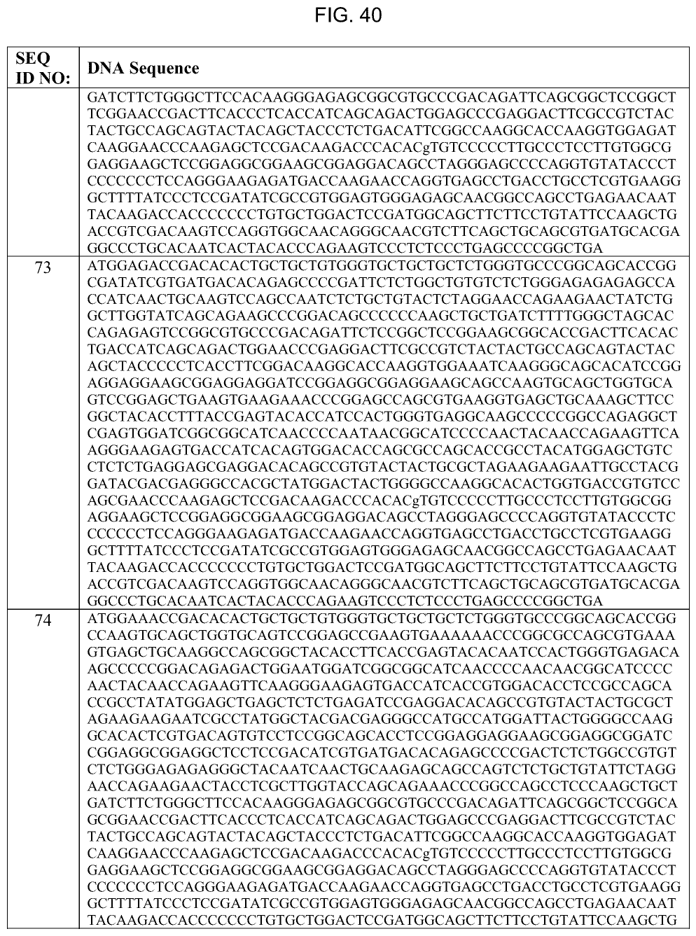

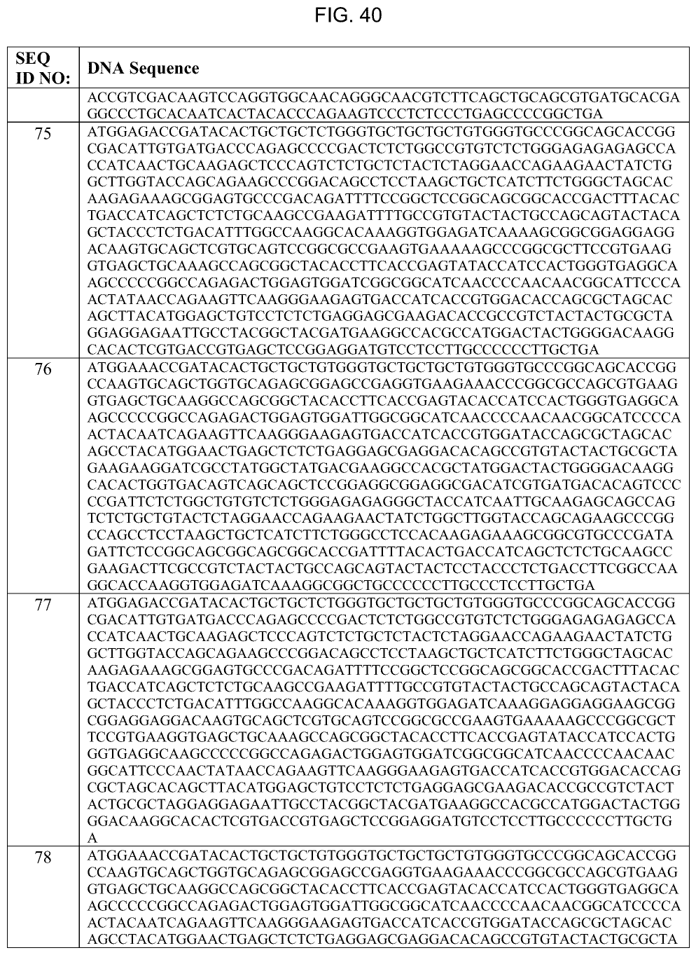

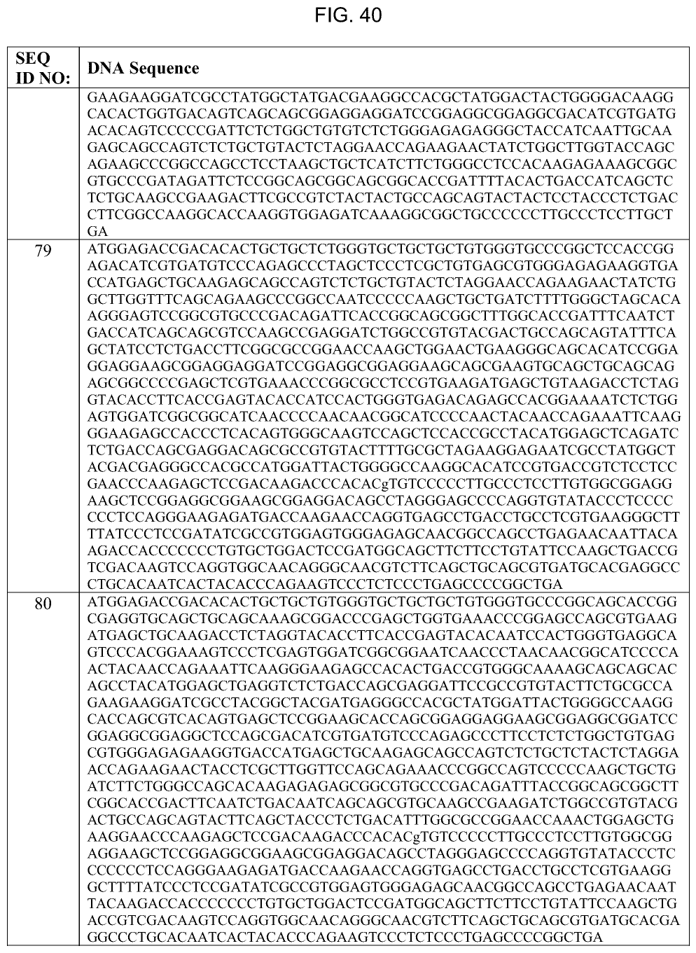

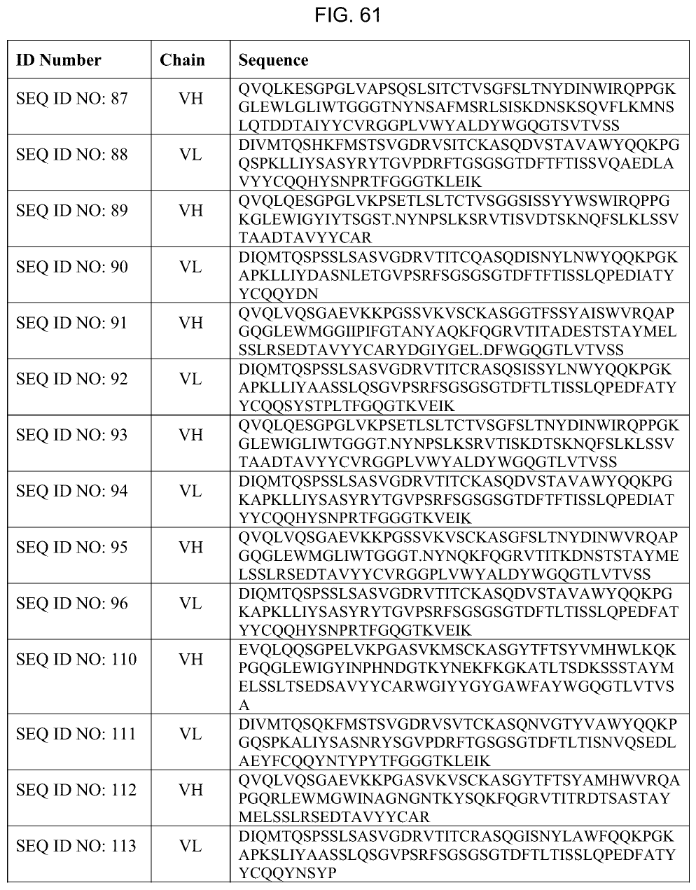

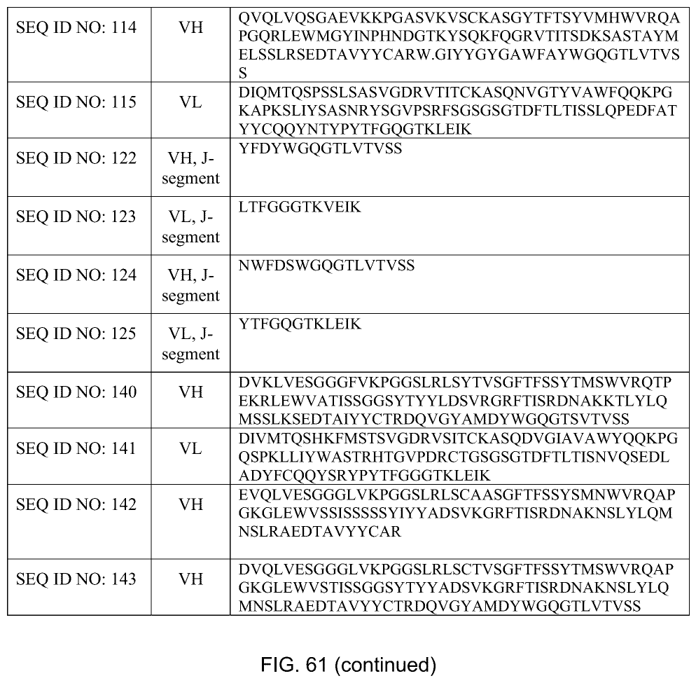

Disclosed herein are novel antigen binding constructs that target FAP. Non-limiting examples of amino acid sequences for the novel antigen binding constructs are given in FIGS. 6-39, . 62-84. Non-limiting examples of nucleic acid sequences for the novel antigen binding constructs are given in FIG. 40. Non-limiting examples of CDR, VH, and VL sequences are given in FIGS. 3-4, 60-61. It will be understood that any of the CDR, VH, and VL regions, alone or in any combination, can be used. In some alternatives, the construct is expressed in a compatible cell with or without further expression sequences, such as a constitutive promoter, inducible promoter, detectable marker, splicing factor, resistance gene, and/or cleavable sequence (FIG. 5). It will be understood that any cell, cell line, tissue, organ, or organ system capable of expressing the antigen binding construct may be used; preferably, a mammalian cell or cell line; most preferably, a human or rodent cell or cell line.

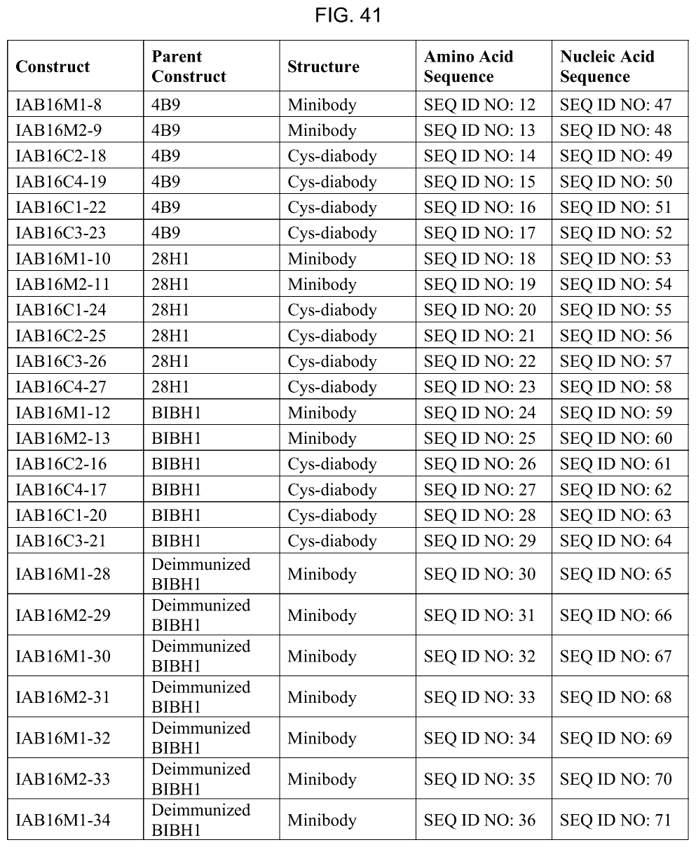

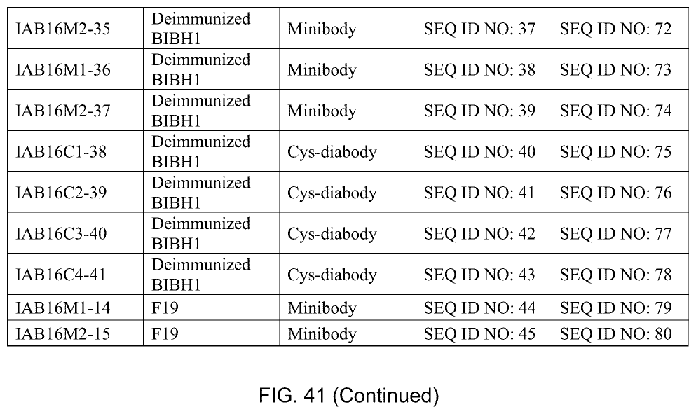

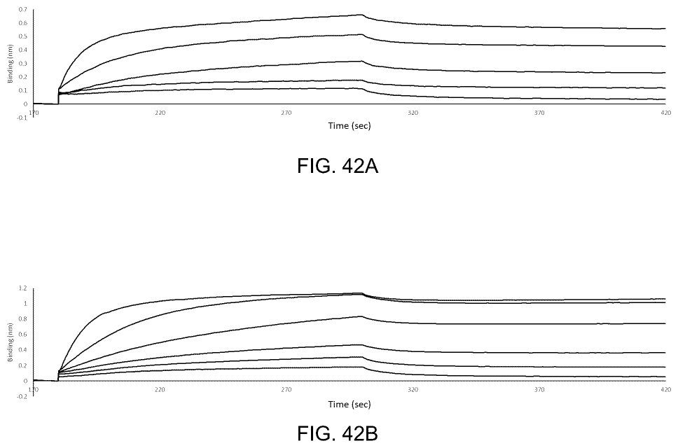

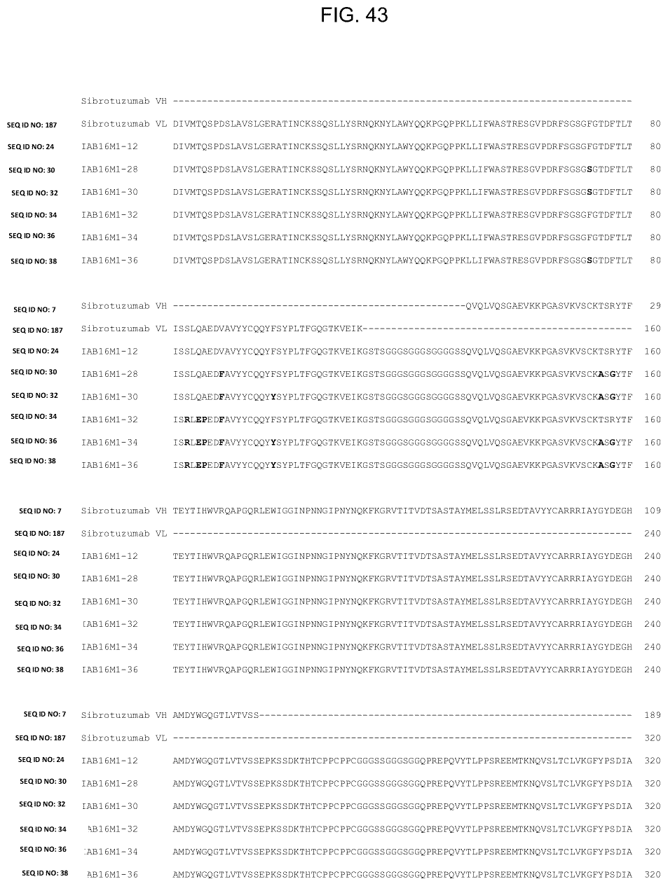

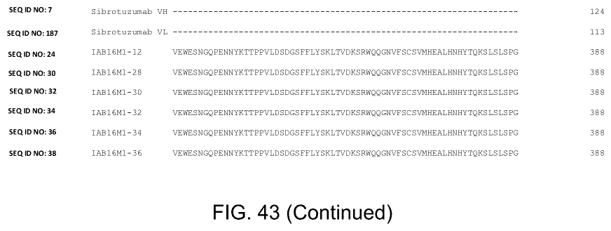

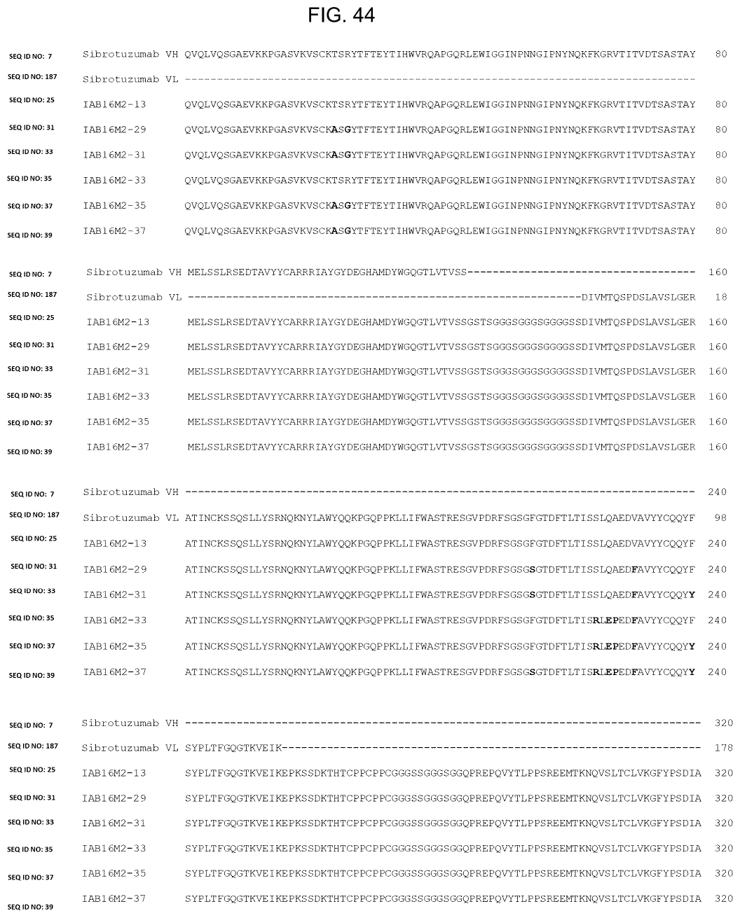

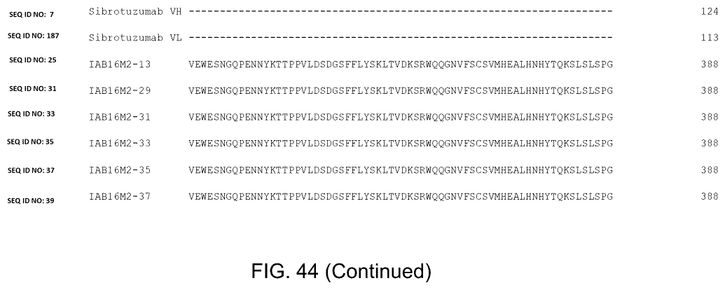

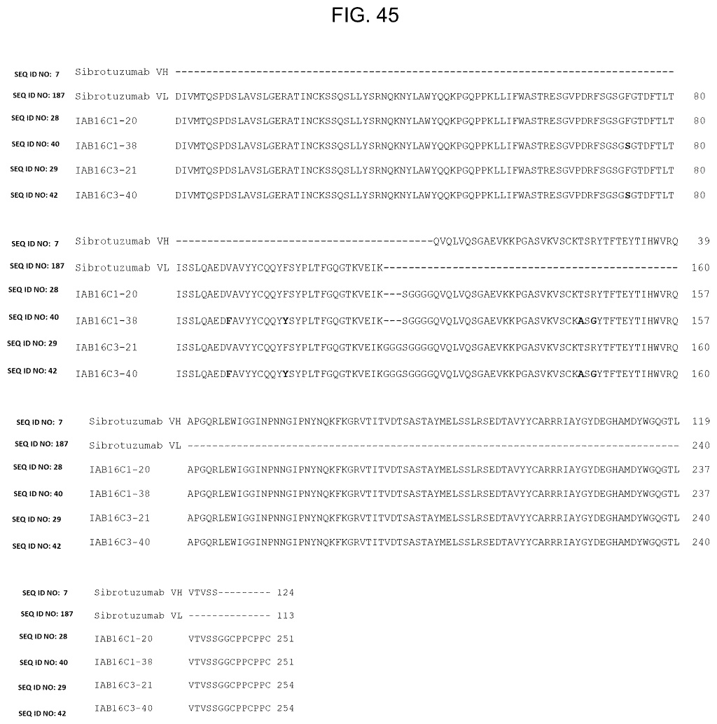

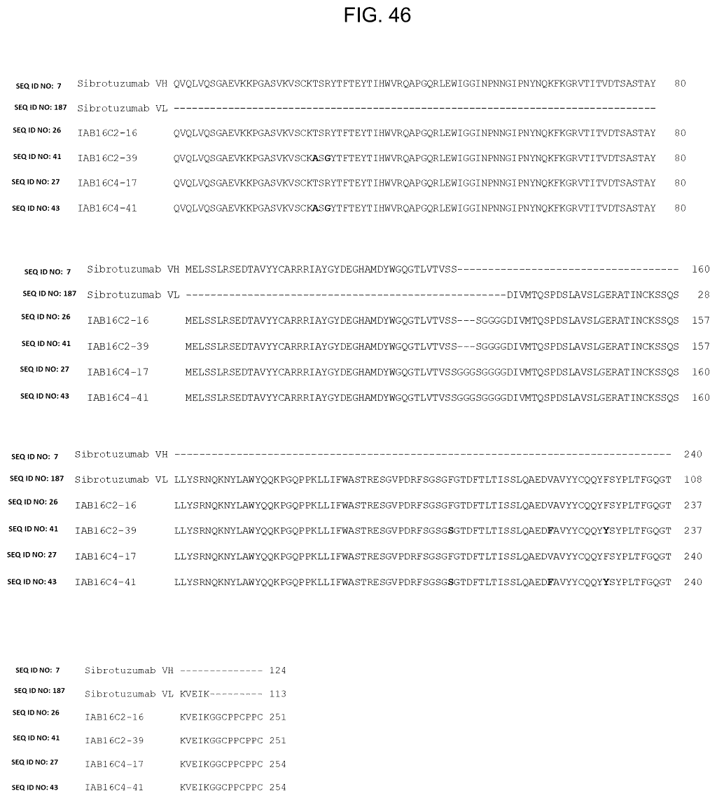

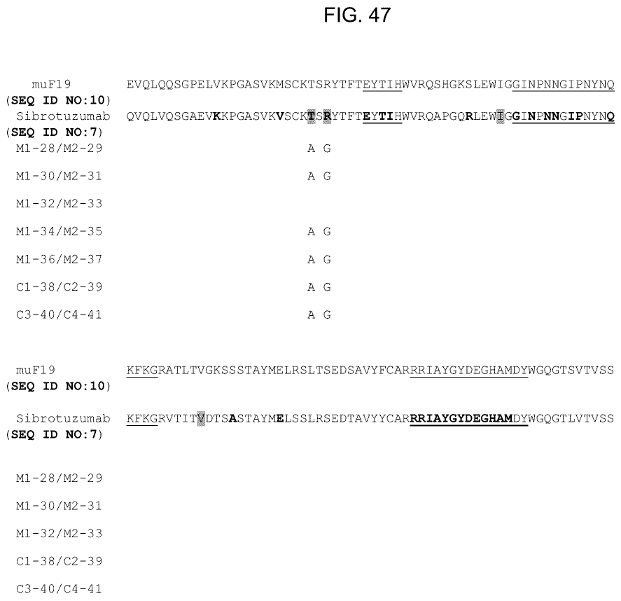

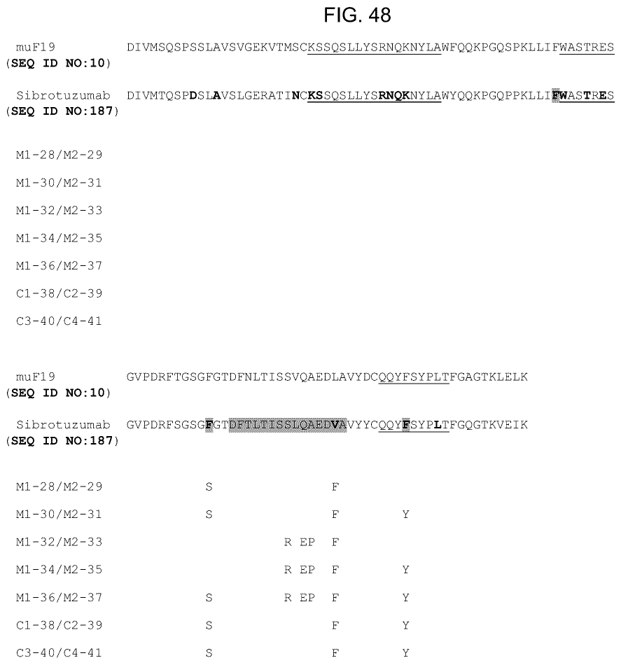

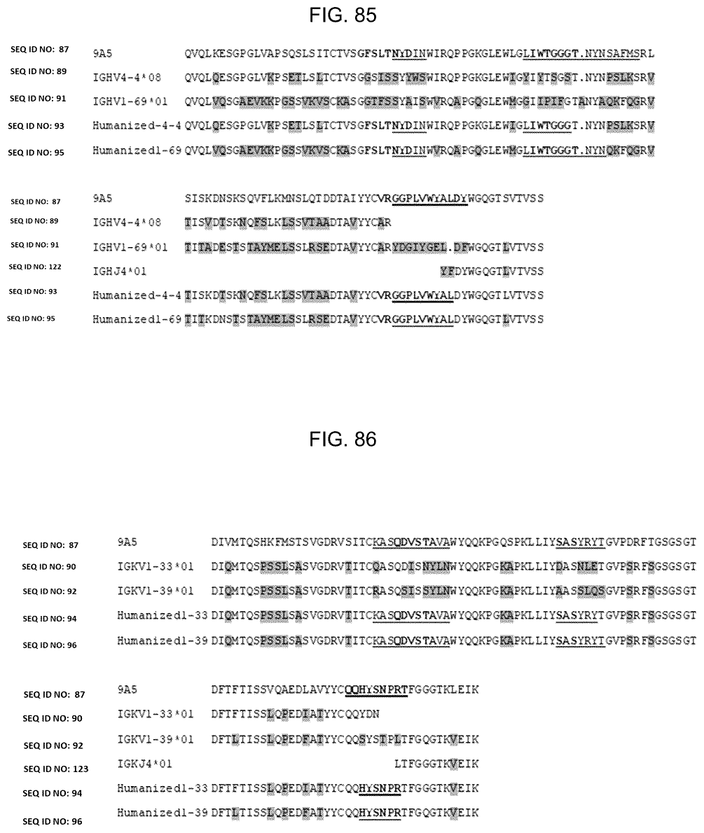

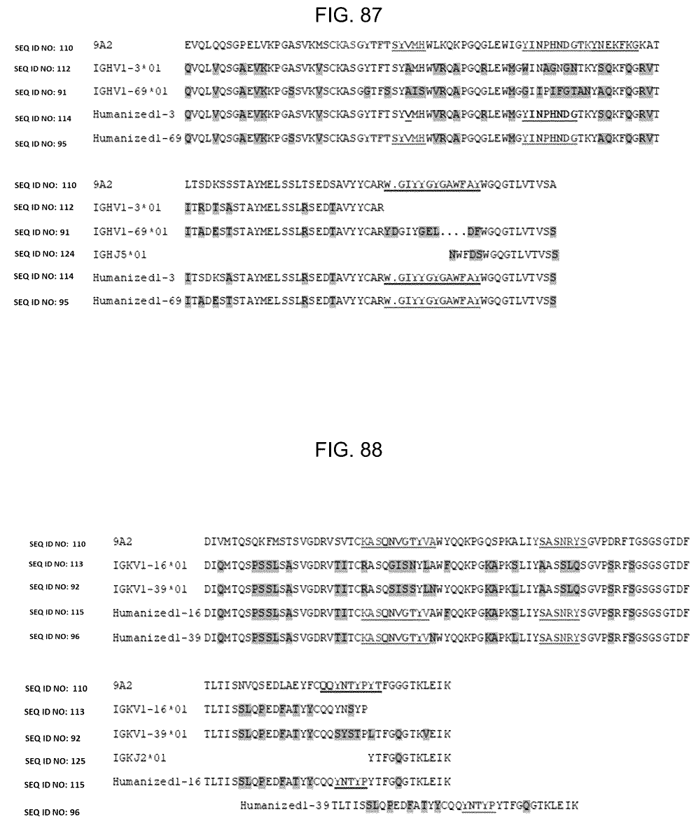

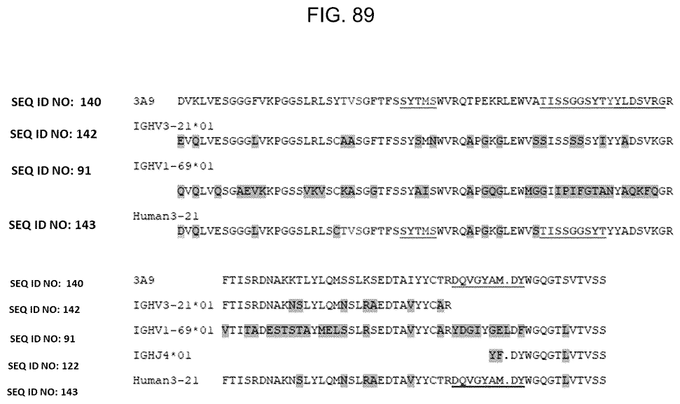

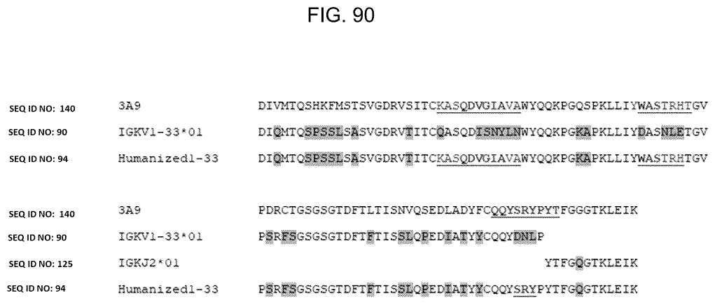

As disclosed herein, the novel antigen binding constructs are derived from parental constructs. In some alternatives, the novel antigen binding construct is a minibody. In some alternatives, the novel antigen binding construct is a cys-diabody. Non-limiting examples of novel antigen binding constructs, their parental antibody construct, and their corresponding sequence ID NOS as referenced herein are given in FIG. 41. Examples of alignments between novel antigen binding constructs and parental antigen binding constructs are given in FIGS. 47-48. Examples of alignments between novel antigen binding constructs and known antibody sequences are given in FIGS. 42-46, and 85-90.

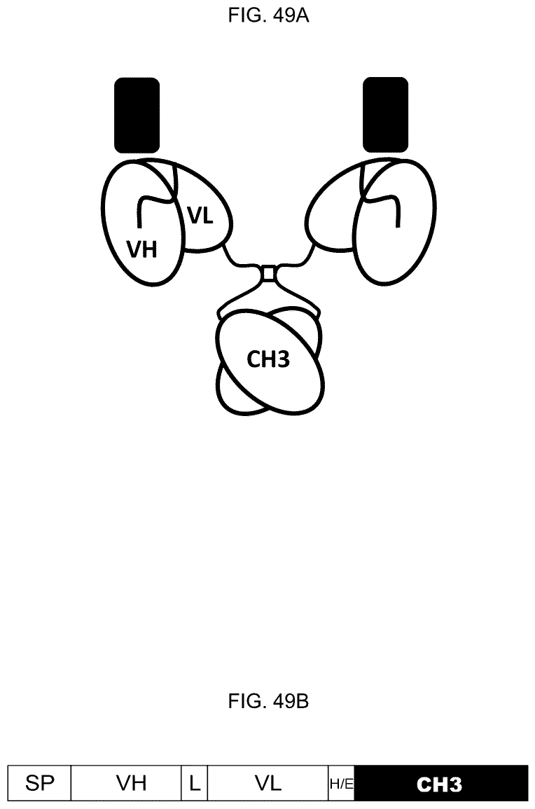

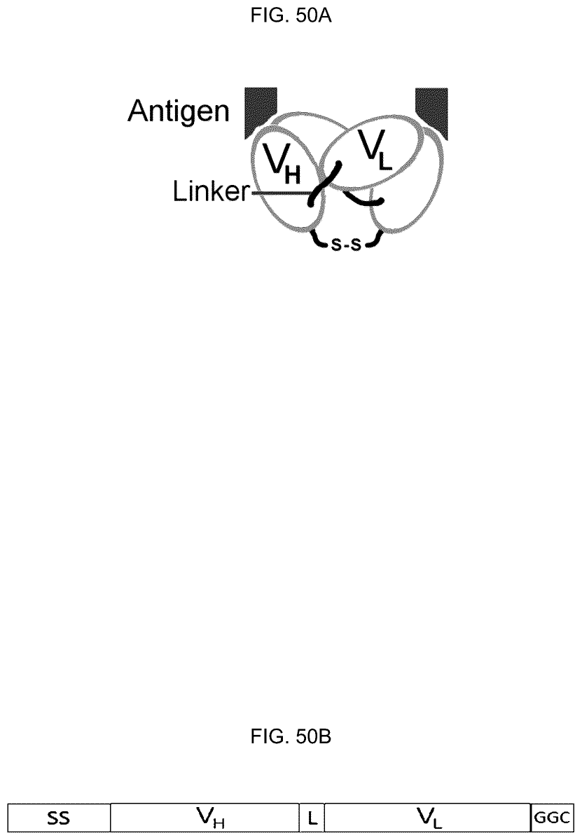

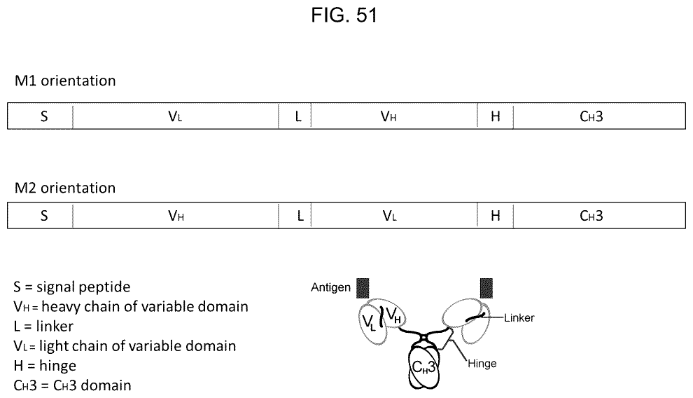

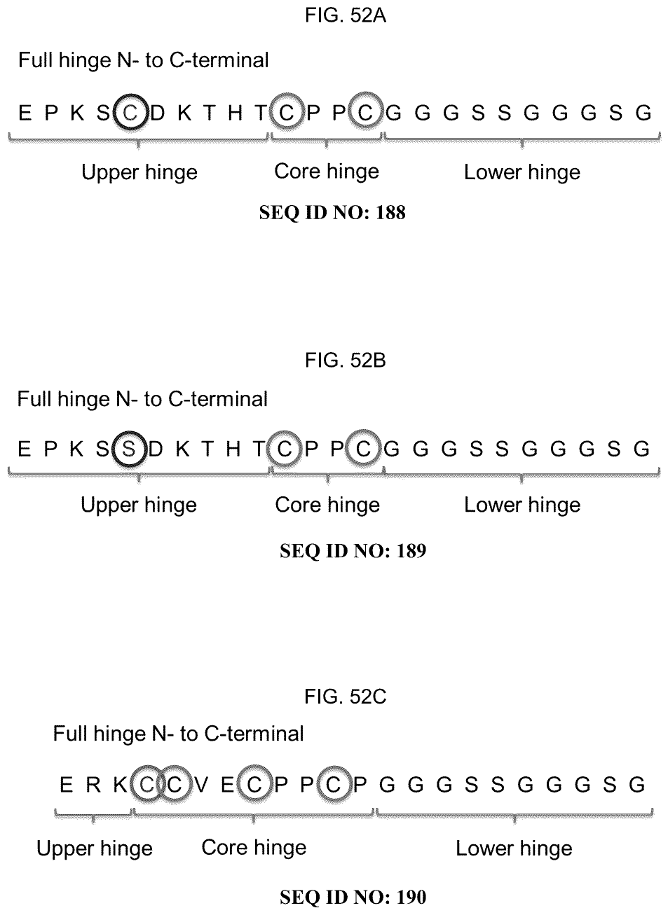

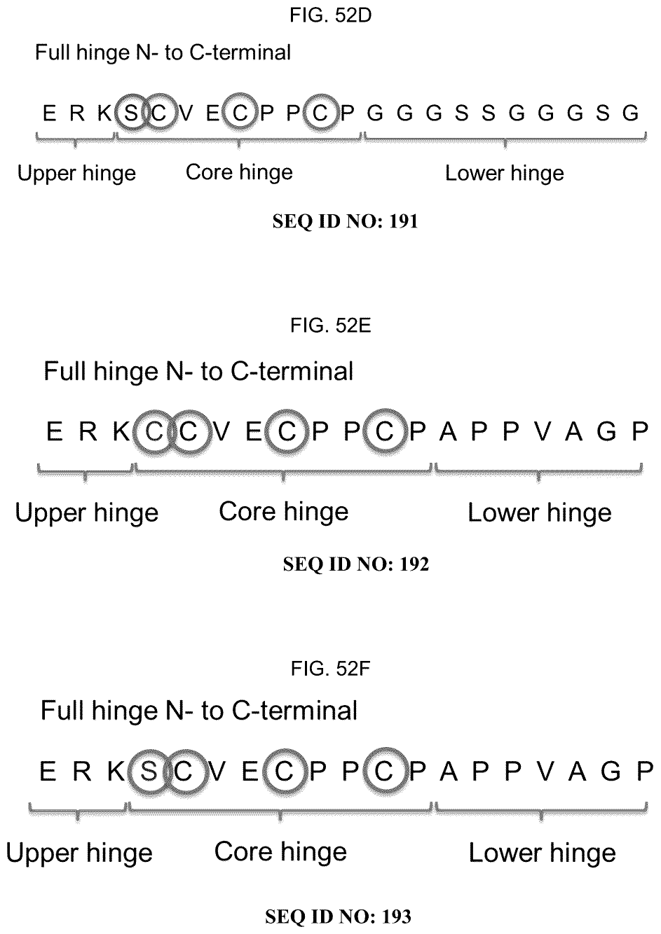

In some alternatives, the novel antigen binding construct comprises at least one VH domain, VL domain, a hinge domain, a linker domain, or any combination thereof (FIGS. 47-51). In some alternatives, the novel antigen binding construct additional comprises at least one signal peptide, CH3 domain, or any combination thereof. Non-limiting examples of hinge sequences are given in FIGS. 52A-52F. In some alternatives, the hinge sequence comprises one or more of an upper hinge region, core hinge region, and/or lower hinge region. It will be understood that any of the disclosed hinge sequences can be used in combination with any of the CDR, VH, and/or VL sequences provided herein. Furthermore, any of the any of the CDR, VH, and/or VL sequences provided herein may be used with or without any of the hinge sequences provided herein in any of the constructs/methods disclosed herein.

As disclosed herein, antigen binding constructs are designed with specific sequences. In some embodiments, the isolated antigen binding construct comprises a HCDR1 comprising amino acids having at least 80% identity with the amino acid sequence of SEQ ID NO: 1 (EYTIH); a HCDR2 comprising amino acids having at least 80% identity with the amino acid sequence of SEQ ID NO: 2 (GINPNNGIPNYNQKFKG); a HCDR3 comprising amino acids having at least 80% identity with the amino acid sequence of SEQ ID NO: 3 (RRIAYGYDEGHAMDY); a LCDR1 comprising amino acids having at least 80% identity with the amino acid sequence of SEQ ID NO: 4 (KSSQSLLYSRNQKNYLA); a LCDR2 comprising amino acids having at least 80% identity with the amino acid sequence of SEQ ID NO: 5 (WASTRES); and a LCDR3 comprising amino acids having the amino acid sequence of SEQ ID NO: 6 (QQYYSYPLT). In some embodiments, the isolated antigen binding construct comprises a heavy chain having at least 80% identity with the amino acid sequence of SEQ ID NO: 7. In some embodiments, the isolated antigen binding construct comprises a heavy chain having at least 90% identity with the amino acid sequence of SEQ ID NO: 7. In some embodiments, the isolated antigen binding construct comprises a heavy chain having at least 95% identity with the amino acid sequence of SEQ ID NO: 7. In some embodiments, the isolated antigen binding construct comprises a light chain having at least 80% identity with the amino acid sequence of SEQ ID NO: 8. In some embodiments, the isolated antigen binding construct comprises a light chain having at least 90% identity with the amino acid sequence of SEQ ID NO: 8. In some embodiments, the isolated antigen binding construct comprises a light chain having at least 95% identity with the amino acid sequence of SEQ ID NO: 8. In some embodiments, the isolated antigen binding construct comprises a light chain having at least 80% identity with the amino acid sequence of SEQ ID NO: 9. In some embodiments, the isolated antigen binding construct comprises a light chain having at least 90% identity with the amino acid sequence of SEQ ID NO: 9. In some embodiments, the isolated antigen binding construct comprises a light chain having at least 95% identity with the amino acid sequence of SEQ ID NO: 9. In some embodiments, the isolated antigen binding construct comprises at least one VH framework residue selected from the group consisting of: an Alanine at position 24 of the sequence in SEQ ID NO: 7; or a Glycine at position 26 of the sequence in SEQ ID NO: 7. In some embodiments, the isolated antigen binding construct comprises at least one VL framework residue selected from the group consisting of: a Serine at position 73 of the sequence in SEQ ID NO: 8; an Arginine at position 83 of the sequence in SEQ ID NO: 8; a Glutamic acid at position 85 of the sequence in SEQ ID NO:8; a Proline at position 86 of the sequence in SEQ ID NO: 8; a Phenylalanine at position 89 of the sequence in SEQ ID NO: 8; or a Tyrosine at position 98 of the sequence in SEQ ID NO: 8. In some embodiments, the isolated antigen binding construct comprises a phenylalanine at position 89 of the light chain sequence in SEQ ID NO: 8. In some embodiments, the isolated antigen binding construct comprises an alanine at position 24 and a glycine at position 26 of the heavy chain sequence in SEQ ID NO: 7. In some embodiments, the isolated antigen binding construct comprises a phenylalanine at position 89 and a serine at position 73 of the sequence of the light chain sequence in SEQ ID NO: 8. In some embodiments, the isolated antigen binding construct comprises a phenylalanine at position 89, a serine at position 73, and a tyrosine at position 98 of the sequence of the light chain sequence in SEQ ID NO: 8. In some embodiments, the isolated antigen binding construct comprises a phenylalanine at position 89 and a tyrosine at position 98 of the sequence of the light chain sequence in SEQ ID NO: 8. In some embodiments, the isolated antigen binding construct comprises a phenylalanine at position 89, an arginine at position 83, a glutamic acid at position 85, a proline at position 86, and a tyrosine at position 98 of the sequence of the light chain sequence in SEQ ID NO: 8. In some embodiments, the isolated antigen binding construct comprises a phenylalanine at position 89, a serine at position 73, an arginine at position 83, a glutamic acid at position 85, and a proline at position 86 of the sequence of the light chain sequence in SEQ ID NO: 8. In some embodiments, the isolated antigen binding construct comprises a phenylalanine at position 89, a serine at position 73, an arginine at position 83, a glutamic acid at position 85, a proline at position 86, and a tyrosine at position 98 of the sequence of the light chain sequence in SEQ ID NO: 8. In some embodiments, the isolated antigen binding construct comprises a phenylalanine at position 89, an arginine at position 83, a glutamic acid at position 85, and a proline at position 86 of the sequence of the light chain sequence in SEQ ID NO: 8. In some embodiments, the isolated antigen binding construct comprises a variable heavy domain (VH) of SEQ ID NO: 7; and a variable light domain (VL) of SEQ ID NO: 8. In some embodiments, the isolated antigen binding construct comprises a variable heavy domain (VH) of SEQ ID NO: 7; and a variable light domain (VL), of SEQ ID NO: 9.

Some aspects of the present disclosure relate to an isolated antigen binding construct specific against FAP alpha comprising a CDR3 comprising amino acids having at least 90% identity with the amino acid sequence of SEQ ID NO: 6 (QQYYSYPLT). Some aspects of the present disclosure relate to an isolated antigen binding construct comprising a light chain CDR3 comprising amino acids having at least 100% identity with the amino acid sequence of SEQ ID NO: 6. Some aspects of the present disclosure relate to an isolated antigen binding construct thereof, comprising: a LCDR1 comprising amino acids having at least 80% identity with the amino acid sequence of SEQ ID NO: 4 (KSSQSLLYSRNQKNYLA); a LCDR2 comprising amino acids having at least 80% identity with the amino acid sequence of SEQ ID NO: 5 (WASTRES); and a LCDR3 comprising amino acids having the amino acid sequence of SEQ ID NO: 6 (QQYYSYPLT). Some aspects of the present disclosure relate to an isolated antigen binding construct comprising a heavy chain having at least 99% identity with the amino acid sequence of SEQ ID NO: 7. Some aspects of the present disclosure relate to an isolated antigen binding construct comprising a heavy chain having at least 80% identity with the amino acid sequence of SEQ ID NO: 7, comprising at least one VH framework residue selected from the group consisting of: an Alanine at position 24 of the sequence in SEQ ID NO: 7; and a Glycine at position 26 of the sequence in SEQ ID NO: 7. Some aspects of the present disclosure relate to an isolated antigen binding construct comprising a light chain having at least 80% identity with the amino acid sequence of SEQ ID NO: 8, comprising at least one VL framework residue selected from the group consisting of: a Serine at position 73 of the sequence in SEQ ID NO: 8; an Arginine at position 83 of the sequence in SEQ ID NO: 8; a Glutamic acid at position 85 of the sequence in SEQ ID NO:8; a Proline at position 86 of the sequence in SEQ ID NO: 8; a Phenylalanine at position 89 of the sequence in SEQ ID NO: 8; and a Tyrosine at position 98 of the sequence in SEQ ID NO: 8. Some aspects of the present disclosure relate to an isolated antigen binding construct comprising a light chain comprising amino acids having at least 95% identity with the amino acid sequence of SEQ ID NO: 9. Some aspects of the present disclosure relate to an isolated antigen binding construct comprising a light chain comprising amino acids having at least 95% identity with the amino acid sequence of SEQ ID NO: 8. Some aspects of the present disclosure relate to an isolated antigen binding construct comprising: a variable heavy domain (VH) comprising amino acids having at least 95% identity with the amino acid sequence of SEQ ID NO: 7; and a variable light domain (VL) comprising amino acids having at least 95% identity with the amino acid sequence of SEQ ID NO: 8. Some aspects of the present disclosure relate to an isolated humanized antigen binding construct comprising: a variable heavy domain (VH) comprising amino acids having at least 95% identity with the amino acid sequence of SEQ ID NO: 7; and a variable light domain (VL) comprising amino acids having at least 89% identity with the amino acid sequence of SEQ ID NO: 9. In some embodiments, the isolated antigen binding construct comprises at least one VH framework residue selected from the group consisting of: an Alanine at position 24 of the sequence in SEQ ID NO: 7; and a Glycine at position 26 of the sequence in SEQ ID NO: 7. In some embodiments, the isolated antigen binding construct comprises at least one VL framework residue selected from the group consisting of: a Serine at position 73 of the sequence in SEQ ID NO: 8; an Arginine at position 83 of the sequence in SEQ ID NO: 8; a Glutamic acid at position 85 of the sequence in SEQ ID NO:8; a Proline at position 86 of the sequence in SEQ ID NO: 8; a Phenylalanine at position 89 of the sequence in SEQ ID NO: 8; and a Tyrosine at position 98 of the sequence in SEQ ID NO: 8. In some embodiments, the isolated antigen binding construct comprises a phenylalanine at position 89 of the light chain sequence in SEQ ID NO: 8. In some embodiments, the isolated antigen binding construct comprises a phenylalanine at position 89 and a serine at position 73 of the sequence of the light chain sequence in SEQ ID NO: 4. In some embodiments, the isolated antigen binding construct comprises a phenylalanine at position 89, a serine at position 73, and a tyrosine at position 98 of the sequence of the light chain sequence in SEQ ID NO: 8. In some embodiments, the isolated antigen binding construct comprises a phenylalanine at position 89 and a tyrosine at position 98 of the sequence of the light chain sequence in SEQ ID NO: 8. In some embodiments, the isolated antigen binding construct comprises a phenylalanine at position 89, an arginine at position 83, a glutamic acid at position 85, a proline at position 86, and a tyrosine at position 98 of the sequence of the light chain sequence in SEQ ID NO: 8. In some embodiments, the isolated antigen binding construct comprises a phenylalanine at position 89, a serine at position 73, an arginine at position 83, a glutamic acid at position 85, and a proline at position 86 of the sequence of the light chain sequence in SEQ ID NO: 8. In some embodiments, the isolated antigen binding construct comprises a phenylalanine at position 89, a serine at position 73, an arginine at position 83, a glutamic acid at position 85, a proline at position 86, and a tyrosine at position 98 of the sequence of the light chain sequence in SEQ ID NO: 8. In some embodiments, the isolated antigen binding construct comprises a phenylalanine at position 89, an arginine at position 83, a glutamic acid at position 85, and a proline at position 86 of the sequence of the light chain sequence in SEQ ID NO: 8.

Some aspects of the present disclosure relate to an isolated antigen binding construct comprising amino acids having at least 80% identity with an amino acid sequence selected from the group of SEQ ID NOS: 23-44. Some aspects of the present disclosure relate to an isolated antigen binding construct comprising amino acids having at least 90% identity with an amino acid sequence selected from the group of SEQ ID NOS: 23-44. Some aspects of the present disclosure relate to an isolated antigen binding construct comprising amino acids having at least 100% identity with an amino acid sequence selected from the group of SEQ ID NOS: 23-44, 12-43, 12-45, 87-96, 110-115, 122-125, or 140-143.

As disclosed herein, antigen binding constructs are designed with specific sequences. In some embodiments, these constructs bind to FAP. In some embodiments, the antigen binding construct comprises one or more of: (1) a HCDR1 comprising the amino acid sequence of SEQ ID NO: 81 (NYDIN), or a sequence that has no more than 3 point mutations thereto and/or a sequence that has at least 80% similarity to SEQ ID NO: 81; (2) a HCDR2 comprising the amino acid sequence of SEQ ID NO: 82 (LIWTGGGTN), or a sequence that has no more than 3 point mutations thereto and/or a sequence that has at least 80% similarity to SEQ ID NO: 82; (3) a HCDR3 comprising the amino acid sequence of SEQ ID NO: 83 (GGPLVWYALDY), or a sequence that has no more than 3 point mutations thereto and/or a sequence that has at least 80% similarity to SEQ ID NO: 83; (4) a LCDR1 comprising the amino acid sequence of SEQ ID NO: 84 (KASQDVSTAVA), or a sequence that has no more than 3 point mutations thereto and/or a sequence that has at least 80% similarity to SEQ ID NO: 84; (5) a LCDR2 comprising the amino acid sequence of SEQ ID NO: 85 (SASYRYT), or a sequence that has no more than 3 point mutations thereto and/or a sequence that has at least 80% similarity to SEQ ID NO: 85; (6) a LCDR3 comprising the amino acid sequence of SEQ ID NO: 86 (QQHYSNPRT), or a sequence that has no more than 3 point mutations thereto and/or a sequence that has at least 80% similarity to SEQ ID NO: 86; (7) or any combination thereof.

In any of the embodiments provided herein, the % similarity or identity can be 80, 85, 90, 95, 96, 97, 98, 99 or 100% to any one or more of the recited sequences (e.g., 1, 2, 3, 4, 5, 6 of the CDRs and/or VH and/or VL provided herein). In some embodiments, any one or more of the protein sections provided herein (e.g., CDR, VH, and/or VL) can include 1, 2, or 3 substitutions that can be, but need not be conservative substitutions.

In some embodiments, an antigen binding construct comprises one or more of: (1) a HCDR1 comprising the amino acid sequence of SEQ ID NO: 81 (NYDIN), or a sequence that has no more than 3 point mutations thereto and/or a sequence that has at least 80% similarity to SEQ ID NO: 81; (2) a HCDR2 comprising the amino acid sequence of SEQ ID NO: 82 (LIWTGGGTN), or a sequence that has no more than 3 point mutations thereto and/or a sequence that has at least 80% similarity to SEQ ID NO: 82; (3) a HCDR3 comprising the amino acid sequence of SEQ ID NO: 83 (GGPLVWYALDY), or a sequence that has no more than 3 point mutations thereto and/or a sequence that has at least 80% similarity to SEQ ID NO: 83; (4) or any combination thereof. Also disclosed herein is an antigen binding construct comprises one or more of: (1) a LCDR1 comprising the amino acid sequence of SEQ ID NO: 84 (KASQDVSTAVA), or a sequence that has no more than 3 point mutations thereto and/or a sequence that has at least 80% similarity to SEQ ID NO: 884; (2) a LCDR2 comprising the amino acid sequence of SEQ ID NO: 5 (SASYRYT), or a sequence that has no more than 3 point mutations thereto and/or a sequence that has at least 80% similarity to SEQ ID NO: 85; (3) a LCDR3 comprising the amino acid sequence of SEQ ID NO: 86 (QQHYSNPRT), or a sequence that has no more than 3 point mutations thereto and/or a sequence that has at least 80% similarity to SEQ ID NO: 86; (4) or any combination thereof.

In some embodiments, an isolated antigen binding construct is specific against FAP comprising a CDR1 comprising the amino acid sequence of SEQ ID NO: 81 (NYDIN), a sequence that has no more than 3 point mutations thereto, and/or a sequence that has at least 80% similarity to SEQ ID NO: 81. In some embodiments, the CDR1 is part of a heavy chain. Also disclosed herein is an isolated antigen binding construct specific against FAP comprising a CDR2 comprising with the amino acid sequence of SEQ ID NO: 82 (LIWTGGGTN), a sequence that has no more than 3 point mutations thereto, and/or a sequence that has at least 80% similarity to SEQ ID NO: 82. In some embodiments, the CDR2 is part of a heavy chain. Also disclosed herein is an isolated antigen binding construct specific against FAP comprising a CDR3 comprising the amino acid sequence of SEQ ID NO: 83 (GGPLVWYALDY), a sequence that has no more than 3 point mutations thereto, and/or a sequence that has at least 80% similarity to SEQ ID NO: 83. In some embodiments, the CDR3 is part of a heavy chain. Also disclosed herein is an isolated antigen binding construct specific against FAP comprising a CDR1 comprising the amino acid sequence of SEQ ID NO: 84 (KASQDVSTAVA), a sequence that has no more than 3 point mutations thereto, and/or a sequence that has at least 80% similarity to SEQ ID NO: 84. In some embodiments, the CDR1 is part of a light chain. Also disclosed herein is an isolated antigen binding construct specific against FAP comprising a CDR2 comprising the amino acid sequence of SEQ ID NO: 85 (SASYRYT), a sequence that has no more than 3 point mutations thereto, and/or a sequence that has at least 80% similarity to SEQ ID NO: 85. In some embodiments, the CDR2 is part of a light chain. Also disclosed herein is an isolated antigen binding construct specific against FAP comprising a CDR3 comprising the amino acid sequence of SEQ ID NO: 86 (QQHYSNPRT), a sequence that has no more than 3 point mutations thereto, and/or a sequence that has at least 80% similarity to SEQ ID NO: 86. In some embodiments, the CDR3 is part of a light chain.

In some embodiments, the antigen binding construct is specific against FAP. In some embodiments, the antigen binding construct is specific against FAP alpha. In some embodiments, the antigen binding construct does not bind DPP4.

In some embodiments, the antigen binding construct contains an at least one CDR region with less than 4 point mutations, less than 3 point mutations, less than 2 point mutations, or less than 1 point mutation from any one of SEQ ID NO: 81-86.

In some embodiments, the antigen binding construct contains an at least one CDR region with at least about 80%, at least about 85%, at least about 90%, at least about 95%, at least about 99%, or about 100% similarity with any one SEQ ID NO: 81-86. In some embodiments, the antigen binding construct comprises one CDR region with at most 3 point mutations and/or at least about 80% similarity to the sequence of any one of SEQ ID NO: 1-6. In some embodiments, the antigen binding construct comprises two CDR regions, each with at most 3 point mutations and/or at least about 80% similarity to the sequence of any one of SEQ ID NO: 81-86. In some embodiments, the antigen binding construct comprises three CDR regions, each with at most 3 point mutations and/or at least about 80% similarity to the sequence of any one of SEQ ID NO: 81-86. In some embodiments, the antigen binding construct comprises four CDR regions, each with at most 3 point mutations and/or at least about 80% similarity to the sequence of any one of SEQ ID NO: 81-86. In some embodiments, the antigen binding construct comprises five CDR regions, each with at most 3 point mutations and/or at least about 80% similarity to the sequence of any one of SEQ ID NO: 81-86. In some embodiments, the antigen binding construct comprises six CDR regions, each with at most 3 point mutations and/or at least about 80% similarity to the sequence of any one of SEQ ID NO: 81-86. In some embodiments, the antigen binding construct comprises a heavy chain having at least about 80%, at least about 85%, at least about 90%, at least about 95%, at least about 99%, and/or at least 100% identity with the amino acid sequence of SEQ ID NO: 87. In some embodiments, the antigen binding construct comprises a light chain having at least about 80%, at least about 85%, at least about 90%, at least about 95%, at least about 99%, and/or at least 100% identity with the amino acid sequence of SEQ ID NO: 88. In some embodiments, the antigen binding construct comprises a variable heavy domain (VH) having at least about 80%, at least about 85%, at least about 90%, at least about 95%, at least about 99%, and/or at least 100% identity with the amino acid sequence of SEQ ID NO: 87, and a variable light domain (VL) having at least about 80%, at least about 85%, at least about 90%, at least about 95%, at least about 99%, and/or at least 100% identity with the amino acid sequence of SEQ ID NO: 88.

In some embodiments, the antigen binding construct has any of the CDRs provided herein and one or more of the FRs provided in any of the VH and/or VL sequences provided herein, including any one of FR1, FR2, FR3, and/or FR4, or any sequences that is at least 60, 70, 80, 90, or 95, 96, 97, 98, or 99% identical or similar thereto.

In some embodiments, the antigen binding construct comprises a heavy chain having at least about 80%, at least about 85%, at least about 90%, at least about 95%, at least about 99%, and/or at least 100% identity with the amino acid sequence of SEQ ID NO: 89. In some embodiments, the antigen binding construct comprises a light chain having at least about 80%, at least about 85%, at least about 90%, at least about 95%, at least about 99%, and/or at least 100% identity with the amino acid sequence of SEQ ID NO: 90. In some embodiments, the antigen binding construct comprises a variable heavy domain (VH) having at least about 80%, at least about 85%, at least about 90%, at least about 95%, at least about 99%, and/or at least 100% identity with the amino acid sequence of SEQ ID NO: 89, and a variable light domain (VL) having at least about 80%, at least about 85%, at least about 90%, at least about 95%, at least about 99%, and/or at least 100% identity with the amino acid sequence of SEQ ID NO: 90.

In some embodiments, the antigen binding construct comprises a heavy chain having at least about 80%, at least about 85%, at least about 90%, at least about 95%, at least about 99%, and/or at least 100% identity with the amino acid sequence of SEQ ID NO: 91. In some embodiments, the antigen binding construct comprises a light chain having at least about 80%, at least about 85%, at least about 90%, at least about 95%, at least about 99%, and/or at least 100% identity with the amino acid sequence of SEQ ID NO: 92. In some embodiments, the antigen binding construct comprises a variable heavy domain (VH) having at least about 80%, at least about 85%, at least about 90%, at least about 95%, at least about 99%, and/or at least 100% identity with the amino acid sequence of SEQ ID NO: 91, and a variable light domain (VL) having at least about 80%, at least about 85%, at least about 90%, at least about 95%, at least about 99%, and/or at least 100% identity with the amino acid sequence of SEQ ID NO: 92.

In some embodiments, the antigen binding construct comprises a heavy chain having at least about 80%, at least about 85%, at least about 90%, at least about 95%, at least about 99%, and/or at least 100% identity with the amino acid sequence of SEQ ID NO: 93. In some embodiments, the antigen binding construct comprises a light chain having at least about 80%, at least about 85%, at least about 90%, at least about 95%, at least about 99%, and/or at least 100% identity with the amino acid sequence of SEQ ID NO: 94. In some embodiments, the antigen binding construct comprises a variable heavy domain (VH) having at least about 80%, at least about 85%, at least about 90%, at least about 95%, at least about 99%, and/or at least 100% identity with the amino acid sequence of SEQ ID NO: 93, and a variable light domain (VL) having at least about 80%, at least about 85%, at least about 90%, at least about 95%, at least about 99%, and/or at least 100% identity with the amino acid sequence of SEQ ID NO: 94.

In some embodiments, the antigen binding construct comprises a heavy chain having at least about 80%, at least about 85%, at least about 90%, at least about 95%, at least about 99%, and/or at least 100% identity with the amino acid sequence of SEQ ID NO: 95. In some embodiments, the antigen binding construct comprises a light chain having at least about 80%, at least about 85%, at least about 90%, at least about 95%, at least about 99%, and/or at least 100% identity with the amino acid sequence of SEQ ID NO: 96. In some embodiments, the antigen binding construct comprises a variable heavy domain (VH) having at least about 80%, at least about 85%, at least about 90%, at least about 95%, at least about 99%, and/or at least 100% identity with the amino acid sequence of SEQ ID NO: 95, and a variable light domain (VL) having at least about 80%, at least about 85%, at least about 90%, at least about 95%, at least about 99%, and/or at least 100% identity with the amino acid sequence of SEQ ID NO: 96.

In some embodiments, the antigen binding construct comprises a heavy chain having at least about 80%, at least about 85%, at least about 90%, at least about 95%, at least about 99%, and/or at least 100% identity with the amino acid sequence of SEQ ID NO: 122. In some embodiments, the antigen binding construct comprises a light chain having at least about 80%, at least about 85%, at least about 90%, at least about 95%, at least about 99%, and/or at least 100% identity with the amino acid sequence of SEQ ID NO: 123. In some embodiments, the antigen binding construct comprises a variable heavy domain (VH) having at least about 80%, at least about 85%, at least about 90%, at least about 95%, at least about 99%, and/or at least 100% identity with the amino acid sequence of SEQ ID NO: 122, and a variable light domain (VL) having at least about 80%, at least about 85%, at least about 90%, at least about 95%, at least about 99%, and/or at least 100% identity with the amino acid sequence of SEQ ID NO: 123.

Also disclosed herein is an antigen binding construct comprising a variable heavy domain (VH) having at least about 80%, at least about 85%, at least about 90%, at least about 95%, at least about 99%, and/or at least 100% identity with the amino acid sequence of SEQ ID NO: 87, and a variable light domain (VL) having at least about 80%, at least about 85%, at least about 90%, at least about 95%, at least about 99%, and/or at least 100% identity with the amino acid sequence of SEQ ID NO: 88. Also disclosed herein is an antigen binding construct comprising a variable heavy domain (VH) having at least about 80%, at least about 85%, at least about 90%, at least about 95%, at least about 99%, and/or at least 100% identity with the amino acid sequence of SEQ ID NO: 89, and/or a variable light domain (VL) having at least about 80%, at least about 85%, at least about 90%, at least about 95%, at least about 99%, and/or at least 100% identity with the amino acid sequence of SEQ ID NO: 90. Also disclosed herein is an antigen binding construct comprising a variable heavy domain (VH) having at least about 80%, at least about 85%, at least about 90%, at least about 95%, at least about 99%, and/or at least 100% identity with the amino acid sequence of SEQ ID NO: 91, and/or a variable light domain (VL) having at least about 80%, at least about 85%, at least about 90%, at least about 95%, at least about 99%, and/or at least 100% identity with the amino acid sequence of SEQ ID NO: 92. Also disclosed herein is an antigen binding construct comprising a variable heavy domain (VH) having at least about 80%, at least about 85%, at least about 90%, at least about 95%, at least about 99%, and/or at least 100% identity with the amino acid sequence of SEQ ID NO: 93, and/or a variable light domain (VL) having at least about 80%, at least about 85%, at least about 90%, at least about 95%, at least about 99%, and/or at least 100% identity with the amino acid sequence of SEQ ID NO: 94. Also disclosed herein is an antigen binding construct comprising a variable heavy domain (VH) having at least about 80%, at least about 85%, at least about 90%, at least about 95%, at least about 99%, and/or at least 100% identity with the amino acid sequence of SEQ ID NO: 95, and/or a variable light domain (VL) having at least about 80%, at least about 85%, at least about 90%, at least about 95%, at least about 99%, and/or at least 100% identity with the amino acid sequence of SEQ ID NO: 96. Also disclosed herein is an antigen binding construct comprising a variable heavy domain (VH) having at least about 80%, at least about 85%, at least about 90%, at least about 95%, at least about 99%, and/or at least 100% identity with the amino acid sequence of SEQ ID NO: 122, and/or a variable light domain (VL) having at least about 80%, at least about 85%, at least about 90%, at least about 95%, at least about 99%, and/or at least 100% identity with the amino acid sequence of SEQ ID NO: 123.

In some embodiments, the antigen binding construct comprises a variable light domain and a variably heavy domain, wherein the order of the variable regions, from N terminus to C terminus of the polypeptide is V_{L}, V_{H}. In some embodiments, the antigen binding construct comprises a variable light domain and a variably heavy domain, wherein the order of the variable regions, from N terminus to C terminus of the polypeptide is V_{H}, V_{L}.

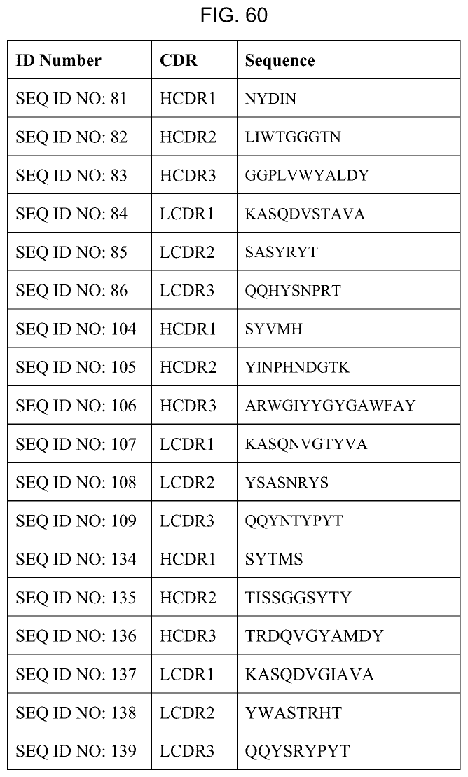

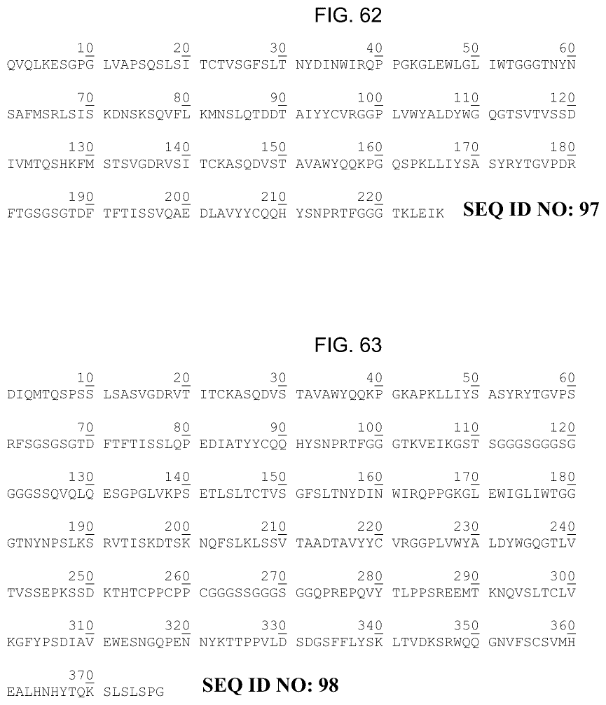

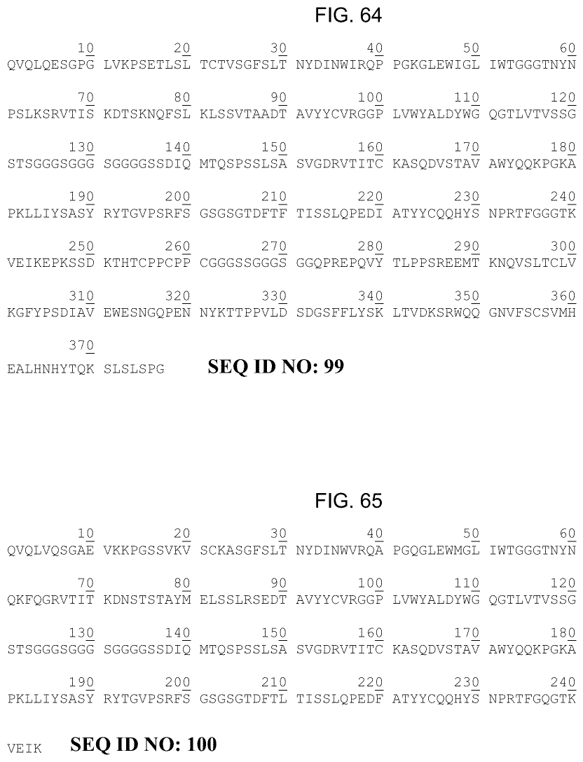

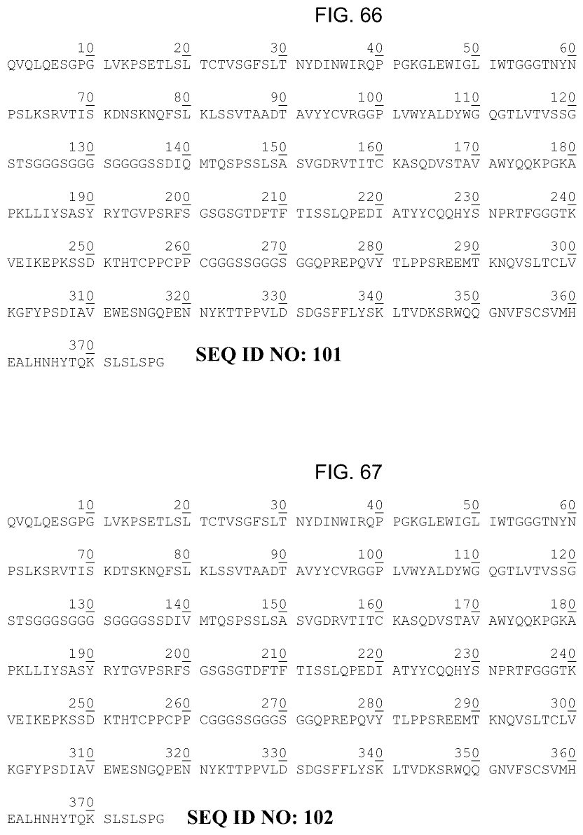

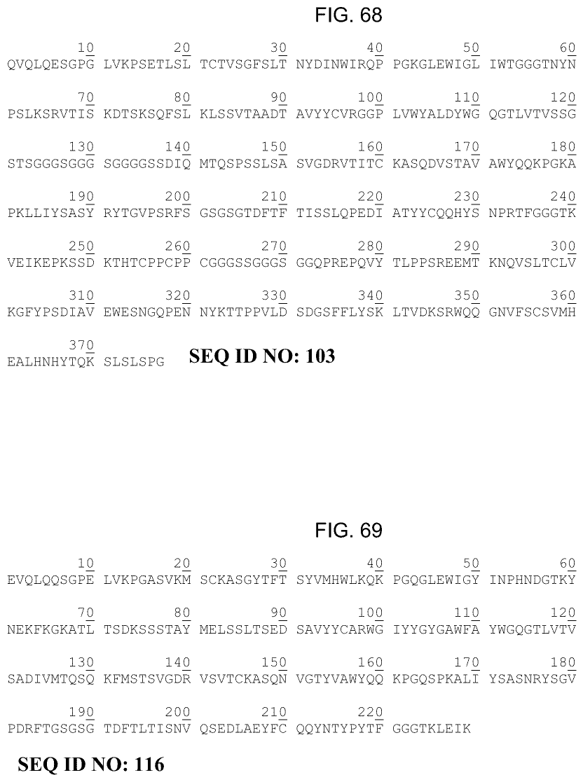

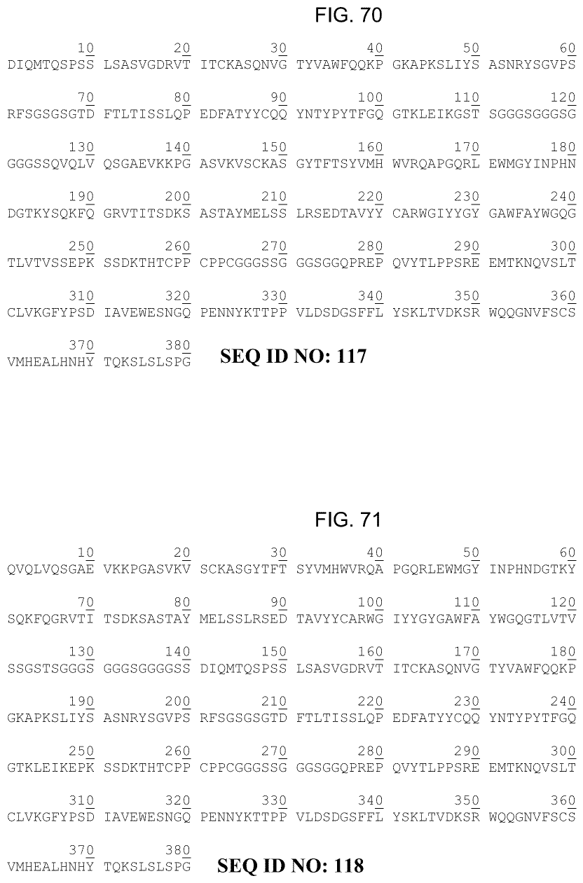

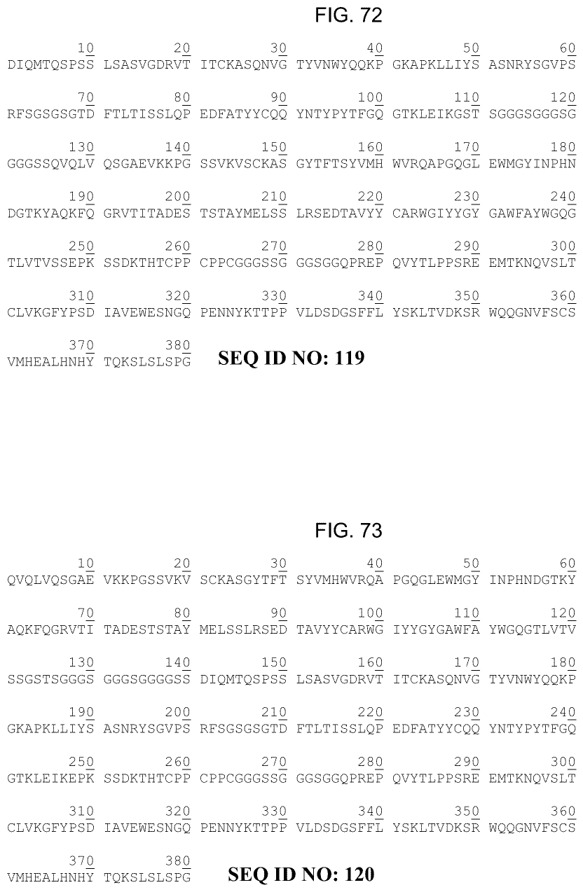

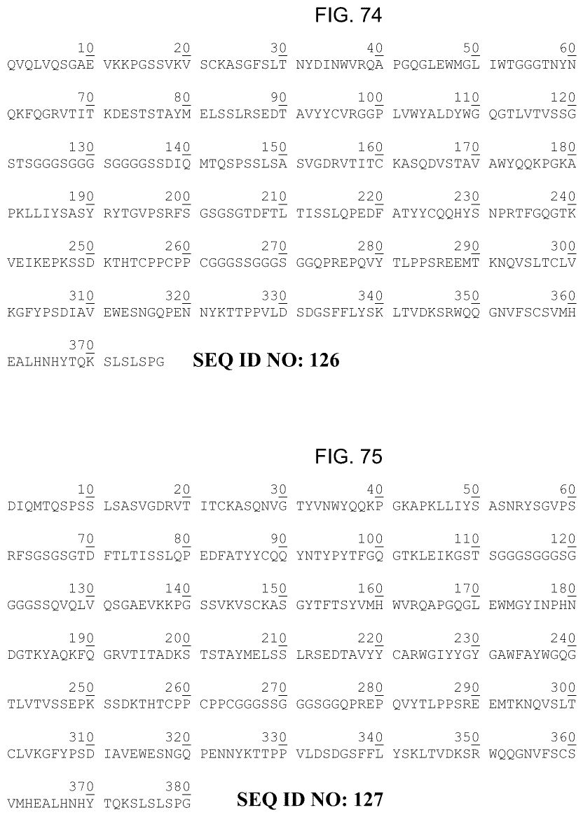

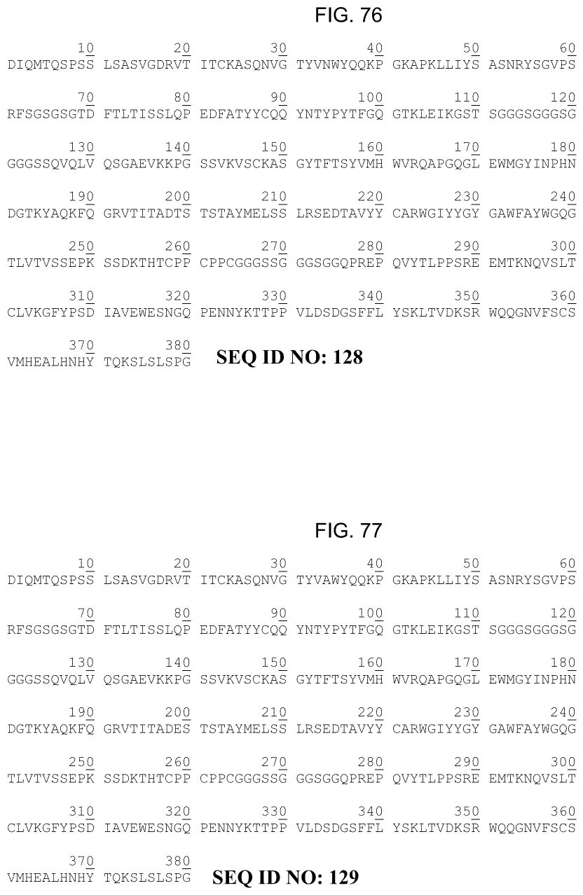

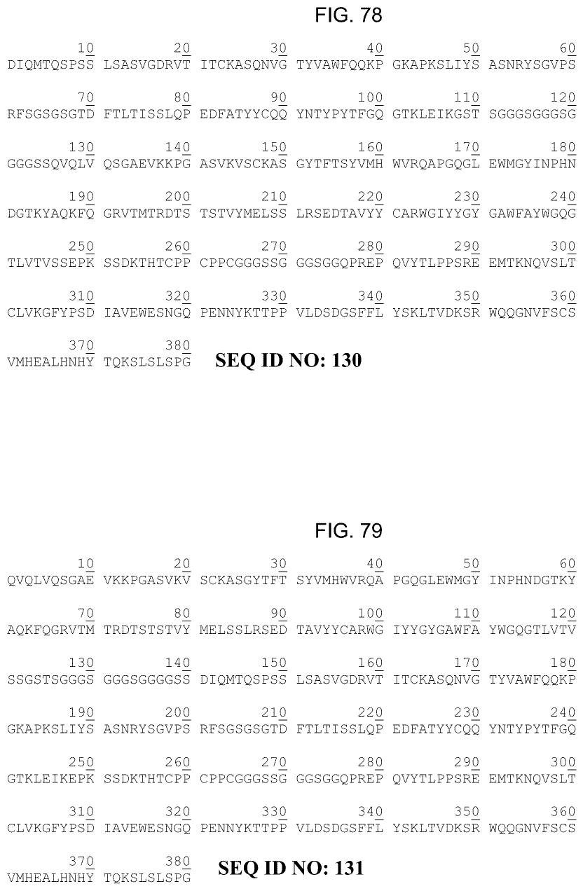

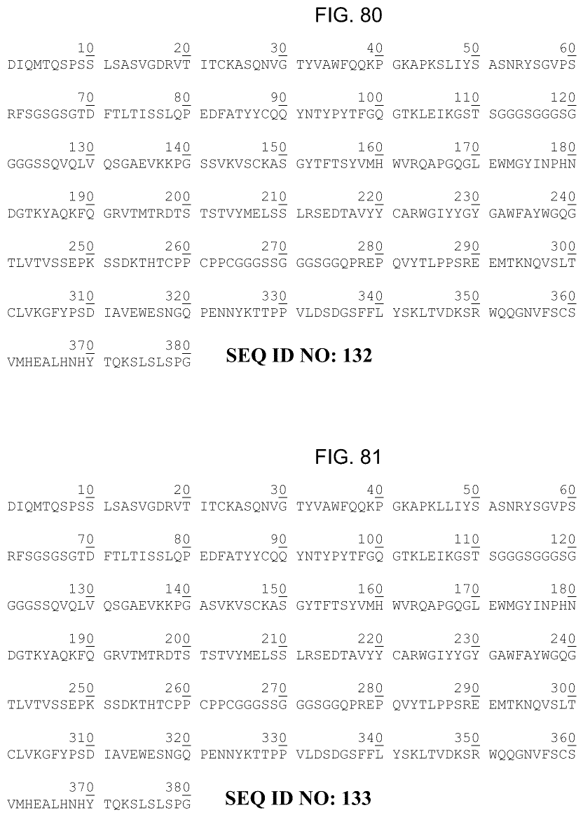

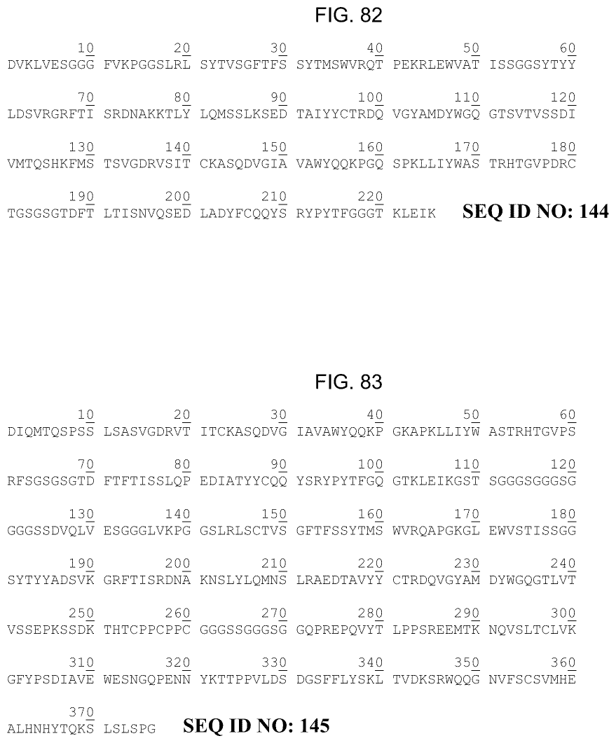

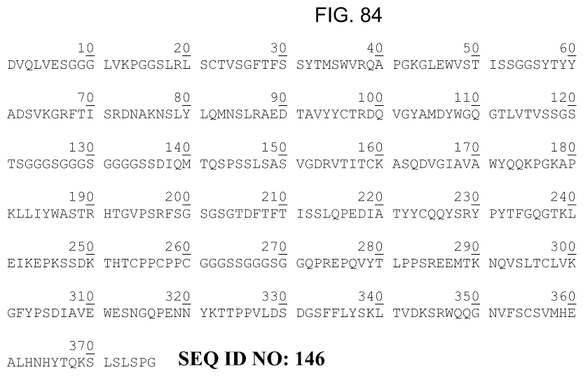

In some embodiments, the antigen binding construct comprises any of the sequences provided in any one or more of the figures provided herein, including variants there of that are at least 60, 70, 80, 90, 95, 96, 97, 98, 99 or greater percent identity thereto or similarity thereto, including those in FIGS. 52A-52F for a hinge region and FIGS. 60-61, and 62-90 for any CDR, VH, and/or VL sequence provided therein.

Also disclosed herein is an isolated antigen binding construct comprising amino acids having at least about 80%, at least about 85%, at least about 90%, at least about 95%, at least about 98%, at least about 99%, and/or 100% identity with at least one amino acid sequence selected from the group of SEQ ID NOS: 97-103 and 126.

Also disclosed herein is an isolated antigen binding construct thereof, comprising at least one of: (1) a HCDR1 comprising the amino acid sequence of SEQ ID NO: 104 (SYVMH), or a sequence that has no more than 3 point mutations thereto and/or a sequence that has at least 80% similarity to SEQ ID NO: 104; (2) a HCDR2 comprising the amino acid sequence of SEQ ID NO: 105 (YINPHNDGTK), or a sequence that has no more than 3 point mutations thereto and/or a sequence that has at least 80% similarity to SEQ ID NO: 105; (3) a HCDR3 comprising the amino acid sequence of SEQ ID NO: 106 (ARWGIYYGYGAWFAY), or a sequence that has no more than 3 point mutations thereto and/or a sequence that has at least 80% similarity to SEQ ID NO: 106; (4) a LCDR1 comprising the amino acid sequence of SEQ ID NO: 107 (KASQNVGTYVA), or a sequence that has no more than 3 point mutations thereto and/or a sequence that has at least 80% similarity to SEQ ID NO: 107; (5) a LCDR2 comprising the amino acid sequence of SEQ ID NO: 108 (YSASNRYS), or a sequence that has no more than 3 point mutations thereto and/or a sequence that has at least 80% similarity to SEQ ID NO: 108; (6) a LCDR3 comprising the amino acid sequence of SEQ ID NO: 109 (QQYNTYPYT), or a sequence that has no more than 3 point mutations thereto and/or a sequence that has at least 80% similarity to SEQ ID NO: 109; (7) or any combination thereof. Also disclosed herein is an antigen binding construct comprising one or more of: (1) a HCDR1 comprising the amino acid sequence of SEQ ID NO: 104 (SYVMH), or a sequence that has no more than 3 point mutations thereto and/or a sequence that has at least 80% similarity to SEQ ID NO: 104; (2) a HCDR2 comprising the amino acid sequence of SEQ ID NO: 105 (YINPHNDGTK), or a sequence that has no more than 3 point mutations thereto and/or a sequence that has at least 80% similarity to SEQ ID NO: 105; (3) a HCDR3 the amino acid sequence of SEQ ID NO: 106 (ARWGIYYGYGAWFAY), or a sequence that has no more than 3 point mutations thereto and/or a sequence that has at least 80% similarity to SEQ ID NO: 106; (4) or any combination thereof. Also disclosed herein is an antigen binding construct comprising one or more of: (1) a LCDR1 comprising the amino acid sequence of SEQ ID NO: 107 (KASQNVGTYVA), or a sequence that has no more than 3 point mutations thereto and/or a sequence that has at least 80% similarity to SEQ ID NO: 107; (2) a LCDR2 comprising the amino acid sequence of SEQ ID NO: 108 (YSASNRYS), or a sequence that has no more than 3 point mutations thereto and/or a sequence that has at least 80% similarity to SEQ ID NO: 108; (3) a LCDR3 comprising the amino acid sequence of SEQ ID NO: 109 (QQYNTYPYT), or a sequence that has no more than 3 point mutations thereto and/or a sequence that has at least 80% similarity to SEQ ID NO: 109; or (4) any combination thereof.

Also disclosed herein is an isolated antigen binding construct specific against FAP comprising a CDR1 comprising the amino acid sequence of SEQ ID NO: 104 (SYVMH), a sequence that has no more than 3 point mutations thereto, and/or a sequence that has at least 80% similarity to SEQ ID NO: 104. In some embodiments, the CDR1 is part of a heavy chain. Also disclosed herein is an isolated antigen binding construct specific against FAP comprising a CDR2 comprising the amino acid sequence of SEQ ID NO: 105 (YINPHNDGTK), a sequence that has no more than 3 point mutations thereto, and/or a sequence that has at least 80% similarity to SEQ ID NO: 105. In some embodiments, the CDR2 is part of a heavy chain. Also disclosed herein is an isolated antigen binding construct specific against FAP comprising a CDR3 comprising the amino acid sequence of SEQ ID NO: 106 (ARWGIYYGYGAWFAY), a sequence that has no more than 3 point mutations thereto, and/or a sequence that has at least 80% similarity to SEQ ID NO: 106. In some embodiments, the CDR3 is part of a heavy chain. Also disclosed herein is an isolated antigen binding construct specific against FAP comprising a CDR1 comprising the amino acid sequence of SEQ ID NO: 107 (KASQNVGTYVA), a sequence that has no more than 3 point mutations thereto, and/or a sequence that has at least 80% similarity to SEQ ID NO: 107. In some embodiments, the CDR1 is part of a light chain. Also disclosed herein is an isolated antigen binding construct specific against FAP comprising a CDR2 comprising the amino acid sequence of SEQ ID NO: 108 (YSASNRYS), a sequence that has no more than 3 point mutations thereto, and/or a sequence that has at least 80% similarity to SEQ ID NO: 108. In some embodiments, the CDR2 is part of a light chain. Also disclosed herein is an isolated antigen binding construct specific against FAP comprising a CDR3 comprising the amino acid sequence of SEQ ID NO: 109 (QQYNTYPYT), a sequence that has no more than 3 point mutations thereto, and/or a sequence that has at least 80% similarity to SEQ ID NO: 109. In some embodiments, the CDR3 is part of a light chain.

In some embodiments, the antigen binding construct is specific against FAP. In some embodiments, the antigen binding construct is specific against FAP alpha. In some embodiments, the antigen binding construct does not bind DPP4. In some embodiments, the antigen binding construct contains an at least one CDR region with less than 4 point mutations, less than 3 point mutations, less than 2 point mutations, or less than 1 point mutation from any one of SEQ ID NO: 104-109. In some embodiments, the antigen binding construct contains an at least one CDR region with at least about 80%, at least about 85%, at least about 90%, at least about 95%, at least about 99%, or about 100% similarity with any one SEQ ID NO: 104-109. In some embodiments, the antigen binding construct comprises one CDR region with at most 3 point mutations and/or at least about 80% similarity to the sequence of any one of SEQ ID NO: 104-109. In some embodiments, the antigen binding construct comprises two CDR regions, each with at most 3 point mutations and/or at least about 80% similarity to the sequence of any one of SEQ ID NO: 104-109. In some embodiments, the antigen binding construct comprises three CDR regions, each with at most 3 point mutations and/or at least about 80% similarity to the sequence of any one of SEQ ID NO: 104-109. In some embodiments, the antigen binding construct comprises four CDR regions, each with at most 3 point mutations and/or at least about 80% similarity to the sequence of any one of SEQ ID NO: 104-109. In some embodiments, the antigen binding construct comprises five CDR regions, each with at most 3 point mutations and/or at least about 80% similarity to the sequence of any one of SEQ ID NO: 104-109. In some embodiments, the antigen binding construct comprises six CDR regions, each with at most 3 point mutations and/or at least about 80% similarity to the sequence of any one of SEQ ID NO: 104-109.

In some embodiments, the antigen binding construct comprises a heavy chain having at least about 80%, at least about 85%, at least about 90%, at least about 95%, at least about 99%, and/or at least 100% identity with the amino acid sequence of SEQ ID NO: 110. In some embodiments, the antigen binding construct comprises a light chain having at least about 80%, at least about 85%, at least about 90%, at least about 95%, at least about 99%, and/or at least 100% identity with the amino acid sequence of SEQ ID NO: 111. In some embodiments, the antigen binding construct comprises a variable heavy domain (VH) having at least about 80%, at least about 85%, at least about 90%, at least about 95%, at least about 99%, and/or at least 100% identity with the amino acid sequence of SEQ ID NO: 110, and/or a variable light domain (VL) having at least about 80%, at least about 85%, at least about 90%, at least about 95%, at least about 99%, and/or at least 100% identity with the amino acid sequence of SEQ ID NO: 111.

In some embodiments, the antigen binding construct comprises a heavy chain having at least about 80%, at least about 85%, at least about 90%, at least about 95%, at least about 99%, and/or at least 100% identity with the amino acid sequence of SEQ ID NO: 112. In some embodiments, the antigen binding construct comprises a light chain having at least about 80%, at least about 85%, at least about 90%, at least about 95%, at least about 99%, and/or at least 100% identity with the amino acid sequence of SEQ ID NO: 113. In some embodiments, the antigen binding construct comprises a variable heavy domain (VH) having at least about 80%, at least about 85%, at least about 90%, at least about 95%, at least about 99%, and/or at least 100% identity with the amino acid sequence of SEQ ID NO: 112, and/or a variable light domain (VL) having at least about 80%, at least about 85%, at least about 90%, at least about 95%, at least about 99%, and/or at least 100% identity with the amino acid sequence of SEQ ID NO: 113.

In some embodiments, the antigen binding construct comprises a heavy chain having at least about 80%, at least about 85%, at least about 90%, at least about 95%, at least about 99%, and/or at least 100% identity with the amino acid sequence of SEQ ID NO: 95. In some embodiments, the antigen binding construct comprises a light chain having at least about 80%, at least about 85%, at least about 90%, at least about 95%, at least about 99%, and/or at least 100% identity with the amino acid sequence of SEQ ID NO: 96. In some embodiments, the antigen binding construct comprises a variable heavy domain (VH) having at least about 80%, at least about 85%, at least about 90%, at least about 95%, at least about 99%, and/or at least 100% identity with the amino acid sequence of SEQ ID NO: 95, and/or a variable light domain (VL) having at least about 80%, at least about 85%, at least about 90%, at least about 95%, at least about 99%, and/or at least 100% identity with the amino acid sequence of SEQ ID NO: 96.

In some embodiments, the antigen binding construct comprises a heavy chain having at least about 80%, at least about 85%, at least about 90%, at least about 95%, at least about 99%, and/or at least 100% identity with the amino acid sequence of SEQ ID NO: 91. In some embodiments, the antigen binding construct comprises a light chain having at least about 80%, at least about 85%, at least about 90%, at least about 95%, at least about 99%, and/or at least 100% identity with the amino acid sequence of SEQ ID NO: 92. In some embodiments, the antigen binding construct comprises a variable heavy domain (VH) having at least about 80%, at least about 85%, at least about 90%, at least about 95%, at least about 99%, and/or at least 100% identity with the amino acid sequence of SEQ ID NO: 91, and/or a variable light domain (VL) having at least about 80%, at least about 85%, at least about 90%, at least about 95%, at least about 99%, and/or at least 100% identity with the amino acid sequence of SEQ ID NO: 92.

In some embodiments, the antigen binding construct comprises a heavy chain having at least about 80%, at least about 85%, at least about 90%, at least about 95%, at least about 99%, and/or at least 100% identity with the amino acid sequence of SEQ ID NO: 114. In some embodiments, the antigen binding construct comprises a light chain having at least about 80%, at least about 85%, at least about 90%, at least about 95%, at least about 99%, and/or at least 100% identity with the amino acid sequence of SEQ ID NO: 115. In some embodiments, the antigen binding construct comprises a variable heavy domain (VH) having at least about 80%, at least about 85%, at least about 90%, at least about 95%, at least about 99%, and/or at least 100% identity with the amino acid sequence of SEQ ID NO: 114, and/or a variable light domain (VL) having at least about 80%, at least about 85%, at least about 90%, at least about 95%, at least about 99%, and/or at least 100% identity with the amino acid sequence of SEQ ID NO: 115.

In some embodiments, the antigen binding construct comprises a heavy chain having at least about 80%, at least about 85%, at least about 90%, at least about 95%, at least about 99%, and/or at least 100% identity with the amino acid sequence of SEQ ID NO: 95. In some embodiments, the antigen binding construct comprises a light chain having at least about 80%, at least about 85%, at least about 90%, at least about 95%, at least about 99%, and/or at least 100% identity with the amino acid sequence of SEQ ID NO: 96. In some embodiments, the antigen binding construct comprises a variable heavy domain (VH) having at least about 80%, at least about 85%, at least about 90%, at least about 95%, at least about 99%, and/or at least 100% identity with the amino acid sequence of SEQ ID NO: 95, and/or a variable light domain (VL) having at least about 80%, at least about 85%, at least about 90%, at least about 95%, at least about 99%, and/or at least 100% identity with the amino acid sequence of SEQ ID NO: 96.

In some embodiments, the antigen binding construct comprises a heavy chain having at least about 80%, at least about 85%, at least about 90%, at least about 95%, at least about 99%, and/or at least 100% identity with the amino acid sequence of SEQ ID NO: 124. In some embodiments, the antigen binding construct comprises a light chain having at least about 80%, at least about 85%, at least about 90%, at least about 95%, at least about 99%, and/or at least 100% identity with the amino acid sequence of SEQ ID NO: 125. In some embodiments, the antigen binding construct comprises a variable heavy domain (VH) having at least about 80%, at least about 85%, at least about 90%, at least about 95%, at least about 99%, and/or at least 100% identity with the amino acid sequence of SEQ ID NO: 124, and/or a variable light domain (VL) having at least about 80%, at least about 85%, at least about 90%, at least about 95%, at least about 99%, and/or at least 100% identity with the amino acid sequence of SEQ ID NO: 125.

In some embodiments, an antigen binding construct comprises a variable heavy domain (VH) having at least about 80%, at least about 85%, at least about 90%, at least about 95%, at least about 99%, and/or at least 100% identity with the amino acid sequence of SEQ ID NO: 110, and/or a variable light domain (VL) having at least about 80%, at least about 85%, at least about 90%, at least about 95%, at least about 99%, and/or at least 100% identity with the amino acid sequence of SEQ ID NO: 111. Also disclosed herein is an antigen binding construct comprising a variable heavy domain (VH) having at least about 80%, at least about 85%, at least about 90%, at least about 95%, at least about 99%, and/or at least 100% identity with the amino acid sequence of SEQ ID NO: 112, and/or a variable light domain (VL) having at least about 80%, at least about 85%, at least about 90%, at least about 95%, at least about 99%, and/or at least 100% identity with the amino acid sequence of SEQ ID NO: 113.

In some embodiments, an antigen binding construct comprises a variable heavy domain (VH) having at least about 80%, at least about 85%, at least about 90%, at least about 95%, at least about 99%, and/or at least 100% identity with the amino acid sequence of SEQ ID NO: 91, and/or a variable light domain (VL) having at least about 80%, at least about 85%, at least about 90%, at least about 95%, at least about 99%, and/or at least 100% identity with the amino acid sequence of SEQ ID NO: 92.

In some embodiments, an antigen binding construct comprises a variable heavy domain (VH) having at least about 80%, at least about 85%, at least about 90%, at least about 95%, at least about 99%, and/or at least 100% identity with the amino acid sequence of SEQ ID NO: 95, and/or a variable light domain (VL) having at least about 80%, at least about 85%, at least about 90%, at least about 95%, at least about 99%, and/or at least 100% identity with the amino acid sequence of SEQ ID NO: 96.

In some embodiments, an antigen binding construct comprises a variable heavy domain (VH) having at least about 80%, at least about 85%, at least about 90%, at least about 95%, at least about 99%, and/or at least 100% identity with the amino acid sequence of SEQ ID NO: 114, and/or a variable light domain (VL) having at least about 80%, at least about 85%, at least about 90%, at least about 95%, at least about 99%, and/or at least 100% identity with the amino acid sequence of SEQ ID NO: 115.

In some embodiments, an antigen binding construct comprises a variable heavy domain (VH) having at least about 80%, at least about 85%, at least about 90%, at least about 95%, at least about 99%, and/or at least 100% identity with the amino acid sequence of SEQ ID NO: 124, and/or a variable light domain (VL) having at least about 80%, at least about 85%, at least about 90%, at least about 95%, at least about 99%, and/or at least 100% identity with the amino acid sequence of SEQ ID NO: 125.

In some embodiments, an antigen binding construct comprises a variable light domain and a variably heavy domain, wherein the order of the variable regions, from N terminus to C terminus of the polypeptide is V_{L}, VH. In some embodiments, the antigen binding construct comprises a variable light domain and a variably heavy domain, wherein the order of the variable regions, from N terminus to C terminus of the polypeptide is VH, V_{L}. Also disclosed herein is an isolated antigen binding construct comprising amino acids having at least 98% identity with at least one amino acid sequence selected from the group of SEQ ID NOS: 116-120, and 127-133.

In some embodiments, an antigen binding construct comprises at least one of: (1) a HCDR1 comprising the amino acid sequence of SEQ ID NO: 134 (SYTMS), or a sequence that has no more than 3 point mutations thereto and/or a sequence that has at least 80% similarity to SEQ ID NO: 134; (2) a HCDR2 comprising the amino acid sequence of SEQ ID NO: 135 (TISSGGSYTY), or a sequence that has no more than 3 point mutations thereto and/or a sequence that has at least 80% similarity to SEQ ID NO: 135; (3) a HCDR3 comprising the amino acid sequence of SEQ ID NO: 136 (TRDQVGYAMDY), or a sequence that has no more than 3 point mutations thereto and/or a sequence that has at least 80% similarity to SEQ ID NO: 136; (4) a LCDR1 comprising the amino acid sequence of SEQ ID NO: 137 (TRDQVGYAMDY), or a sequence that has no more than 3 point mutations thereto and/or a sequence that has at least 80% similarity to SEQ ID NO: 137; (5) a LCDR2 comprising the amino acid sequence of SEQ ID NO: 138 (YWASTRHT), or a sequence that has no more than 3 point mutations thereto and/or a sequence that has at least 80% similarity to SEQ ID NO: 138; (6) a LCDR3 comprising the amino acid sequence of SEQ ID NO: 139 (QQYSRYPYT), or a sequence that has no more than 3 point mutations thereto and/or a sequence that has at least 80% similarity to SEQ ID NO: 139; (7) or any combination thereof.

In some embodiments, an antigen binding construct comprises one or more of: (1) a HCDR1 comprising the amino acid sequence of SEQ ID NO: 134, or a sequence that has no more than 3 point mutations thereto and/or a sequence that has at least 80% similarity to SEQ ID NO: 134; (2) a HCDR2 comprising the amino acid sequence of SEQ ID NO: 135, or a sequence that has no more than 3 point mutations thereto and/or a sequence that has at least 80% similarity to SEQ ID NO: 135; (3) a HCDR3 the amino acid sequence of SEQ ID NO: 136, or a sequence that has no more than 3 point mutations thereto and/or a sequence that has at least 80% similarity to SEQ ID NO: 136; (4) or any combination thereof.

In some embodiments, an antigen binding construct comprises one or more of: (1) a LCDR1 comprising the amino acid sequence of SEQ ID NO: 137, or a sequence that has no more than 3 point mutations thereto and/or a sequence that has at least 80% similarity to SEQ ID NO: 137; (2) a LCDR2 comprising the amino acid sequence of SEQ ID NO: 138, or a sequence that has no more than 3 point mutations thereto and/or a sequence that has at least 80% similarity to SEQ ID NO: 138; (3) a LCDR3 comprising the amino acid sequence of SEQ ID NO: 139, or a sequence that has no more than 3 point mutations thereto and/or a sequence that has at least 80% similarity to SEQ ID NO: 139; or (4) any combination thereof.

In some embodiments, the an isolated antigen binding construct is specific against FAP and comprises a CDR1 comprising the amino acid sequence of SEQ ID NO: 134, a sequence that has no more than 3 point mutations thereto, and/or a sequence that has at least 80% similarity to SEQ ID NO: 134. In some embodiments, the CDR1 is part of a heavy chain. In some embodiments, an isolated antigen binding construct is specific against FAP comprising a CDR2 comprising the amino acid sequence of SEQ ID NO: 135, a sequence that has no more than 3 point mutations thereto, and/or a sequence that has at least 80% similarity to SEQ ID NO: 135. In some embodiments, the CDR2 is part of a heavy chain.

In some embodiments, an isolated antigen binding construct specific against FAP comprises a CDR3 comprising the amino acid sequence of SEQ ID NO: 136, a sequence that has no more than 3 point mutations thereto, and/or a sequence that has at least 80% similarity to SEQ ID NO: 136. In some embodiments, the CDR3 is part of a heavy chain. In some embodiments, an isolated antigen binding construct specific against FAP comprises a CDR1 comprising the amino acid sequence of SEQ ID NO: 137, a sequence that has no more than 3 point mutations thereto, and/or a sequence that has at least 80% similarity to SEQ ID NO: 137. In some embodiments, the CDR1 is part of a light chain. In some embodiments, an isolated antigen binding construct specific against FAP comprises a CDR2 comprising the amino acid sequence of SEQ ID NO: 138, a sequence that has no more than 3 point mutations thereto, and/or a sequence that has at least 80% similarity to SEQ ID NO: 138. In some embodiments, the CDR2 is part of a light chain. In some embodiments, an isolated antigen binding construct specific against FAP comprising a CDR3 comprising the amino acid sequence of SEQ ID NO: 139, a sequence that has no more than 3 point mutations thereto, and/or a sequence that has at least 80% similarity to SEQ ID NO: 139. In some embodiments, the CDR3 is part of a light chain.

In some embodiments, the antigen binding construct is specific against FAP. In some embodiments, the antigen binding construct is specific against FAP alpha. In some embodiments, the antigen binding construct does not bind DPP4. In some embodiments, the antigen binding construct contains an at least one CDR region with less than 4 point mutations, less than 3 point mutations, less than 2 point mutations, or less than 1 point mutation from any one of SEQ ID NO: 134-139. In some embodiments, the antigen binding construct contains an at least one CDR region with at least about 80%, at least about 85%, at least about 90%, at least about 95%, at least about 99%, or about 100% similarity with any one SEQ ID NO: 134-139. In some embodiments, the antigen binding construct comprises one CDR region with at most 3 point mutations and/or at least about 80% similarity to the sequence of any one of SEQ ID NO: 134-139. In some embodiments, the antigen binding construct comprises two CDR regions, each with at most 3 point mutations and/or at least about 80% similarity to the sequence of any one of SEQ ID NO: 134-139. In some embodiments, the antigen binding construct comprises three CDR regions, each with at most 3 point mutations and/or at least about 80% similarity to the sequence of any one of SEQ ID NO: 134-139. In some embodiments, the antigen binding construct comprises four CDR regions, each with at most 3 point mutations and/or at least about 80% similarity to the sequence of any one of SEQ ID NO: 134-139. In some embodiments, the antigen binding construct comprises five CDR regions, each with at most 3 point mutations and/or at least about 80% similarity to the sequence of any one of SEQ ID NO: 134-139. In some embodiments, the antigen binding construct comprises six CDR regions, each with at most 3 point mutations and/or at least about 80% similarity to the sequence of any one of SEQ ID NO: 134-139.

In some embodiments, the antigen binding construct comprises a heavy chain having at least about 80%, at least about 85%, at least about 90%, at least about 95%, at least about 99%, and/or at least 100% identity with the amino acid sequence of SEQ ID NO: 140. In some embodiments, the antigen binding construct comprises a light chain having at least about 80%, at least about 85%, at least about 90%, at least about 95%, at least about 99%, and/or at least 100% identity with the amino acid sequence of SEQ ID NO: 141. In some embodiments, the antigen binding construct comprises a variable heavy domain (VH) having at least about 80%, at least about 85%, at least about 90%, at least about 95%, at least about 99%, and/or at least 100% identity with the amino acid sequence of SEQ ID NO: 140, and/or a variable light domain (VL) having at least about 80%, at least about 85%, at least about 90%, at least about 95%, at least about 99%, and/or at least 100% identity with the amino acid sequence of SEQ ID NO: 141.

In some embodiments, the antigen binding construct comprises a heavy chain having at least about 80%, at least about 85%, at least about 90%, at least about 95%, at least about 99%, and/or at least 100% identity with the amino acid sequence of SEQ ID NO: 142. In some embodiments, the antigen binding construct comprises a light chain having at least about 80%, at least about 85%, at least about 90%, at least about 95%, at least about 99%, and/or at least 100% identity with the amino acid sequence of SEQ ID NO: 90. In some embodiments, the antigen binding construct comprises a variable heavy domain (VH) having at least about 80%, at least about 85%, at least about 90%, at least about 95%, at least about 99%, and/or at least 100% identity with the amino acid sequence of SEQ ID NO: 142, and/or/or a variable light domain (VL) having at least about 80%, at least about 85%, at least about 90%, at least about 95%, at least about 99%, and/or at least 100% identity with the amino acid sequence of SEQ ID NO: 90.

In some embodiments, the antigen binding construct comprises a heavy chain having at least about 80%, at least about 85%, at least about 90%, at least about 95%, at least about 99%, and/or at least 100% identity with the amino acid sequence of SEQ ID NO: 143. In some embodiments, the antigen binding construct comprises a light chain having at least about 80%, at least about 85%, at least about 90%, at least about 95%, at least about 99%, and/or at least 100% identity with the amino acid sequence of SEQ ID NO: 94. In some embodiments, the antigen binding construct comprises a variable heavy domain (VH) having at least about 80%, at least about 85%, at least about 90%, at least about 95%, at least about 99%, and/or at least 100% identity with the amino acid sequence of SEQ ID NO: 143, and/or a variable light domain (VL) having at least about 80%, at least about 85%, at least about 90%, at least about 95%, at least about 99%, and/or at least 100% identity with the amino acid sequence of SEQ ID NO: 94.

In some embodiments, the antigen binding construct comprises a variable heavy domain (VH) having at least about 80%, at least about 85%, at least about 90%, at least about 95%, at least about 99%, and/or at least 100% identity with the amino acid sequence of SEQ ID NO: 140, and/or a variable light domain (VL) having at least about 80%, at least about 85%, at least about 90%, at least about 95%, at least about 99%, and/or at least 100% identity with the amino acid sequence of SEQ ID NO: 141. In some embodiments, the antigen binding construct comprises a variable heavy domain (VH) having at least about 80%, at least about 85%, at least about 90%, at least about 95%, at least about 99%, and/or at least 100% identity with the amino acid sequence of SEQ ID NO: 142, and/or a variable light domain (VL) having at least about 80%, at least about 85%, at least about 90%, at least about 95%, at least about 99%, and/or at least 100% identity with the amino acid sequence of SEQ ID NO: 90. In some embodiments, the antigen binding construct comprises a variable heavy domain (VH) having at least about 80%, at least about 85%, at least about 90%, at least about 95%, at least about 99%, and/or at least 100% identity with the amino acid sequence of SEQ ID NO: 143, and/or a variable light domain (VL) having at least about 80%, at least about 85%, at least about 90%, at least about 95%, at least about 99%, and/or at least 100% identity with the amino acid sequence of SEQ ID NO: 94.

In some embodiments, the antigen binding construct comprises a variable light domain and a variably heavy domain, wherein the order of the variable regions, from N terminus to C terminus of the polypeptide is V_{L}, V_{H}. In some embodiments, the antigen binding construct comprises a variable light domain and a variably heavy domain, wherein the order of the variable regions, from N terminus to C terminus of the polypeptide is V_{H}, V_{L}. In some embodiments, the antigen binding construct comprises amino acids having at least 98% identity with at least one amino acid sequence selected from the group of SEQ ID NOS: 144-146.

In some embodiments, the isolated antigen binding construct of any one of the above embodiments is an antibody. In some embodiments, the isolated antigen binding construct is a minibody. In some embodiments, the isolated antigen binding construct is a cys-diabody.

Some aspects of the present disclosure relate to a minibody antigen binding construct comprising amino acids having at least about 80%, at least about 90%, or at least about 99% identity with an amino acid sequence selected from the group of SEQ ID NOS: 11-22. Some aspects of the present disclosure relate to a cys-diabody antigen binding construct comprising amino acids having at least about 80%, at least about 90%, or at least about 99% identity with an amino acid sequence selected from the group of SEQ ID NOS: 11-22.

As disclosed herein, the antigen binding construct can be isolated using any conventional method known to one skilled in the art. It will also be understood that the term “isolated antigen binding construct” can refer to a purified construct, a construct in solution, a construct expressed on the surface of a cell, a construct expressed inside a cell, a construct expressed on the surface of a tissue, a construct expressed inside a tissue, a construct expressed in a system, and/or a construct expressed in an organism. In some embodiments, the antigen binding construct is mammalian in origin. In some embodiments, the antigen binding construct is murine in origin. In some embodiments, the antigen binding construct is human in origin. In some embodiments, the antigen binding construct is humanized. In some embodiments, the antigen binding construct is expressed in a mammalian cell, mammalian cell line, mammalian tissue, mammalian organ, mammalian organ system, and/or mammalian organism. In some embodiments, the antigen binding construct is expressed in a murine cell, murine cell line, murine tissue, murine organ, murine organ system, and/or murine organism. In some embodiments, the antigen binding construct is expressed in a human cell, human cell line, human tissue, human organ, human organ system, and/or human organism.

In some embodiments, the antigen binding construct is an antibody. In some embodiments, the antigen binding construct is an antibody fragment. In some embodiments, the antigen binding construct is an scFv. In some embodiments, the antigen binding construct is a Fab. In some embodiments, the antigen binding construct is a Fab2. In some embodiments, the antigen binding construct is a nanobody. In some embodiments, the antigen binding construct is a minibody. In some embodiments, the antigen binding construct is a diabody. In some embodiments, the antigen binding construct is a cys-diabody. In some embodiments, the antigen binding construct is a combination of two or more of any of the above as a composition and/or a bispecific arrangement.

In some embodiments, a minibody antigen binding construct comprising amino acids having at least about 80%, at least about 90%, or at least about 99% identity with at least one of an amino acid sequence selected from the group of SEQ ID NOS: 97-103, 116-120, 126-133, and 144-146. In some embodiments, a cys-diabody antigen binding construct comprising amino acids having at least about 80%, at least about 90%, or at least about 99% identity with at least one of an amino acid sequence selected from the group of SEQ ID NOS: 97-103, 116-120, 126-133, and 144-146.

In some embodiments, the isolated antigen binding construct is specific against human FAP. In some embodiments, the antigen binding construct is specific against FAP alpha. In some embodiments, the isolated antigen binding construct of any one of the above embodiments is specific against FAP alpha (FIG. 54). In some embodiments, the isolated antigen binding construct does not bind DPP4. In some embodiments, the antigen binding construct does not bind DPP4. In some embodiments, the antigen binding construct has a higher binding affinity to FAP and/or FAP alpha than to DPP4. In some embodiments, the isolated antigen binding construct has an expression in mammalian cells that is at least about 2-fold, at least about 3-fold, at least about 4-fold, at least about 5-fold, at least about 6-fold, at least about 7-fold, at least about 8-fold, at least about 9-fold, or at least about 10-fold, greater than the expression of Sibrotuzumab minibodies. In some embodiments, the isolated antigen binding construct has a KD of less than about 3×10\hat{ }-9M, less than about 2.5×10\hat{ }-9M, less than about 2×10\hat{ }-9 M, less than about 1.5×10\hat{ }-9M, less than about 1×10\hat{ }-9M, or less than about 0.5×10\hat{ }-9M. In some embodiments, the isolated antigen binding construct has an on-rate (kₒₙ) greater than about 7.0 (l/Ms), greater than about 8.0 (l/Ms), greater than about 9.0 (l/Ms), or greater than about 10.0 (l/Ms). In some embodiments, the isolated antigen binding construct has an on-rate (kₒₙ) greater than 9.0 (l/Ms). In some embodiments, the isolated antigen binding construct has an off-rate (k_{off}) lesser than about 3×10\hat{ }-3 (l/s), lesser than about 2.5×10\hat{ }-3 (l/s), lesser than about 2×10\hat{ }-3 (l/s), lesser than about 1.5×10\hat{ }-3 (l/s), or lesser than about 1.0×10\hat{ }-3 (l/s).

In some embodiments, the order of the variable regions, from N terminus to C terminus of the polypeptide is V_{L}, V_{H}. In some embodiments, the order of the variable regions, from N terminus to C terminus of the polypeptide is V_{H}, V_{L}. In some embodiments, the isolated antigen binding construct further comprises a payload. In some embodiments, the payload is fluorescent. In some embodiments, the payload is luminescent. In some embodiments, the payload is colorimetric. In some embodiments, the payload is radioactive. In some embodiments, the payload is non-radioactive. In some embodiments, the payload is ADC. In some embodiments, the isolated antigen binding construct is humanized. In some embodiments, the payload is chemically reactive. In some embodiments, the payload is a detectable marker.

Some aspects of the present disclosure relate to a composition comprising the amino acid sequence of any one of embodiments 1-66, further comprising at least one payload, such as a label or therapeutic ion, selected from at least one of a group consisting of ¹⁸F, ¹⁸F-FAC, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁵Sc, ⁷⁷As, ⁸⁶Y, ⁹⁰Y, ⁸⁹S, ⁸⁹Zr, ⁹⁴Tc, ⁹⁴Tc, ⁹⁹mTc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁴⁹Tb, ¹⁵³Sm, ¹⁵⁴⁻¹⁵⁸Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁷Th and ²²⁵Ac, or any combination thereof. Some aspects of the present disclosure relate to an expression vector capable of expressing the sequence of any one of the embodiments disclosed herein. In some embodiments, the vector is a viral vector selected from a lentiviral vector or an adenoviral vector. In some embodiments, the vector comprises a sequence encoding the cleavable signal peptide having at least about 80%, at least about 85%, at least about 90%, at least about 95%, at least about 99%, and/or at least about 100% identity with the amino acid sequence of SEQ ID NOs: 46 and 121 (METDTLLLWVLLLWVPGSTG).

In some embodiments, the amino acid sequence of the antigen binding construct further comprises at least one metal binding site. In some embodiments, the amino acid sequence of the antigen binding construct further comprises a histidine (His) tag sequence. In some embodiments, the amino acid sequence of the antigen binding construct further comprises at ^{99m}Tc-carbonyl radiolabel.

Some aspects of the present disclosure relate to a method of transferring the amino acid sequence or expression vector of any of the antigen binding constructs and/or expression vectors encoding them into a host cell, comprising performing electroporation, viral infection, and/or at least one chemical method. Some aspects of the present disclosure relate to a host cell comprising the amino acid sequence or expression vector of any one of the above embodiments disclosed herein.

Some aspects of the present disclosure relate to a composition comprising the amino acid sequence of any one of the above embodiments disclosed herein and at least one chelator. In some embodiments, the at least one chelator is selected from a group consisting of ethylenediaminetetraacetic acid (EDTA), diethylenetriaminepentaacetic acid (DTPA), DOTA, NOTA, NOGADA, NETA, deferoxamine (DFO), porphyrins, polyamines, crown ethers, bis-thiosemicarbazones, polyoximes, dimercaprol, penicillamine, trientine, zinc, deferasirox, deferiprone, deferoxamine, succimer, pyrophosphoric acid, tripolyphosphoric acid, citric acid, tartaric acid, glycine, DMPS, DMSA, NTA, calcium, sodium, desferioxamine, dicobalt EDTA, dimercarpol, BAL, and demercaptosuccinic acid, or any combination thereof. In some embodiments, the composition further comprises dual chelators. In some embodiments, the dual chelators are ⁶⁴Cu/⁶⁷Cu. In some embodiments, the dual chelators are ⁸⁹Zr/¹⁷⁷Lu. In some embodiments, the at least one chelator is configured to capture an isotope. In some embodiments, the composition further comprises at least one payload. In some embodiments, the at least one payload is selected from a group consisting of ¹⁸F, ¹⁸F-FAC, ³²P, ³³P, ⁴⁵Ti, ⁴⁷SC, ⁵²Fe, ⁵⁹Fe, ⁶²Cu, ⁶⁴Cu, ⁶⁷Cu, ⁶⁷Ga, ⁶⁸Ga, ⁷⁵Sc, ⁷⁷As, ⁸⁶Y, ⁹⁰Y, ⁸⁹Sr, ⁸⁹Zr, ⁹⁴Tc, ⁹⁴Tc, ⁹⁹mTc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁴⁹Tb, ¹⁵³Sm, ¹⁵⁴⁻¹⁵⁸Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁷Th and ²²⁵Ac, or any combination thereof.

Some aspects of the present disclosure relate to a use of the composition disclosed herein as a medicament. Some aspects of the present disclosure relate to a use of the composition disclosed herein in the manufacture of a medicament for administration to a subject. Some aspects of the present disclosure relate to a use of the composition disclosed herein for imaging a cell, tissue, organ, and/or subject. Some aspects of the present disclosure relate to a use of the composition disclosed herein for identifying a disease in a subject. In some embodiments, the disease is a cancer or tumor. In some embodiments, the disease is a solid tumor. In some embodiments, the disease is fibrosis. In some embodiments, the disease is an autoimmune disease. In some embodiments, the disease is cardiovascular.

Some aspects of the present disclosure relate to a method of identifying a disease in a subject, comprising: administering at least one of the antibody, antigen binding construct, minibody, and/or cys-diabody of any one of the embodiments disclosed herein to the subject; screening for the binding of the at least one antibody, antigen binding construct, minibody, and/or cys-diabody to FAP; and determining whether the subject has a disease thereof based upon the presence or absence of binding to FAP. In some embodiments, FAP is FAP alpha. In some embodiments, wherein the disease is a cancer or tumor. In some embodiments, the disease is a solid tumor. In some embodiments, the disease is fibrosis. In some embodiments, the disease is an autoimmune disease. In some embodiments, the disease is cardiovascular. In some embodiments, the subject is mammalian or human. In some embodiments, the subject is a mouse or rat.

In some embodiments, the at least one of the antibody, antigen binding construct, minibody, and/or cys-diabody binds to a tumor stroma. In some embodiments, the at least one of the antibody, antigen binding construct, minibody, and/or cys-diabody binds to a fibroblast. In some embodiments, the at least one of the antibody, antigen binding construct, minibody, and/or cys-diabody binds to a cancer-associated fibroblast. In some embodiments, the disease is epithelial. In some embodiments, the least one antibody, antigen binding construct, minibody, and/or cys-diabody further comprises a payload. In some embodiments, the payload (which can be a marker or label) is selected from a group consisting of ¹⁸F, ¹⁸F-FAC, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁵Sc, ⁷⁷As, ⁸⁶Y, ⁹⁰Y, ⁸⁹Sr, ⁸⁹Zr, ⁹⁴Tc, ⁹⁴Tc, ⁹⁹mTc, 99Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁷Th and ²²⁵Ac. In some embodiments, the payload is used to determine the binding of the at least one antibody, antigen binding construct, minibody, and/or cys-diabody to FAP. In some embodiments, the binding of the at least one antibody, antigen binding construct, minibody, and/or cys-diabody to FAP is determined through a PET scan. In some embodiments, the binding of the at least one antibody, antigen binding construct, minibody, and/or cys-diabody to FAP is determined through an in vitro testing of a tissue or cell sample removed from the subject. In some embodiments, the method further comprises identifying the subject as negative for having a disease upon the absence of significant binding of the at least one antibody, antigen binding construct, minibody, and/or cys-diabody to FAP. In some embodiments, the method further comprises identifying the subject having a disease upon the significant binding of the at least one antibody, antigen binding construct, minibody, and/or cys-diabody to FAP.

Some aspects provide a pharmaceutical composition comprising an amount of the at least one antibody, antigen binding construct, minibody, and/or cys-diabody thereof of any one of the embodiments disclosed herein, that is effective for treating a subject with cancer and/or a tumor; and a pharmaceutically acceptable carrier. In some embodiments, the amount of the at least one antibody, antigen binding construct, minibody, and/or cys-diabody is from about 0.01 mg/kg to about 25 mg/kg. In some embodiments, the amount of the at least one antibody, antigen binding construct, minibody, and/or cys-diabody is from about 1 mg/kg to about 20 mg/kg.

In some embodiments, the pharmaceutical composition further comprises an eluent, diluent, carrier, and/or other molecule to aid in the delivery and effectiveness of the composition. In some embodiments, the pharmaceutical composition is formulated for oral delivery. In some embodiments, the pharmaceutical composition is formulated for nasal delivery. In some embodiments, the pharmaceutical composition is formulated for intravenous delivery. In some embodiments, the pharmaceutical composition is formulated for a one-time dose. In some embodiments, the pharmaceutical composition is formulated for multiple dosage delivery. In some embodiments, the pharmaceutical composition further comprises an at least one known small molecule, therapeutic, or antigen binding construct effective in treating a disease. In some embodiments, the at least one known small molecule, therapeutic, or antigen binding construct effective in treating a disease is an antibody. In some embodiments, the at least one known small molecule, therapeutic, or antigen binding construct effective in treating a disease is used as part of chemotherapy. In some embodiments, the at least one known small molecule, therapeutic, or antigen binding construct effective in treating a disease is an immuno-oncology drug. In some embodiments, the at least one known small molecule, therapeutic, or antigen binding construct effective in treating a disease is an immune-oncology drug. In some embodiments, the at least one known small molecule, therapeutic, or antigen binding construct effective in treating a disease is a DNA repair inhibitor. In some embodiments, the at least one known small molecule, therapeutic, or antigen binding construct effective in treating a disease is used as part of photodynamic therapy. In some embodiments, the at least one known small molecule, therapeutic, or antigen binding construct effective in treating a cancer and/or tumor is selected from the group consisting of an alkylating agent, a metabolic inhibitor, a radiosensitizer agent, an antimetabolite, an anti-tumor antibiotic, a topoisomerase inhibitor, a mitotic inhibitor, a nitrosourea, a corticosteroid, an anti-angiogenic, an apoptosis inducer, an anti-microtubule agent, a vinca alkaloid, a taxane, an anthracycline, an anti-androgen, a VEGF pathway inhibitor, a VEGF pathway inhibitor, a MAPK/Ras/Raf pathway inhibitor, and an EGFR pathway inhibitor. In some embodiments, the disease is fibrosis. In some embodiments, the disease is a cancer or tumor. In some embodiments, the disease is a solid tumor. In some embodiments, the disease is an autoimmune disease. In some embodiments, the disease is cardiovascular.

Some aspects of the present disclosure relate to a method of treating, inhibiting, or ameliorating a disease in a subject, comprising: administering the pharmaceutical composition of any one of embodiments disclosed herein to the subject in need thereof. In some embodiments, the subject is mammalian. In some embodiments, the subject is a human. In some embodiments, the disease is fibrosis. In some embodiments, the disease is a cancer or tumor. In some embodiments, the disease is a solid tumor. In some embodiments, the disease is an autoimmune disease. In some embodiments, the disease is cardiovascular. In some embodiments, the method further comprises imaging the disease using the composition of any of embodiments disclosed herein. In some embodiments, the method further comprises imaging the disease using any of the antigen binding constructs disclosed herein.

Some aspects of the present disclosure relate to a method of targeting FAP protein on a fibroblast in a subject, comprising: administering the pharmaceutical composition of any one of embodiments disclosed herein to the subject. In some embodiments, the subject is mammalian. In some embodiments, the subject is human. In some embodiments, the fibroblast is a cancer-associated fibroblast In some embodiments, the cancer is in a solid tumor. In some embodiments, a cancer cell and/or a tumor associated macrophage are damaged or killed following the targeting of the fibroblast.

Some aspects of the present disclosure relate to a method of inhibiting, ameliorating, damaging, or inducing apoptosis in a cancer or tumor associated macrophage in a subject. In some embodiments, the method comprises administering the pharmaceutical composition of any one of the embodiments disclosed herein to the subject in need thereof.

In some embodiments, the subject is mammalian or human.

In some embodiments, the method further comprises administering radiation therapy, photodynamic therapy, and/or chemotherapy to a subject.

In some embodiments, the disease is a solid tumor. In some embodiments, the disease is epithelial cancer. In some embodiments, the cancer or tumor is selected from the group consisting of bone cancer, bone sarcoma, breast cancer, triple negative breast cancer, carcinoid, cervical cancer, colon cancer, colorectal cancer, endometrial carcinoma, epithelial ovarian cancer, esophageal cancer, gastric cancer, gastrointestinal cancer, glioma, glioblastoma, brain cancer, head and neck cancer, hepatocellular cancer, kidney cancer, leukemia, liver cancer, lung cancer, lymphoma, medullary thyroid carcinoma, melanoma, non-small cell lung cancer, small cell lung cancer, osteosarcoma, oral squamous cell carcinoma, oral cancer, ovarian carcinoma, ovarian cancer, pancreatic adenocarcinoma, pancreatic cancer, prostate cancer, rectal cancer, renal cancer, anal cancer, skin cancer, stomach cancer, testicular cancer, thyroid cancer, anaplastic thyroid cancer and urothelial cancer.

### Therapeutic Agents and Compositions

In some embodiments, therapy comprises administering a composition of at least one therapeutic agent, such as any of the appropriate antigen binding constructs provided herein, optionally with other excipients.

Proper formulation of treatment can be dependent upon the route of administration chosen. Techniques for formulation and administration of the compounds described herein are known to those skilled in the art. Multiple techniques of administering a compound exist in the art including, but not limited to, enteral, oral, rectal, topical, sublingual, buccal, intraaural, epidural, epicutaneous, aerosol, parenteral delivery, including intramuscular, subcutaneous, intra-arterial, intravenous, continuous infusion, intraportal, intra-articular, intradermal, peritoneal, intramedullary injections, intrathecal, direct intraventricular, intraperitoneal, intranasal or intraocular injections, subcutaneous, intracranial injection, injection in surgically created resection cavities (SCRCs), injection through Ommaya reservoir, injections through Rickham reservoir. Formulations suitable for parenteral administration include aqueous and non-aqueous isotonic sterile injection solutions which may contain, for example, antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose sealed containers, for example, ampoules and vials, and may be stored in a freeze-dried (lyophilized) condition requiring only the addition of the sterile liquid carrier, for example water for injections, immediately prior to use. Injection solutions and suspensions may be prepared from sterile powders, granules and tablets of the kind previously described.

In some embodiments unit dosage formulations contain a daily dose or unit, daily sub-dose, or an appropriate fraction thereof, of a drug. It will be understood, however, that the specific dose level for any particular patient will depend on a variety of factors including the activity of the specific compound employed; the age, body weight, general health, sex and diet of the individual being treated; the time and route of administration; the rate of excretion; other drugs which have previously been administered; and the severity of the particular disease undergoing therapy, as is well understood by those skilled in the art.

Alternatively, the formulations may be presented in a form suitable for once-daily, once-weekly or once-monthly administration; for example, an insoluble salt of the active compound may be adapted to provide a preparation for intramuscular injection. The pharmaceutical formulations described herein can be administered to a patient per se, or in pharmaceutical formulations where they are mixed with other active ingredients, as in combination therapy, or suitable pharmaceutically acceptable carriers or excipient(s). Techniques for formulation and administration of the compounds of the instant application may be found in “Remington's Pharmaceutical Sciences,” Mack Publishing Co., Easton, PA, 18th edition, 1990.

Pharmaceutical formulations for parenteral administration, e.g., by bolus injection or continuous infusion, include aqueous solutions of the active compounds in water-soluble form. Additionally, suspensions of the active compounds may be prepared as appropriate oily injection suspensions. Suitable lipophilic solvents or vehicles include fatty oils such as sesame oil, or other organic oils such as soybean, grapefruit or almond oils, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions. Formulations for injection may be presented in unit dosage form, e.g., in ampoules or in multi-dose containers, with an added preservative. The formulations may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use. In some embodiments, the formulation includes an at least one agent that acts to reduce radiolysis (also known as “radioprotectors”). Non-limiting examples of radiolysis reducing agents include Acetylcholine, AET, ACE inhibitors, acteoside, alpha-tocopherol acetate, amifostine, ascorbic acid, aspirin, atorvastatin, beta-carotene, Bowman-Birk proteinase inhibitor, Caffeic acid, Captopril, carbaminoylcholine, Carvacrol, Celecoxib, coenzyme Q10, COX2 inhibitors/NSAIDs, curcumin, cysteine, cysteamine, cystamine, dendrodine analog, Dithiolthione, Dopamine, enalapril, epigallocatechin-3-gallate, Epinephrine, 17-β-estradiol, GANRA-5, Genistein, green tea abstract, growth factors, guanine nucleotides, Halofuginone, Hmg-CoA reductase inhibitors (statins), heroin, histamine, ibuprofen, in a poyl-E-glucoside, Isoflavone, isofraxidin, kukoamine A, lactoferrin amifostine, lipoic acid, lovastatin, luteolin-7-O-(2-apiosyl)-glucoside, 2-mercaptoethylguanidine, melatonin, methacholine, morphine, N-acetyl cysteine, Oltipraz, palifermin, phenethyl ester, polyphenols, pravastatin, protease inhibitors, quercetin-3-O-rhamnoside-7-O-glucoside, quercetin-3-O-rhamnoside, ramipril, Resveratrol, rutin, serotonin, Simvastatin, Sodium ascorbate, superoxide dismutase, TGF-signaling inhibitors, tocopherols, vitamin C, vitamin E, watermelon juice, black grape juice, and thiols such as glutathione.

The dosage regimen utilizing the compounds of the present embodiments is selected in accordance with a variety of factors including type, species, age, weight, sex and medical condition of the patient; the severity of the condition to be treated; the route of administration; the renal and hepatic function of the patient; and the particular compound thereof employed. A physician or veterinarian of ordinary skill can readily determine and prescribe the effective amount of the drug required to prevent, counter or arrest the progress of the condition. Optimal precision in achieving concentration of drug within the range that yields efficacy without toxicity requires a regimen based on the kinetics of the pharmaceutical formulation's availability to target sites. This involves a consideration of the distribution, equilibrium, and elimination of the compounds. Advantageously, compounds of the present embodiments may be administered, for example, in a single daily dose, or the total daily dosage may be administered in divided doses of two, three or four times daily.

In some embodiments, the pharmaceutical formulations herein described in detail are typically administered in accordance with conventional pharmaceutical practices. Moreover, when desired or necessary, suitable pharmaceutically acceptable carriers can also be incorporated into the mixture.

The dosage of the products may be varied over a wide range. An effective amount of the instant compounds is ordinarily supplied at a dosage level of from about 0.01 mg/kg to about 25 mg/kg of body weight per dose. In some embodiments, dosage is administered daily. In some embodiments, dosage is administered as a single dosage. In some embodiments, dosage is administered as a fractionated dosage repeated at intervals of once every about 1 week, about 2 weeks, about 3 weeks, about 4 weeks, about 6 weeks, or about 10 weeks.

Embodiments of the present disclosure provided herein are described by way of the following exemplary numbered arrangements:
1. An isolated antigen binding construct thereof, comprising:
   a HCDR1 comprising amino acids having at least 80% identity with the amino acid sequence of SEQ ID NO: 1 (EYTIH);
   a HCDR2 comprising amino acids having at least 80% identity with the amino acid sequence of SEQ ID NO: 2 (GINPNNGIPNYNQKFKG);
   a HCDR3 comprising amino acids having at least 80% identity with the amino acid sequence of SEQ ID NO: 3 (RRIAYGYDEGHAMDY);
   a LCDR1 comprising amino acids having at least 80% identity with the amino acid sequence of SEQ ID NO: 4 (KSSQSLLYSRNQKNYLA);
   a LCDR2 comprising amino acids having at least 80% identity with the amino acid sequence of SEQ ID NO: 5 (WASTRES); and
   a LCDR3 comprising amino acids having the amino acid sequence of SEQ ID NO: 6 (QQYYSYPLT).
2. The isolated antigen binding construct of arrangement 1, comprising a heavy chain having at least 80% identity with the amino acid sequence of SEQ ID NO: 7.
3. The isolated antigen binding construct of arrangement 1, comprising a heavy chain having at least 90% identity with the amino acid sequence of SEQ ID NO: 7.
4. The isolated antigen binding construct of arrangement 1, comprising a heavy chain having at least 95% identity with the amino acid sequence of SEQ ID NO: 7.
5. The isolated antigen binding construct of any one of arrangements 1-4, comprising a light chain having at least 80% identity with the amino acid sequence of SEQ ID NO: 8.
6. The isolated antigen binding construct of any one of arrangements 1-4, comprising a light chain having at least 90% identity with the amino acid sequence of SEQ ID NO: 8.
7. The isolated antigen binding construct of any one of arrangements 1-4, comprising a light chain having at least 95% identity with the amino acid sequence of SEQ ID NO: 8.
8. The isolated antigen binding construct of any one of arrangements 1-4, comprising a light chain having at least 80% identity with the amino acid sequence of SEQ ID NO: 9.
9. The isolated antigen binding construct of any one of arrangements 1-4, comprising a light chain having at least 90% identity with the amino acid sequence of SEQ ID NO: 9.
10. The isolated antigen binding construct of any one of arrangements 1-4, comprising a light chain having at least 95% identity with the amino acid sequence of SEQ ID NO: 9.
11. The isolated antigen binding construct of any one of arrangements 1-10, comprising at least one VH framework residue selected from the group consisting of:
   an Alanine at position 24 of the sequence in SEQ ID NO: 7; or
   a Glycine at position 26 of the sequence in SEQ ID NO: 7.
12. The isolated antigen binding construct of any one of arrangements 1-11, comprising at least one VL framework residue selected from the group consisting of:
   a Serine at position 73 of the sequence in SEQ ID NO: 8;
   an Arginine at position 83 of the sequence in SEQ ID NO: 8;
   a Glutamic acid at position 85 of the sequence in SEQ ID NO:8;
   a Proline at position 86 of the sequence in SEQ ID NO: 8;
   a Phenylalanine at position 89 of the sequence in SEQ ID NO: 8; or
   a Tyrosine at position 98 of the sequence in SEQ ID NO: 8.
13. The isolated antigen binding construct of any one of arrangements 1-12, comprising a phenylalanine at position 89 of the light chain sequence in SEQ ID NO: 8.
14. The isolated antigen binding construct of any one of arrangements 1-13, comprising an alanine at position 24 and a glycine at position 26 of the heavy chain sequence in SEQ ID NO: 7.
15. The isolated antigen binding construct of any one of arrangements 1-14, comprising a phenylalanine at position 89 and a serine at position 73 of the sequence of the light chain sequence in SEQ ID NO: 8.
16. The isolated antigen binding construct of any one of arrangements 1-15, comprising a phenylalanine at position 89, a serine at position 73, and a tyrosine at position 98 of the sequence of the light chain sequence in SEQ ID NO: 8.
17. The isolated antigen binding construct of any one of arrangements 1-16, comprising a phenylalanine at position 89 and a tyrosine at position 98 of the sequence of the light chain sequence in SEQ ID NO: 8.
18. The isolated antigen binding construct of any one of arrangements 1-17, comprising a phenylalanine at position 89, an arginine at position 83, a glutamic acid at position 85, a proline at position 86, and a tyrosine at position 98 of the sequence of the light chain sequence in SEQ ID NO: 8.
19. The isolated antigen binding construct of any one of arrangements 1-18, comprising a phenylalanine at position 89, a serine at position 73, an arginine at position 83, a glutamic acid at position 85, and a proline at position 86 of the sequence of the light chain sequence in SEQ ID NO: 8.
20. The isolated antigen binding construct of any one of arrangements 1-19, comprising a phenylalanine at position 89, a serine at position 73, an arginine at position 83, a glutamic acid at position 85, a proline at position 86, and a tyrosine at position 98 of the sequence of the light chain sequence in SEQ ID NO: 8.
21. The isolated antigen binding construct of any one of arrangements 1-20, comprising a phenylalanine at position 89, an arginine at position 83, a glutamic acid at position 85, and a proline at position 86 of the sequence of the light chain sequence in SEQ ID NO: 8.
22. The antigen binding construct of any of Arrangements 1-21, comprising:
   a variable heavy domain (VH) of SEQ ID NO: 7; and
   a variable light domain (VL) of SEQ ID NO: 8.
23. The antigen binding construct of any of Arrangements 1-21, comprising:
   a variable heavy domain (VH) of SEQ ID NO: 7; and
   a variable light domain (VL), of SEQ ID NO: 9.
24. An isolated antigen binding construct specific against FAP alpha comprising a CDR3 comprising amino acids having at least 90% identity with the amino acid sequence of SEQ ID NO: 6 (QQYYSYPLT).
25. An isolated antigen binding construct comprising a light chain CDR3 comprising amino acids having at least 100% identity with the amino acid sequence of SEQ ID NO: 6.
26. An isolated antigen binding construct thereof, comprising:
   a LCDR1 comprising amino acids having at least 80% identity with the amino acid sequence of SEQ ID NO: 4 (KSSQSLLYSRNQKNYLA);
   a LCDR2 comprising amino acids having at least 80% identity with the amino acid sequence of SEQ ID NO: 5 (WASTRES); and
   a LCDR3 comprising amino acids having the amino acid sequence of SEQ ID NO: 6 (QQYYSYPLT).
   27. An isolated antigen binding construct comprising a heavy chain having at least 99% identity with the amino acid sequence of SEQ ID NO: 7.
28. An isolated antigen binding construct comprising a heavy chain having at least 80% identity with the amino acid sequence of SEQ ID NO: 7, comprising at least one VH framework residue selected from the group consisting of:
   an Alanine at position 24 of the sequence in SEQ ID NO: 7;
   a Glycine at position 26 of the sequence in SEQ ID NO: 7.
29. An isolated antigen binding construct comprising a light chain having at least 80% identity with the amino acid sequence of SEQ ID NO: 8, comprising at least one VL framework residue selected from the group consisting of:
   a Serine at position 73 of the sequence in SEQ ID NO: 8;
   an Arginine at position 83 of the sequence in SEQ ID NO: 8;
   a Glutamic acid at position 85 of the sequence in SEQ ID NO:8;
   a Proline at position 86 of the sequence in SEQ ID NO: 8;
   a Phenylalanine at position 89 of the sequence in SEQ ID NO: 8;
   a Tyrosine at position 98 of the sequence in SEQ ID NO: 8.
30. An isolated antigen binding construct comprising a light chain comprising amino acids having at least 95% identity with the amino acid sequence of SEQ ID NO: 9.
31. An isolated antigen binding construct comprising a light chain comprising amino acids having at least 95% identity with the amino acid sequence of SEQ ID NO: 8.
32. An isolated antigen binding construct comprising:
   a variable heavy domain (VH) comprising amino acids having at least 95% identity with the amino acid sequence of SEQ ID NO: 7; and
   a variable light domain (VL) comprising amino acids having at least 95% identity with the amino acid sequence of SEQ ID NO: 8.
33. An isolated humanized antigen binding construct comprising:
   a variable heavy domain (VH) comprising amino acids having at least 95% identity with the amino acid sequence of SEQ ID NO: 7; and
   a variable light domain (VL) comprising amino acids having at least 89% identity with the amino acid sequence of SEQ ID NO: 9.
34. The isolated antigen binding construct of any one of arrangements 27, 32, or 33, comprising at least one VH framework residue selected from the group consisting of:
   an Alanine at position 24 of the sequence in SEQ ID NO: 7;
   a Glycine at position 26 of the sequence in SEQ ID NO: 7.
35. The isolated antigen binding construct of any one of arrangements 29, 31, or 32, comprising at least one VL framework residue selected from the group consisting of:
   a Serine at position 73 of the sequence in SEQ ID NO: 8;
   an Arginine at position 83 of the sequence in SEQ ID NO: 8;
   a Glutamic acid at position 85 of the sequence in SEQ ID NO:8;
   a Proline at position 86 of the sequence in SEQ ID NO: 8;
   a Phenylalanine at position 89 of the sequence in SEQ ID NO: 8;
   a Tyrosine at position 98 of the sequence in SEQ ID NO: 8.
36. The isolated antigen binding construct of any one of arrangements 29, 31, or 32, comprising a phenylalanine at position 89 of the light chain sequence in SEQ ID NO: 8.
37. The isolated antigen binding construct of any one of arrangements 29, 31, or 32, comprising a phenylalanine at position 89 and a serine at position 73 of the sequence of the light chain sequence in SEQ ID NO: 4.
38. The isolated antigen binding construct of any one of arrangements 29, 31, or 32, comprising a phenylalanine at position 89, a serine at position 73, and a tyrosine at position 98 of the sequence of the light chain sequence in SEQ ID NO: 8.
39. The isolated antigen binding construct of any one of arrangements 29, 31, or 32, comprising a phenylalanine at position 89 and a tyrosine at position 98 of the sequence of the light chain sequence in SEQ ID NO: 8.
40. The isolated antigen binding construct of any one of arrangements 29, 31, or 32, comprising a phenylalanine at position 89, an arginine at position 83, a glutamic acid at position 85, a proline at position 86, and a tyrosine at position 98 of the sequence of the light chain sequence in SEQ ID NO: 8.
41. The isolated antigen binding construct of any one of arrangements 29, 31, or 32, comprising a phenylalanine at position 89, a serine at position 73, an arginine at position 83, a glutamic acid at position 85, and a proline at position 86 of the sequence of the light chain sequence in SEQ ID NO: 8.
42. The isolated antigen binding construct of any one of arrangements 29, 31, or 32, comprising a phenylalanine at position 89, a serine at position 73, an arginine at position 83, a glutamic acid at position 85, a proline at position 86, and a tyrosine at position 98 of the sequence of the light chain sequence in SEQ ID NO: 8.
43. The isolated antigen binding construct of any one of arrangements 29, 31, or 32, comprising a phenylalanine at position 89, an arginine at position 83, a glutamic acid at position 85, and a proline at position 86 of the sequence of the light chain sequence in SEQ ID NO: 8.
44. An isolated antigen binding construct comprising amino acids having at least 80% identity with an amino acid sequence selected from the group of SEQ ID NOS: 23-44.
45. An isolated antigen binding construct comprising amino acids having at least 90% identity with an amino acid sequence selected from the group of SEQ ID NOS: 23-44.
46. An isolated antigen binding construct comprising amino acids having at least 100% identity with an amino acid sequence selected from the group of SEQ ID NOS: 23-44, 12-43, 12-45, 87-96, 110-115, 122-125, or 140-143.
47. An isolated antigen binding construct thereof, comprising:
   a HCDR1 comprising the amino acid sequence of SEQ ID NO: 81 (NYDIN), or a sequence that has no more than 3 point mutations thereto;
   a HCDR2 comprising the amino acid sequence of SEQ ID NO: 82 (LIWTGGGTN), or a sequence that has no more than 3 point mutations thereto;
   a HCDR3 comprising the amino acid sequence of SEQ ID NO: 83 (GGPLVWYALDY), or a sequence that has no more than 3 point mutations thereto;
   a LCDR1 comprising the amino acid sequence of SEQ ID NO: 84 (KASQDVSTAVA), or a sequence that has no more than 3 point mutations thereto;
   a LCDR2 comprising the amino acid sequence of SEQ ID NO: 85 (SASYRYT), or a sequence that has no more than 3 point mutations thereto; and/or
   a LCDR3 comprising the amino acid sequence of SEQ ID NO: 86 (QQHYSNPRT), or a sequence that has no more than 3 point mutations thereto.
48. An isolated antigen binding construct thereof, comprising:
   a HCDR1 comprising the amino acid sequence of SEQ ID NO: 81 (NYDIN), or a sequence that has no more than 3 point mutations thereto;
   a HCDR2 comprising the amino acid sequence of SEQ ID NO: 82 (LIWTGGGTN), or a sequence that has no more than 3 point mutations thereto; and/or
   a HCDR3 comprising the amino acid sequence of SEQ ID NO: 83 (GGPLVWYALDY), or a sequence that has no more than 3 point mutations thereto.
49. An isolated antigen binding construct thereof, comprising:
   a LCDR1 comprising the amino acid sequence of SEQ ID NO: 84 (KASQDVSTAVA), or a sequence that has no more than 3 point mutations thereto;
   a LCDR2 comprising the amino acid sequence of SEQ ID NO: 85 (SASYRYT), or a sequence that has no more than 3 point mutations thereto; and/or
   a LCDR3 comprising the amino acid sequence of SEQ ID NO: 86 (QQHYSNPRT), or a sequence that has no more than 3 point mutations thereto.
50. An isolated antigen binding construct specific against FAP comprising a CDR1 comprising the amino acid sequence of SEQ ID NO: 81 (NYDIN), or a sequence that has no more than 3 point mutations thereto.
51. The isolated antigen binding construct of arrangement 50, wherein the CDR1 is part of a heavy chain.
52. An isolated antigen binding construct specific against FAP comprising a CDR2 comprising with the amino acid sequence of SEQ ID NO: 82 (LIWTGGGTN), or a sequence that has no more than 3 point mutations thereto.
53. The isolated antigen binding construct of arrangement 52, wherein the CDR2 is part of a heavy chain.
54. An isolated antigen binding construct specific against FAP comprising a CDR3 comprising the amino acid sequence of SEQ ID NO: 83 (GGPLVWYALDY), or a sequence that has no more than 3 point mutations thereto.
55. The isolated antigen binding construct of arrangement 54, wherein the CDR3 is part of a heavy chain.
56. An isolated antigen binding construct specific against FAP comprising a CDR1 comprising the amino acid sequence of SEQ ID NO: 84 (KASQDVSTAVA), or a sequence that has no more than 3 point mutations thereto.
57. The isolated antigen binding construct of arrangement 56, wherein the CDR1 is part of a light chain.
58. An isolated antigen binding construct specific against FAP comprising a CDR2 comprising the amino acid sequence of SEQ ID NO: 85 (SASYRYT), or a sequence that has no more than 3 point mutations thereto.
59. The isolated antigen binding construct of arrangement 58, wherein the CDR2 is part of a light chain.
60. An isolated antigen binding construct specific against FAP comprising a CDR3 comprising the amino acid sequence of SEQ ID NO: 86 (QQHYSNPRT), or a sequence that has no more than 3 point mutations thereto.
61. The isolated antigen binding construct of arrangement 60, wherein the CDR3 is part of a light chain.
62. The isolated antigen binding construct of any one of arrangements 47-61, wherein there are no more than 2 point mutations.
63. The isolated antigen binding construct of any one of arrangements 47-61, wherein there is no more than 1 point mutation.
64. The isolated antigen binding construct of any one of arrangements 47-61, wherein there are no point mutations.
65. The isolated antigen binding construct of any one of arrangements 47-64, further comprising a heavy chain having at least 80% identity with the amino acid sequence of SEQ ID NO: 87.
66. The isolated antigen binding construct of any one of arrangements 47-65, further comprising a light chain having at least 80% identity with the amino acid sequence of SEQ ID NO: 88.
67. The antigen binding construct of any of arrangements 47-66, further comprising:
   a variable heavy domain (VH) having at least 80% identity with the amino acid sequence of SEQ ID NO: 87; and/or
   a variable light domain (VL) chain having at least 80% identity with the amino acid sequence of SEQ ID NO: 88.
68. An isolated antigen binding construct thereof, comprising:
   a variable heavy domain (VH) having at least 80% identity with the amino acid sequence of SEQ ID NO: 87; and/or
   a variable light domain (VL) chain having at least 80% identity with the amino acid sequence of SEQ ID NO: 88.
69. The antigen binding construct of any of arrangements 47-66, further comprising:
   a variable heavy domain (VH) having at least 80% identity with the amino acid sequence of SEQ ID NO: 89; and/or
   a variable light domain (VL) chain having at least 80% identity with the amino acid sequence of SEQ ID NO: 90.
70. An isolated antigen binding construct thereof, comprising:
   a variable heavy domain (VH) having at least 80% identity with the amino acid sequence of SEQ ID NO: 89; and/or
   a variable light domain (VL) chain having at least 80% identity with the amino acid sequence of SEQ ID NO: 90.
71. The antigen binding construct of any of arrangements 47-66, further comprising:
   a variable heavy domain (VH) having at least 80% identity with the amino acid sequence of SEQ ID NO: 91; and/or
   a variable light domain (VL) chain having at least 80% identity with the amino acid sequence of SEQ ID NO: 92.
72. An isolated antigen binding construct thereof, comprising:
   a variable heavy domain (VH) having at least 80% identity with the amino acid sequence of SEQ ID NO: 91; and/or
   a variable light domain (VL) chain having at least 80% identity with the amino acid sequence of SEQ ID NO: 92.
73. The antigen binding construct of any of arrangements 47-66, further comprising:
   a variable heavy domain (VH) having at least 80% identity with the amino acid sequence of SEQ ID NO: 93 and/or
   a variable light domain (VL) chain having at least 80% identity with the amino acid sequence of SEQ ID NO: 94.
74. An isolated antigen binding construct thereof, comprising:
   a variable heavy domain (VH) having at least 80% identity with the amino acid sequence of SEQ ID NO: 93; and/or
   a variable light domain (VL) chain having at least 80% identity with the amino acid sequence of SEQ ID NO: 94.
75. The antigen binding construct of any of arrangements 47-66, further comprising:
   a variable heavy domain (VH) having at least 80% identity with the amino acid sequence of SEQ ID NO: 95 and/or
   a variable light domain (VL) chain having at least 80% identity with the amino acid sequence of SEQ ID NO: 96.
76. An isolated antigen binding construct thereof, comprising:
   a variable heavy domain (VH) having at least 80% identity with the amino acid sequence of SEQ ID NO: 95; and/or
   a variable light domain (VL) chain having at least 80% identity with the amino acid sequence of SEQ ID NO: 96.
77. The antigen binding construct of any of arrangements 47-66, further comprising:
   a variable heavy domain (VH) having at least 80% identity with the amino acid sequence of SEQ ID NO: 122 and/or
   a variable light domain (VL) chain having at least 80% identity with the amino acid sequence of SEQ ID NO: 123.
78. An isolated antigen binding construct thereof, comprising:
   a variable heavy domain (VH) having at least 80% identity with the amino acid sequence of SEQ ID NO: 122; and/or
   a variable light domain (VL) chain having at least 80% identity with the amino acid sequence of SEQ ID NO: 23.
79. An isolated antigen binding construct thereof, comprising:
   a HCDR1 comprising the amino acid sequence of SEQ ID NO: 104 (SYVMH), or a sequence that has no more than 3 point mutations thereto;
   a HCDR2 comprising the amino acid sequence of SEQ ID NO: 105 (YINPHNDGTK), or a sequence that has no more than 3 point mutations thereto;
   a HCDR3 comprising the amino acid sequence of SEQ ID NO: 106 (ARWGIYYGYGAWFAY), or a sequence that has no more than 3 point mutations thereto;
   a LCDR1 comprising the amino acid sequence of SEQ ID NO: 107 (KASQNVGTYVA), or a sequence that has no more than 3 point mutations thereto;
   a LCDR2 comprising the amino acid sequence of SEQ ID NO: 108 (YSASNRYS), or a sequence that has no more than 3 point mutations thereto; and/or
   a LCDR3 comprising the amino acid sequence of SEQ ID NO: 109 (QQYNTYPYT), or a sequence that has no more than 3 point mutations thereto.
80. An isolated antigen binding construct thereof, comprising:
   a HCDR1 comprising the amino acid sequence of SEQ ID NO: 104 (SYVMH), or a sequence that has no more than 3 point mutations thereto;
   a HCDR2 comprising the amino acid sequence of SEQ ID NO: 105 (YINPHNDGTK), or a sequence that has no more than 3 point mutations thereto; and/or
   a HCDR3 the amino acid sequence of SEQ ID NO: 106 (ARWGIYYGYGAWFAY), or a sequence that has no more than 3 point mutations thereto.
81. An isolated antigen binding construct thereof, comprising:
   a LCDR1 comprising the amino acid sequence of SEQ ID NO: 107 (KASQNVGTYVA), or a sequence that has no more than 3 point mutations thereto;
   a LCDR2 comprising the amino acid sequence of SEQ ID NO: 108 (YSASNRYS), or a sequence that has no more than 3 point mutations thereto; and/or
   a LCDR3 comprising the amino acid sequence of SEQ ID NO: 109 (QQYNTYPYT), or a sequence that has no more than 3 point mutations thereto.
82. An isolated antigen binding construct specific against FAP comprising a CDR1 comprising the amino acid sequence of SEQ ID NO: 104 (SYVMH), or a sequence that has no more than 3 point mutations thereto.
83. The isolated antigen binding construct of arrangement 82, wherein the CDR1 is part of a heavy chain.
84. An isolated antigen binding construct specific against FAP comprising a CDR2 comprising the amino acid sequence of SEQ ID NO: 105 (YINPHNDGTK), or a sequence that has no more than 3 point mutations thereto.
85. The isolated antigen binding construct of arrangement 84, wherein the CDR2 is part of a heavy chain.
86. An isolated antigen binding construct specific against FAP comprising a CDR3 comprising the amino acid sequence of SEQ ID NO: 106 (ARWGIYYGYGAWFAY), or a sequence that has no more than 3 point mutations thereto.
87. The isolated antigen binding construct of arrangement 86, wherein the CDR3 is part of a heavy chain.
88. An isolated antigen binding construct specific against FAP comprising a CDR1 comprising the amino acid sequence of SEQ ID NO: 107 (KASQNVGTYVA), or a sequence that has no more than 3 point mutations thereto.
89. The isolated antigen binding construct of arrangement 88, wherein the CDR1 is part of a light chain.
90. An isolated antigen binding construct specific against FAP comprising a CDR2 comprising the amino acid sequence of SEQ ID NO: 108 (YSASNRYS), or a sequence that has no more than 3 point mutations thereto.
91. The isolated antigen binding construct of arrangement 90, wherein the CDR2 is part of a light chain.
92. An isolated antigen binding construct specific against FAP comprising a CDR3 comprising the amino acid sequence of SEQ ID NO: 109 (QQYNTYPYT), or a sequence that has no more than 3 point mutations thereto.
93. The isolated antigen binding construct of arrangement 92, wherein the CDR3 is part of a light chain.
94. The isolated antigen binding construct of any one of arrangements 79-93, wherein there are no more than 2 point mutations thereto.
95. The isolated antigen binding construct of any one of arrangements 79-93, wherein there are no more than 1 point mutation thereto.
96. The isolated antigen binding construct of any one of arrangements 79-93, wherein there are no point mutations.
97. The isolated antigen binding construct of any one of arrangements 79-96, further comprising a heavy chain having at least 80% identity with the amino acid sequence of SEQ ID NO: 110.
98. The isolated antigen binding construct of any one of arrangements 79-97, further comprising a light chain having at least 80% identity with the amino acid sequence of SEQ ID NO: 111.
99. The antigen binding construct of any of arrangements 79-98, further comprising:
   a variable heavy domain (VH) having at least 80% identity with the amino acid sequence of SEQ ID NO: 110; and/or
   a variable light domain (VL) chain having at least 80% identity with the amino acid sequence of SEQ ID NO: 111.
100. An isolated antigen binding construct thereof, comprising:
   a variable heavy domain (VH) having at least 80% identity with the amino acid sequence of SEQ ID NO: 110; and/or
   a variable light domain (VL) chain having at least 80% identity with the amino acid sequence of SEQ ID NO: 111.
101. The antigen binding construct of any of arrangements 79-98, further comprising:
   a variable heavy domain (VH) having at least 80% identity with the amino acid sequence of SEQ ID NO: 112; and/or
   a variable light domain (VL) chain having at least 80% identity with the amino acid sequence of SEQ ID NO: 113.
102. An isolated antigen binding construct thereof, comprising:
   a variable heavy domain (VH) having at least 80% identity with the amino acid sequence of SEQ ID NO: 112; and/or
   a variable light domain (VL) chain having at least 80% identity with the amino acid sequence of SEQ ID NO: 113.
103. The antigen binding construct of any of arrangements 79-98, further comprising:
   a variable heavy domain (VH) having at least 80% identity with the amino acid sequence of SEQ ID NO: 91; and/or
   a variable light domain (VL) chain having at least 80% identity with the amino acid sequence of SEQ ID NO: 92.
104. The antigen binding construct of any of arrangements 79-98, further comprising:
   a variable heavy domain (VH) having at least 80% identity with the amino acid sequence of SEQ ID NO: 114 and/or
   a variable light domain (VL) chain having at least 80% identity with the amino acid sequence of SEQ ID NO: 115.
105. An isolated antigen binding construct thereof, comprising:
   a variable heavy domain (VH) having at least 80% identity with the amino acid sequence of SEQ ID NO: 114; and/or
   a variable light domain (VL) chain having at least 80% identity with the amino acid sequence of SEQ ID NO: 115.
106. The antigen binding construct of any of arrangements 79-98, further comprising:
   a variable heavy domain (VH) having at least 80% identity with the amino acid sequence of SEQ ID NO: 95 and/or
   a variable light domain (VL) chain having at least 80% identity with the amino acid sequence of SEQ ID NO: 96.
107. The antigen binding construct of any of arrangements 79-98, further comprising:
   a variable heavy domain (VH) having at least 80% identity with the amino acid sequence of SEQ ID NO: 124 and/or
   a variable light domain (VL) chain having at least 80% identity with the amino acid sequence of SEQ ID NO: 125.
108. An isolated antigen binding construct thereof, comprising:
   a variable heavy domain (VH) having at least 80% identity with the amino acid sequence of SEQ ID NO: 124; and/or
   a variable light domain (VL) chain having at least 80% identity with the amino acid sequence of SEQ ID NO: 125.
109. An isolated antigen binding construct thereof, comprising:
   a HCDR1 comprising the amino acid sequence of SEQ ID NO: 134 (SYTMS), or a sequence that has no more than 3 point mutations thereto;
   a HCDR2 comprising the amino acid sequence of SEQ ID NO: 135 (TISSGGSYTY), or a sequence that has no more than 3 point mutations thereto;
   a HCDR3 comprising the amino acid sequence of SEQ ID NO: 136 (TRDQVGYAMDY), or a sequence that has no more than 3 point mutations thereto;
   a LCDR1 comprising the amino acid sequence of SEQ ID NO: 137 (TRDQVGYAMDY), or a sequence that has no more than 3 point mutations thereto;
   a LCDR2 comprising the amino acid sequence of SEQ ID NO: 138 (YWASTRHT), or a sequence that has no more than 3 point mutations thereto; and/or
   a LCDR3 comprising the amino acid sequence of SEQ ID NO: 139 (QQYSRYPYT), or a sequence that has no more than 3 point mutations thereto.
110. An isolated antigen binding construct thereof, comprising:
   a HCDR1 comprising the amino acid sequence of SEQ ID NO: 134 (SYTMS), or a sequence that has no more than 3 point mutations thereto;
   a HCDR2 comprising the amino acid sequence of SEQ ID NO: 135 (TISSGGSYTY), or a sequence that has no more than 3 point mutations thereto; and/or
   a HCDR3 comprising the amino acid sequence of SEQ ID NO: 136 (TRDQVGYAMDY), or a sequence that has no more than 3 point mutations thereto.
111. An isolated antigen binding construct thereof, comprising:
   a LCDR1 comprising the amino acid sequence of SEQ ID NO: 137 (TRDQVGYAMDY), or a sequence that has no more than 3 point mutations thereto;
   a LCDR2 comprising the amino acid sequence of SEQ ID NO: 138 (YWASTRHT), or a sequence that has no more than 3 point mutations thereto; and/or
   a LCDR3 comprising the amino acid sequence of SEQ ID NO: 139 (QQYSRYPYT), or a sequence that has no more than 3 point mutations thereto.
112. An isolated antigen binding construct specific against FAP comprising a CDR1 comprising the amino acid sequence of SEQ ID NO: 134 (SYTMS), or a sequence that has no more than 3 point mutations thereto.
113. The isolated antigen binding construct of arrangement 112, wherein the CDR1 is part of a heavy chain.
114. An isolated antigen binding construct specific against FAP comprising a CDR2 comprising the amino acid sequence of SEQ ID NO: 135 (TISSGGSYTY), or a sequence that has no more than 3 point mutations thereto.
115. The isolated antigen binding construct of arrangement 114, wherein the CDR2 is part of a heavy chain.
116. An isolated antigen binding construct specific against FAP comprising a CDR3 comprising the amino acid sequence of SEQ ID NO: 136 (TRDQVGYAMDY), or a sequence that has no more than 3 point mutations thereto.
117. The isolated antigen binding construct of arrangement 116, wherein the CDR3 is part of a heavy chain.
118. An isolated antigen binding construct specific against FAP comprising a CDR1 comprising the amino acid sequence of SEQ ID NO: 137 (TRDQVGYAMDY), or a sequence that has no more than 3 point mutations thereto.
119. The isolated antigen binding construct of arrangement 118, wherein the CDR1 is part of a light chain.
120. An isolated antigen binding construct specific against FAP comprising a CDR2 comprising the amino acid sequence of SEQ ID NO: 138 (YWASTRHT), or a sequence that has no more than 3 point mutations thereto.
121. The isolated antigen binding construct of arrangement 120, wherein the CDR2 is part of a light chain.
122. An isolated antigen binding construct specific against FAP comprising a CDR3 comprising the amino acid sequence of SEQ ID NO: 139 (QQYSRYPYT), or a sequence that has no more than 3 point mutations thereto.
123. The isolated antigen binding construct of arrangement 122, wherein the CDR3 is part of a light chain.
124. The isolated antigen binding construct of any one of arrangements 109-123, wherein there are no more than 2 point mutations thereto.
125. The isolated antigen binding construct of any one of arrangements 109-123, wherein there are no more than 1 point mutation thereto.
126. The isolated antigen binding construct of any one of arrangements 109-123, wherein there are no point mutations.
127. The isolated antigen binding construct of any one of arrangements 109-126, further comprising a heavy chain having at least 80% identity with the amino acid sequence of SEQ ID NO: 140.
128. The isolated antigen binding construct of any one of arrangements 109-127, further comprising a light chain having at least 80% identity with the amino acid sequence of SEQ ID NO: 11.
129. The antigen binding construct of any of arrangements 109-128, further comprising:
   a variable heavy domain (VH) having at least 80% identity with the amino acid sequence of SEQ ID NO: 140; and/or
   a variable light domain (VL) chain having at least 80% identity with the amino acid sequence of SEQ ID NO: 141.
130. An isolated antigen binding construct thereof, comprising:
   a variable heavy domain (VH) having at least 80% identity with the amino acid sequence of SEQ ID NO: 140; and/or
   a variable light domain (VL) chain having at least 80% identity with the amino acid sequence of SEQ ID NO: 141.
131. The antigen binding construct of any of arrangements 109-128, further comprising:
   a variable heavy domain (VH) having at least 80% identity with the amino acid sequence of SEQ ID NO: 142; and/or
   a variable light domain (VL) chain having at least 80% identity with the amino acid sequence of SEQ ID NO: 90.
132. An isolated antigen binding construct thereof, comprising:
   a variable heavy domain (VH) having at least 80% identity with the amino acid sequence of SEQ ID NO: 142; and/or
   a variable light domain (VL) chain having at least 80% identity with the amino acid sequence of SEQ ID NO: 90.
133. The antigen binding construct of any of arrangements 109-128, further comprising:
   a variable heavy domain (VH) having at least 80% identity with the amino acid sequence of SEQ ID NO: 143; and/or
   a variable light domain (VL) chain having at least 80% identity with the amino acid sequence of SEQ ID NO: 94.
134. An isolated antigen binding construct thereof, comprising:
   a variable heavy domain (VH) having at least 80% identity with the amino acid sequence of SEQ ID NO: 143 and/or
   a variable light domain (VL) chain having at least 80% identity with the amino acid sequence of SEQ ID NO: 94.
135. The isolated antigen binding construct of any one of arrangements 47-134, wherein the order of the variable regions, from N terminus to C terminus of the polypeptide is V_{L}, V_{H}.
136. The isolated antigen binding construct of any one of arrangements 47-134, wherein the order of the variable regions, from N terminus to C terminus of the polypeptide is V_{H}, V_{L}.
137. An isolated antigen binding construct comprising amino acids having at least 98% identity with at least one amino acid sequence selected from the group of SEQ ID NOS: 97-103 and 126.
138. An isolated antigen binding construct comprising amino acids having at least 98% identity with at least one amino acid sequence selected from the group of SEQ ID NOS: 116-120, and 127-133.
139. An isolated antigen binding construct comprising amino acids having at least 98% identity with at least one amino acid sequence selected from the group of SEQ ID NOS: 144-146.
140. The isolated antigen binding construct of any one of arrangements 47-139, wherein the antigen binding construct is mammalian.
141. The isolated antigen binding construct of arrangement 140, wherein the antigen binding construct is murine.
142. The isolated antigen binding construct of arrangement 140, wherein the antigen binding construct is human.
143. The isolated antigen binding construct of any one of arrangements 1-139, wherein the antigen binding construct is an antibody.
144. The isolated antigen binding construct of any one of arrangements 1-139, wherein the humanized antigen binding construct is selected from the group consisting of: a scFv, an Fab, an Fab2, a nanobody, a minibody, a cys-diabody, or any combination thereof.
145. The isolated antigen binding construct of any one of arrangements 1-144, wherein the humanized antigen binding construct is a minibody.
146. The isolated antigen binding construct of any one of arrangements 1-144, wherein the humanized antigen binding construct is a cys-diabody.
147. A minibody antigen binding construct comprising amino acids having at least about 80%, at least about 90%, or at least about 99% identity with an amino acid sequence selected from the group of SEQ ID NOS: 11-22.
148. A cys-diabody antigen binding construct comprising amino acids having at least about 80%, at least about 90%, or at least about 99% identity with an amino acid sequence selected from the group of SEQ ID NOS: 11-22.
149. A minibody antigen binding construct comprising amino acids having at least about 80%, at least about 90%, or at least about 99% identity with at least one of an amino acid sequence selected from the group of SEQ ID NOS: 97-103, 116-120, 126-133, and 144-146.
150. A cys-diabody antigen binding construct comprising amino acids having at least about 80%, at least about 90%, or at least about 99% identity with at least one of an amino acid sequence selected from the group of SEQ ID NOS: 97-103, 116-120, 126-133, and 144-146.
151. The isolated antigen binding construct of any one of arrangements 47-146 or 149-150, wherein the antigen binding construct is specific against human FAP.
152. The isolated antigen binding construct of any one of arrangements 1-150, wherein the antigen binding construct is specific against FAP alpha.
153. The isolated antigen binding construct of any one of arrangements 1-150, wherein the antigen binding construct does not bind DPP4.
154. The isolated antigen binding construct of any one of arrangements 1-46, 147-148 or 152-153, wherein the antigen binding construct has an expression in mammalian cells that is at least 2-fold greater than the expression of Sibrotuzumab minibodies (IAB16M1-12 and IAB16M2-13).
155. The isolated antigen binding construct of any one of arrangements 1-46, 147-148 or 152-154, wherein the antigen binding construct has an expression in mammalian cells that is at least 6-fold greater than the expression of Sibrotuzumab minibodies (IAB16M1-12 and IAB16M2-13).
156. The isolated antigen binding construct of any one of arrangements 1-46, 147-148 or 152-155, wherein the antigen binding construct has a KD of less than 2×10\hat{ }-9 M.
157. The isolated antigen binding construct of any one of arrangements 1-46, 147-148 or 152-156, wherein the antigen binding construct has a KD of less than 1×10\hat{ }-9 M.
158. The isolated antigen binding construct of any one of arrangements 1-46, 147-148 or 152-157, wherein the antigen binding construct has an on-rate (kon) greater than 8.0 (l/Ms).
159. The isolated antigen binding construct of any one of arrangements 1-46, 147-148 or 152-158, wherein the antigen binding construct has an on-rate (kon) greater than 9.0 (l/Ms).
160. The isolated antigen binding construct of any one of arrangements 1-46, 147-148 or 152-159, wherein the antigen binding construct has an off-rate (koff) lesser than 2.5×10\hat{ }-3 (l/s).
161. The isolated antigen binding construct of any one of arrangements 1-46, 147-148 or 152-160, wherein the antigen binding construct has an off-rate (koff) lesser than 1.5×10\hat{ }-3 (l/s).
162. The isolated antigen binding construct of any one of arrangements 1-46, 147-148 or 152-161, wherein the order of the variable regions, from N terminus to C terminus of the polypeptide is VL, VH.
163. The isolated antigen binding construct of any one of arrangements 1-46, 147-148 or 152-161, wherein the order of the variable regions, from N terminus to C terminus of the polypeptide is VH, VL.
164. The isolated antigen binding construct of any one of arrangements 1-163, further comprising a payload.
165. The isolated antigen binding construct of arrangement 164, wherein the payload is fluorescent.
166. The isolated antigen binding construct of arrangement 164, wherein the payload is luminescent.
167. The isolated antigen binding construct of arrangement 164, wherein the payload is colorimetric.
168. The isolated antigen binding construct of arrangement 164, wherein the payload is radioactive.
169. The isolated antigen binding construct of arrangement 164, wherein the payload is non-radioactive.
170. The isolated antigen binding construct of arrangement 164, wherein the payload is ADC.
171. The isolated antigen binding construct of arrangement 164, wherein the payload is chemically reactive.
172. The isolated antigen binding construct of any one of arrangements 1-114, wherein the payload is a detectable marker.
173. The isolated antigen binding construct of any one of arrangements 1-172, wherein the isolated antigen binding construct is humanized.
174. A composition comprising the amino acid sequence of any one of arrangements 1-173, further comprising at least one payload selected from a group consisting of ¹⁸F, ¹⁸F-FAC, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁵Sc, ⁷⁷As, ⁸⁶Y, ⁹⁰Y, ⁸⁹Sr, ⁸⁹Zr, ⁹⁴Tc, ⁹⁴Tc, ⁹⁹mTc, 99Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁷Th and ²²⁵Ac, or any combination thereof.
175. The isolated antigen binding construct of any one of arrangements 47-146, 149-153 or 164-174, wherein the amino acid sequence further comprises at least one metal binding site.
176. The isolated antigen binding construct of any one of arrangements 47-146, 149-153 or 164-175, wherein the amino acid sequence further comprises a histidine (His) tag sequence.
177. The isolated antigen binding construct of any one of arrangements 47-146, 149-153 or 164-176, wherein the amino acid sequence further comprises at 99mTc-carbonyl radiolabel.
178. An expression vector configured to express the sequence of any one of arrangements 1-177.
179. An expression vector capable of expressing the sequence of any one of arrangements 1-177.
180. The expression vector of arrangement 179, wherein the vector is a vector for transfection in mammalian cells.
181. The expression vector of arrangements 178-179, wherein the vector is a viral vector selected from a lentiviral vector or an adenoviral vector.
182. The expression vector of any one of arrangements 178-181, wherein the vector comprises a sequence encoding the cleavable signal peptide having at least 99% identity with the amino acid sequence of SEQ ID NOS: 46 and 121 (METDTLLLWVLLLWVPGSTG).
183. A method of transferring the amino acid sequence or expression vector of any of the preceding arrangements into a host cell, comprising performing electroporation, viral infection, and/or at least one chemical method.
184. A host cell comprising the amino acid sequence or expression vector of any one of arrangements 1-182.
185. A method of use of the amino acid sequence or expression vector of any of the preceding arrangements as a pre-targeting modality, comprising:
   adding a non-radioactive sequence, vector, or antigen binding construct to a system; and
   adding a fast-clearing radiolabeled product that recognizes a protein product of the sequence or vector.
186. The method of arrangement 185, wherein the fast-clearing radiolabeled product comprises a small molecule and/or a peptide.
187. A host cell comprising the amino acid sequence or expression vector of any one of arrangements 1-182.
188. A composition comprising the amino acid sequence of any one of arrangements 1-177 and at least one chelator.
189. The composition of arrangement 188, wherein the at least one chelator is selected from a group consisting of ethylenediaminetetraacetic acid (EDTA), diethylenetriaminepentaacetic acid (DTPA), DOTA, NOTA, NOGADA, NETA, deferoxamine (DfO), porphyrins, polyamines, crown ethers, bis-thiosemicarbazones, polyoximes, dimercaprol, penicillamine, trientine, zinc, deferasirox, deferiprone, deferoxamine, succimer, pyrophosphoric acid, tripolyphosphoric acid, citric acid, tartaric acid, glycine, DMPS, DMSA, NTA, calcium, sodium, desferioxamine, dicobalt EDTA, dimercarpol, BAL, and demercaptosuccinic acid, or any combination thereof.
190. The composition of any one of arrangements 188 or 189, further comprising dual or triple chelators.
191. The composition of arrangement 190, wherein the dual or triple chelators are 64Cu/67Cu, 89Zr/177Lu, or 89Zr/227Th.
192. The composition of any one of arrangements 188-191, wherein the at least one chelator is configured to capture an isotope.
193. The composition of any one of 188-192, further comprising an optical probe.
194. The composition of any one of arrangements 188-193, further comprising at least one payload.
195. The composition of arrangement 194, wherein the at least one payload is selected from a group consisting of ¹⁸F, ¹⁸F-FAC, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁵Sc, ⁷⁷As, ⁸⁶Y, ⁹⁰Y, ⁸⁹Sr, ⁸⁹Zr, ⁹⁴Tc, ⁹⁴Tc, ⁹⁹mTc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁷Th and ²²⁵Ac, or any combination thereof.
196. A use of the composition of any one of arrangements 188-195 as a medicament.
197. A use of the composition of any one of arrangements 188-195 in the manufacture of a medicament for administration to a subject.
198. A use of the composition of any one of arrangements 188-195 for imaging a cell, tissue, organ, and/or subject.
199. A use of the composition of any one of arrangements 188-195 for identifying a disease in a subject.
200. The use of arrangement 199, wherein the disease is a cancer or tumor.
201. The use of arrangement 199, wherein the disease is a solid tumor.
202. The use of arrangement 199, wherein the disease is fibrosis.
203. The use of arrangement 199, wherein the disease is an autoimmune disease.
204. The use of arrangement 199, wherein the disease is cardiovascular.
205. A method of identifying a disease in a subject, comprising:
   administering at least one of the antibody, antigen binding construct, minibody, and/or cys-diabody of any one of the arrangements 1-118 to the subject;
   screening for the binding of the at least one antibody, antigen binding construct, minibody, and/or cys-diabody to FAP; and
   determining whether the subject has a disease thereof based upon the presence or absence of binding to FAP.
206. The method of arrangement 205, wherein the disease is a cancer or tumor.
207. The method of arrangement 205, wherein the disease is a solid tumor.
208. The method of arrangement 205, wherein the disease is fibrosis.
209. The method of arrangement 205, wherein the disease is an autoimmune disease.
210. The method of arrangement 205, wherein the disease is cardiovascular.
211. The method of any one of arrangements 205-210, wherein the subject is mammalian or human.
212. The method of any one of arrangements 205-211, wherein the at least one of the antibody, antigen binding construct, minibody, and/or cys-diabody binds to a tumor stroma.
213. The method of any one of arrangements 205-212, wherein the at least one of the antibody, antigen binding construct, minibody, and/or cys-diabody binds to a fibroblast.
214. The method of any one of arrangements 205-213, wherein the at least one of the antibody, antigen binding construct, minibody, and/or cys-diabody binds to a cancer-associated fibroblast.
215. The method of any one of arrangements 205-214, wherein the disease is epithelial.
216. The method of any one of arrangements 205-215, wherein the least one antibody, antigen binding construct, minibody, and/or cys-diabody further comprises a payload.
217. The method of arrangement 216, wherein the payload is selected from a group consisting of ¹⁸F, ¹⁸F-FAC, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁵Sc, ⁷⁷As, ⁸⁶Y, ⁹⁰Y, ⁸⁹Sr, ⁸⁹Zr, ⁹⁴Tc, ⁹⁴Tc, ⁹⁹mTc, ⁹⁹Mo, ¹⁰⁵Pd, ¹⁰⁵Rh, ¹¹¹Ag, ¹¹¹In, ¹²³I, ¹²⁴I ¹²⁵I, ¹³¹I, ¹⁴²Pr, ¹⁴³Pr, ¹⁴⁹Pm, ¹⁵³Sm, ¹⁵⁴⁻¹⁵⁸Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁷Th and ²²⁵Ac.
218. The method of any one of arrangements 216 or 217, wherein the payload is used to determine the binding of the at least one antibody, antigen binding construct, minibody, and/or cys-diabody to FAP.
219. The method of arrangement 218, wherein the binding of the at least one antibody, antigen binding construct, minibody, and/or cys-diabody to FAP is determined through a PET scan.
220. The method of arrangement 218, wherein the binding of the at least one antibody, antigen binding construct, minibody, and/or cys-diabody to FAP is determined through at least one of a group consisting of: MR imaging, optical probe, magnetic nanoparticles, spectroscopy, and/or photoacoustics.
221. The method of arrangement 218, wherein the binding of the at least one antibody, antigen binding construct, minibody, and/or cys-diabody to FAP is determined through an in vitro testing of a tissue or cell sample removed from the subject.
222. The method of any one of arrangements 205-221, further comprising identifying the subject as negative for having a disease upon the absence of significant binding of the at least one antibody, antigen binding construct, minibody, and/or cys-diabody to FAP.
223. The method of any one of arrangements 205-221, further comprising identifying the subject having a disease upon the significant binding of the at least one antibody, antigen binding construct, minibody, and/or cys-diabody to FAP.
224. A pharmaceutical composition comprising an amount of the at least one antibody, antigen binding construct, minibody, and/or cys-diabody thereof of any one of the arrangements 1-177, that is effective for treating a subject with cancer and/or a tumor; and a pharmaceutically acceptable carrier.
225. The pharmaceutical composition of arrangement 224, wherein the amount of the at least one antibody, antigen binding construct, minibody, and/or cys-diabody is from about 1 mg/kg to about 20 mg/kg.
226. The pharmaceutical composition of arrangement 224, wherein the amount of the at least one antibody, antigen binding construct, minibody, and/or cys-diabody is from about 0.01 mg/kg to about 25 mg/kg.
227. The pharmaceutical composition of any one of arrangements 223-226, further comprising an at least one known small molecule, therapeutic, or antigen binding construct effective in treating a disease.
228. The pharmaceutical composition of arrangement 227, wherein the at least one known small molecule, therapeutic, or antigen binding construct effective in treating a disease is an antibody.
229. The pharmaceutical composition of any one of arrangements 227 or 228, wherein the at least one known small molecule, therapeutic, or antigen binding construct effective in treating a disease is used as part of chemotherapy.
230. The pharmaceutical composition of any one of arrangements 227-229, wherein the at least one known small molecule, therapeutic, or antigen binding construct effective in treating a disease is an immune-oncology drug.
231. The pharmaceutical composition of any one of arrangements 227-230, wherein the at least one known small molecule, therapeutic, or antigen binding construct effective in treating a disease is a DNA repair inhibitor.
232. The pharmaceutical composition of any one of arrangements 227-231, wherein the at least one known small molecule, therapeutic, or antigen binding construct effective in treating a disease is used as part of photodynamic therapy.
233. The pharmaceutical composition of any one of arrangements 227-232, wherein the at least one known small molecule, therapeutic, or antigen binding construct effective in treating a cancer and/or tumor is selected from the group consisting of an alkylating agent, an antimetabolite, an anti-tumor antibiotic, a topoisomerase inhibitor, a mitotic inhibitor, a nitrosourea, a corticosteroid, an anti-angiogenic, an apoptosis inducer, an anti-microtubule agent, a vinca alkaloid, a taxane, an anthracycline, an anti-androgen, a VEGF pathway inhibitor, a VEGF pathway inhibitor, a MAPK/Ras/Raf pathway inhibitor, and an EGFR pathway inhibitor.
234. The pharmaceutical composition of any one of arrangements 227-233, wherein the disease is fibrosis.
235. The pharmaceutical composition of any one of arrangements 227-233, wherein the disease is a cancer or tumor.
236. The pharmaceutical composition of any one of arrangements 227-233, wherein the disease is a solid tumor.
237. The pharmaceutical composition of any one of arrangements 227-233, wherein the disease is an autoimmune disease.
238. The pharmaceutical composition of any one of arrangements 227-233, wherein the disease is cardiovascular.
239. A method of treating, inhibiting, or ameliorating a disease in a subject, comprising: administering the pharmaceutical composition of any one of arrangements 227-238 to the subject in need thereof.
240. The method of arrangement 239, wherein the disease is fibrosis.
241. The method of arrangement 239, wherein the disease is a cancer or tumor.
242. The method of arrangement 239, wherein the disease is an autoimmune disease.
243. The method of arrangement 239, wherein the disease is cardiovascular.
244. The method of arrangement 239, wherein the disease is a solid tumor.
245. The method of any one of arrangements 239-244, further comprising imaging the disease using the composition of any of arrangements 188-193.
246. The method of any one of arrangements 239-245, further comprising imaging the disease using the antigen binding construct of any one of arrangements 1-177.
247. A method of targeting FAP protein on a fibroblast in a subject, comprising: administering the pharmaceutical composition of any one of arrangements 224-238 to the subject.
248. The method of arrangement 247, wherein the fibroblast is a cancer-associated fibroblast or tumor associate macrophage.
249. The method of any one of arrangements 247 or 248, wherein a cancer cell and/or a tumor associated macrophage are damaged or killed following the targeting of the fibroblast.
250. A method of inhibiting, ameliorating, damaging, or inducing apoptosis in a cancer or tumor associated macrophage in a subject, comprising: administering the pharmaceutical composition of any one of arrangements 224-238 to the subject in need thereof.
251. The method of any one of arrangements 239-250, wherein the subject is mammalian or human.
252. The method of any one of arrangements 239-251, further comprising administering radiation therapy, photodynamic therapy, and/or chemotherapy to a subject.
253. The method of any one of arrangements 239-252, wherein the disease is a solid tumor.
254. The method of any one of arrangements 239-253, wherein the disease is epithelial.
255. The method of any one of arrangements 239-254, wherein the cancer or tumor is selected from the group consisting of bone cancer, bone sarcoma, breast cancer, carcinoid, cervical cancer, colon cancer, colorectal cancer, endometrial carcinoma, epithelial ovarian cancer, esophageal cancer, gastric cancer, gastrointestinal cancer, glioma, head and neck cancer, hepatocellular cancer, kidney cancer, leukemia, liver cancer, lung cancer, lymphoma, medullary thyroid carcinoma, melanoma, non-small cell lung cancer, osteosarcoma, oral squamous cell carcinoma, oral cancer, ovarian carcinoma, ovarian cancer, pancreatic adenocarcinoma, pancreatic cancer, prostate cancer, rectal cancer, renal cancer, skin cancer, stomach cancer, testis cancer, thyroid cancer, and urothelial cancer.
256. The isolated antigen binding construct or composition of any one of arrangements 1-177 or 188-195, wherein the antigen binding construct is humanized.
257. A use of the composition of any one of arrangements 188-195 in the manufacture of a medicament for administration to a subject.
258. A use of the composition or antigen binding construct of any one of arrangements 1-174 or 224-238 for photodynamic therapy.
259. A use of the composition or antigen binding construct of any one of arrangements 1-174 or 224-238 for theranostics.
260. A use of the composition or antigen binding construct of any one of arrangements 1-174 or 224-238 for molecular imaging and/or therapy.
261. The use of arrangement 260, wherein the molecular imaging comprises one or more of the group consisting of: photoacoustics, MR imaging, magnetic nanoparticles, spectroscopy, optical probes, and/or any other standard method of imaging.
262. A use of the composition or antigen binding construct of any one of arrangements 1-174 or 224-238 for diagnosing cancer in a subject.
263. A use of the composition or antigen binding construct of any one of arrangements 1-174 or 224-238 for determining a subject's stratification for a therapy.
264. A use of the composition or antigen binding construct of any one of arrangements 1-174 or 224-238 for monitoring a subject's response to a therapy.
265. A use of the composition or antigen binding construct of any one of arrangements 1-174 or 224-238 to inform on a change in therapy decision for a subject in need thereof.
266. A use of the composition or antigen binding construct of any one of arrangements 1-174 or 224-238 for assisted surgery.
267. The composition or antigen binding construct of any one of arrangements 1-174 or 224-238, wherein the antigen binding construct is humanized.

### EXAMPLES

Embodiments of the present invention are further defined in the following Examples. It should be understood that these Examples are given by way of illustration only. From the above discussion and these Examples, one skilled in the art can ascertain the essential characteristics of this invention, and without departing from the spirit and scope thereof, can make various changes and modifications of the embodiments of the invention to adapt it to various usages and conditions. Thus, various modifications of the embodiments of the invention, in addition to those shown and described herein, will be apparent to those skilled in the art from the foregoing description. Such modifications are also intended to fall within the scope of the appended claims. The disclosure of each reference set forth herein is incorporated herein by reference in its entirety, and for the disclosure referenced herein.

### Example 1A

### Isolation of Novel Antigen Binding Constructs

The isolation of a series of novel antigen binding constructs as described herein can also be performed using any standard methodology known to one skilled in the art.

As disclosed herein, a series of antigen binding constructs specific against FAP were designed and isolated (SEQ ID NOS: 11-44). Each antigen binding construct was either human or murine, and either an antibody, a minibody, or a cys-diabody. In one example, the variable region from a parental antibody was converted to a scFv and engrafted in a minibody or cy-diabody scaffold. In another example, the variable region from a parental antibody tested for immunogenic epitopes through in-silico methods known in the art, converted to a scFv and engrafted in a minibody or cy-diabody scaffold. Furthermore, each construct comprised a heavy chain and light chain, with a HCDR1 of SEQ ID NO: 1, a HCDR2 of SEQ ID NO: 2, a HCDR3 of SEQ ID NO:3, a LCDR1 of SEQ ID NO: 4, a LCDR2 of SEQ ID NO: 5, and a LCDR3 of SEQ ID NO: 6. Each antigen binding construct varied in sequence; for example, some contained an alanine at position 24 of the VH framework, a glycine at position 26 of the VH framework, a Serine at position 73 of the VL framework, an arginine at position 83 of the VL framework, a glutamic acid at position 85 of the VL framework, a proline at position 86 of the VL framework, a phenylalanine at position 89 of the VL framework, and/or a tyrosine at position 98 of the VL framework. None of these constructs bound DPP4.

These antigen binding constructs where then inserted into Expi293™ cell-line using a liposome transfection protocol (Thermo Exp293 kit). The vector contained a cleavable signal peptide of SEQ ID NOS: 46 and 121. In other alternatives, the sequence of the antigen binding constructs can be inserted into any cell line using any conventional method, such as electroporation.

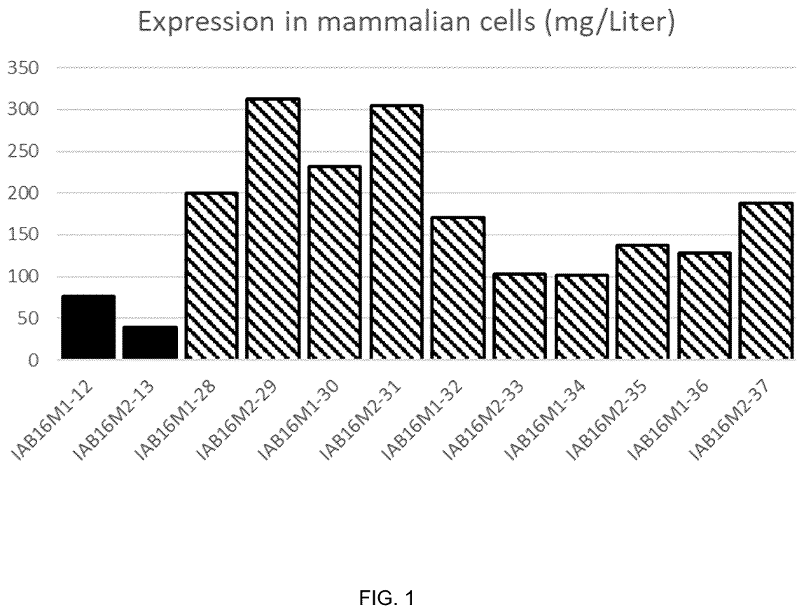

Expression of the constructs was measured compared to the Sibrotuzumab minibodies IAB16M1-12 and IAB16M2-13. As shown in FIG. 1, the novel antigen binding constructs disclosed herein had anywhere from 1.5 to 7-fold greater expression in Expi293™ mammalian cells compared to the Sibrotuzumab minibodies.

Binding kinetics of the novel antigen binding constructs were also determined (TABLE 1). The novel antigen binding construct had a stronger KD, faster on-rate, and slower off-rate, compared with the control (FIGS. 42A and 42B). Additionally, binding of minibodies to the His-tagged extracellular domain of human FAP was also determined (FIG. 53).

TABLE 1
Binding kinetics of antigen binding constructs

| Construct | KD (M) | Ka (1/Ms) | Kd (1/s) |
|---|---|---|---|
| IAB16M1-12 | 3.338E−9 | 7.688E+5 | 2.566E−3 |
| IAB16M2-37 | 1.154E−9 | 9.348E+5 | 1.078E−3 |

Binding kinetics of the novel antigen binding constructs to a purified human FAP antigen was assessed using ELISA. For this assay, a 96 well ELISA plate was coated with the human-FAP antigen diluted to a concentration of 2 mg/mL in coating buffer (0.05M sodium carbonate-bicarbonate buffer, pH 9.6). 100 μl of this coating solution was added to each well in the plate and incubate at 4° C. overnight. The plate was washed 3 times with 300 μl per well of washing buffer (PBS with 0.05% Tween-20). After the last wash, the plate was blocked by adding to each well 200 μl of blocking buffer (PBS with 1% BSA) warmed to room temperature. The plate was then incubated at room temperature for 1 hour under gentle agitation. After blocking, the plate was washed 3 times with 300 μl per well of washing buffer. Dilution samples for each minibody were created by making 2.5× dilutions from a starting sample at 100 ng/mL, or 250 ng/mL, or 1000 ng/mL or 3000 ng/mL in 100 μL sample buffer (PBS with 1% BSA). The dilution samples were transferred to the assay plate and incubated at room temperature for 1 hour under gentle agitation. The plate was then washed 3 times with 300 μl per well of washing buffer.

Detection was performed by adding 100 μl of HRP labeled detection secondary antibody at an appropriate dilution and incubating the plate at room temperature for 1 hour under gentle agitation. The plate was then washed 3 times with 300 μl per well of washing buffer. For color development, 100 μl of TMB substrate regent, pre-warmed to room temperature, was added to the plate and incubated in the dark at room temperature for 15 min. The reaction was then stopped by adding 100 μl of 650 nm stop solution to the plate and mixed for 1 min at room temperature under gentle agitation. The plate is recorded by reading OD at 650 nm in a plate reader within 20 min after terminating the reaction.

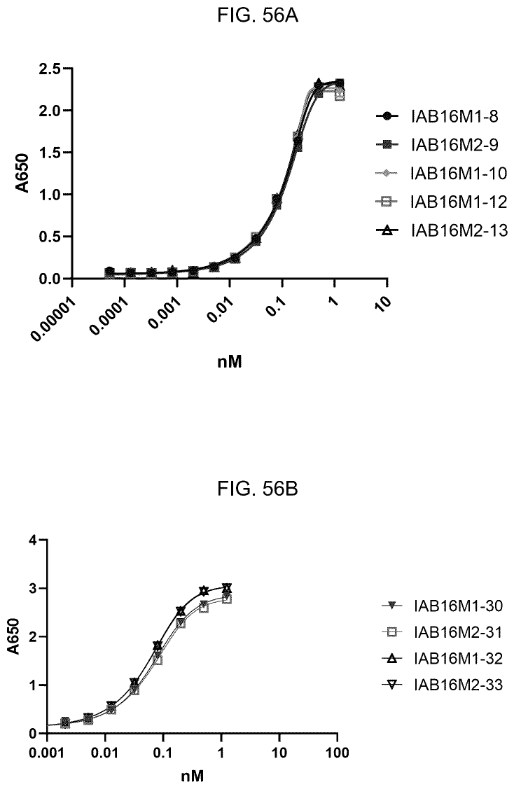

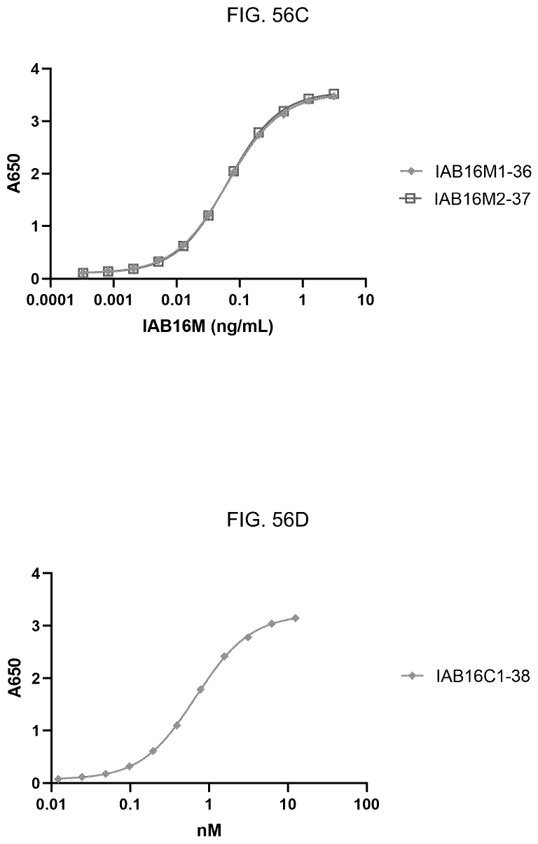

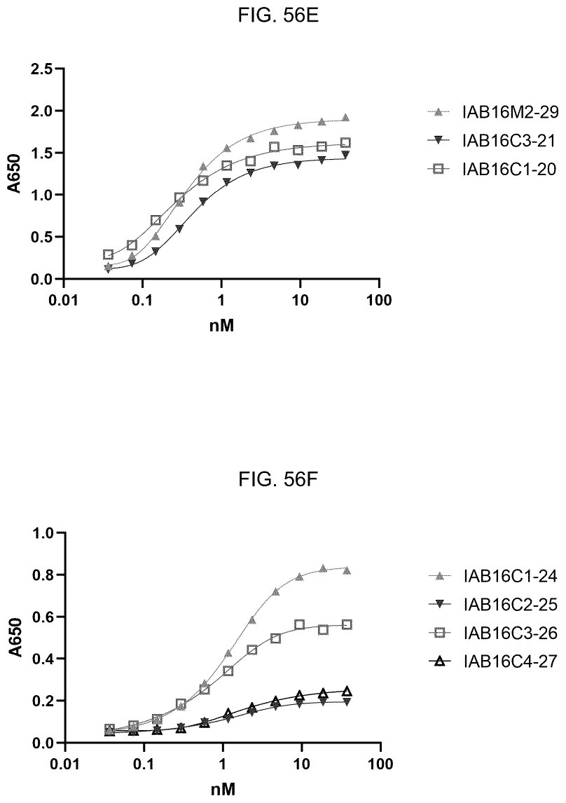

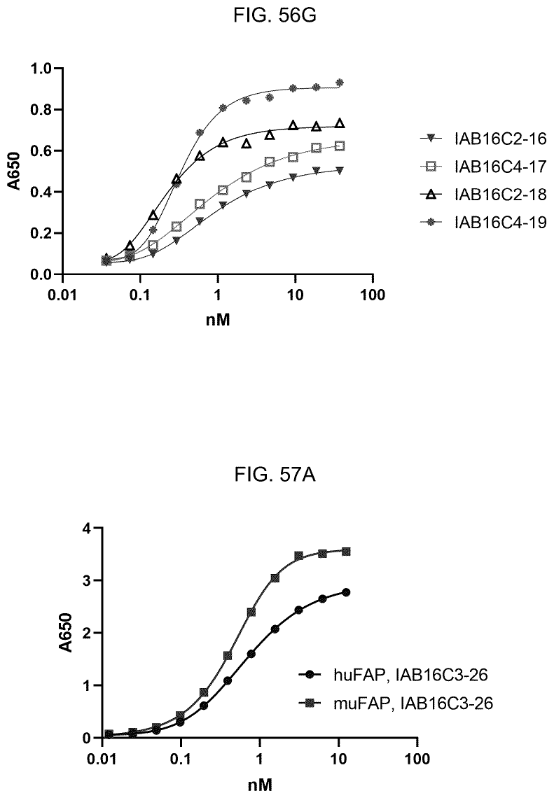

The KD of IAB16M1-8, IAB16M2-9, IAB16M1-10, IAB16M1-12, IAB16M2-13, IAB16M1-30, IAB16M2-31, IAB16M1-32, IAB16M2-33, IAB16M1-36, and IAB16M2-37 were each around 0.1 nM (FIG. 56A-56C). The KD of IAB16M2-29, IAB16C1-20, IAB16C3-21, and IAB16C1-38 were each around 1 nM (FIG. 56D-56E). Constructs IAB16C1-24, IAB16C2-16, IAB16C2-18 IAB16C2-25, IAB16C3-26, IAB16C4-17, IAB164-19, and IAB16C4-27 each had a KD of approximately 1 nM, and different total binding values (FIG. 56F-56G).

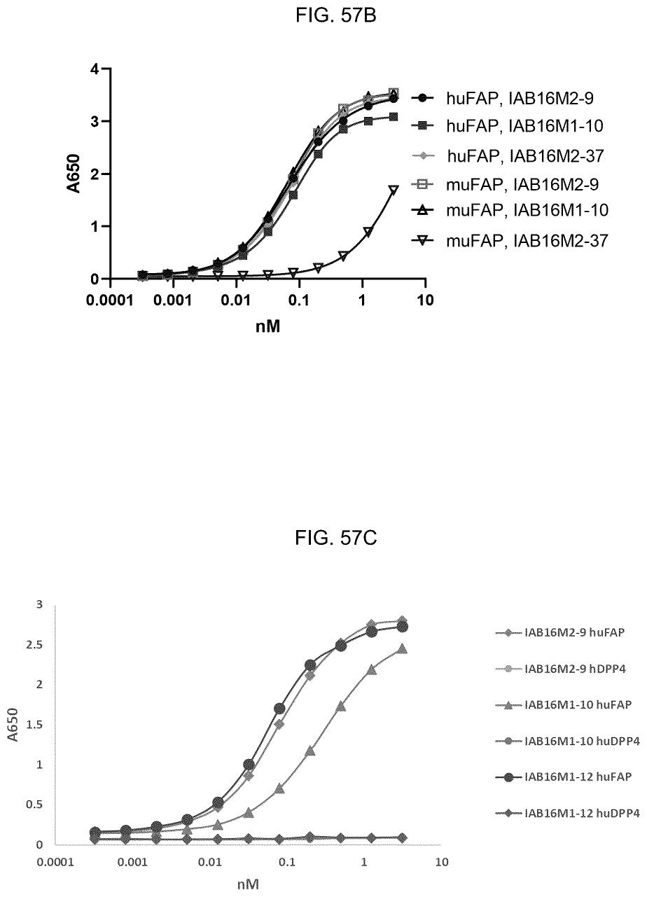

The cross-reactivity of antigen binding constructs to other targets was also assessed using ELISA. Construct IAB16C3-26 was found to have a smaller KD for human FAP compared to murine FAP (FIG. 57A). Similarly, construct IAB16M2-37 had around a 10-fold higher KD for murine FAP compare with human FAP (FIG. 57B). Strikingly, the constructs tested had little to no significant binding to the human DPP4 off-target protein (FIG. 57C).

As disclosed herein, the binding of constructs to cellular targets was assessed using flow cytometry. Stock of 2.5× serial dilution of minibodies were created starting from a concentration of 100 nM in 180 l FACS buffer (PBS 2% FBS). Cells were added to a Corning V-bottom polypropylene cell culture plate at 200,000 cells/well. The cells were centrifuged at 100 rpm for 3 minutes and the supernatant discarded. The minibody dilution series were transferred to the plate containing cell pellets and incubated for 1 hr at 4° C. The plate was then washed 3 times with FACS buffer at 200 l/well, discarding supernatant after each wash by centrifugation at 1000 rpm. 50 l/well of detection antibody R&D AlexaFluor-647 Goat anti Human (H+L) was added at a 1:100 dilution in FACS buffer and incubated at 4° C. 1 hr. The plate was washed 3 times with FACS buffer at 200 l/well, discarding supernatant after each wash by centrifuging at 1000 rpm. Cells were then fixed by adding 100 l of 1% paraformaldehyde in PBS and incubating at room temperature for 15 minutes. 100 l of PBS were added to each well and the plate was analyzed by flow cytometry using the allophycocyanin fluorescence setting.

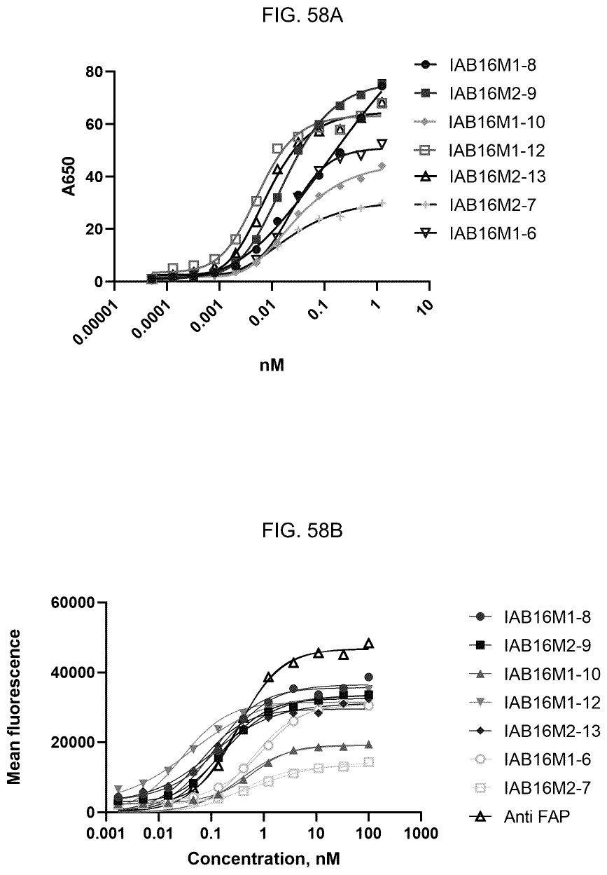

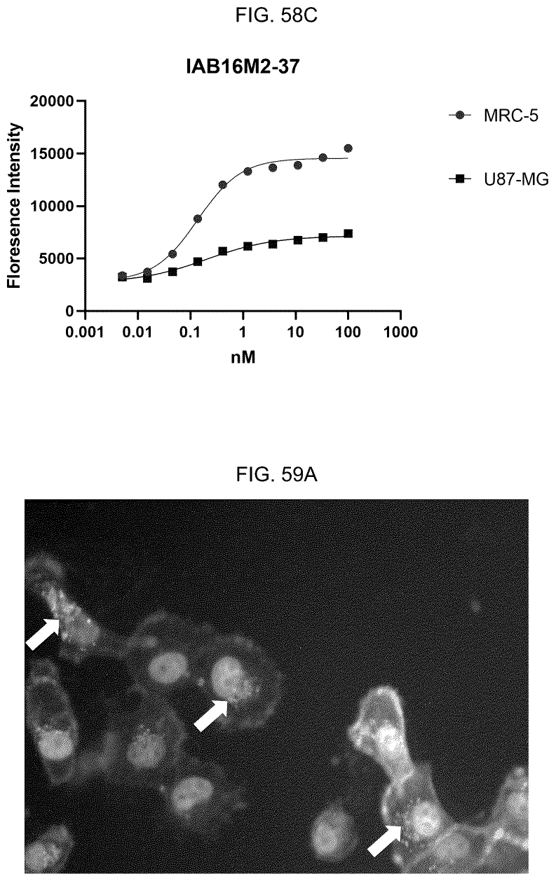

The novel antigen binding constructs disclosed herein had a high affinity for MRC-5 fibroblasts, with KDs between 0.01-0.1 nM (FIG. 58A). The KDs of the constructs to U87-MG glioblastoma cells ranged around 0.1 nM (FIG. 58B). The general trend of antigen binding constructs having a lower KD for MRC-5 fibroblasts than U87-MG glioblastoma cells is also depicted in FIG. 58C.

As disclosed herein, the internalization of antigen binding constructs into cancer cells was visualized using fluorescence microscopy. Glass Slides (Falcon) were coated with poly D Lysine, by adding 100 ml/well of 0.1 mg/ml poly D-lysine to the well and incubating at room temp for 2 hrs. The coating solution was aspirated from the chambers and allowed to dry completely. The slide was then washed three times with sterile water.

The cells were plated at 5000 cells per chamber and incubated overnight at 37° C. in 5% CO2. The minibodies were then added at 100 nM concentration in each chamber in culture media and incubated at 37° C. in 5% CO2 for 3 hrs. As control, a separate slide was not added with any minibody. The control slide was removed from the incubator, and minibody added to the chambers and incubated on ice for 1 hr. The chambers were then washed 3 times with PBS 2% FBS buffer.

To stain the non-internalized minibody bound to the cell membranes, the chambers were treated with 1:100 dilution of Invitrogen AlexaFluor-488 Anti Human H+L for 1 hr on ice. The chambers were washed 3 times with PBS 2% FBS buffer. Then the cells were fixed with 200 ml/chamber of Becton Dickinson Cytofix/CytoPerm solution, and washed 2 times with Becton Dickinson Perm/Wash Solution.

To stain the internalized minibody, a 1:100 dilution of R&D AlexaFluor-594 Goat anti human (H+L) in BD Perm Wash with 300 nM DAPI was prepared. The cell culture was then stained by adding 100 ml/chamber of this staining solution and incubating on ice 1 hr. The chambers were washed 3 time with 500 ml of BD Perm/Wash solution, then 200 ml PBS was added and photographs were recorded under a fluorescent microscope.

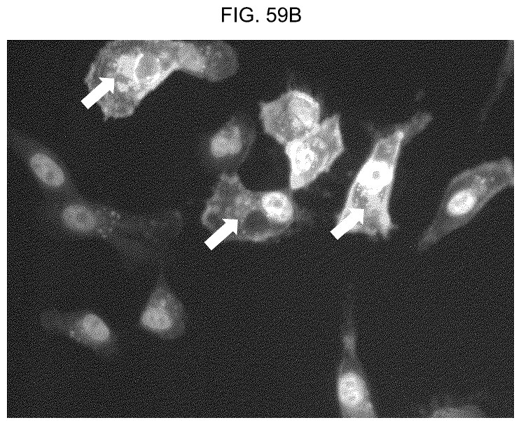

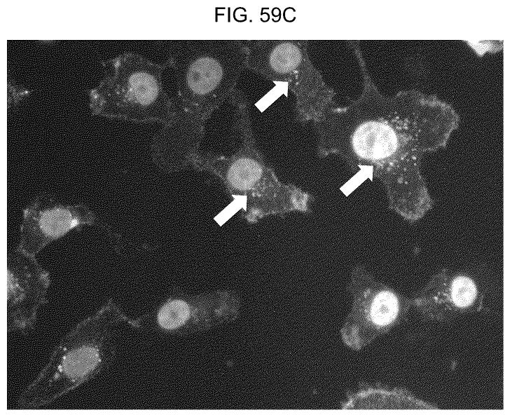

The rapid internalization of the minibodies in cancer cells, and localization to the late endosomes located at the juxtanuclear region was visualized using fluorescence microscopy. Constructs were visualized inside HT1080-hFAP cells after 1 hour (white arrows), indicating that they were successfully recognized, bound to, and localized inside cancer cells (FIG. 59A-59C).

While AlexaFluor-488 was used above, in the alternative, the compound can comprise any payload, including detectable marker such as ⁸⁹Zr, a nonradioactive payload such as ADC, and/or radioactive payload such as one or more of ¹⁸F, ¹⁸F-FAC, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁵Sc, ⁷⁷As, ⁸⁶Y, ⁹⁰Y, ⁸⁹Sr, ⁸⁹Zr, ⁹⁴Tc, ⁹⁴Tc, ⁹⁹mTc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁴⁹Tb, ¹⁵³Sm, ¹⁵⁴⁻¹⁵⁸Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁷Th and ²²⁵Ac. The dual chelators ⁶⁴Cu/⁶⁷Cu, ⁸⁹Zr/¹⁷⁷Lu, ⁸⁹Zr/²²⁷Th and the payload ¹⁸F will also be added to the composition. In other embodiments, at least one of any chelator may be added to the composition, such as EDTA, diethylenetriaminepentaacetic acid, DOTA, DTPA, NOTA, NOGADA, NETA, DFO, porphyrins, polyamines, crown ethers, bis-thiosemicarbazones, polyoximes, dimercaprol, penicillamine, trientine, zinc, deferasirox, deferiprone, deferoxamine, succimer, pyrophosphoric acid, tripolyphosphoric acid, citric acid, tartaric acid, glycine, DMPS, DMSA, NTA, calcium, sodium, desferioxamine, dicobalt EDTA, dimercarpol, BAL, and demercaptosuccinic acid, or any combination thereof. In other embodiments, the at least one chelator is configured to capture an isotope. In other alternatives, the payload can be ADC. In other embodiments, the at least one payload can be any radioactive isotope, including ¹⁸F, ¹⁸F-FAC, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁵Sc, ⁷⁷As, ⁸⁶Y, ⁹⁰Y, ⁸⁹Sr, ⁸⁹Zr, ⁹⁴Tc, ⁹⁴Tc, ⁹⁹mTc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁴⁹Tb, ¹⁵³Sm, ¹⁵⁴⁻¹⁵⁸Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁷Th and ²²⁵Ac, or any combination thereof.

### Example 1B

### Isolation of Novel Antigen Binding Constructs

The isolation of a series of novel antigen binding constructs as described herein can also be performed using any standard methodology known to one skilled in the art.

As disclosed herein, a series of antigen binding constructs specific against FAP were designed and isolated (SEQ ID NOS: 97-103, 116-120, 126-133, and 144-146.). Each antigen binding construct was either human or murine, and either an antibody, a minibody, or a cys-diabody. In one example, the variable region from a parental antibody was converted to a scFv and engrafted in a minibody or cy-diabody scaffold. In another example, the variable region from a parental antibody tested for immunogenic epitopes through in-silico methods known in the art, converted to a scFv and engrafted in a minibody or cy-diabody scaffold. Furthermore, each construct comprised a heavy chain and light chain, with a HCDR1 of SEQ ID NO: 81, 104, or 134; a HCDR2 of SEQ ID NO: 82, 105, or 135; a HCDR3 of SEQ ID NO: 83, 106, or 136; a LCDR1 of SEQ ID NO: 84, 107, or 137; a LCDR2 of SEQ ID NO: 85, 108, or 138; and a LCDR3 of SEQ ID NO: 86, 109, or 139. None of these constructs bound DPP4.

Binding kinetics of the novel antigen binding constructs to a purified human FAP antigen was assessed using ELISA. For this assay, a 96 well ELISA plate was coated with the human-FAP antigen diluted to a concentration of 2 mg/mL in coating buffer (0.05M sodium carbonate-bicarbonate buffer, pH 9.6). 100 μl of this coating solution was added to each well in the plate and incubate at 4° C. overnight. The plate was washed 3 times with 300 μl per well of washing buffer (PBS with 0.05% Tween-20). After the last wash, the plate was blocked by adding to each well 200 μl of blocking buffer (PBS with 1% BSA) warmed to room temperature. The plate was then incubated at room temperature for 1 hour under gentle agitation. After blocking, the plate was washed 3 times with 300 μl per well of washing buffer. Dilution samples for each minibody were created by making 2.5× dilutions from a starting sample at 100 ng/mL, or 250 ng/mL, or 1000 ng/mL or 3000 ng/mL in 100 μL sample buffer (PBS with 1% BSA). The dilution samples were transferred to the assay plate and incubated at room temperature for 1 hour under gentle agitation. The plate was then washed 3 times with 300 μl per well of washing buffer.

Detection was performed by adding 100 μl of HRP labeled detection secondary antibody at an appropriate dilution and incubating the plate at room temperature for 1 hour under gentle agitation. The plate was then washed 3 times with 300 μl per well of washing buffer. For color development, 100 μl of TMB substrate regent, pre-warmed to room temperature, was added to the plate and incubated in the dark at room temperature for 15 min. The reaction was then stopped by adding 100 μl of 650 nm stop solution to the plate and mixed for 1 min at room temperature under gentle agitation. The plate is recorded by reading OD at 650 nm in a plate reader within 20 min after terminating the reaction.

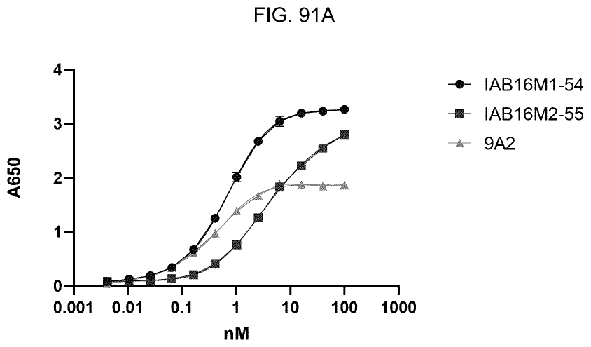

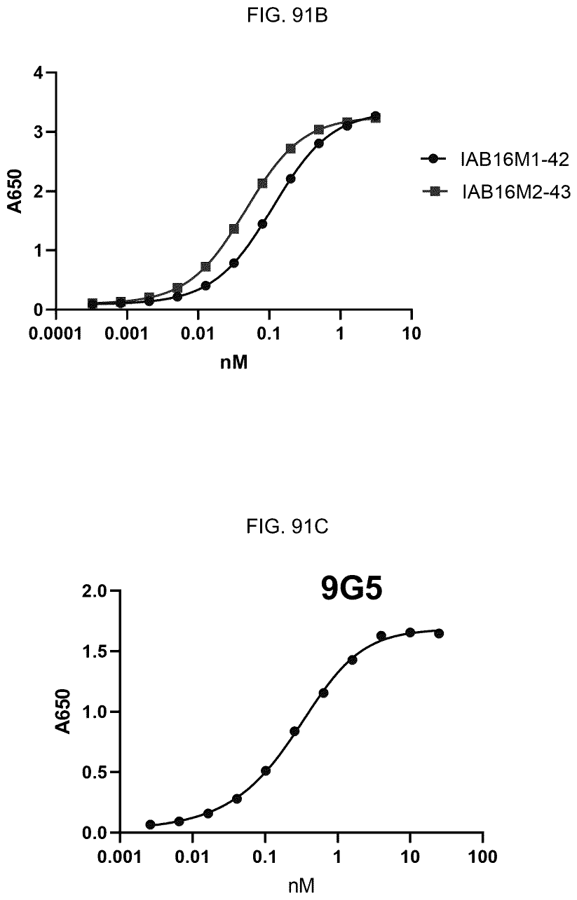

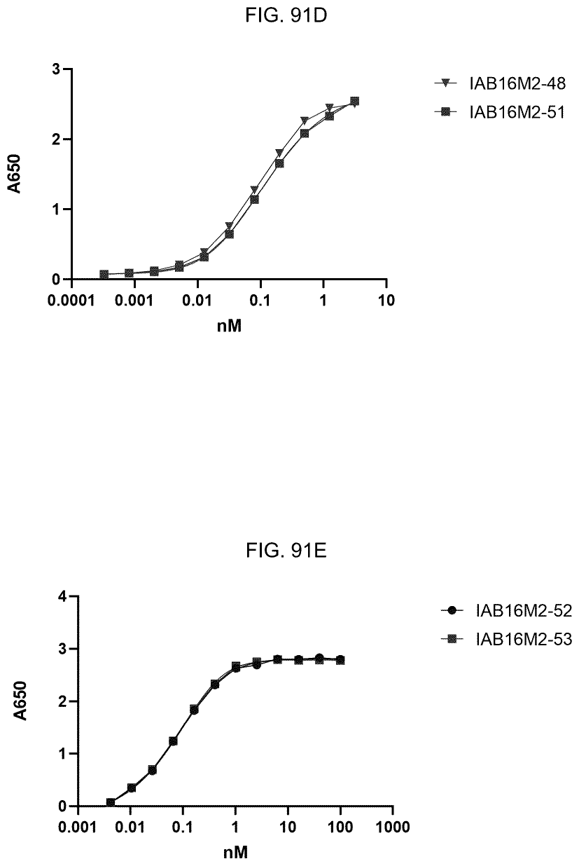

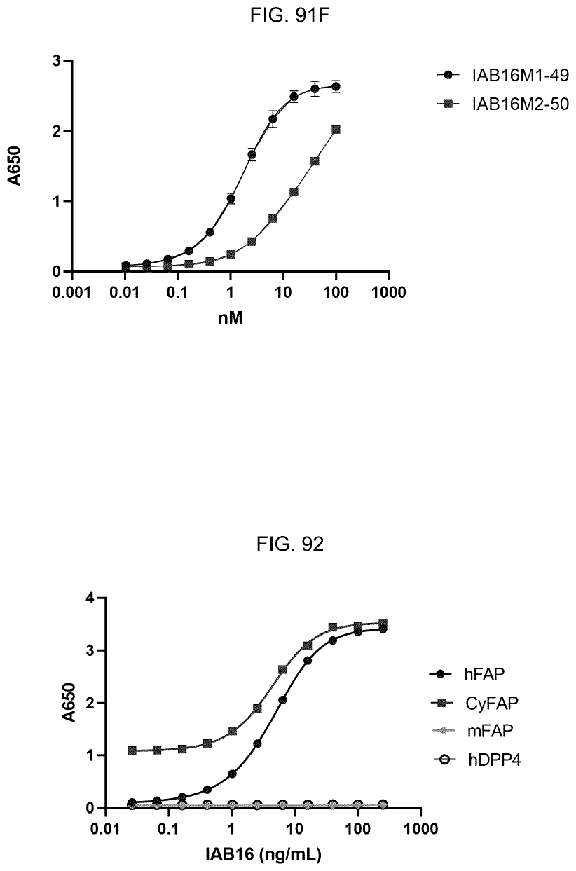

The KD of 9A2, IAB16M1-54, IAB16M2-55, 9G5, and IAB16M1-49 were each around 1 nM (FIGS. 91A, 91C, and 91F). The KD of IAB16M1-42, IAB16M2-43, IAB16M2-48, IAB16M2-51, IAB16M2-52, and IAB16M2-53 were each around 0.1 nM (FIGS. 91B, and 91D-91E). The KD of IAB16M2-50 was around 10 nM (FIG. 91F).

The cross-reactivity of antigen binding constructs to other targets was also assessed using ELISA. Construct IAB16M2-43 was found to have a smaller KD for cynomolgus FAP and human FAP compared to murine FAP (FIG. 92). Strikingly, the construct had little to no significant binding to the human DPP4 off-target protein.

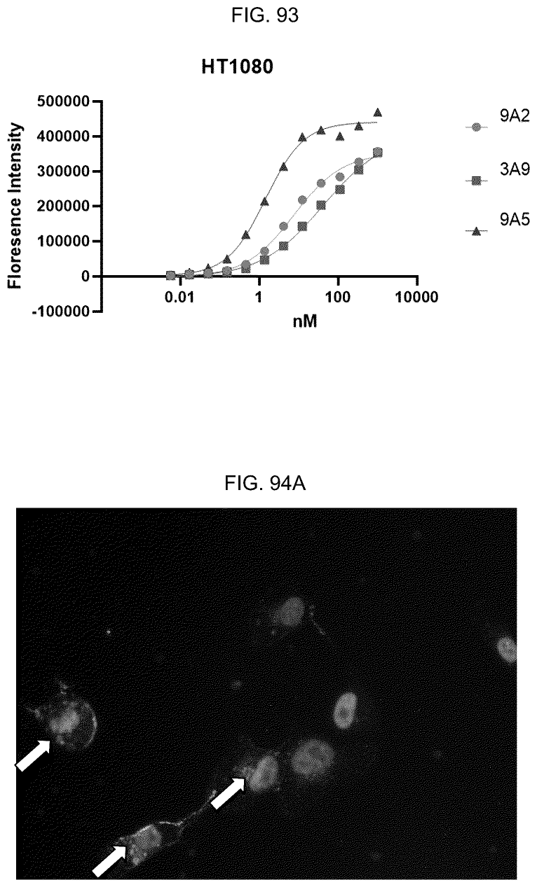

As disclosed herein, the binding of constructs to cellular targets was assessed using flow cytometry. Stock of 2.5× serial dilution of minibodies were created starting from a concentration of 100 nM in 180 μl FACS buffer (PBS 2% FBS). Cells were added to a Corning V-bottom polypropylene cell culture plate at 200,000 cells/well. The cells were centrifuged at 100 rpm for 3 minutes and the supernatant discarded. The minibody dilution series were transferred to the plate containing cell pellets and incubated for 1 hr at 4° C. The plate was then washed 3 times with FACS buffer at 200 μl/well, discarding supernatant after each wash by centrifugation at 1000 rpm. 50 μl/well of detection antibody R&D AlexaFluor-647 Goat anti Human (H+L) was added at a 1:100 dilution in FACS buffer and incubated at 4° C. 1 hr. The plate was washed 3 times with FACS buffer at 200 l/well, discarding supernatant after each wash by centrifuging at 1000 rpm. Cells were then fixed by adding 100 μl 1 of 1% paraformaldehyde in PBS and incubating at room temperature for 15 minutes. 100 μl of PBS were added to each well and the plate was analyzed by flow cytometry using the allophycocyanin fluorescence setting. The antigen binding constructs 9A2, 3A9, and 9A5 disclosed herein had KDs to HT1080-human FAP cells of approximately between 0.1-20 nM (FIG. 93).

As disclosed herein, the internalization of antigen binding constructs into cancer cells was visualized using fluorescence microscopy. Glass Slides (Falcon) were coated with poly D Lysine, by adding 100 ml/well of 0.1 mg/ml poly D-lysine to the well and incubating at room temp for 2 hrs. The coating solution was aspirated from the chambers and allowed to dry completely. The slide was then washed three times with sterile water.

The cells were plated at 5000 cells per chamber and incubated overnight at 37° C. in 5% CO2. The minibodies were then added at 100 nM concentration in each chamber in culture media and incubated at 37° C. in 5% CO2 for 3 hrs. As control, a separate slide was not added with any minibody. The control slide was removed from the incubator, and minibody added to the chambers and incubated on ice for 1 hr. The chambers were then washed 3 times with PBS 2% FBS buffer.

To stain the non-internalized minibody bound to the cell membranes, the chambers were treated with 1:100 dilution of Invitrogen AlexaFluor-488 Anti Human H+L for 1 hr on ice. The chambers were washed 3 times with PBS 2% FBS buffer. Then the cells were fixed with 200 ml/chamber of Becton Dickinson Cytofix/CytoPerm solution, and washed 2 times with Becton Dickinson Perm/Wash Solution.

To stain the internalized minibody, a 1:100 dilution of R&D AlexaFluor-594 Goat anti human (H+L) in BD Perm Wash with 300 nM DAPI was prepared. The cell culture was then stained by adding 100 ml/chamber of this staining solution and incubating on ice 1 hr. The chambers were washed 3 time with 500 ml of BD Perm/Wash solution, then 200 ml PBS was added and photographs were recorded under a fluorescent microscope.

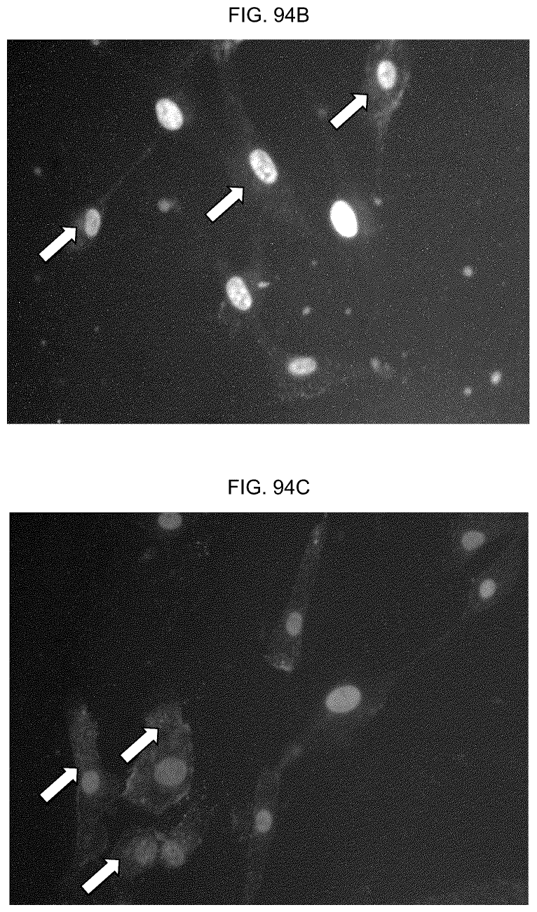

The rapid internalization of the minibodies in cancer cells, and localization to the late endosomes located at the juxtanuclear region was visualized using fluorescence microscopy. The construct IAB16M2-43 were successfully visualized inside HT1080-hFAP cells, MRC5 cells, and U87-MG cells (white arrows), indicating that they were successfully recognized, bound to, and localized inside cancer cells (FIG. 94A-94C).

While AlexaFluor-488 was used above, in the alternative, the compound can comprise any payload, including detectable marker such as ⁸⁹Zr, a nonradioactive payload such as ADC, and/or radioactive payload such as one or more of ¹⁸F, ¹⁸F-FAC, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁵Sc, ⁷⁷As, ⁸⁶Y, ⁹⁰Y, ⁸⁹Sr, ⁸⁹Zr, ⁹⁴Tc, ⁹⁴Tc, ⁹⁹mTc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁴⁹Tb, ¹⁵³Sm, ¹⁵⁴⁻¹⁵⁸Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁷Th and ²²⁵Ac. The dual chelators ⁶⁴Cu/⁶⁷Cu, ⁸⁹Zr/¹⁷⁷Lu, ⁸⁹Zr/²²⁷Th and the payload ¹⁸F will also be added to the composition.

In other embodiments, at least one of any chelator may be added to the composition, such as EDTA, diethylenetriaminepentaacetic acid, DOTA, DTPA, NOTA, NOGADA, NETA, DFO, porphyrins, polyamines, crown ethers, bis-thiosemicarbazones, polyoximes, dimercaprol, penicillamine, trientine, zinc, deferasirox, deferiprone, deferoxamine, succimer, pyrophosphoric acid, tripolyphosphoric acid, citric acid, tartaric acid, glycine, DMPS, DMSA, NTA, calcium, sodium, desferioxamine, dicobalt EDTA, dimercarpol, BAL, and demercaptosuccinic acid, or any combination thereof. In other embodiments, the at least one chelator is configured to capture an isotope.

In other alternatives, the payload can be ADC. In other embodiments, the at least one payload can be any radioactive isotope, including ¹⁸F, ¹⁸F-FAC, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁵Sc, ⁷⁷As, ⁸⁶Y, ⁹⁰Y, ⁸⁹Sr, ⁸⁹Zr, ⁹⁴Tc, ⁹⁴Tc, ⁹⁹mTc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁴⁹Tb, ¹⁵³Sm, ¹⁵⁴⁻¹⁵⁸Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁷Th and ²²⁵Ac, or any combination thereof.

### Example 2

### Use of the Antigen Binding Constructs for Diagnostics

The methods of utilizing the novel antigen binding constructs described herein for diagnostics thereof may confer a benefit to a subject suspected of having a disease.

As disclosed herein, the novel antigen binding construct will be used for imaging the tissue of a human subject suspected of having cancer.

In other alternatives, the antigen binding constructs can be used for imaging a cell, cultured cell line, fraction of tissue, organ, organ sectional, multi-tissue, multi-organ, or whole subject.

In other alternatives, the subject can be screened for a tumor, fibrosis, autoimmune disease, cardiovascular disease, or any other disease or abnormality associated with FAP.

In other alternatives, the subject can be any mammal, including mice, rats, and non-human primates.

The antigen binding construct will be administered to the epithelial tissue of a subject and screened for binding to FAP. The subject will be evaluated for the presence of FAP by methods of in-vivo diagnostic medical imaging such as, positron emission tomography (PET), or Single-photon emission computed tomography (SPECT). The binding of the antigen binding construct to FAP will indicate a high likelihood for the presence of cancer in the tissue. In other alternatives, the absence of binding of the antigen binding construct to FAP will indicate a low likelihood for the presence of cancer in the tissue.

In other alternatives, the antigen binding construct may bind to a tumor stroma, fibroblast, macrophage, tumor associated macrophage, or cancer-associated fibroblast.

In other alternatives, the binding of the antigen binding construct to FAP is determined by a payload added to the antigen binding construct, such as ¹⁸F, ¹⁸F-FAC, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁵Sc, ⁷⁷As, ⁸⁶Y, ⁹⁰Y, ⁸⁹Sr, ⁸⁹Zr, ⁹⁴Tc, ⁹⁴Tc, ⁹⁹mTc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁷Th or ²²⁵Ac.

In other alternatives, the binding of the antigen binding construct to FAP is determined through a PET scan.

### Example 3A

### Use of Antigen Binding Constructs for Reducing Uptake in Kidneys

The methods of modifying the antigen binding constructs described herein can confer a benefit of reduced kidney uptake of radiolabeled minibodies in the subject.

As disclose herein, a minibody was made through humanization of a mouse hybridoma. The humanization of the antibody was based on germlines: IGHV1-69*01/IGHV1-39*01.

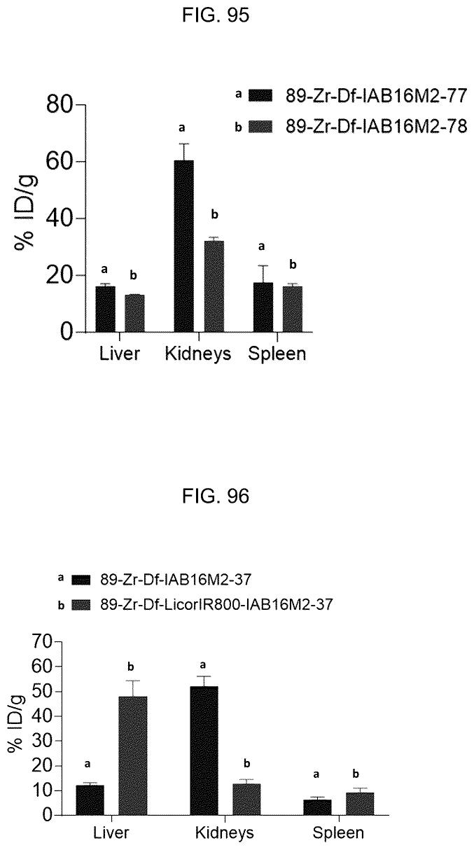

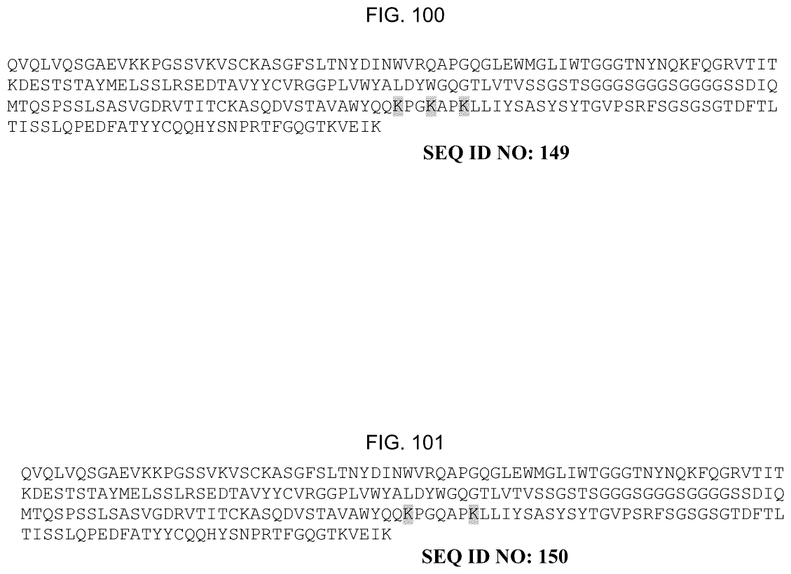

Positively charged patch was identified on the germline IGKV1-39*01 which is responsible for accumulating this minibody in the kidney As disclosed herein, the patch maps to sequence KPGKAPK which contains 3 charged lysine residues (FIG. 100 and SEQ ID NO: 149). Mutations on the framework 2 of this germline (FIG. 101 and SEQ ID NO: 150) were identified that reduce kidney uptake of radiolabeled minibodies (FIG. 95).

As disclosed herein, several human germlines have a pattern of three positively charged residues at framework 2.

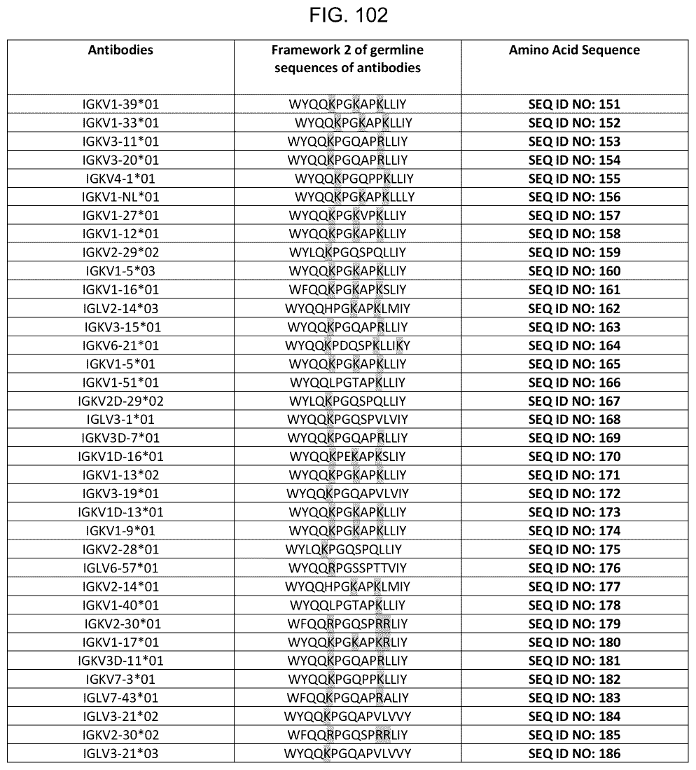

In some embodiments, the positively charged residue is a lysine (K) and/or arginine (R). In some embodiments, the germline IGKV1-39*01 is modified where the framework 2 is substituted with similar sequence from germlines that do not contain this charge pattern (SEQ ID NOS: 151-186 and FIG. 102). A non-limiting example for such substitution is framework 2 from IGKV3-20*01 (SEQ ID NO:154—KPGQAPR) or IGKV2-28*01 (SEQ ID NO:175—KPGQSPQ).

### Example 3B

### Use of Antigen Binding Constructs for Modulating Biodistribution

The methods of modifying the antigen binding constructs described herein may confer a benefit of modulating the biodistribution between the renal and the hepatic routes in the subject.

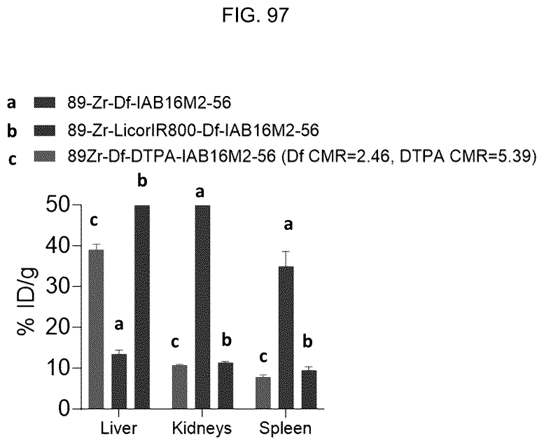

As disclosed herein, chemical modifications of minibodies consisting in conjugating minibodies with negatively charged organic moieties drastically redirected the clearance from the renal route to the hepatic route. The modifications tested consisted in conjugation with Licor IRDye800 (FIGS. 96 and 97).

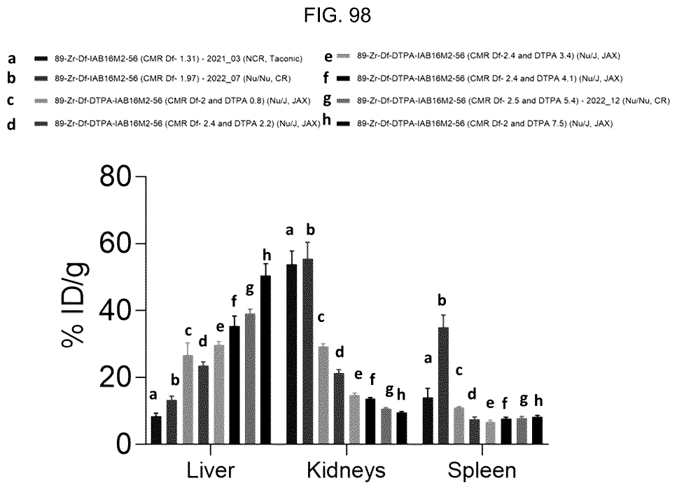

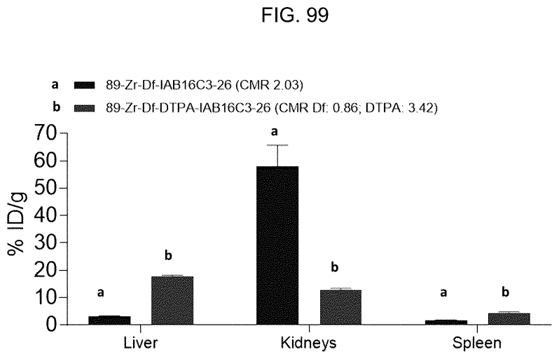

Conjugation of minibodies at high Chelator-to-Minibody Ratio (CMR >=5) with metal chelators DTPA and DOTA, also triggered the same effect (FIGS. 97-99).

### Example 4

### Use of the Antigen Binding Constructs for Treatment of a Subject in Need Thereof

The methods of utilizing the novel antigen binding constructs described herein as a medicament to a subject in need thereof may confer a benefit to the subject.

As disclosed herein, a pharmaceutical composition will be made comprising 10 mg/kg a novel antigen binding construct against FAP, alone or in combination with an immuno-oncology drug, and a pharmaceutically acceptable carrier. In other alternatives, the dosage is a radioactive dose. In other alternatives, the dosage is not fixed. In other alternatives, the dosage is provided as fractionated doses. In other alternatives, the dosage is measured in mCi or MBq.

In other alternatives, the composition can have more than one antigen binding constructs.

In other alternatives, the antigen binding construct can be an antibody, minibody, cys-diabody, or another combination thereof. In other alternatives, the antigen binding construct may be present in the composition at any pharmaceutically effective concentration, such as about 0.01 mg/kg to about 25 mg/kg.

In other alternatives, the immuno-oncology drug may be omitted from the composition.

In other alternatives, the immuno-oncology drug may be replaced by one or more small molecule, therapeutic, or antigen binding construct effective in treating a disease, such as an antibody, a chemotherapy drug, a DNA repair inhibitor, an alkylating agent, a metabolic inhibitor, a radiosensitizer agent, an anti-tumor antibiotic, a topoisomerase inhibitor, a mitotic inhibitor, a nitrosourea, a corticosteroid, an anti-angiogenic, an apoptosis inducer, an anti-microtubule agent, a vinca alkaloid, a taxane, an anthracycline, an anti-androgen, a VEGF pathway inhibitor, a VEGF pathway inhibitor, a MAPK/Ras/Raf pathway inhibitor, and an EGFR pathway inhibitor.

As disclosed herein, the composition will be administered to a human subject at a pharmaceutically acceptable dose for treating epithelial cancer.

In other alternatives, the disease may be fibrosis, cancer, a tumor, a solid tumor, an autoimmune disease, cardiovascular disease, metabolic disease, bone cancer, bone sarcoma, breast cancer, carcinoid, cervical cancer, colon cancer, colorectal cancer, endometrial carcinoma, epithelial ovarian cancer, esophageal cancer, gastric cancer, gastrointestinal cancer, glioma, head and neck cancer, hepatocellular cancer, kidney cancer, leukemia, liver cancer, lung cancer, lymphoma, medullary thyroid carcinoma, melanoma, non-small cell lung cancer, osteosarcoma, oral squamous cell carcinoma, oral cancer, ovarian carcinoma, ovarian cancer, pancreatic adenocarcinoma, pancreatic cancer, prostate cancer, rectal cancer, renal cancer, skin cancer, stomach cancer, testis cancer, thyroid cancer, urothelial cancer, or any other disease associated with changing FAP expression. In other alternatives, the composition is administered to a subject for inhibiting or ameliorating the disease. In other alternatives, the subject can be any mammal, including mice, rats, and non-human primates. In other alternatives, the composition is used to target FAP on a fibroblast or cancer-associated fibroblast, or on a macrophage or tumor associated macrophage.

After administration, the disease will be monitored through the imaging of relevant cells, tissues, or organs in the subject using the method outlined in Example 2. As seen through the imaging, the cancer cells, or the tumor associated fibroblasts, or the tumor associated macrophages are damaged following administration of the composition to the subject. In other alternatives, the cancer cell, fibroblast, or macrophage is tumor associated. In other alternatives, the cancer cell, fibroblast, or macrophage in inhibited, ameliorated, damaged, killed, or has induced apoptosis in response to contact with the composition.

### Example 5

### Use of the Antigen Binding Constructs for Theranostics in a Subject in Need Thereof

The methods of utilizing the novel antigen binding constructs described herein in theranostics to a subject in need thereof may confer a benefit to the subject.

As disclosed herein, a pharmaceutical composition will be made comprising 10 μg/kg of a novel antigen binding construct against FAP, alone or in combination with a detectable marker. In other alternatives, the antigen binding construct will be administered at a dosage from 0.1 ug-1 mg/kg or 0.2 ug-1 mg/mL. This composition will be used in the diagnosis of a suspect suspected of having a disease. In other alternatives, the disease will be a cancer, tumor, solid tumor, fibrosis, epithelial disease, autoimmune disease, cardiovascular disease, or any other disease or abnormality associated with FAP.

As disclosed herein, the diagnosis will comprise utilizing the novel antigen binding construct in imaging the tissue of a human subject suspected of having cancer. In other alternatives, the antigen binding constructs can be used for imaging a cell, cultured cell line, fraction of tissue, organ, organ sectional, multi-tissue, multi-organ, or whole subject. In other alternatives, the subject can be any mammal, including mice, rats, and non-human primates.

The antigen binding construct will be administered to the epithelial tissue of a subject and screened for binding to FAP.

In other alternatives, the antigen binding construct will be screened for binding to FAP alpha. In other alternatives, the antigen binding construct will be screened for binding to DPP4 as a negative control. The subject will be evaluated for the presence of FAP by methods of in-vivo diagnostic medical imaging such as, positron emission tomography (PET), or Single-photon emission computed tomography (SPECT). The binding of the antigen binding construct to FAP will indicate a high likelihood for the presence of cancer in the tissue.

In other alternatives, the absence of binding of the antigen binding construct to FAP will indicate a low likelihood for the presence of cancer in the tissue.

In other alternatives, the antigen binding construct may bind to a tumor stroma, fibroblast, macrophage, tumor associated macrophage, or cancer-associated fibroblast.

In other alternatives, the binding of the antigen binding construct to FAP is determined by a payload added to the antigen binding construct, such as ¹⁸F, ¹⁸F-FAC, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁵Sc, ⁷⁷As, ⁸⁶Y, ⁹⁰Y, ⁸⁹Sr, ⁸⁹Zr, ⁹⁴Tc, ⁹⁴Tc, ⁹⁹mTc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁷Th or ²²⁵Ac. In other alternatives, the binding of the antigen binding construct to FAP is determined through a PET scan.

After the subject is diagnosed as having the disease, the novel antigen binding construct will be administered conjugated to a cytotoxic agent at a therapeutic dose. In other alternatives, the antigen binding construct is conjugated to an immuno-oncology drug, medicament, or toxic radiolabel. In other alternatives, the antigen binding construct is administered along with a pharmaceutically acceptable carrier. As disclosed herein, a pharmaceutical composition will be made comprising 10 mg/kg a novel antigen binding construct against FAP, alone or in combination with an immuno-oncology drug, and a pharmaceutically acceptable carrier. In other alternatives, the dosage is a radioactive dose. In other alternatives, the dosage is not fixed. In other alternatives, the dosage is provided as fractionated doses. In other alternatives, the dosage is measured in mCi or MBq. In other alternatives, the composition can have more than one antigen binding constructs. In other alternatives, the antigen binding construct can be an antibody, minibody, cys-diabody, or another combination thereof. In other alternatives, the antigen binding construct may be present in the composition at any pharmaceutically effective concentration, such as about 0.01 mg/kg to about 25 mg/kg. In other alternatives, the immuno-oncology drug may be omitted from the composition. In other alternatives, the immuno-oncology drug may be replaced by one or more small molecule, therapeutic, or antigen binding construct effective in treating a disease, such as an antibody, a chemotherapy drug, a DNA repair inhibitor, an alkylating agent, a metabolic inhibitor, a radiosensitizer agent, an anti-tumor antibiotic, a topoisomerase inhibitor, a mitotic inhibitor, a nitrosourea, a corticosteroid, an anti-angiogenic, an apoptosis inducer, an anti-microtubule agent, a vinca alkaloid, a taxane, an anthracycline, an anti-androgen, a VEGF pathway inhibitor, a VEGF pathway inhibitor, a MAPK/Ras/Raf pathway inhibitor, and an EGFR pathway inhibitor.

As disclosed herein, the administration of the compound will serve as a therapy to reduce, alleviate, limit, or treat the occurrence and/or symptoms of the disease.

### Example 6

A subject is identified as potentially having a disorder relating to FAP levels. After the subject is diagnosed as having a FAP related disease, an antigen binding construct will be administered conjugated to a cytotoxic agent at a therapeutic dose.

As used herein, the section headings are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose, including the disclosures specifically referenced herein. When definitions of terms in incorporated references appear to differ from the definitions provided in the present teachings, the definition provided in the present teachings shall control. It will be appreciated that there is an implied “about” prior to the temperatures, concentrations, times, etc. discussed in the present teachings, such that slight and insubstantial deviations are within the scope of the present teachings herein.

Although this invention has been disclosed in the context of certain embodiments and examples, those skilled in the art will understand that the present invention extends beyond the specifically disclosed embodiments to other alternative embodiments and/or uses of the invention and obvious modifications and equivalents thereof. In addition, while several variations of the invention have been shown and described in detail, other modifications, which are within the scope of this invention, will be readily apparent to those of skill in the art based upon this disclosure. It is also contemplated that various combinations or sub-combinations of the specific features and aspects of the embodiments may be made and still fall within the scope of the invention. It should be understood that various features and aspects of the disclosed embodiments can be combined with, or substituted for, one another in order to form varying modes or embodiments of the disclosed invention. Thus, it is intended that the scope of the present invention herein disclosed should not be limited by the particular disclosed embodiments described above.

It should be understood, however, that this detailed description, while indicating preferred embodiments of the invention, is given by way of illustration only, since various changes and modifications within the spirit and scope of the invention will become apparent to those skilled in the art.

The terminology used in the description presented herein is not intended to be interpreted in any limited or restrictive manner. Rather, the terminology is simply being utilized in conjunction with a detailed description of embodiments of the systems, methods and related components. Furthermore, embodiments may comprise several novel features, no single one of which is solely responsible for its desirable attributes or is believed to be essential to practicing the inventions herein described.

1-54. (canceled)

55. An isolated antigen binding construct comprising:
a HCDR1 comprising the amino acid sequence of SEQ ID NO: 81 (NYDIN), or a sequence that has no more than 3 point mutations thereto;
a HCDR2 comprising the amino acid sequence of SEQ ID NO: 82 (LIWTGGGTN), or a sequence that has no more than 3 point mutations thereto; and/or
a HCDR3 comprising the amino acid sequence of SEQ ID NO: 83 (GGPLVWYALDY), or a sequence that has no more than 3 point mutations thereto.

56. The isolated antigen binding construct of claim 55, further comprising:
a LCDR1 comprising the amino acid sequence of SEQ ID NO: 84 (KASQDVSTAVA), or a sequence that has no more than 3 point mutations thereto;
a LCDR2 comprising the amino acid sequence of SEQ ID NO: 85 (SASYRYT), or a sequence that has no more than 3 point mutations thereto; and/or
a LCDR3 comprising the amino acid sequence of SEQ ID NO: 86 (QQHYSNPRT), or a sequence that has no more than 3 point mutations thereto.

57. The isolated antigen binding construct of claim 55, wherein there are no more than 2 point mutations in HCDR1, HCDR2 and/or HCDR3.

58. The isolated antigen binding construct of claim 55, further comprising a variable heavy domain (VH) having the amino acid sequence of SEQ ID NO: 87.

59. The isolated antigen binding construct of claim 56, further comprising a variable light domain (VL) having the amino acid sequence of SEQ ID NO: 88.

60. The isolated antigen binding construct of claim 55, wherein the antigen binding construct is specific against FAP alpha.

61. The isolated antigen binding construct of claim 55, wherein the antigen binding construct does not bind DPP4.

62. The isolated antigen binding construct of claim 55, further comprising a payload.

63. The isolated antigen binding construct of claim 62, wherein the payload is selected from among a moiety that is fluorescent, luminescent, colorimetric, radioactive, non-radioactive, a chemotherapeutic agent, a chemically reactive agent and a detectable marker.

64. A composition comprising the isolated antigen binding construct of claim 55, further comprising at least one payload selected from a group consisting of ¹⁸F, ¹⁸F-FAC, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁵Sc, ⁷⁷As, ⁸⁶Y, ⁹⁰Y, ⁸⁹Sr, ⁸⁹Zr, ⁹⁴Tc, ⁹⁴Tc, ⁹⁹mTc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁴⁹Tb, ¹⁵³Sm, ¹⁵⁴⁻¹⁵⁸Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁷Th and ²²⁵Ac, or any combination thereof.

65. A composition comprising the isolated antigen binding construct of claim 55 and at least one chelator.

66. A pharmaceutical composition comprising an amount of the isolated antigen binding construct of claim 55 that is effective for treating a subject with cancer and/or a tumor; and a pharmaceutically acceptable carrier.

67. A method of treating, inhibiting, or ameliorating a disease in a subject, comprising: administering a pharmaceutical composition comprising the isolated antigen binding construct of claim 55 to the subject in need thereof.

68. A method of targeting FAP protein on a fibroblast in a subject, comprising: administering a pharmaceutical composition comprising the isolated antigen binding construct of claim 55 to the subject.

69. A method of inhibiting, ameliorating, damaging, or inducing apoptosis in a cancer or tumor associated macrophage in a subject, comprising: administering a pharmaceutical composition comprising the isolated antigen binding construct of claim 55 to the subject in need thereof.

70. A method of identifying a disease in a subject, comprising:
administering a pharmaceutical composition comprising the isolated antigen binding construct of claim 55 to the subject;
screening for the binding of the isolated antigen binding construct to FAP; and
determining whether the subject has a disease thereof based upon the presence or absence of binding to FAP.

71. An isolated antigen binding construct, comprising:
a HCDR1 comprising amino acids having at least 80% identity with the amino acid sequence of SEQ ID NO: 1 (EYTIH);
a HCDR2 comprising amino acids having at least 80% identity with the amino acid sequence of SEQ ID NO: 2 (GINPNNGIPNYNQKFKG);
a HCDR3 comprising amino acids having at least 80% identity with the amino acid sequence of SEQ ID NO: 3 (RRIAYGYDEGHAMDY);
a LCDR1 comprising amino acids having at least 80% identity with the amino acid sequence of SEQ ID NO: 4 (KSSQSLLYSRNQKNYLA);
a LCDR2 comprising amino acids having at least 80% identity with the amino acid sequence of SEQ ID NO: 5 (WASTRES); and
a LCDR3 comprising amino acids having the amino acid sequence of SEQ ID NO: 6 (QQYYSYPLT).

72. The antigen binding construct of claim 71, comprising:
a variable heavy domain (VH) of SEQ ID NO: 7; and
a variable light domain (VL) of SEQ ID NO: 8 or 9.

73. The isolated antigen binding construct of claim 72, comprising at least one VH framework residue selected from the group consisting of:
an Alanine at position 24 of the sequence in SEQ ID NO: 7; or
a Glycine at position 26 of the sequence in SEQ ID NO: 7 and
at least one VL framework residue selected from the group consisting of:
a Serine at position 73 of the sequence in SEQ ID NO: 8;
an Arginine at position 83 of the sequence in SEQ ID NO: 8;
a Glutamic acid at position 85 of the sequence in SEQ ID NO:8;
a Proline at position 86 of the sequence in SEQ ID NO: 8;
a Phenylalanine at position 89 of the sequence in SEQ ID NO: 8; or
a Tyrosine at position 98 of the sequence in SEQ ID NO: 8.

74. An isolated antigen binding construct comprising amino acids having 100% identity with an amino acid sequence selected from the any one of the sequences listed in SEQ ID NOS: 23-44, 12-43, 12-45, 87-96, 110-115, 122-125, or 140-143.
